(19) United States
(12) Patent Application Publication
(10) Pub. No.: US 2026/0167969 A1
(43) Pub. Date: Jun. 18, 2026

(54) INHIBITORY NUCLEIC ACIDS FOR ITFG1

(71) Applicant: LERNA BIOPHARMA PTE. LTD., Amnios (SG)

(72) Inventors: Yann Chong TAN, Amnios (SG); Si Hui TAN, Amnios (SG); Binxia YANG, Amnios (SG); Xi LIN, Amnios (SG); Mei Jia TAN, Amnios (SG); Audrey Shimei WANG, Amnios (SG)

(73) Assignee: LERNA BIOPHARMA PTE. LTD., Amnios (SG)

(21) Appl. No.: 18/876,389

(22) PCT Filed: Jun. 28, 2023
(86) PCT No.: PCT/EP2023/067668

§ 371 Date: Dec. 18, 2024

(30) Foreign Application Priority Data
Jun. 28, 2022 (GB) 2209462.7
Feb. 14, 2023 (GB) 2302067.0

(51) Int. Cl. C12N 15/113; A61K 9/00; A61K 31/712; A61K 31/7125; C12N 15/63

(52) U.S. Cl. CPC C12N 15/1138; A61K 9/0019; A61K 31/712; A61K 31/7125; C12N 15/63; C12N 2310/11; C12N 2310/14; C12N 2310/315; C12N 2310/321; C12N 2310/322

(57) ABSTRACT

Provided herein are nucleic acids for decreasing the expression of ITFG1, and methods for using the same, including methods of medical treatment and prophylaxis.

This application claims priority from GB 2209462.7 filed 28 Jun. 2022 and from GB 2302067.0 filed 14 Feb. 2023, the contents and elements of which are herein incorporated by reference for all purposes.

### TECHNICAL FIELD

The present disclosure relates to the fields of molecular biology, more specifically nucleic acid technology. The present disclosure also relates to methods of medical treatment and prophylaxis.

### BACKGROUND

The rising incidence of acute and chronic liver failure, which causes more than 1.3 million deaths per year worldwide (World Health Organization, 2018), represents a major global health concern. The main underlying causes of end-stage liver disease are hepatitis virus infections (especially hepatitis B and C), drug- and alcohol-induced liver damage, and non-alcoholic fatty liver disease (NAFLD; associated with obesity and progressing to non-alcoholic steatohepatitis (NASH)). Asia has an especially high burden of hepatitis virus infections (WHO), and an increased incidence of NAFLD. Despite advances in the prevention and treatment of viral hepatitis (hepatitis B vaccination and hepatitis C combination therapies) the number of people with end-stage liver disease is expected to rise, mainly fuelled by the obesity epidemic and aging societies.

Currently, the only curative treatment for end-stage liver disease is liver transplantation. However, donor organs are limited, and end-stage liver disease patients may also experience complications that render them unfit for major surgery. Therefore, alternative strategies to hold off or reverse end-stage liver disease are being pursued. These include cell transplantation, artificial liver devices, and enhancing the organ's endogenous regenerative capacity.

The liver is the only visceral organ that possesses the remarkable capacity to regenerate. It is known that as little as 25% of the original liver mass can regenerate back to its full size. Adult hepatocytes are long-lived and normally do not undergo cell division (GO). However, upon liver damage, they have the ability to enter the cell cycle and proliferate. Once cell proliferation is completed, the newly divided cells undergo restructuring, and other regeneration-related processes such as angiogenesis and reformation of extracellular matrix to complete the regeneration process.

Despite this amazing ability, the regenerative capacity of the liver seems limited, especially under chronic damaging conditions. The ability of the liver to regenerate is central to liver homeostasis. Because the liver is the main site of drug detoxification, it is exposed to many chemicals in the body which may potentially induce cell death and injury. Furthermore, through the enterohepatic circulation, it is exposed to microbiota related metabolites. The liver can regenerate damaged tissue rapidly thereby preventing functional failure. Liver regeneration is also critical for patients with partial removal of the liver due to tumor resection or living-donor transplantation.

WO 2022/025827 A1 identifies ITFG1 as a target for inhibition in order to promote proliferation/expansion of cells including hepatocytes, lung cells and myoblasts, wound healing and regeneration of liver tissue, and for treatment/prevention of disease characterised by fibrosis, e.g. fibrosis of the liver.

### SUMMARY

In a first aspect, the present disclosure provides an inhibitory nucleic acid for reducing gene and/or protein expression of ITFG1.

The present disclosure also provides an inhibitory nucleic acid for reducing gene and/or protein expression of ITFG1, wherein the inhibitory nucleic acid comprises or encodes antisense nucleic acid targeting a nucleotide sequence comprising, or consisting of, one of SEQ ID NOs:26, 33, 13 to 25, 27 to 32, 33 to 44, 128, 135, 115 to 127, 129 to 134, or 136 to 146.

In some embodiments, the inhibitory nucleic acid comprises or encodes antisense nucleic acid targeting a nucleotide sequence comprising, or consisting of, SEQ ID NO:26, 33, 128 or 135.

In some embodiments, the inhibitory nucleic acid comprises or encodes antisense nucleic acid comprising or consisting of a nucleotide sequence having at least 75% sequence identity to one of SEQ ID NOs:58, 65, 45 to 57, 59 to 64, 66 to 76, 160, 167, 147 to 159, 161 to 166, or 168 to 178.

In some embodiments, the inhibitory nucleic acid comprises or encodes antisense nucleic acid comprising or consisting of a nucleotide sequence having at least 75% sequence identity to SEQ ID NO:58, 65, 160 or 167. In some embodiments, the inhibitory nucleic acid comprises: (i) nucleic acid comprising the nucleotide sequence of one of SEQ ID NOs:58, 65, 45 to 57, 59 to 64, 66 to 76, 160, 167, 147 to 159, 161 to 166, or 168 to 178, or a nucleotide sequence having at least 75% sequence identity to one of SEQ ID NOs:58, 65, 45 to 57, 59 to 64, 66 to 76, 160, 167, 147 to 159, 161 to 166, or 168 to 178; and (ii) nucleic acid comprising a nucleotide sequence having the reverse complement of the nucleotide sequence of (i), or having at least 75% sequence identity to the reverse complement of the nucleotide sequence of (i).

In some embodiments, the inhibitory nucleic acid comprises, or consists of: (i) nucleic acid comprising a nucleotide sequence indicated in column A of Table I, and (ii) nucleic acid comprising a nucleotide sequence indicated in column B of Table I, wherein the sequences of columns A and B are selected from the same row of Table I.

In some embodiments, the inhibitory nucleic acid comprises, or consists of: (i) nucleic acid comprising a nucleotide sequence indicated in column A of Table II, and (ii) nucleic acid comprising a nucleotide sequence indicated in column B of Table II, wherein the sequences of columns A and B are selected from the same row of Table II.

In some embodiments, the inhibitory nucleic acid comprises one or more modified nucleotides selected from: 2′-O-methyluridine-3′-phosphate, 2′-O-methyladenosine-3′-phosphate, 2′-O-methylguanosine-3′-phosphate, 2′-O-methylcytidine-3′-phosphate, 2′-O-methyluridine-3′-phosphorothioate, 2′-O-methyladenosine-3′-phosphorothioate, 2′-O-methylguanosine-3′-phosphorothioate, 2′-O-methylcytidine-3′-phosphorothioate, 2′-fluorouridine-3′-phosphate, 2′-fluoroadenosine-3′-phosphate, 2′-fluoroguanosine-3-phosphate, 2′-fluorocytidine-3′-phosphate, 2′-fluorocytidine-3′-phosphorothioate, 2′-fluoroguanosine-3′-phosphorothioate, 2′-fluoroadenosine-3′-phosphorothioate, and 2′-fluorouridine-3′-phosphorothioate.

In some embodiments, the inhibitory nucleic acid comprises, or consists of: (i) nucleic acid comprising a nucleotide sequence (including the modifications thereto) indicated in column A of Table III, and (ii) nucleic acid comprising a nucleotide sequence (including the modifications thereto) indicated in column B of Table III, wherein the sequences of columns A and B are selected from the same row of Table III.

In some embodiments, the inhibitory nucleic acid comprises, or consists of (i) SEQ ID NO:215 (including the modifications thereto), and (ii) SEQ ID NO:235 (including the modifications thereto). In some embodiments, the inhibitory nucleic acid comprises, or consists of (i) SEQ ID NO:215 (including the modifications thereto), and (ii) SEQ ID NO:241 (including the modifications thereto). In some embodiments, the inhibitory nucleic acid comprises, or consists of (i) SEQ ID NO:215 (including the modifications thereto), and (ii) SEQ ID NO:254 (including the modifications thereto). In some embodiments, the inhibitory nucleic acid comprises, or consists of (i) SEQ ID NO:215 (including the modifications thereto), and (ii) SEQ ID NO:255 (including the modifications thereto). In some embodiments, the inhibitory nucleic acid comprises, or consists of (i) SEQ ID NO:215 (including the modifications thereto), and (ii) SEQ ID NO:256 (including the modifications thereto). In some embodiments, the inhibitory nucleic acid comprises, or consists of (i) SEQ ID NO:215 (including the modifications thereto), and (ii) SEQ ID NO:257 (including the modifications thereto). In some embodiments, the inhibitory nucleic acid comprises, or consists of (i) SEQ ID NO:216 (including the modifications thereto), and (ii) SEQ ID NO:235 (including the modifications thereto). In some embodiments, the inhibitory nucleic acid comprises, or consists of (i) SEQ ID NO:226 (including the modifications thereto), and (ii) SEQ ID NO:240 (including the modifications thereto).

In some embodiments, the inhibitory nucleic acid comprises a moiety facilitating uptake of the inhibitory nucleic acid by hepatocytes, optionally wherein the moiety facilitating uptake of the inhibitory nucleic acid by hepatocytes is or comprises a N-acetylgalactosamine (GalNAc) moiety.

In some embodiments, the inhibitory nucleic acid is an siRNA.

The present disclosure also provides a nucleic acid, optionally isolated, encoding an inhibitory nucleic acid according to the present disclosure.

The present disclosure also provides an expression vector, comprising a nucleic acid according to the present disclosure.

The present disclosure also provides a composition comprising an inhibitory nucleic acid, nucleic acid or expression vector according to the present disclosure, and a pharmaceutically acceptable carrier, diluent, excipient or adjuvant.

The present disclosure also provides a cell comprising an inhibitory nucleic acid, nucleic acid or expression vector according to the present disclosure.

The present disclosure also provides an in vitro or in vivo method for reducing gene and/or protein expression of ITFG1 in a cell, comprising introducing an inhibitory nucleic acid, nucleic acid or expression vector according to the present disclosure into a cell.

The present disclosure also provides the use of an inhibitory nucleic acid, nucleic acid, expression vector or composition according to the present disclosure to reduce gene and/or protein expression of ITFG1 in vitro or in vivo.

The present disclosure also provides an inhibitory nucleic acid, nucleic acid, expression vector or composition according to the present disclosure for use in a method of medical treatment or prophylaxis.

The present disclosure also provides an inhibitory nucleic acid, nucleic acid, expression vector or composition according to the present disclosure for use in a method of treating or preventing a disease/condition selected from: a disease/condition characterised by fibrosis, a disease/condition characterised by damage to and/or death of cells of the liver, chronic liver disease, liver fibrosis, cirrhosis, non-alcoholic fatty liver disease (NAFLD), hepatitis, steatohepatitis, non-alcoholic steatohepatitis (NASH), alcoholic liver disease (ALD), alcoholic fatty liver (AFL), alcoholic hepatitis, alcoholic steatohepatitis (ASH), primary biliary cholangitis (PBC), schistosomal liver disease, congenital liver disease, liver cancer, hepatocellular carcinoma (HCC), acute liver injury (ALI), acute liver failure, acute liver disease, viral hepatitis, liver ischemia-reperfusion injury (IRI), warm ischemia-reperfusion (WIR), radiation-induced liver disease (RILD), drug-induced liver injury (DILI), acetaminophen-induced liver injury, autoimmune liver injury, liver transplantation, extended hepatectomy, small-for-size syndrome, split liver grafts, cholestatic liver disease, pulmonary fibrosis, interstitial lung disease (ILD), idiopathic interstitial pneumonia (IIP), idiopathic pulmonary fibrosis (IPF), cystic fibrosis, progressive massive fibrosis, scleroderma, obliterative bronchiolitis, Hermansky-Pudlak syndrome, asbestosis, silicosis, sarcoidosis, tumor stroma in lung disease, chronic obstructive pulmonary disease (COPD), emphysema, pneumonia, pulmonary edema, chronic bronchitis, asthma, hypertrophic cardiomyopathy (HCM), dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertension, hypertensive heart disease, arrhythmogenic right ventricular cardiomyopathy (ARVC), atherosclerosis, chronic pulmonary hypertension, AIDS-associated pulmonary hypertension, varicose veins, cerebral infarcts, tubulointerstitial fibrosis, glomerular fibrosis, renal fibrosis, nephritic syndrome, Alport's syndrome, HIV-associated nephropathy, polycystic kidney disease, Fabry's disease, diabetic nephropathy, chronic glomerulonephritis, nephritis associated with systemic lupus, pancreatic fibrosis, cystic fibrosis, chronic pancreatitis, gliosis, Alzheimer's disease, multiple sclerosis, muscular dystrophy, Duchenne muscular dystrophy (DMD), Becker's muscular dystrophy (BMD), fibrotic myopathy, inflammatory bowel disease (IBD), Crohn's disease, microscopic colitis, primary sclerosing cholangitis (PSC), scleroderma, nephrogenic systemic fibrosis, Dupuytren's contracture, cutis keloid, Grave's ophthalmopathy, epiretinal fibrosis, retinal fibrosis, subretinal fibrosis, subretinal fibrosis associated with macular degeneration (e.g. wet age-related macular degeneration (AMD)), diabetic retinopathy, glaucoma, corneal fibrosis, post-surgical fibrosis (e.g. of the posterior capsule following cataract surgery, or of the bleb following trabeculectomy for glaucoma), conjunctival fibrosis, subconjunctival fibrosis, arthrofibrosis, arthritis, adhesive capsulitis, progressive systemic sclerosis (PSS), chronic graft versus host disease (GVHD), fibrotic pre-neoplastic, fibrotic neoplastic disease, fibrosis induced by chemical or environmental insult, gastric cancer, esophageal cancer, lung cancer, head and neck cancer, colorectal cancer, pancreatic cancer, cervical cancer, vulvar cancer, mediastinal fibrosis, retroperitoneal fibrosis, myelofibrosis, Peyronie's disease, renal tubular disorder, renal tubular acidosis, hypokalemia, hyperkalemia, acute tubular necrosis, Fanconi syndrome, Liddle syndrome, nephrogenic diabetes insipidus, pseudohypoaldosteronism type I, chronic kidney disease, acute kidney injury, a neurodegenerative disease, amyotrophic lateral sclerosis, epilepsy, frontotemporal dementia, Parkinson's disease, Huntington's disease, multiple system atrophy, a prion disease, ulcerative colitis, inflammatory bowel syndrome, a chronic inflammatory condition of the gastrointestinal tract or coeliac disease.

The present disclosure also provides the use of an inhibitory nucleic acid, nucleic acid, expression vector or composition according to the present disclosure in the manufacture of a medicament for treating or preventing a disease/condition selected from: a disease/condition characterised by fibrosis, a disease/condition characterised by damage to and/or death of cells of the liver, chronic liver disease, liver fibrosis, cirrhosis, non-alcoholic fatty liver disease (NAFLD), hepatitis, steatohepatitis, non-alcoholic steatohepatitis (NASH), alcoholic liver disease (ALD), alcoholic fatty liver (AFL), alcoholic hepatitis, alcoholic steatohepatitis (ASH), primary biliary cholangitis (PBC), schistosomal liver disease, congenital liver disease, liver cancer, hepatocellular carcinoma (HCC), acute liver injury (ALI), acute liver failure, acute liver disease, viral hepatitis, liver ischemia-reperfusion injury (IRI), warm ischemia-reperfusion (WIR), radiation-induced liver disease (RILD), drug-induced liver injury (DILI), acetaminophen-induced liver injury, autoimmune liver injury, liver transplantation, extended hepatectomy, small-for-size syndrome, split liver grafts, cholestatic liver disease, pulmonary fibrosis, interstitial lung disease (ILD), idiopathic interstitial pneumonia (IIP), idiopathic pulmonary fibrosis (IPF), cystic fibrosis, progressive massive fibrosis, scleroderma, obliterative bronchiolitis, Hermansky-Pudlak syndrome, asbestosis, silicosis, sarcoidosis, tumor stroma in lung disease, chronic obstructive pulmonary disease (COPD), emphysema, pneumonia, pulmonary edema, chronic bronchitis, asthma, hypertrophic cardiomyopathy (HCM), dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertension, hypertensive heart disease, arrhythmogenic right ventricular cardiomyopathy (ARVC), atherosclerosis, chronic pulmonary hypertension, AIDS-associated pulmonary hypertension, varicose veins, cerebral infarcts, tubulointerstitial fibrosis, glomerular fibrosis, renal fibrosis, nephritic syndrome, Alport's syndrome, HIV-associated nephropathy, polycystic kidney disease, Fabry's disease, diabetic nephropathy, chronic glomerulonephritis, nephritis associated with systemic lupus, pancreatic fibrosis, cystic fibrosis, chronic pancreatitis, gliosis, Alzheimer's disease, multiple sclerosis, muscular dystrophy, Duchenne muscular dystrophy (DMD), Becker's muscular dystrophy (BMD), fibrotic myopathy, inflammatory bowel disease (IBD), Crohn's disease, microscopic colitis, primary sclerosing cholangitis (PSC), scleroderma, nephrogenic systemic fibrosis, Dupuytren's contracture, cutis keloid, Grave's ophthalmopathy, epiretinal fibrosis, retinal fibrosis, subretinal fibrosis, subretinal fibrosis associated with macular degeneration (e.g. wet age-related macular degeneration (AMD)), diabetic retinopathy, glaucoma, corneal fibrosis, post-surgical fibrosis (e.g. of the posterior capsule following cataract surgery, or of the bleb following trabeculectomy for glaucoma), conjunctival fibrosis, subconjunctival fibrosis, arthrofibrosis, arthritis, adhesive capsulitis, progressive systemic sclerosis (PSS), chronic graft versus host disease (GVHD), fibrotic pre-neoplastic, fibrotic neoplastic disease, fibrosis induced by chemical or environmental insult, gastric cancer, esophageal cancer, lung cancer, head and neck cancer, colorectal cancer, pancreatic cancer, cervical cancer, vulvar cancer, mediastinal fibrosis, retroperitoneal fibrosis, myelofibrosis, Peyronie's disease, renal tubular disorder, renal tubular acidosis, hypokalemia, hyperkalemia, acute tubular necrosis, Fanconi syndrome, Liddle syndrome, nephrogenic diabetes insipidus, pseudohypoaldosteronism type I, chronic kidney disease, acute kidney injury, a neurodegenerative disease, amyotrophic lateral sclerosis, epilepsy, frontotemporal dementia, Parkinson's disease, Huntington's disease, multiple system atrophy, a prion disease, ulcerative colitis, inflammatory bowel syndrome, a chronic inflammatory condition of the gastrointestinal tract or coeliac disease.

The present disclosure also provides a method of treating or preventing a disease or condition in a subject, comprising administering to a subject a therapeutically- or prophylactically-effective amount of an inhibitory nucleic acid, nucleic acid, expression vector or composition according to the present disclosure, wherein the disease or condition is selected from: a disease/condition characterised by fibrosis, a disease/condition characterised by damage to and/or death of cells of the liver, chronic liver disease, liver fibrosis, cirrhosis, non-alcoholic fatty liver disease (NAFLD), hepatitis, steatohepatitis, non-alcoholic steatohepatitis (NASH), alcoholic liver disease (ALD), alcoholic fatty liver (AFL), alcoholic hepatitis, alcoholic steatohepatitis (ASH), primary biliary cholangitis (PBC), schistosomal liver disease, congenital liver disease, liver cancer, hepatocellular carcinoma (HCC), acute liver injury (ALI), acute liver failure, acute liver disease, viral hepatitis, liver ischemia-reperfusion injury (IRI), warm ischemia-reperfusion (WIR), radiation-induced liver disease (RILD), drug-induced liver injury (DILI), acetaminophen-induced liver injury, autoimmune liver injury, liver transplantation, extended hepatectomy, small-for-size syndrome, split liver grafts, cholestatic liver disease, pulmonary fibrosis, interstitial lung disease (ILD), idiopathic interstitial pneumonia (IIP), idiopathic pulmonary fibrosis (IPF), cystic fibrosis, progressive massive fibrosis, scleroderma, obliterative bronchiolitis, Hermansky-Pudlak syndrome, asbestosis, silicosis, sarcoidosis, tumor stroma in lung disease, chronic obstructive pulmonary disease (COPD), emphysema, pneumonia, pulmonary edema, chronic bronchitis, asthma, hypertrophic cardiomyopathy (HCM), dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertension, hypertensive heart disease, arrhythmogenic right ventricular cardiomyopathy (ARVC), atherosclerosis, chronic pulmonary hypertension, AIDS-associated pulmonary hypertension, varicose veins, cerebral infarcts, tubulointerstitial fibrosis, glomerular fibrosis, renal fibrosis, nephritic syndrome, Alport's syndrome, HIV-associated nephropathy, polycystic kidney disease, Fabry's disease, diabetic nephropathy, chronic glomerulonephritis, nephritis associated with systemic lupus, pancreatic fibrosis, cystic fibrosis, chronic pancreatitis, gliosis, Alzheimer's disease, multiple sclerosis, muscular dystrophy, Duchenne muscular dystrophy (DMD), Becker's muscular dystrophy (BMD), fibrotic myopathy, inflammatory bowel disease (IBD), Crohn's disease, microscopic colitis, primary sclerosing cholangitis (PSC), scleroderma, nephrogenic systemic fibrosis, Dupuytren's contracture, cutis keloid, Grave's ophthalmopathy, epiretinal fibrosis, retinal fibrosis, subretinal fibrosis, subretinal fibrosis associated with macular degeneration (e.g. wet age-related macular degeneration (AMD)), diabetic retinopathy, glaucoma, corneal fibrosis, post-surgical fibrosis (e.g. of the posterior capsule following cataract surgery, or of the bleb following trabeculectomy for glaucoma), conjunctival fibrosis, subconjunctival fibrosis, arthrofibrosis, arthritis, adhesive capsulitis, progressive systemic sclerosis (PSS), chronic graft versus host disease (GVHD), fibrotic pre-neoplastic, fibrotic neoplastic disease, fibrosis induced by chemical or environmental insult, gastric cancer, esophageal cancer, lung cancer, head and neck cancer, colorectal cancer, pancreatic cancer, cervical cancer, vulvar cancer, mediastinal fibrosis, retroperitoneal fibrosis, myelofibrosis, Peyronie's disease, renal tubular disorder, renal tubular acidosis, hypokalemia, hyperkalemia, acute tubular necrosis, Fanconi syndrome, Liddle syndrome, nephrogenic diabetes insipidus, pseudohypoaldosteronism type I, chronic kidney disease, acute kidney injury, a neurodegenerative disease, amyotrophic lateral sclerosis, epilepsy, frontotemporal dementia, Parkinson's disease, Huntington's disease, multiple system atrophy, a prion disease, ulcerative colitis, inflammatory bowel syndrome, a chronic inflammatory condition of the gastrointestinal tract or coeliac disease.

In some embodiments in accordance with various aspects of the present disclosure, the disease/condition is a disease/condition characterised by fibrosis, or a disease/condition characterised by damage to and/or death of cells of the liver.

In some embodiments, the disease/condition characterised by fibrosis is selected from: chronic liver disease, liver fibrosis, cirrhosis, non-alcoholic fatty liver disease (NAFLD), hepatitis, steatohepatitis, non-alcoholic steatohepatitis (NASH), alcoholic liver disease (ALD), alcoholic fatty liver (AFL), alcoholic hepatitis, alcoholic steatohepatitis (ASH), primary biliary cholangitis (PBC), schistosomal liver disease, congenital liver disease, liver cancer, hepatocellular carcinoma (HCC), acute liver injury (ALI), acute liver failure, acute liver disease, viral hepatitis, liver ischemia-reperfusion injury (IRI), warm ischemia-reperfusion (WIR), radiation-induced liver disease (RILD), drug-induced liver injury (DILI), acetaminophen-induced liver injury, autoimmune liver injury, liver transplantation, extended hepatectomy, small-for-size syndrome, split liver grafts, cholestatic liver disease, pulmonary fibrosis, interstitial lung disease (ILD), idiopathic interstitial pneumonia (IIP), idiopathic pulmonary fibrosis (IPF), cystic fibrosis, progressive massive fibrosis, scleroderma, obliterative bronchiolitis, Hermansky-Pudlak syndrome, asbestosis, silicosis, sarcoidosis, tumor stroma in lung disease, chronic obstructive pulmonary disease (COPD), emphysema, pneumonia, pulmonary edema, chronic bronchitis, asthma, hypertrophic cardiomyopathy (HCM), dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertension, hypertensive heart disease, arrhythmogenic right ventricular cardiomyopathy (ARVC), atherosclerosis, chronic pulmonary hypertension, AIDS-associated pulmonary hypertension, varicose veins, cerebral infarcts, tubulointerstitial fibrosis, glomerular fibrosis, renal fibrosis, nephritic syndrome, Alport's syndrome, HIV-associated nephropathy, polycystic kidney disease, Fabry's disease, diabetic nephropathy, chronic glomerulonephritis, nephritis associated with systemic lupus, pancreatic fibrosis, cystic fibrosis, chronic pancreatitis, gliosis, Alzheimer's disease, multiple sclerosis, muscular dystrophy, Duchenne muscular dystrophy (DMD), Becker's muscular dystrophy (BMD), fibrotic myopathy, inflammatory bowel disease (IBD), Crohn's disease, microscopic colitis, primary sclerosing cholangitis (PSC), scleroderma, nephrogenic systemic fibrosis, Dupuytren's contracture, cutis keloid, Grave's ophthalmopathy, epiretinal fibrosis, retinal fibrosis, subretinal fibrosis, subretinal fibrosis associated with macular degeneration (e.g. wet age-related macular degeneration (AMD)), diabetic retinopathy, glaucoma, corneal fibrosis, post-surgical fibrosis (e.g. of the posterior capsule following cataract surgery, or of the bleb following trabeculectomy for glaucoma), conjunctival fibrosis, subconjunctival fibrosis, arthrofibrosis, arthritis, adhesive capsulitis, progressive systemic sclerosis (PSS), chronic graft versus host disease (GVHD), fibrotic pre-neoplastic, fibrotic neoplastic disease, fibrosis induced by chemical or environmental insult, gastric cancer, esophageal cancer, lung cancer, head and neck cancer, colorectal cancer, pancreatic cancer, cervical cancer, vulvar cancer, mediastinal fibrosis, retroperitoneal fibrosis, myelofibrosis and Peyronie's disease.

In some embodiments, the disease/condition characterised by damage to and/or death of cells of the liver is selected from: chronic liver disease, liver fibrosis, cirrhosis, non-alcoholic fatty liver disease (NAFLD), hepatitis, steatohepatitis, non-alcoholic steatohepatitis (NASH), alcoholic liver disease (ALD), alcoholic fatty liver (AFL), alcoholic hepatitis, alcoholic steatohepatitis (ASH), primary biliary cholangitis (PBC), primary sclerosing cholangitis (PSC), schistosomal liver disease, congenital liver disease, liver cancer, hepatocellular carcinoma (HCC), acute liver injury (ALI), acute liver failure, acute liver disease, viral hepatitis, liver ischemia-reperfusion injury (IRI), warm ischemia-reperfusion (WIR), radiation-induced liver disease (RILD), drug-induced liver injury (DILI), acetaminophen-induced liver injury, autoimmune liver injury, liver transplantation, extended hepatectomy, small-for-size syndrome, split liver grafts, and cholestatic liver disease.

### Description

### ITFG1

The present disclosure relates particularly to inhibition of gene and/or protein expression of integrin alpha FG-GAP repeat containing 1 (ITFG1; also known as TIP, Linkin/LNKN-1). Human ITFG1 isoform 1 is a 612 amino acid protein comprising a FG-GAP repeat (SEQ ID NO:2), an N-terminal signal peptide (SEQ ID NO:3) and a transmembrane region proximal to the C-terminus (SEQ ID NO:4). The amino acid sequence of mature human ITFG1 isoform 1—after processing to remove the signal peptide—is shown in SEQ ID NO:5. The amino acid sequence of human ITFG1 isoform 2 is shown in SEQ ID NO:6.

ITGF1 is a highly conserved protein, which is thought to possess immunomodulatory function. ITFG1 has been reported to promote the secretion of IFNγ, TNFα and IL-10 from primary human and murine T cells in vitro, and to ameliorate pathology in vivo in a mouse model of acute graft-versus-host disease (GVHD; Fiscella et al., Nat Biotechnol. (2003) 21(3):302-7). Interaction between ITFG1 and ATPase RUVBL1 is reported to be important for breast cancer cell invasion and progression (Fan et al., Biochim. Biophys. Acta. Gen. Subj. (2017) 1861(7):1788-1800). ITFG1 is found in recurrent amplifications in hepatocellular carcinoma (Rudalska et al., Nat. Med. (2014) 20(10):1138-1146). As noted above, WO 2022/025827 A1 identifies ITFG1 as a target for inhibition in order to promote proliferation/expansion of cells including hepatocytes, lung cells and myoblasts, wound healing and regeneration of liver tissue, and for treatment/prevention of disease characterised by fibrosis, e.g. fibrosis of the liver. A functional genetic in vivo RNAi screen in the FAH−/− mouse determined that shRNA targeting Itfg1 promoted hepatocyte proliferation/expansion, and thus regeneration of liver tissue (Example 1 of WO 2022/025827 A1). shRNA targeting Itfg1 was found to accelerate wound healing and liver regeneration in vitro and in vivo (Example 2 of WO 2022/025827 A1). RNAi-mediated knockdown of Itfg1 was moreover shown to attenuate the development of fibrosis in a murine model of non-alcoholic fatty liver disease (NAFLD; Example 2 of WO 2022/025827 A1). RNAi-mediated knockdown of Itfg1 was also found to enhance proliferation of lung cells and myoblasts (Example 2 of WO 2022/025827 A1).

In this specification, reference to ‘ITFG1’ encompasses: human ITFG1 isoform 1, isoforms of human ITFG1 isoform 1 (e.g. human ITFG1 isoform 2), homologues of human ITFG1 isoform 1 (i.e. encoded by the genome of a non-human animal), and variants thereof. In some embodiments, ITFG1 according to the present disclosure comprises or consists of an amino acid sequence having at 70% or greater amino acid sequence identity, preferably one of 75%, 80%, 85%, 90%, 91%, 92%, 93%, 94%, 95%, 96%, 97%, 98%, 99% or 100% amino acid sequence identity to the amino acid sequence of SEQ ID NO:1, 5 or 6. In some embodiments, ITFG1 comprises or consists of an amino acid sequence having at 70% or greater amino acid sequence identity, preferably one of 75%, 80%, 85%, 90%, 91%, 92%, 93%, 94%, 95%, 96%, 97%, 98%, 99% or 100% amino acid sequence identity to the amino acid sequence of SEQ ID NO:1.

A homologue of human ITFG1 isoform 1 may be from any animal. In some embodiments, a homologue of human ITFG1 isoform 1 may be from a mammal. In some embodiments, the mammal may be a non-human mammal, e.g. a primate (e.g. a non-human primate, e.g. an animal of the genus Macaca (e.g. Macaca fascicularis, Macaca mulatta), e.g. a non-human hominid (e.g. Pan troglodytes)). In some embodiments, the mammal may be a rabbit, guinea pig, rat, mouse or animal of the order Rodentia, cat, dog, pig, sheep, goat, an animal of the order Bos (e.g. cattle), an animal of the family Equidae (e.g. horse) or donkey.

Homologues of human ITFG1 isoform 1 may optionally be characterised as having 70% or greater amino acid sequence identity, preferably one of 75%, 80%, 85%, 90%, 91%, 92%, 93%, 94%, 95%, 96%, 97%, 98%, 99% or 100% amino acid sequence identity to the amino acid sequence of SEQ ID NO:1. Variants of human ITFG1 isoform 1 may optionally be characterised as having 70% or greater amino acid sequence identity, preferably one of 75%, 80%, 85%, 90%, 91%, 92%, 93%, 94%, 95%, 96%, 97%, 98%, 99% or greater amino acid sequence identity to the amino acid sequence of SEQ ID NO:1.

The amino acid sequence of Macaca fascularis has 99.3% sequence identity (608/612 amino acids) to SEQ ID NO:1, and is shown in SEQ ID NO:7. The amino acid sequence of Mus musculus ITFG1 has 89.2% sequence identity (546/612 amino acids) to SEQ ID NO:1, and is shown in SEQ ID NO:8.

### Inhibitory Nucleic Acids and Related Articles

Aspects and embodiments of the present disclosure relate to inhibitory nucleic acids. As used herein, an ‘inhibitory nucleic acid’ refers to a nucleic acid capable of reducing or preventing the gene and/or protein expression of one or more given target gene(s)/protein(s).

Inhibitory nucleic acids according to the present disclosure are suitable for reducing gene and/or protein expression of ITFG1. It will be appreciated that where an inhibitory nucleic acid is described as reducing gene expression of ITFG1, inhibition of expression of the gene encoding ITFG1 is intended. That is, reference herein to inhibition of gene expression of ITFG1 contemplates inhibition of expression of ITFG1.

In some embodiments, an inhibitory nucleic acid according to the present disclosure may: reduce/prevent expression of a gene encoding ITFG1;
reduce/prevent transcription of nucleic acid encoding ITFG1 (e.g. from DNA encoding ITFG1 to RNA encoding ITFG1);
reduce the level of RNA encoding ITFG1;
increase degradation of RNA encoding ITFG1;
reduce the level of ITFG1 protein;
reduce/prevent normal splicing of pre-mRNA encoding ITFG1;
reduce/prevent translation of mRNA encoding ITFG1;
reduce the level of a function of ITFG1; and/or
Increase cell proliferation/population expansion (e.g. of ITFG1-expressing cells, e.g. hepatocytes).

It will be appreciated that a given nucleic acid may display more than one of the properties recited in the preceding paragraph. A given nucleic acid may be evaluated for the properties recited in the preceding paragraph using suitable assays. The assays may be e.g. in vitro assays, optionally cell-based assays or cell-free assays. The assays may be e.g. in vivo assays, i.e. performed in non-human animals.

Where assays are cell-based assays, they may comprise treating cells with a nucleic acid in order to determine whether the nucleic acid displays one or more of the recited properties. Assays may employ species labelled with detectable entities in order to facilitate their detection. Assays may comprise evaluating the recited properties following treatment of cells separately with a range of quantities/concentrations of a given nucleic acid (e.g. a dilution series). It will be appreciated that the cells are preferably cells that express ITFG1, e.g. liver cells (e.g. HepG2 cells or HuH7 cells).

Analysis of the results of such assays may comprise determining the concentration at which 50% of the maximal level of the relevant activity is attained. The concentration of nucleic acid at which 50% of the maximal level of the relevant activity is attained may be referred to as the ‘half-maximal effective concentration’ of the nucleic acid in relation to the relevant activity, which may also be referred to as the ‘EC₅₀’. By way of illustration, the EC₅₀ of a given inhibitory nucleic acid for increasing degradation of RNA encoding ITFG1 may be the concentration at which 50% of the maximal level of degradation of RNA encoding ITFG1 is achieved.

Depending on the property, the EC₅₀ may also be referred to as the ‘half-maximal inhibitory concentration’ or ‘IC₅₀’, this being the concentration of nucleic acid at which 50% of the maximal level of inhibition of a given property is observed. By way of illustration, the IC₅₀ of a given inhibitory nucleic acid for reducing expression of a gene encoding ITFG1 may be the concentration at which 50% of the maximal level of inhibition of expression of the gene is achieved.

Analysis of the results of such assays may comprise determining the concentration at which a specific effect is produced in 50% of a treated population, which may also be referred to as the ‘ED50’. Analysis of the results of such assays may comprise determining the concentration at which a specific effect is produced in 80% of a treated population, which may also be referred to as the ‘ED80’. By way of illustration, the ED50 or ED80 of a given inhibitory nucleic acid for increasing degradation of RNA encoding ITFG1 may be the concentration at which 50% or 80% (respectively) of the maximal level of degradation of RNA encoding ITFG1 is achieved.

Nucleic acids capable of reducing/preventing gene expression of ITFG1 and/or reducing/preventing transcription of nucleic acid encoding ITFG1 and/or reducing the level of RNA encoding ITFG1 and/or increasing degradation of RNA encoding ITFG1 may be identified using assays comprising detecting and/or quantifying the level of RNA encoding ITFG1. Such assays may comprise quantifying RNA encoding ITFG1 by RT-qPCR (a technique well known to the skilled person). The methods may employ primers and/or probes for the detection and/or quantification of RNA encoding ITFG1. Such assays may comprise introducing (e.g. by transfection) into cells that express ITFG1 in in vitro culture (i) a putative inhibitory nucleic acid, or (ii) a control nucleic acid (e.g. a nucleic acid known not to influence the level of RNA encoding ITFG1), and subsequently (e.g. after an appropriate period of time, i.e. a period of time sufficient for a reduction in the level of gene expression of ITFG1/transcription of nucleic acid encoding ITFG1/level of RNA encoding ITFG1 or an increase in the level of degradation of RNA encoding ITFG1 to be observed) measuring the level of RNA encoding ITFG1 in cells according to (i) and (ii), and (iii) comparing the level of RNA encoding ITFG1 detected to determine whether the putative inhibitory nucleic acid reduces/prevents gene expression of ITFG1/transcription of nucleic acid encoding ITFG1, and/or reduces the level of RNA encoding ITFG1, and/or increases degradation of RNA encoding ITFG1.

In particular embodiments, a nucleic acid may be evaluated for its ability to reduce/prevent gene expression of ITFG1 as described in Example 2 herein.

Nucleic acids capable of reducing/preventing normal splicing of pre-mRNA encoding ITFG1 may be identified using assays comprising detecting and/or quantifying the level of RNA (e.g. mature mRNA) encoding one or more isoforms of ITFG1. Such assays may comprise quantifying RNA (e.g. mature mRNA) encoding one or more isoforms of ITFG1 by RT-qPCR. The methods may employ primers and/or probes for the detection and/or quantification of mature mRNA produced by canonical splicing of pre-mRNA transcribed from a gene encoding ITFG1, and/or primers and/or probes for the detection and/or quantification of mature mRNA produced by alternative splicing of pre-mRNA transcribed from a gene encoding ITFG1. Mature mRNA produced by canonical splicing of pre-mRNA transcribed from a gene encoding ITFG1 may be mature mRNA encoding the major isoform produced by expression of the gene encoding ITFG1. The major isoform may be the most commonly produced/detected isoform. For example, mature mRNA produced by canonical splicing of pre-mRNA transcribed from human ITFG1 may be mature mRNA encoding human ITFG1 isoform 1 (i.e. having the amino acid sequence shown in SEQ ID NO:1). Mature mRNA produced by alternative splicing of pre-mRNA transcribed from a gene encoding ITFG1 may be mature mRNA encoding an isoform other than the major isoform produced by expression of the gene encoding ITFG1. For example, mature mRNA produced by alternative splicing of pre-mRNA transcribed from human ITFG1 may be mature mRNA encoding an isoform of human ITFG1 other than isoform 1 (i.e. having an amino acid sequence non-identical to SEQ ID NO:1); e.g. mature mRNA encoding human ITFG1 isoform 2 (i.e. having the amino acid sequence shown in SEQ ID NO:6). Such assays may comprise introducing (e.g. by transfection) into cells that express ITFG1 in in vitro culture (i) a putative inhibitory nucleic acid, or (ii) a control nucleic acid (e.g. a nucleic acid known not to influence splicing of pre-mRNA encoding ITFG1), and subsequently (e.g. after an appropriate period of time, i.e. a period of time sufficient for an effect on splicing of pre-mRNA encoding ITFG1 to be observed) measuring the level of mature mRNA encoding one or more isoforms of ITFG1 in cells according to (i) and (ii), and (iii) comparing the level of mature mRNA encoding the isoform(s) to determine whether the putative inhibitory nucleic acid reduces/prevents normal splicing of pre-mRNA encoding ITFG1.

Nucleic acids capable of reducing the level of ITFG1 protein and/or reducing/preventing translation of mRNA encoding ITFG1 may be identified using assays comprising detecting the level of ITFG1 protein, e.g. using techniques well known to the skilled person, such as antibody/reporter-based methods (western blot, ELISA, immunohisto/cytochemistry, etc.). The methods may employ antibodies specific for ITFG1. Such assays may comprise introducing (e.g. by transfection) into cells that express ITFG1 in in vitro culture (i) a putative inhibitory nucleic acid, or (ii) a control nucleic acid (e.g. a nucleic acid known not to influence the level of ITFG1 protein), and subsequently (e.g. after an appropriate period of time, i.e. a period of time sufficient for a reduction in the level of ITFG1 protein to be observed) measuring the level of ITFG1 protein in cells according to (i) and (ii), and (iii) comparing the level of ITFG1 protein detected to determine whether the putative inhibitory nucleic acid reduces the level of ITFG1 protein and/or reduces/prevents translation of mRNA encoding ITFG1.

In particular embodiments, a nucleic acid may be evaluated for its ability to reduce/prevent the level of ITFG1 protein as described in Example 6 herein.

Nucleic acids capable of reducing the level of a function of ITFG1 (e.g. a function of ITFG1 described herein) may be identified using assays comprising detecting the level of the relevant function. Such assays may comprise introducing (e.g. by transfection) into cells that express ITFG1 in in vitro culture (i) a putative inhibitory nucleic acid, or (ii) a control nucleic acid (e.g. a nucleic acid known not to influence ITFG1 function), and subsequently (e.g. after an appropriate period of time, i.e. a period of time sufficient for a reduction in the level of a function of ITFG1 to be observed) measuring the level of a function of ITFG1 in cells according to (i) and (ii), and (iii) comparing the level of the function of ITFG1 detected to determine whether the putative inhibitory nucleic acid reduces the level of a function of ITFG1.

Cell proliferation and/or population expansion of a given cell type (e.g. ITFG1-expressing cells, e.g. hepatocytes) or population thereof can be evaluated in vitro by measuring or monitoring the number or proportion of such cells overtime.

Cell proliferation/expansion can be investigated by analysing cell division or the number of cells over a period of time. Cell division can be analysed, for example, by in vitro analysis of incorporation of 3H-thymidine or by CFSE dilution assay, e.g. as described in Fulcher and Wong, Immunol Cell Biol (1999) 77(6): 559-564, hereby incorporated by reference in entirety. Proliferating cells can also be identified by analysis of incorporation of 5-ethynyl-2′-deoxyuridine (EdU) by an appropriate assay, as described e.g. in Buck et a., Biotechniques. 2008 June; 44(7):927-9, and Sali and Mitchison, PNAS USA 2008 Feb. 19; 105(7): 2415-2420, both hereby incorporated by reference in their entirety. Other suitable assays include assays for quantifying viable cells, e.g. the CellTiter-Glo Luminescent Cell Viability Assay (Promega) or the CCK8 Assay (Dojindo).

Nucleic acids capable of increasing cell proliferation and/or population expansion of a given cell type (e.g. ITFG1-expressing cells, e.g. hepatocytes) may be identified using assays comprising introducing (e.g. by transfection) into the cells in in vitro culture (i) a putative inhibitory nucleic acid, or (ii) a control nucleic acid (e.g. a nucleic acid known not to increase cell proliferation/population expansion of the given cell type), and subsequently (e.g. after an appropriate period of time, i.e. a period of time sufficient for an increase in cell proliferation/population expansion to be observed) measuring the number/proportion of, or some other correlate cell proliferation/population expansion of, cells according to (i) and (ii), and (iii) comparing the number/proportion/proliferation/population expansion detected to determine whether the putative inhibitory nucleic acid increases cell proliferation and/or population expansion.

In particular embodiments, a nucleic acid may be evaluated for its ability to increase cell proliferation/population expansion (e.g. of ITFG1-expressing cells, e.g. hepatocytes) as described in Example 3 or 7 herein.

In some embodiments, an inhibitory nucleic acid according to the present disclosure may reduce expression of a gene encoding ITFG1 to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of expression observed in the absence of the inhibitory nucleic acid, or in the presence of the same quantity of a control nucleic acid known not to inhibit expression of the relevant gene, in a given assay. In some embodiments, an inhibitory nucleic acid according to the present disclosure may be capable of reducing expression of a gene encoding ITFG1 to less than 100%, e.g. one of ≤99%, ≤95%, ≤90%, ≤85%, ≤80%, ≤75%, ≤70%, ≤65%, ≤60%, ≤55%, ≤50%, ≤45%, ≤40%, ≤35%, ≤30%, ≤25%, ≤20%, ≤15%, ≤10%, ≤5%, or ≤1% of the level of expression observed in the absence of the inhibitory nucleic acid, or in the presence of the same quantity of a control nucleic acid known not to inhibit expression of the relevant gene, in a given assay.

In some embodiments, an inhibitory nucleic acid according to the present disclosure may be capable of reducing the level of RNA encoding ITFG1 to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level observed in the absence of the inhibitory nucleic acid, or in the presence of the same quantity of a control nucleic acid known not to reduce the level of RNA encoding ITFG1, in a given assay. In some embodiments, an inhibitory nucleic acid according to the present disclosure may be capable of reducing the level of RNA encoding ITFG1 to less than 100%, e.g. one of ≤99%, ≤95%, ≤90%, ≤85%, ≤80%, ≤75%, ≤70%, ≤65%, ≤60%, ≤55%, ≤50%, ≤45%, ≤40%, ≤35%, ≤30%, ≤25%, ≤20%, ≤15%, ≤10%, ≤5%, or ≤1% of the level observed in the absence of the inhibitory nucleic acid, or in the presence of the same quantity of a control nucleic acid known not to reduce the level of RNA encoding ITFG1, in a given assay.

In some embodiments, an inhibitory nucleic acid according to the present disclosure may be capable of reducing the level of transcription of nucleic acid encoding ITFG1 to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level observed in the absence of the inhibitory nucleic acid, or in the presence of the same quantity of a control nucleic acid known not to reduce transcription of nucleic acid encoding ITFG1, in a given assay. In some embodiments, an inhibitory nucleic acid according to the present disclosure may be capable of reducing the level of transcription of nucleic acid encoding ITFG1 to less than 100%, e.g. one of ≤99%, ≤95%, ≤90%, ≤85%, ≤80%, ≤75%, ≤70%, ≤65%, ≤60%, ≤55%, ≤50%, ≤45%, ≤40%, ≤35%, ≤30%, ≤25%, ≤20%, ≤15%, ≤10%, ≤5%, or ≤1% of the level observed in the absence of the inhibitory nucleic acid, or in the presence of the same quantity of a control nucleic acid known not to reduce transcription of nucleic acid encoding ITFG1, in a given assay.

In some embodiments, an inhibitory nucleic acid according to the present disclosure may be capable of reducing the level of ITFG1 protein to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level observed in the absence of the inhibitory nucleic acid, or in the presence of the same quantity of a control nucleic acid known not to reduce the level of ITFG1 protein, in a given assay.

In some embodiments, an inhibitory nucleic acid according to the present disclosure may be capable of reducing the level of ITFG1 protein to less than 100%, e.g. one of ≤99%, ≤95%, ≤90%, ≤85%, ≤80%, ≤75%, ≤70%, ≤65%, ≤60%, ≤55%, ≤50%, ≤45%, ≤40%, ≤35%, ≤30%, ≤25%, ≤20%, ≤15%, ≤10%, ≤5%, or ≤1% of the level observed in the absence of the inhibitory nucleic acid, or in the presence of the same quantity of a control nucleic acid known not to reduce the level of ITFG1 protein, in a given assay.

In some embodiments, an inhibitory nucleic acid according to the present disclosure may be capable of reducing the level of a function of ITFG1 to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level observed in the absence of the inhibitory nucleic acid, or in the presence of the same quantity of a control nucleic acid known not to reduce the level of the function of ITFG1, in a given assay. In some embodiments, an inhibitory nucleic acid according to the present disclosure may be capable of reducing the level of a function of ITFG1 to less than 100%, e.g. one of ≤99%, ≤95%, ≤90%, ≤85%, ≤80%, ≤75%, ≤70%, ≤65%, ≤60%, ≤55%, ≤50%, ≤45%, ≤40%, ≤35%, ≤30%, ≤25%, ≤20%, ≤15%, ≤10%, ≤5%, or ≤1% of the level observed in the absence of the inhibitory nucleic acid, or in the presence of the same quantity of a control nucleic acid known not to reduce the level of the function of ITFG1, in a given assay.

In some embodiments, an inhibitory nucleic acid according to the present disclosure may be capable of reducing normal splicing of pre-mRNA encoding ITFG1 to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level observed in the absence of the inhibitory nucleic acid, or in the presence of the same quantity of a control nucleic acid known not to reduce normal splicing of pre-mRNA encoding ITFG1, in a given assay. In some embodiments, an inhibitory nucleic acid according to the present disclosure may be capable of reducing the level of normal splicing of pre-mRNA encoding ITFG1 to less than 100%, e.g. one of ≤99%, ≤95%, ≤90%, ≤85%, ≤80%, ≤75%, ≤70%, ≤65%, ≤60%, ≤555%, ≤50%, ≤45%, ≤40%, ≤35%, ≤30%, ≤25%, ≤20%, ≤15%, ≤10%, ≤5%, or ≤51% of the level observed in the absence of the inhibitory nucleic acid, or in the presence of the same quantity of a control nucleic acid known not to reduce normal splicing of pre-mRNA encoding ITFG1, in a given assay.

In some embodiments, an inhibitory nucleic acid according to the present disclosure may be capable of reducing translation of mRNA encoding ITFG1 to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level observed in the absence of the inhibitory nucleic acid, or in the presence of the same quantity of a control nucleic acid known not to reduce translation of mRNA encoding ITFG1, in a given assay. In some embodiments, an inhibitory nucleic acid according to the present disclosure may be capable of reducing translation of mRNA encoding ITFG1 to less than 100%, e.g. one of 599%, ≤95%, ≤90%, ≤85%, ≤80%, ≤75%, ≤70%, ≤65%, ≤60%, ≤55%, ≤50%, ≤45%, ≤40%, ≤35%, ≤30%, ≤25%, ≤20%, ≤15%, ≤10%, ≤5%, or ≤1% of the level observed in the absence of the inhibitory nucleic acid, or in the presence of the same quantity of a control nucleic acid known not to reduce translation of mRNA encoding ITFG1, in a given assay.

Preferred levels of reduction in accordance with the preceding seven paragraphs are reduction to less than 0.5 times/≤50%, e.g. one of less than 0.4 times/≤40%, less than 0.3 times/≤30%, less than 0.2 times/≤20%, less than 0.15 times/≤15%, or less than 0.1 times/≤10%.

In some embodiments, an inhibitory nucleic acid according to the present disclosure may be capable of increasing degradation of RNA encoding ITFG1 to more than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥1.5 times, ≥1.6 times, ≥21.7 times, ≥21.8 times, ≥21.9 times, ≥2 times, ≥23 times, ≥4 times, ≥25 times, ≥26 times, ≥27 times, ≥8 times, ≥9 times or ≥10 times the level observed in the absence of the inhibitory nucleic acid, or in the presence of the same quantity of a control nucleic acid known not to increase degradation of RNA encoding ITFG1, in a given assay.

In some embodiments, an inhibitory nucleic acid according to the present disclosure may be capable of increasing cell proliferation/population expansion (e.g. of ITFG1-expressing cells, e.g. hepatocytes) to more than 1 times, e.g. one of ≥1.01 times, ≥1.02 times, ≥1.03 times, ≥1.04 times, ≥21.05 times, ≥1.1 times, ≥1.2 times, ≥1.3 times, ≥1.4 times, ≥1.5 times, ≥1.6 times, ≥1.7 times, ≥1.8 times, ≥1.9 times, ≥2 times, ≥3 times, ≥24 times, ≥5 times, ≥6 times, ≥27 times, ≥8 times, ≥9 times or ≥10 times the level observed in the absence of the inhibitory nucleic acid, or in the presence of the same quantity of a control nucleic acid known not to increase cell proliferation/population expansion of the relevant cell type, in a given assay.

In some embodiments, an inhibitory nucleic acid according to the present disclosure prevents or silences expression of a gene encoding ITFG1. In some embodiments, an inhibitory nucleic acid according to the present disclosure prevents or silences expression of ITFG1 at the protein level. As used herein, expression of a given gene/protein may be considered to be ‘prevented’ or ‘silenced’ where the level of expression is reduced to less than 0.1 times/≤10% of the level observed in the absence of the inhibitory nucleic acid, or in the presence of the same quantity of a control nucleic acid known not to be an inhibitor of expression of the relevant gene(s)/protein(s).

In preferred embodiments, an inhibitory nucleic acid (e.g. an siRNA) according to the present disclosure inhibits greater than 50%, e.g. one of ≥60%, ≥61%, ≥62%, ≥63%, ≥64%, ≥65%, ≥66%, ≥67%, ≥68%, ≥69%, ≥70%, ≥71%, ≥72%, ≥73%, ≥74%, ≥75%, ≥76%, ≥77%, ≥78%, ≥79%, ≥80%, ≥81%, ≥82%, ≥83%, ≥84%, ≥85%, 86%, ≥87%, ≥88%, ≥89%, ≥90%, ≥91%, ≥92%, ≥93%, ≥94%, ≥95%, ≥96%, ≥97%, ≥98%, ≥99% or 100% of the gene and/or protein expression of ITFG1 observed in the absence of the inhibitory nucleic acid, or in the presence of the same quantity of a control nucleic acid known not to inhibit gene and/or protein expression of ITFG1, in a given assay.

In preferred embodiments, an inhibitory nucleic acid (e.g. an siRNA) according to the present disclosure inhibits greater than 50%, e.g. one of ≥60%, ≥61%, ≥62%, ≥63%, ≥64%, ≥65%, ≥66%, ≥67%, ≥68%, ≥69%, ≥70%, ≥71%, ≥72%, ≥73%, ≥74%, ≥75%, ≥76%, ≥77%, ≥78%, ≥79%, ≥80%, ≥81%, ≥82%, ≥83%, ≥84%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% of the gene expression of ITFG1 (e.g. as determined by qRT-PCR) observed in the absence of the inhibitory nucleic acid, or in the presence of the same quantity of a control nucleic acid known not to inhibit gene and/or protein expression of ITFG1, in a given assay (e.g. the assay described in Example 2 herein).

In preferred embodiments, an inhibitory nucleic acid (e.g. an siRNA) according to the present disclosure inhibits greater than 50%, e.g. one of ≥60%, ≥61%, ≥62%, ≥63%, ≥64%, ≥65%, ≥66%, ≥67%, ≥68%, ≥69%, ≥70%, ≥71%, ≥72%, ≥73%, ≥74%, ≥75%, ≥76%, ≥77%, ≥78%, ≥79%, ≥80%, ≥81%, ≥82%, ≥83%, 84%, ≥85%, ≥86%, ≥87%, ≥88%, ≥89%, ≥90%, ≥91%, ≥92%, ≥93%, ≥94%, ≥95%, ≥96%, ≥97%, ≥98%, ≥99% or 100% of the protein expression of ITFG1 (e.g. as determined by ELISA) observed in the absence of the inhibitory nucleic acid, or in the presence of the same quantity of a control nucleic acid known not to inhibit gene and/or protein expression of ITFG1, in a given assay (e.g. the assay described in Example 2 herein).

In some embodiments, an inhibitory nucleic acid (e.g. an siRNA) according to the present disclosure may inhibit gene and/or protein expression of ITFG1 with an IC₅₀ of 51 μM, e.g. one of ≤500 nM, ≤100 nM, ≤75 nM, ≤50 nM, ≤40 nM, ≤30 nM, ≤20 nM, ≤15 nM, ≤12.5 nM, ≤10 nM, ≤9 nM, ≤8 nM, ≤7 nM, ≤6 nM, ≤5 nM, ≤4 nM≤3 nM, ≤2 nM, ≤1 nM, ≤900 pM, ≤800 pM, ≤700 pM, ≤600 pM, ≤500 pM, ≤400 pM, ≤300 pM, ≤200 pM, ≤100 pM, ≤50 pM, ≤40 pM, ≤30 pM, ≤20 pM, ≤10 pM or ≤1 pM.

In some embodiments an inhibitory nucleic acid according to the present disclosure (e.g. an siRNA) may inhibit gene expression of ITFG1 (e.g. as determined by qRT-PCR) with an IC₅₀ of ≤1 nM, ≤900 pM, ≤800 pM, ≤700 pM, ≤600 pM, ≤500 pM, ≤400 pM, ≤300 pM, ≤200 pM, ≤100 pM, ≤50 pM, ≤40 pM, ≤30 pM, ≤20 pM, ≤10 pM or ≤1 pM.

In some embodiments an inhibitory nucleic acid according to the present disclosure (e.g. an siRNA) may inhibit protein expression of ITFG1 (e.g. as determined by ELISA) with an IC₅₀ of ≤1 nM, ≤900 pM, ≤800 pM, ≤700 pM, ≤600 pM, ≤500 pM, ≤400 pM, ≤300 pM, ≤200 pM, ≤100 pM, ≤50 pM, ≤40 pM, ≤30 pM, ≤20 pM, ≤10 pM or ≤1 pM.

WO 2022/025827 A1 (hereby incorporated by reference in its entirety) describes inhibitory nucleic acids directed against ITFG1. In some embodiments, an inhibitory nucleic acid described in WO 20221025827 Al may be selected from: an siRNA having a guide strand/antisense sequence according to one of SEQ ID NOs:457 to 1482 of WO 2022/025827 A1 (see Table 4 thereof); an siRNA having a guide strand/antisense sequence according to SEQ ID NO:7 or 8 of WO 2022/025827 A1 (see Table 1 thereof); an siRNA formed by SEQ ID NOs:7095 and 7144 of WO 2022/025827 A1 (see Table 11 thereof); an siRNA formed by SEQ ID NOs:7096 and 7145 of WO 2022/025827 A1 (see Table 11 thereof); an siRNA formed by SEQ ID NOs:7149 and 7154 of WO 2022/025827 A1 (see Table 11 thereof); an siRNA formed by SEQ ID NOs:7150 and 7155 of WO 2022/025827 A1 (see Table 11 thereof); an shRNA encoded by the nucleotide sequence according to one of SEQ ID NOs:7109 to 7114 of WO 2022/025827 A1 (see Table 12 thereof); and an shRNA encoded by the nucleotide sequence according to one of SEQ ID NOs:7131 to 7140 of WO 2022/025827 A1 (see Table 13 thereof).

An inhibitory nucleic acid according to the present disclosure may be non-identical to an inhibitory nucleic acid disclosed in WO 2022/025827 A1. In preferred embodiments, the inhibitory nucleic acids of the present disclosure possess novel and/or improved properties compared to an inhibitory nucleic acid disclosed in WO 2022/025827 A1.

In some embodiments, an inhibitory nucleic acid according to the present disclosure:
reduces expression of a gene encoding ITFG1 to a greater extent than an inhibitory nucleic acid described in WO 2022/025827 A1;
reduces transcription of nucleic acid encoding ITFG1 (e.g. from DNA encoding ITFG1 to RNA encoding ITFG1) to a greater extent than an inhibitory nucleic acid described in WO 2022/025827 A1;
reduces the level of RNA encoding ITFG1 to a greater extent than an inhibitory nucleic acid described in WO 20221025827 Al;
increases degradation of RNA encoding ITFG1 to a greater extent than an inhibitory nucleic acid described in WO 2022/025827 A1;
reduces the level of ITFG1 protein to a greater extent than an inhibitory nucleic acid described in WO 2022/025827 A1;
reduces normal splicing of pre-mRNA encoding ITFG1 to a greater extent than an inhibitory nucleic acid described in WO 2022/025827 A1;
reduces translation of mRNA encoding ITFG1 to a greater extent than an inhibitory nucleic acid described in WO 2022/025827 A1;
reduces the level of a function of ITFG1 to a greater extent than an inhibitory nucleic acid described in WO 2022/025827 A1;
increases cell proliferation/population expansion (e.g. of ITFG1-expressing cells, e.g. hepatocytes) to a greater extent than an inhibitory nucleic acid described in WO 2022/025827 A1; and/or
displays reduced toxicity (e.g. cytotoxicity in vitro, e.g. toxicity in vivo to a subject administered the inhibitory nucleic acid) than an inhibitory nucleic acid described in WO 2022/025827 A1.

In some embodiments, an inhibitory nucleic acid according to the present disclosure reduces expression of a gene encoding ITFG1 to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level to which an inhibitory nucleic acid described in WO 2022/025827 A1 reduces expression of the relevant gene in a given assay, at a comparable concentration.

In some embodiments, an inhibitory nucleic acid according to the present disclosure reduces transcription of nucleic acid encoding ITFG1 to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or 50.01 times the level to which an inhibitory nucleic acid described in WO 2022/025827 A1 reduces transcription of the nucleic acid in a given assay, at a comparable concentration.

In some embodiments, an inhibitory nucleic acid according to the present disclosure reduces the level of RNA encoding ITFG1 to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level to which an inhibitory nucleic acid described in WO 2022/025827 A1 reduces the level of the RNA in a given assay, at a comparable concentration.

In some embodiments, an inhibitory nucleic acid according to the present disclosure increases degradation of RNA encoding ITFG1 to more than 1 times, e.g. one of ≥1.01 times, ≥1.02 times, ≥1.03 times, ≥1.04 times, ≥1.05 times, ≥1.1 times, ≥1.2 times, ≥1.3 times, ≥1.4 times, ≥21.5 times, ≥21.6 times, ≥1.7 times, ≥1.8 times, ≥1.9 times, ≥2 times, ≥3 times, ≥4 times, ≥5 times, ≥6 times, ≥7 times, ≥8 times, ≥9 times or ≥10 times the level to which an inhibitory nucleic acid described in WO 20221025827 Al increases degradation of the RNA in a given assay, at a comparable concentration.

In some embodiments, an inhibitory nucleic acid according to the present disclosure reduces the level of ITFG1 protein to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level to which an inhibitory nucleic acid described in WO 2022/025827 A1 reduces the level of ITFG1 protein in a given assay, at a comparable concentration.

In some embodiments, an inhibitory nucleic acid according to the present disclosure reduces normal splicing of pre-mRNA encoding ITFG1 to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level to which an inhibitory nucleic acid described in WO 2022/025827 A1 reduces the level of normal splicing of pre-mRNA encoding ITFG1 in a given assay, at a comparable concentration.

In some embodiments, an inhibitory nucleic acid according to the present disclosure reduces translation of mRNA encoding ITFG1 to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level to which an inhibitory nucleic acid described in WO 2022/025827 A1 reduces the level of translation of mRNA encoding ITFG1 in a given assay, at a comparable concentration.

In some embodiments, an inhibitory nucleic acid according to the present disclosure reduces the level of a function of ITFG1 to less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≥0.25 times, ≤0.2 times, ≤0.15 times, ≤0.1 times, ≤0.05 times, or ≤0.01 times the level to which an inhibitory nucleic acid described in WO 2022/025827 A1 reduces the level of the relevant function in a given assay, at a comparable concentration.

In some embodiments, an inhibitory nucleic acid according to the present disclosure increases cell proliferation/population expansion (e.g. of ITFG1-expressing cells, e.g. hepatocytes) to more than 1 times, e.g. one of ≥1.01 times, ≥1.02 times, ≥1.03 times, ≥1.04 times, ≥1.05 times, ≥1.1 times, ≥1.2 times, ≥1.3 times, ≥21.4 times, ≥21.5 times, ≥1.6 times, ≥1.7 times, ≥1.8 times, ≥21.9 times, ≥2 times, ≥3 times, ≥4 times, ≥5 times, ≥6 times, ≥7 times, ≥8 times, ≥9 times or ≥10 times the level to which an inhibitory nucleic acid described in WO 2022/025827 A1 increases cell proliferation/population expansion of such cells in a given assay, at a comparable concentration. Toxicity of a given nucleic acid molecule can be investigated by analysing correlates of toxicity following treatment of cells or a subject with the nucleic acid molecule. Cytotoxicity can be evaluated by contacting cells, e.g. in vitro with the nucleic acid molecule, and subsequently detecting and/or quantifying the number proportion of viable and/or dead/dying cells, after a suitable period of time. Such analysis may comprise analysing the level of expression or activity of one or more caspases, and/or may comprise detecting and/or quantifying live, dead and/or apoptotic cells. Detecting/quantifying apoptosis may comprise detecting and/or quantifying one or markers of apoptosis (e.g. phosphatidylserine (PS) exposure, Bcl-2 family protein (e.g. Bax, Bak, Bid) activation, ROS production, caspase activation, mitochondrial membrane permeabilization or DNA fragmentation).

Cytotoxicity can be investigated, for example, using any of the methods reviewed in Zaritskaya et al., Expert Rev Vaccines (2011), 9(6):601-616, hereby incorporated by reference in its entirety. Examples of in vitro assays of cytotoxicity/cell killing include release assays such as the ⁵¹Cr release assay, the lactate dehydrogenase (LDH) release assay, the 3-(4,5-dimethylthiazol-2-yl)-2,5-diphenyl tetrazolium bromide (MTT) release assay, and the calcein-acetoxymethyl (calcein-AM) release assay. These assays measure cytotoxicity based on the detection of factors released from lysed cells. Other suitable assays include assays for quantifying viable cells, e.g. the CellTiter-Glo Luminescent Cell Viability Assay (Promega) or the CCK8 Assay (Dojindo).

In particular embodiments, a nucleic acid may be evaluated for its cytotoxicity (e.g. to ITFG1-expressing cells, e.g. hepatocytes) as described in Example 8 herein.

Toxicity to a subject treated with a given nucleic acid molecule can be evaluated by analysing the level of one or more correlates of damage to a cells/tissue (e.g. in a sample obtained from the subject), after a suitable period of time following administration of the nucleic acid molecule to the subject. A correlate of damage to a cells/tissue may be a correlate of an inflammatory immune response, or a biomarker of damage to, or impairment of the function of, a cell type/tissue/organ. In some embodiments, the organ may be the liver, and a correlate of damage to cells and/or tissue the liver may be the level of aspartate transaminase (AST) and/or alanine aminotransferase (ALT) in the serum of the subject.

In particular embodiments, a nucleic acid may be evaluated for its toxicity to a recipient subject as described in Example 11, 17 or 19 herein.

In some embodiments, an inhibitory nucleic acid according to the present disclosure displays a level of cytotoxicity (e.g. in vitro, e.g. to cells expressing ITFG1, e.g. hepatocytes) which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≥0.15 times, ≤0.1 times, ≤0.05 times, or 0.01 times the level of cytotoxicity displayed by an inhibitory nucleic acid described in WO 2022/025827 A1 in a given assay, at a comparable concentration.

In some embodiments, an inhibitory nucleic acid according to the present disclosure displays a level of toxicity (e.g. in vivo, e.g. in a subject administered the inhibitory nucleic acid) which is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of toxicity displayed by an inhibitory nucleic acid described in WO 2022/025827 A1 in a given assay, at a comparable concentration.

In some embodiments, the level of AST and/or ALT in the serum of a subject administered the inhibitory nucleic acid is less than 1 times, e.g. one of ≤0.99 times, ≤0.95 times, ≤0.9 times, ≤0.85 times, ≤0.8 times, ≤0.75 times, ≤0.7 times, ≤0.65 times, ≤0.6 times, ≤0.55 times, ≤0.5 times, ≤0.45 times, ≤0.4 times, ≤0.35 times, ≤0.3 times, ≤0.25 times, ≤0.2 times, ≤0.15 times, ≤0.1 times, ≤0.05 times, or ≤0.01 times the level of the relevant correlate of liver damage in the serum of a subject administered a comparable concentration of an inhibitory nucleic acid described in WO 2022/025827 A1, in a given assay.

Inhibitory nucleic acids according to the present disclosure may comprise or consist of DNA and/or RNA. Inhibitory nucleic acids may be single-stranded (e.g. in the case of antisense oligonucleotides (e.g. gapmers)). Inhibitory nucleic acids may be double-stranded or may comprise double-stranded region(s) (e.g. in the case of siRNA, shRNA, etc.). Inhibitory nucleic acids may comprise both double-stranded and single-stranded regions (e.g. in the case of shRNA and pre-miRNA molecules, which are double-stranded in the stem region of the hairpin structure, and single-stranded in the loop region of the hairpin structure).

In some embodiments, an inhibitory nucleic acid according to the present disclosure may be an antisense nucleic acid as described herein. In some embodiments, an inhibitory nucleic acid may comprise an antisense nucleic acid as described herein. In some embodiments, an inhibitory nucleic acid may encode an antisense nucleic acid as described herein.

As used herein, an ‘antisense nucleic acid’ refers to a nucleic acid (e.g. DNA or RNA) that is complementary to at least a portion of a target nucleotide sequence (e.g. of RNA encoding ITFG1). Antisense nucleic acids according to the present disclosure are preferably single-stranded nucleic acids, and bind via complementary Watson-Crick base-pairing to a target nucleotide sequence. Complementary base-pairing may involve hydrogen bonding between complementary base pairs. Antisense nucleic acids may be provided as single-stranded molecules, as for example in the case of antisense oligonucleotides, or may be comprised in double-stranded molecular species, as for example in the case of siRNA, shRNA and pre-miRNA molecules.

Complementary base-pairing between the antisense nucleic acid and its target nucleotide sequence may be complete. In such embodiments the antisense nucleic acid comprises, or consists of, the reverse complement of its target nucleotide sequence, and complementary base-pairing occurs between each nucleotide of the target nucleotide sequence and complementary nucleotides in the antisense nucleic acid. Alternatively, complementary base-pairing between the antisense nucleic acid and its target nucleotide sequence may be incomplete/partial. In such embodiments complementary base-pairing occurs between some, but not all, nucleotides of the target nucleotide sequence and complementary nucleotides in the antisense nucleic acid.

Such binding between nucleic acids through complementary base pairing may be referred to as ‘hybridisation’. Through binding to its target nucleotide sequence, an antisense nucleic acid may form a nucleic acid complex comprising (i) the antisense nucleic acid and (ii) a target nucleic acid comprising the target nucleotide sequence.

The nucleotide sequence of an antisense nucleic acid is sufficiently complementary to its target nucleotide sequence such that it binds or hybridises to the target nucleotide sequence. It will be appreciated that an antisense nucleic acid preferably has a high degree of sequence identity to the reverse complement of its target nucleotide sequence. In some embodiments, the antisense nucleic acid comprises or consists of a nucleotide sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of its target nucleotide sequence.

In some embodiments, an antisense nucleic acid according to the present disclosure comprises: a nucleotide sequence which is the reverse complement of its target nucleotide sequence, or a nucleotide sequence comprising 1 to 10 (e.g. one of 1, 2, 3, 4, 5, 6, 7, 8, 9 or 10) substitutions relative to the reverse complement of its target nucleotide sequence.

In some embodiments, the target nucleotide sequence for an antisense nucleic acid according to the present disclosure comprises, or consists of, 5 to 100 nucleotides, e.g. one of 10 to 80, 12 to 50, or 15 to nucleotides (e.g. 20 to 27, e.g. ˜21). In some embodiments, the target nucleotide sequence for an antisense nucleic acid according to the present disclosure comprises or consists of DNA and/or RNA. In some embodiments, the target nucleotide sequence for an antisense nucleic acid according to the present disclosure comprises or consists of RNA.

In some embodiments, the antisense nucleic acid reduces/prevents transcription of nucleic acid comprising its target nucleotide sequence. In some embodiments, the antisense nucleic acid reduces/prevents association of factors required for normal transcription (e.g. enhancers, RNA polymerase) with nucleic acid comprising its target nucleotide sequence.

In some embodiments, the antisense nucleic acid increases/potentiates degradation of nucleic acid comprising its target nucleotide sequence, e.g. through RNA interference. In some embodiments, the antisense nucleic acid reduces/prevents translation of nucleic acid comprising its target nucleotide sequence, e.g. through RNA interference or antisense degradation via Rnase H.

RNA interference is described e.g. in Agrawal et al., Microbiol. Mol. Bio. Rev. (2003) 67(4): 657-685 and Hu et al., Sig. Transduc. Tar. Ther. (2020) 5(101), both of which are hereby incorporated by reference in their entirety. Briefly, double-stranded RNA molecules are recognised by the argonaute component of the RNA-induced silencing complex (RISC). The double-stranded RNAs are separated into single strands and integrated into an active RISC, by the RISC-Loading Complex (RLC). The RISC-integrated strands bind to their target RNA through complementary base pairing, and depending on the identity of the RISC-integrated RNA and degree of complementarity to the target RNA, the RISC then either cleaves the target RNA resulting in its degradation, or otherwise blocks access of ribosomes thereby preventing its translation. RNAi based therapeutics have been approved for a number of indications (Kim, Chonnam Med J. (2020) 56(2): 87-93).

In some embodiments, the antisense nucleic acid reduces/prevents normal post-transcriptional processing (e.g. splicing and/or translation) of nucleic acid comprising its target nucleotide sequence. In some embodiments, the antisense nucleic acid reduces or alters splicing of pre-mRNA comprising its target nucleotide sequence to mature mRNA. In some embodiments, the antisense nucleic acid reduces translation of mRNA comprising its target nucleotide sequence to protein.

In some embodiments, the antisense nucleic acid reduces/prevents association of factors required for normal post-transcriptional processing (e.g. components of the spliceosome) with nucleic acid comprising its target nucleotide sequence. In such instances, the antisense nucleic may be referred to as a ‘splice-switching’ nucleic acid.

Splice-switching nucleic acids are reviewed e.g. in Haves and Hastings, Nucleic Acids Res. (2016) 44(14): 6549-6563, which is hereby incorporated by reference in its entirety. Splice-switching nucleic acids include e.g. splice-switching oligonucleotides (SSOs). They disrupt the normal splicing of target RNA transcripts by blocking the RNA:RNA base-pairing and/or protein:RNA binding interactions that occur between components of the splicing machinery and pre-mRNA. Splice-switching nucleic acids may be employed to alter the number/proportion of mature mRNA transcripts encoding ITFG1. Splice-switching nucleic acids may be designed to target a specific region of the target transcript, e.g. to effect skipping of exon(s) of interest, e.g. exons encoding domains/regions of interest. SSOs often comprise alterations to oligonucleotide sugar-phosphate backbones in order to reduce/prevent RNAse H degradation, such as e.g. phosphorothioate linkages, phosphorodiamidate linkages such as phosphorodiamidate morpholino (PMOs), and may comprise e.g. peptide nucleic acids (PNAs), locked nucleic acids (LNAs), methoxyethyl nucleotide modifications, e.g. 2′O-methyl (2′Ome) and 2′-O-methoxyethyl (MOE) ribose modifications and/or 5′-methylcytosine modifications.

In some embodiments, the antisense nucleic acid inhibits/reduces translation of nucleic acid comprising its target nucleotide sequence. In some embodiments, the antisense nucleic acid reduces/prevents association of factors required for translation (e.g. ribosomes) with nucleic acid comprising its target nucleotide sequence.

It will be appreciated that the target nucleotide sequence to which an antisense nucleic acid binds is a nucleotide sequence encoding a protein which it is desired to inhibit expression of. Accordingly, in aspects and embodiments of the present disclosure, the target nucleotide sequence for an antisense nucleic acid is a nucleotide sequence of a gene encoding ITFG1.

In some embodiments, the target nucleotide sequence is a nucleotide sequence of RNA encoded by a gene encoding ITFG1. In some embodiments, the target nucleotide sequence is a nucleotide sequence of RNA encoding ITFG1. In some embodiments, the target nucleotide sequence comprises one or more nucleotides of an exon of RNA encoding ITFG1. In some embodiments, the target nucleotide sequence is a nucleotide sequence of an exon of RNA encoding ITFG1.

In some embodiments, the target nucleotide sequence is a nucleotide sequence of SEQ ID NO:9, which is the corresponding RNA sequence of NCBI Reference Sequence: NM_030790.5, which is a cDNA sequence encoding human ITFG1 isoform. In some embodiments, the target nucleotide sequence is a nucleotide sequence of SEQ ID NO:10, which is the corresponding RNA sequence of NCBI Reference Sequence: NM_001305002.2, which is a cDNA sequence encoding human ITFG1 isoform 2.

In some embodiments, the target nucleotide sequence is a nucleotide sequence from between positions 1 and 226 (inclusive) of SEQ ID NO:9. In some embodiments, the target nucleotide sequence is a nucleotide sequence from between positions 227 and 299 (inclusive) of SEQ ID NO:9. In some embodiments, the target nucleotide sequence is a nucleotide sequence from between positions 300 and 445 (inclusive) of SEQ ID NO:9. In some embodiments, the target nucleotide sequence is a nucleotide sequence from between positions 446 and 503 (inclusive) of SEQ ID NO:9. In some embodiments, the target nucleotide sequence is a nucleotide sequence from between positions 504 and 578 (inclusive) of SEQ ID NO:9. In some embodiments, the target nucleotide sequence is a nucleotide sequence from between positions 579 and 673 (inclusive) of SEQ ID NO:9. In some embodiments, the target nucleotide sequence is a nucleotide sequence from between positions 674 and 738 (inclusive) of SEQ ID NO:9. In some embodiments, the target nucleotide sequence is a nucleotide sequence from between positions 739 and 820 (inclusive) of SEQ ID NO:9. In some embodiments, the target nucleotide sequence is a nucleotide sequence from between positions 821 and 915 (inclusive) of SEQ ID NO:9. In some embodiments, the target nucleotide sequence is a nucleotide sequence from between positions 916 and 1088 (inclusive) of SEQ ID NO:9. In some embodiments, the target nucleotide sequence is a nucleotide sequence from between positions 1089 and 1239 (inclusive) of SEQ ID NO:9. In some embodiments, the target nucleotide sequence is a nucleotide sequence from between positions 1240 and 1348 (inclusive) of SEQ ID NO:9. In some embodiments, the target nucleotide sequence is a nucleotide sequence from between positions 1349 and 1392 (inclusive) of SEQ ID NO:9. In some embodiments, the target nucleotide sequence is a nucleotide sequence from between positions 1393 and 1471 (inclusive) of SEQ ID NO:9. In some embodiments, the target nucleotide sequence is a nucleotide sequence from between positions 1472 and 1596 (inclusive) of SEQ ID NO:9. In some embodiments, the target nucleotide sequence is a nucleotide sequence from between positions 1597 and 1679 (inclusive) of SEQ ID NO:9. In some embodiments, the target nucleotide sequence is a nucleotide sequence from between positions 1680 and 1797 (inclusive) of SEQ ID NO:9. In some embodiments, the target nucleotide sequence is a nucleotide sequence from between positions 1798 and 3170 (inclusive) of SEQ ID NO:9.

In some embodiments, the target nucleotide sequence is, or comprises, the nucleotide sequence of one of SEQ ID NOs:13 to 44. In some embodiments, the target nucleotide sequence is, or comprises, the nucleotide sequence of one of SEQ ID NOs:115 to 146. In some embodiments, the target nucleotide sequence is, or comprises, the nucleotide sequence of SEQ ID NO:26 or 33. In some embodiments, the target nucleotide sequence is, or comprises, the nucleotide sequence of SEQ ID NO:128 or 135.

In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of one of SEQ ID NOs:13 to 44. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of one of SEQ ID NOs:115 to 146. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:26 or 33. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of SEQ ID NO:128 or 135.

In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to one of SEQ ID NOs:45 to 76. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to one of SEQ ID NOs:147 to 178. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:58 or 65. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:160 or 167.

In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to one of SEQ ID NOs:211 to 230. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to one of SEQ ID NOs:211 to 220. In some embodiments, the antisense nucleic acid comprises or consists of a sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to one of SEQ ID NOs:221 to 230.

In some embodiments, an inhibitory nucleic acid is selected from: an siRNA, dsiRNA, miRNA, shRNA, pri-miRNA, pre-miRNA, saRNA, snoRNA, or antisense oligonucleotide (e.g. a gapmer), or a nucleic acid encoding the same. In some embodiments, an inhibitory nucleic acid is selected from: an siRNA, dsiRNA, miRNA, shRNA. In some embodiments, an inhibitory nucleic acid is an siRNA.

In some embodiments, an inhibitory nucleic acid may comprise an antisense nucleic acid described herein, e.g. as part of a larger nucleic acid species. For example, in some embodiments, an inhibitory nucleic acid may be an siRNA, dsiRNA, miRNA, shRNA, pri-miRNA, pre-miRNA, saRNA or snoRNA comprising an antisense nucleic acid described herein.

In some embodiments, an inhibitory nucleic acid is a small interfering RNA (siRNA). As used herein, ‘siRNA’ refers to a double-stranded RNA molecule having a length between 17 to 30 (e.g. 20 to 27, e.g. ˜21) base pairs, which is capable of engaging the RNA interference (RNAi) pathway for the targeted degradation of target RNA. Double-stranded siRNA molecules may be formed as a nucleic acid complex of RNA strands having a high degree of complementarity. In some embodiments, siRNA molecules comprise symmetric 3′ overhangs, e.g. comprising one or two nucleotides (e.g. a ‘UU’ 3′ overhang). The strand of the double-stranded siRNA molecule having complementarity to a target nucleotide sequence (i.e. the antisense nucleic acid) may be referred to as the ‘guide’ strand, and the other strand may be referred to as the ‘passenger’ strand. The structure and function of siRNAs is described e.g. in Kim and Rossi, Biotechniques. 2008 April; 44(5): 613-616.

In some embodiments, the guide strand of an siRNA according to the present disclosure may comprise or consist of an antisense nucleic acid according to an embodiment of an antisense nucleic acid described herein.

In some embodiments, an inhibitory nucleic acid is a dicer small interfering RNA (dsiRNA). As used herein, ‘dsiRNA’ refers to a double-stranded RNA molecule having a length of ˜27 base pairs, which is processed by Dicer to siRNA for RNAi-mediated degradation of target RNA. DsiRNAs are described e.g. in Raja et al., Asian J Pharm Sci. (2019) 14(5): 497-510, which is hereby incorporated by reference in their entirety. DsiRNAs are optimised for Dicer processing and may have increased potency compared with 21-mer siRNAs (see e.g. Kim et al., Nat Biotechnol. (2005) 23(2):222-226), which may be related to the link between Dicer-mediated nuclease activity and RISC loading.

In some embodiments, an inhibitory nucleic acid is a micro RNA (miRNA), or a precursor thereof (e.g. a pri-miRNA or a pre-miRNA). miRNA molecules have a similar structure to siRNA molecules, but are encoded endogenously, and derived from processing of short hairpin RNA molecules. They are initially expressed as long primary transcripts (pri-miRNAs), which are processed within the nucleus into 60 to 70 nucleotide hairpins (pre-miRNAs), which are further processed in the cytoplasm into smaller species that interact with RISC and target mRNA. miRNAs comprise ‘seed sequences’ that are essential for binding to target mRNA. Seed sequences usually comprise six nucleotides and are situated at positions 2 to 7 at the miRNA 5′ end.

In some embodiments, an inhibitory nucleic acid is a short hairpin RNA (shRNA). shRNA molecules comprise sequences of nucleotides having a high degree of complementarity that associate with one another through complementary base pairing to form the stem region of the hairpin. The sequences of nucleotides having a high degree of complementarity may be linked by one or more nucleotides that form the loop region of the hairpin. shRNA molecules may be processed (e.g. via catalytic cleavage by DICER) to form siRNA or miRNA molecules. shRNA molecules may have a length of between 35 to 100 (e.g. 40 to 70) nucleotides. The stem region of the hairpin may have a length between 17 to 30 (e.g. 20 to 27, e.g. ˜21) base pairs. The stem region may comprise G-U pairings to stabilise the hairpin structure.

siRNA, dsiRNA, miRNAs and shRNAs for the targeted inhibition of gene and/or protein expression of ITFG1 may be identified/designed in accordance with principles and/or using tools well known to the skilled person. Parameters and tools for designing siRNA and shRNA molecules are described e.g. in Fakhr et al., Cancer Gene Therapy (2016) 23:73-82 (hereby incorporated by reference in its entirety). Software that may be used by the skilled person for the design of such molecules is summarised in Table 1 of Fakhr et al., Cancer Gene Therapy (2016) 23:73-82, and includes e.g. siRNA Wizard (InvivoGen). Details for making such molecules can be found in the websites of commercial vendors such as Ambion, Dharmacon, GenScript, Invitrogen and OligoEngine.

In some embodiments, an inhibitory nucleic acid is an antisense oligonucleotide (ASO). ASOs are single-stranded nucleic acid molecules comprising or consisting of an antisense nucleic acid to a target nucleotide sequence. An antisense oligonucleotide according to the present disclosure may comprise or consist of an antisense nucleic acid as described herein.

ASOs can modify expression of RNA molecules comprising their target nucleotide sequence by altering splicing, or by recruiting Rnase H to degrade RNA comprising the target nucleotide sequence. Rnase H recognises nucleic acid complex molecules formed when the ASO binds to RNA comprising its target nucleotide sequence. ASOs according to the present disclosure may comprise or consist of an antisense nucleic acid according to the present disclosure. ASOs may comprise 17 to 30 (e.g. 20 to 27, e.g. ˜21) nucleotides in length. Many ASOs are designed as chimeras, comprising a mix of bases with different chemistries, or as gapmers, comprising a central DNA portion surrounded by ‘wings’ of modified nucleotides. ASOs are described in e.g. Scoles et al, Neurol Genet. 2019 April; 5(2): e323. ASOs sometimes comprise alterations to the sugar-phosphate backbone in order to increase their stability and/or reduce/prevent RNAse H degradation, such as e.g. phosphorothicate linkages, phosphorodiamidate linkages such as phosphorodiamidate morpholino (PMOs), and may comprise e.g. peptide nucleic acids (PNAs), locked nucleic acids (LNAs), methoxyethyl nucleotide modifications, e.g. 2′O-methyl (2′Ome) and 2′-O-methoxyethyl (MOE) ribose modifications and/or 5′-methylcytosine modifications.

In some embodiments, an inhibitory nucleic acid according to the present disclosure comprises: (i) nucleic acid comprising the nucleotide sequence of one of SEQ ID NOs: 45 to 76, or a nucleotide sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to one of SEQ ID NOs:45 to 76; and (ii) nucleic acid comprising a nucleotide sequence having the reverse complement of the nucleotide sequence of (i), or having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of the nucleotide sequence of (i).

In some embodiments, an inhibitory nucleic acid according to the present disclosure comprises: (i) nucleic acid comprising the nucleotide sequence of one of SEQ ID NOs:147 to 178, or a nucleotide sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to one of SEQ ID NOs:147 to 178; and (ii) nucleic acid comprising a nucleotide sequence having the reverse complement of the nucleotide sequence of (i), or having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of the nucleotide sequence of (i).

In some embodiments, an inhibitory nucleic acid according to the present disclosure comprises: (i) nucleic acid comprising the nucleotide sequence of SEQ ID NO:58 or 65, or a nucleotide sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:58 or 65; and (ii) nucleic acid comprising a nucleotide sequence having the reverse complement of the nucleotide sequence of (i), or having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of the nucleotide sequence of (i).

In some embodiments, an inhibitory nucleic acid according to the present disclosure comprises: (i) nucleic acid comprising the nucleotide sequence of SEQ ID NO:160 or 167, or a nucleotide sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to SEQ ID NO:160 or 167; and (ii) nucleic acid comprising a nucleotide sequence having the reverse complement of the nucleotide sequence of (i), or having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of the nucleotide sequence of (i).

In some embodiments, an inhibitory nucleic acid according to the present disclosure comprises: (i) nucleic acid comprising the nucleotide sequence of one of SEQ ID NOs:211 to 230, or a nucleotide sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to one of SEQ ID NOs:211 to 230; and (ii) nucleic acid comprising a nucleotide sequence having the reverse complement of the nucleotide sequence of (i), or having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of the nucleotide sequence of (i).

In some embodiments, an inhibitory nucleic acid according to the present disclosure comprises: (i) nucleic acid comprising the nucleotide sequence of one of SEQ ID NOs:211 to 220, or a nucleotide sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to one of SEQ ID NOs:211 to 220; and (ii) nucleic acid comprising a nucleotide sequence having the reverse complement of the nucleotide sequence of (i), or having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of the nucleotide sequence of (i).

In some embodiments, an inhibitory nucleic acid according to the present disclosure comprises: (i) nucleic acid comprising the nucleotide sequence of one of SEQ ID NOs:221 to 230, or a nucleotide sequence having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to one of SEQ ID NOs:221 to 230; and (ii) nucleic acid comprising a nucleotide sequence having the reverse complement of the nucleotide sequence of (i), or having at least 75% sequence identity (e.g. one of at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater sequence identity) to the reverse complement of the nucleotide sequence of (i).

In some embodiments in accordance with the preceding seven paragraphs, the nucleotide sequence of (i) and the nucleotide sequence of (ii) may be provided on different nucleic acids (i.e. separate oligonucleotides). As such, the nucleic acid of (i) and (ii) may be different nucleic acids. In such embodiments, the inhibitory nucleic acid may comprise or consist of a nucleic acid duplex formed by complementary base pairing between the different nucleic acids comprising the nucleotide sequences of (i) and (ii).

Alternatively, in some embodiments the nucleotide sequence of (i) and the nucleotide sequence of (ii) may be provided on the same nucleic acid (i.e. a single oligonucleotide). That is, the nucleic acid of (i) and (ii) may be the same nucleic acid. In such embodiments, the nucleotide sequence of (i) and the nucleotide sequence of (ii) may be connected by one or more linker nucleotides. The inhibitory nucleic acid may comprise a nucleic acid duplex region formed by complementary base pairing between the nucleotide sequences of (i) and (ii), and the linker regions may form a single-stranded loop region.

Inhibitory nucleic acids according to the present disclosure may comprise chemically modified nucleotides, e.g. in which the phosphonate and/or ribose and/or base is/are chemically modified. Such modifications may influence the activity, specificity and/or stability of nucleic acid. One or more (e.g. one of 1,2, 3,4,5,6,7, 8,9,10,11,12,13,14,15,16,17,18,19,20,21,22,23,24, 25,26,27,28, 29,30 or all) nucleotides of an inhibitory nucleic acid may comprise such chemical modification.

The term “including the modifications thereto” as used herein may refer to any nucleotide modifications (e.g. methylation), backbone modifications (e.g. phosphorothioate) and/or targeting modifications (e.g. a monomer described herein).

Modifications contemplated in accordance with inhibitory nucleic acids according to the present disclosure include those described in Hu et al., Sig. Transduc. Tar. Ther. (2020) 5(101) (incorporated by reference hereinabove), in particular those shown in FIG. 2 of Hu et al., Sig. Transduc. Tar. Ther. (2020) 5(101). Further modifications contemplated in accordance with inhibitory nucleic acids according to the present disclosure include those described in Selvam et al., Chem Biol Drug Des. (2017) 90(5): 665-67B, which is hereby incorporated by reference in its entirety).

In some embodiments, an inhibitory nucleic acid according to the present disclosure comprises one or more nucleotides comprising a phosphonate modification. In some embodiments, the phosphonate modification(s) may be selected from: phosphorothioate (e.g. Rp isomer, Sp isomer), phosphorodithioate, methylphosphonate, methoxypropylphosphonate, 5′-(E)-vinylphosphonate, 5′-methylphosphonate, (S)-5′-C-methyl with phosphate, 5′-phosphorothioate, and peptide nucleic acid. In some embodiments, an inhibitory nucleic acid comprises one or more nucleotides comprising phosphorothioate modification.

In some embodiments, an inhibitory nucleic acid according to the present disclosure comprises one or more nucleotides comprising a ribose modification. In some embodiments, the ribose modification(s) may be selected from: 2′-O-methyl, 2′-O-methoxyethyl, 2′-fluoro, 2′-deoxy-2′-fluoro, 2′-methoxyethyl, 2′-O-alkyl, 2′-O-allyl, 2′-C-allyl, 2′-deoxy, 2′-hydroxyl, 2′-arabino-fluoro, 2′-O-benzyl, 2′-O-methyl-4-pyridine, locked nucleic acid, (S)-cEt-BNA, tricyclo-DNA, PMO, unlocked nucleic acid, hexitol nucleic acid and glycol nucleic acid. In some embodiments, an inhibitory nucleic acid comprises one or more nucleotides comprising 2′-O-methyl modification. In some embodiments, an inhibitory nucleic acid comprises one or more nucleotides comprising 2′-fluoro modification.

In some embodiments, an inhibitory nucleic acid according to the present disclosure comprises one or more nucleotides comprising a base modification. In some embodiments, the base modification(s) may be selected from: pseudouridine, 2′-thiouridine, N6′-methyladenosine, 5′-methylcytidine, 5′-fluoro-2′-deoxyuridine, N-ethylpiperidine 7′-EAA triazole-modified adenine, N-ethylpiperidine 6′-triazole-modified adenine, 6′-phenylpyrrolo-cytosine, 2′,4′-difluorotoluyl ribonucleoside and 5′-nitroindole.

In some embodiments, an inhibitory nucleic acid according to the present disclosure comprises: one or more nucleotides comprising phosphorothioate modification, one or more nucleotides comprising 2′-O-methyl modification, and one or more nucleotides comprising 2′-fluoro modification.

In some embodiments, an inhibitory nucleic acid according to the present disclosure comprises one or more modified nucleotides selected from: 2′-O-methyluridine-3′-phosphate, 2′-O-methyladenosine-3′-phosphate, 2′-O-methylguanesine-3′-phosphate, 2′-O-methylcytidine-3′-phosphate, 2′-O-methyluridine-3′-phosphorothioate, 2′-O-methyladenosine-3′-phosphorothioate, 2′-O-methylguanosine-3′-phosphorothioate, 2′-O-methylcytidine-3′-phosphorothioate, 2′-fluorouridine-3′-phosphate, 2′-fluoroadenosine-3′-phosphate, 2′-fluoroguanosine-3′-phosphate, 2′-fluorocytidine-3′-phosphate, 2′-fluorocytidine-3′-phosphorothioate, 2′-fluoroguanosine-3′-phosphorothioate, 2′-fluoroadenosine-3′-phosphorothioate, and 2′-fluorouridine-3′-phosphorothioate.

In some embodiments, an inhibitory nucleic acid according to the present disclosure comprises a nucleotide sequence comprising 3 to 10 (e.g. one of 3, 4, 5, 6, 7, 8, 9 or 10) nucleotides comprising 2′-fluoro modification. In some embodiments, an inhibitory nucleic acid according to the present disclosure comprises 4 to 15 (e.g. one of 4, 5, 6, 7, 8, 9, 10, 11, 12, 13, 14 or 15) nucleotides comprising 2′-fluoro modification. In some embodiments, an inhibitory nucleic acid according to the present disclosure comprises a nucleotide sequence comprising 2 to 6 (e.g. one of 2, 3, 4, 5 or 6) nucleotides comprising phosphorothioate modification. In some embodiments, an inhibitory nucleic acid according to the present disclosure comprises 5 to 20 (e.g. one of 2, 6, 7, 8, 9, 10, 11, 12, 13, 14, 15, 16, 17, 18, 19 or 20) nucleotides comprising 2′-O-methyl modification. In some embodiments, an inhibitory nucleic acid according to the present disclosure comprises a nucleotide sequence comprising 2 to 6 (e.g. one of 2, 3, 4, 5 or 6) nucleotides comprising 2′-O-methyl and phosphorothioate modification. In some embodiments, an inhibitory nucleic acid according to the present disclosure comprises a nucleotide sequence comprising 1 to 4 (e.g. one of 1, 2, 3 or 4) nucleotides comprising 2′-fluoro and phosphorothioate modification.

In various aspects and embodiments, the present disclosure provides an inhibitory nucleic acid comprising a pattern of modified nucleotides.

In some embodiments, an inhibitory nucleic acid according to the present disclosure comprises a (one or more) nucleotide sequence(s) having nucleotides comprising a pattern of modifications according to one of rows 1 to 29 of Table A below. In some embodiments, an inhibitory nucleic acid according to the present disclosure comprises a (one or more) nucleotide sequence(s) having nucleotides comprising a pattern of modifications according to one of rows 1 to 24 of Table A below. In some embodiments, an inhibitory nucleic acid according to the present disclosure comprises an antisense nucleic acid (e.g. a guide strand) having a (one or more) nucleotide sequence(s) having nucleotides comprising a pattern of modifications according to one of rows 1 to 24 of Table A below.

In some embodiments, an inhibitory nucleic acid according to the present disclosure comprises a (one or more) nucleotide sequence(s) having nucleotides comprising a pattern of modifications according to one of rows 25 to 29 of Table A below. In some embodiments, an inhibitory nucleic acid according to the present disclosure comprises a sense nucleic acid (e.g. a sense strand) having (one or more) a nucleotide sequence(s) having nucleotides comprising a pattern of modifications according to one of rows 25 to 29 of Table A below.

In some embodiments, an inhibitory nucleic acid according to the present disclosure comprises (i) an antisense nucleic acid (e.g. a guide strand) having a nucleotide sequence having nucleotides comprising a pattern of modifications according to one of rows 1 to 24 of Table A below; and (ii) a sense nucleic acid (e.g. a sense strand) having a nucleotide sequence having nucleotides comprising a pattern of modifications according to one of rows 25 to 29 of Table A below.

TABLE A
modification patterns.

| Single | |
|---|---|
| stranded RNA | |
| modification | |
| ID | Sequence (5′->3′) |
| ss001 | mNpsfNpsmNfNmNfNmNfNmNfNmNfNmNfNmNfNmNfNmNfNmNpsfNpsmN |
| ss002 | mNpsfNpsmNfNmNfNmNfNmNfNmNfNmNfNmNfNmNmNmNmNmNpsmNpsmN |
| ss003 | mNpsfNpsmNfNmNfNmNfNmNmNmNfNmNfNmNfNmNmNmNmNmNpsmNpsmN |
| ss004 | mNpsfNpsmNfNmNfNmNfNmNfNmNmNmNfNmNfNmNmNmNmNmNpsmNpsmN |
| ss005 | mNpsfNpsmNfNmNfNmNfNmNfNmNfNmNmNmNfNmNmNmNmNmNpsmNpsmN |
| ss006 | mNpsfNpsmNfNmNfNmNfNmNmNmNmNmNfNmNfNmNmNmNmNmNpsmNpsmN |
| ss007 | mNpsfNpsmNfNmNfNmNfNmNmNmNfNmNmNmNfNmNmNmNmNmNpsmNpsmN |
| ss008 | mNpsfNpsmNfNmNfNmNfNmNfNmNmNmNmNmNfNmNmNmNmNmNpsmNpsmN |
| ss009 | mNpsfNpsmNfNmNfNmNfNmNmNmNmNmNmNmNfNmNmNmNmNmNpsmNpsmN |
| ss010 | mNpsmNpsmNfNmNfNmNfNmNfNmNfNmNfNmNfNmNmNmNmNmNpsmNpsmN |
| ss011 | mNpsfNpsmNmNmNfNmNfNmNfNmNfNmNfNmNfNmNmNmNmNmNpsmNpsmN |
| ss012 | mNpsfNpsmNfNmNmNmNfNmNfNmNfNmNfNmNfNmNmNmNmNmNpsmNpsmN |
| ss013 | mNpsfNpsmNfNmNfNmNmNmNfNmNfNmNfNmNfNmNmNmNmNmNpsmNpsmN |
| ss014 | mNpsmNpsmNmNmNfNmNfNmNfNmNfNmNfNmNfNmNmNmNmNmNpsmNpsmN |
| ss015 | mNpsmNpsmNfNmNmNmNfNmNfNmNfNmNfNmNfNmNmNmNmNmNpsmNpsmN |
| ss016 | mNpsmNpsmNfNmNfNmNmNmNfNmNfNmNfNmNfNmNmNmNmNmNpsmNpsmN |
| ss017 | mNpsfNpsmNmNmNmNmNfNmNfNmNfNmNfNmNfNmNmNmNmNmNpsmNpsmN |
| ss018 | mNpsfNpsmNmNmNfNmNmNmNfNmNfNmNfNmNfNmNmNmNmNmNpsmNpsmN |
| ss019 | mNpsmNpsmNmNfNmNfNmNmNfNmNfNmNfNmNfNmNmNmNmNmNpsmNpsmN |
| ss020 | mNpsmNpsmNmNmNmNmNfNmNfNmNfNmNfNmNfNmNmNmNmNmNpsmNpsmN |
| ss021 | mNpsmNpsmNmNmNfNmNmNmNfNmNfNmNfNmNfNmNmNmNmNmNpsmNpsmN |
| ss022 | mNpsmNpsmNfNmNmNmNmNmNfNmNfNmNfNmNfNmNmNmNmNmNpsmNpsmN |
| ss023 | mNpsfNpsmNmNmNmNmNmNmNfNmNfNmNfNmNfNmNmNmNmNmNpsmNpsmN |
| ss024 | mNpsmNpsmNmNmNmNmNmNmNfNmNfNmNfNmNfNmNmNmNmNmNpsmNpsmN |
| ss025 | fNpsmNpsfNmNfNmNfNmNfNmNfNmNfNmNfNmNfNmNfNpsmNpsfN |
| ss026 | mNpsmNpsmNmNmNmNfNmNfNfNfNmNmNmNmNmNmNmNmNpsmNpsmN |
| ss027 | fNpsmNpsfNmNfNmNfNmNfNfNfNmNmNmNmNmNmNmNmNpsmNpsmN |
| ss028 | fNpsmNpsfNmNfNmNfNmNfNfNfNmNmNmNfNmNfNmNfNmNfN |
| ss029 | mNpsmNpsmNmNmNmNfNmNfNfNfNmNmNmNmNmNmNmNmNmNmN-[ligand |
| | (optional)] |

In the table above, mN=nucleotide comprising 2′-O-methyl modification, fN=nucleotide comprising 2′-deoxy-2′-fluoro modification, ps=phosphorothioate linkage. If not specified, there are phosphate linkages between two nucleotides. [ligand]=representing any ligand conjugation, for example which includes at least one GalNAc moiety, such as ‘GalNAc monomer 3′ps′GalNAc monomer 3′ps′GalNAc monomer 3’.

In some embodiments, an inhibitory nucleic acid according to the present disclosure comprises (i) an antisense nucleic acid (e.g. a guide strand) having a nucleotide sequence having nucleotides comprising a pattern of modifications according to one of rows 1 to 24 of Table A; and (ii) a sense nucleic acid (e.g. a sense strand) having a nucleotide sequence having nucleotides comprising a pattern of modifications according to one of rows 25 to 29 of the Table A, following the pairs of patterns in Table B below.

In some embodiments, the inhibitory nucleic acid comprises, or consists of: (i) nucleic acid (e.g. antisense nucleic acid/guide strand) comprising a nucleotide sequence having nucleotides comprising a pattern of modifications indicated in column 1 of Table B, and (ii) nucleic acid (e.g. a sense strand) comprising a nucleotide sequence having nucleotides comprising a pattern of modifications indicated in column 2 of Table B, wherein the sequences of columns 1 and 2 are selected from the same row of Table B. The modification patterns are described in Table A above.

TABLE B
combinations of modification patterns from Table A.

| siRNA | Antisense strand | |
|---|---|---|
| modification | modification ID | Sense strand modification ID |
| pattern ID | (column 1) | (column 2) |
| PAT001 | ss001 | ss026 |
| PAT002 | ss002 | ss026 |
| PAT003 | ss003 | ss026 |
| PAT004 | ss004 | ss026 |
| PAT005 | ss005 | ss026 |
| PAT006 | ss006 | ss026 |
| PAT007 | ss007 | ss026 |
| PAT008 | ss008 | ss026 |
| PAT009 | ss009 | ss026 |
| PAT010 | ss010 | ss026 |
| PAT011 | ss011 | ss026 |
| PAT012 | ss012 | ss026 |
| PAT013 | ss013 | ss026 |
| PAT014 | ss014 | ss026 |
| PAT015 | ss015 | ss026 |
| PAT016 | ss016 | ss026 |
| PAT017 | ss017 | ss026 |
| PAT018 | ss018 | ss026 |
| PAT019 | ss019 | ss026 |
| PAT020 | ss020 | ss026 |
| PAT021 | ss021 | ss026 |
| PAT022 | ss022 | ss026 |
| PAT023 | ss023 | ss026 |
| PAT024 | ss024 | ss026 |
| PAT025 | ss001 | ss025 |
| PAT026 | ss001 | ss027 |
| PAT027 | ss001 | ss028 |
| PAT028 | ss011 | ss029 |
| PAT029 | ss001 | ss029 |

In some embodiments, an inhibitory nucleic acid according to the present disclosure comprises (i) an antisense nucleic acid (e.g. a guide strand) having a nucleotide sequence having nucleotides comprising a pattern of modifications according to row 1 of Table A; and/or (ii) a sense nucleic acid (e.g. a sense strand) having a nucleotide sequence having nucleotides comprising a pattern of modifications according to row 26 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to row 2 of Table A; and/or (ii) a sense nucleic acid (e.g. a sense strand) having a nucleotide sequence having nucleotides comprising a pattern of modifications according to row 26 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to row 3 of Table A; and/or (ii) a sense nucleic acid (e.g. a sense strand) having a nucleotide sequence having nucleotides comprising a pattern of modifications according to row 26 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to row 4 of Table A; and/or (ii) a sense nucleic acid (e.g. a sense strand) having a nucleotide sequence having nucleotides comprising a pattern of modifications according to row 26 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to row 5 of Table A; and/or (ii) a sense nucleic acid (e.g. a sense strand) having a nucleotide sequence having nucleotides comprising a pattern of modifications according to row 26 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to row 6 of Table A; and/or (ii) a sense nucleic acid (e.g. a sense strand) having a nucleotide sequence having nucleotides comprising a pattern of modifications according to row 26 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to row 7 of Table A; and/or (ii) a sense nucleic acid (e.g. a sense strand) having a nucleotide sequence having nucleotides comprising a pattern of modifications according to row 26 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to row 8 of Table A; and/or (ii) a sense nucleic acid (e.g. a sense strand) having a nucleotide sequence having nucleotides comprising a pattern of modifications according to row 26 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to row 9 of Table A; and/or (ii) a sense nucleic acid (e.g. a sense strand) having a nucleotide sequence having nucleotides comprising a pattern of modifications according to row 26 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to row 10 of Table A; and/or (ii) a sense nucleic acid (e.g. a sense strand) having a nucleotide sequence having nucleotides comprising a pattern of modifications according to row 26 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to row 11 of Table A; and/or (ii) a sense nucleic acid (e.g. a sense strand) having a nucleotide sequence having nucleotides comprising a pattern of modifications according to row 26 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to row 12 of Table A; and/or (ii) a sense nucleic acid (e.g. a sense strand) having a nucleotide sequence having nucleotides comprising a pattern of modifications according to row 26 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to row 13 of Table A; and/or (ii) a sense nucleic acid (e.g. a sense strand) having a nucleotide sequence having nucleotides comprising a pattern of modifications according to row 26 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to row 14 of Table A; and/or (ii) a sense nucleic acid (e.g. a sense strand) having a nucleotide sequence having nucleotides comprising a pattern of modifications according to row 26 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to row 15 of Table A; and/or (ii) a sense nucleic acid (e.g. a sense strand) having a nucleotide sequence having nucleotides comprising a pattern of modifications according to row 26 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to row 16 of Table A; and/or (ii) a sense nucleic acid (e.g. a sense strand) having a nucleotide sequence having nucleotides comprising a pattern of modifications according to row 26 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to row 17 of Table A; and/or (ii) a sense nucleic acid (e.g. a sense strand) having a nucleotide sequence having nucleotides comprising a pattern of modifications according to row 26 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to row 18 of Table A; and/or (ii) a sense nucleic acid (e.g. a sense strand) having a nucleotide sequence having nucleotides comprising a pattern of modifications according to row 26 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to row 19 of Table A; and/or (ii) a sense nucleic acid (e.g. a sense strand) having a nucleotide sequence having nucleotides comprising a pattern of modifications according to row 26 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to row 20 of Table A; and/or (ii) a sense nucleic acid (e.g. a sense strand) having a nucleotide sequence having nucleotides comprising a pattern of modifications according to row 26 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to row 21 of Table A; and/or (ii) a sense nucleic acid (e.g. a sense strand) having a nucleotide sequence having nucleotides comprising a pattern of modifications according to row 26 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to row 22 of Table A; and/or (ii) a sense nucleic acid (e.g. a sense strand) having a nucleotide sequence having nucleotides comprising a pattern of modifications according to row 26 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to row 23 of Table A; and/or (ii) a sense nucleic acid (e.g. a sense strand) having a nucleotide sequence having nucleotides comprising a pattern of modifications according to row 26 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to row 24 of Table A; and/or (ii) a sense nucleic acid (e.g. a sense strand) having a nucleotide sequence having nucleotides comprising a pattern of modifications according to row 26 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to any one of rows 1-24 of Table A; and/or (ii) a sense nucleic acid (e.g. a sense strand) having a nucleotide sequence having nucleotides comprising a pattern of modifications according to row 25 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to any one of rows 1-24 of Table A; and/or (ii) a sense nucleic acid (e.g. a sense strand) having a nucleotide sequence having nucleotides comprising a pattern of modifications according to row 27 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to any one of rows 1-14 of Table A; and/or (ii) a sense nucleic acid (e.g. a sense strand) having a nucleotide sequence having nucleotides comprising a pattern of modifications according to row 28 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to any one of rows 1-24 of Table A; and/or (ii) a sense nucleic acid (e.g. a sense strand) having a nucleotide sequence having nucleotides comprising a pattern of modifications according to row 29 of Table A.

In some embodiments, an inhibitory nucleic acid according to the present disclosure comprises (i) an antisense nucleic acid (e.g. a guide strand) having a nucleotide sequence having nucleotides comprising a pattern of modifications according to row 11 of Table A; and/or (ii) a sense nucleic acid (e.g. a sense strand) having a nucleotide sequence having nucleotides comprising a pattern of modifications according to row 29 of Table A.

Modified nucleic acids according to the present disclosure may have advantageous properties, including one or more of increased potency, increased bioavailability, decreased toxicity, and decreased off-target effects, increased stability e.g. against nuclease degradation, improved affinities, expanded chemical functionality, and/or improved targeting e.g. to organ or tissue of interest, e.g. compared to an unmodified nucleic acid.

In embodiments wherein inhibitory nucleic acids comprise nucleotides comprising chemical modification as described herein, the nucleotide sequence is nevertheless evaluated for the purposes of sequence comparison in accordance with the present disclosure as if the equivalent unmodified nucleotide were instead present.

Nucleic acids comprising nucleotide(s) comprising a modified phosphonate group are evaluated for the purposes of nucleotide sequence comparison as if nucleotide(s) comprising a modified phosphonate group instead comprise the equivalent unmodified phosphonate group. Nucleic acids comprising nucleotide(s) comprising a modified ribose group are evaluated for the purposes of nucleotide sequence comparison as if nucleotide(s) comprising a modified ribose group instead comprise the equivalent unmodified ribose group. Nucleic acids comprising nucleotide(s) comprising a modified base group are evaluated for the purposes of nucleotide sequence comparison as if nucleotide(s) comprising a modified base group instead comprise the equivalent unmodified base group.

By way of illustration, nucleic acids comprising nucleotides comprising pseudouridine, 2-thiouridine and/or 5′-fluoro-2′-deoxyuridine are evaluated for the purposes of nucleotide sequence comparison as if nucleotides comprising uridine were instead present at their respective positions. By way of illustration, nucleic acids comprising nucleotides comprising N6′-methyladenosine, N-ethylpiperidine 7′-EAA triazole-modified adenine and/or N-ethylpiperidine 6′-triazole-modified adenine are evaluated for the purposes of nucleotide sequence comparison as if nucleotides comprising adenine were instead present at their respective positions. By way of illustration, nucleic acids comprising nucleotides comprising 5′-methylcytidine and/or 6′-phenylpyrrolo-cytosine are evaluated for the purposes of nucleotide sequence comparison as if nucleotides comprising cytosine were instead present at their respective positions.

In some embodiments, an inhibitory nucleic acid according to the present disclosure comprises nucleic acid comprising the nucleotide sequence (including the modifications thereto) shown in one of SEQ ID NOs:211 to 241 or 254 to 257.

In some embodiments, an inhibitory nucleic acid according to the present disclosure comprises nucleic acid comprising the nucleotide sequence (including the modifications thereto) shown in one of SEQ ID NOs:211 to 220. In some embodiments, an inhibitory nucleic acid according to the present disclosure comprises nucleic acid comprising the nucleotide sequence (including the modifications thereto) shown in one of SEQ ID NOs:221 to 230.

In some embodiments, an inhibitory nucleic acid according to the present disclosure comprises nucleic acid comprising the nucleotide sequence (including the modifications thereto) shown in one of SEQ ID NOs:231 to 241 or 254 to 257. In some embodiments, an inhibitory nucleic acid according to the present disclosure comprises nucleic acid comprising the nucleotide sequence (including the modifications thereto) shown in one of SEQ ID NOs:231 to 235, 241, or 254 to 257. In some embodiments, an inhibitory nucleic acid according to the present disclosure comprises nucleic acid comprising the nucleotide sequence (including the modifications thereto) shown in one of SEQ ID NOs:236 to 240. In some embodiments, an inhibitory nucleic acid according to the present disclosure comprises nucleic acid comprising the nucleotide sequence (including the modifications thereto) shown in one of SEQ ID NOs:235, 241, 255, 256 or 257.

In some embodiments, an inhibitory nucleic acid according to the present disclosure comprises: (i) nucleic acid comprising the nucleotide sequence (including the modifications thereto) shown in one of SEQ ID NOs:211 to 230; and (ii) nucleic acid comprising the nucleotide sequence (including the modifications thereto) shown in one of SEQ ID NOs:231 to 241 or 254 to 257.

In some embodiments, an inhibitory nucleic acid according to the present disclosure comprises: (i) nucleic acid comprising the nucleotide sequence (including the modifications thereto) shown in one of SEQ ID NOs:211 to 220; and (ii) nucleic acid comprising the nucleotide sequence (including the modifications thereto) shown in one of SEQ ID NOs:231 to 235, 241, or 254 to 257. In some embodiments, an inhibitory nucleic acid according to the present disclosure comprises: (i) nucleic acid comprising the nucleotide sequence (including the modifications thereto) shown in one of SEQ ID NOs:221 to 230; and (ii) nucleic acid comprising the nucleotide sequence (including the modifications thereto) shown in one of SEQ ID NOs:236 to 240.

In some embodiments, an inhibitory nucleic acid comprises, or consists of: (i) nucleic acid comprising a nucleotide sequence indicated in column A of Table I (below), and (ii) nucleic acid comprising a nucleotide sequence indicated in column B of Table I, wherein the sequences of columns A and B are selected from the same row of Table I.

In some embodiments, an inhibitory nucleic acid comprises, or consists of: (i) nucleic acid comprising a nucleotide sequence indicated in column A of Table II (below), and (ii) nucleic acid comprising a nucleotide sequence indicated in column B of Table II, wherein the sequences of columns A and B are selected from the same row of Table II.

In some embodiments, an inhibitory nucleic acid comprises, or consists of: (i) nucleic acid comprising a nucleotide sequence (including the modifications thereto) indicated in column A of Table III (below), and (ii) nucleic acid comprising a nucleotide sequence (including the modifications thereto) indicated in column B of Table III, wherein the sequences of columns A and B are selected from the same row of Table Ill.

By way of illustration, where the nucleotide sequences of (i) and (ii) are selected from row 1 of Table I, the inhibitory nucleic acid comprises, or consists of: (i) nucleic acid comprising the nucleotide sequence of SEQ ID NO:45; and (ii) nucleic acid comprising the nucleotide sequence of SEQ ID NO:77. By way of further illustration, the inhibitory nucleic acid designated ‘CG_200135’ described in Example 1 herein is formed of (i) an oligonucleotide having the nucleotide sequence of SEQ ID NO:147, and (ii) an oligonucleotide having the nucleotide sequence of SEQ ID NO:179; this embodiment corresponds to row 1 of Table II. By way of further illustration, the inhibitory nucleic acid designated ‘CG_200197’ described in Example 1 herein is formed of (i) an oligonucleotide having the nucleotide sequence (including the modifications thereto) of SEQ ID NO:211, and (ii) an oligonucleotide having the nucleotide sequence (including the modifications thereto) of SEQ ID NO:231; this embodiment corresponds to row 1 of Table III.

TABLE I

| | Column A | Column B |
|---|---|---|
| 1 | SEQ ID NO: 45 | SEQ ID NO: 77 |
| 2 | SEQ ID NO: 46 | SEQ ID NO: 78 |
| 3 | SEQ ID NO: 47 | SEQ ID NO: 79 |
| 4 | SEQ ID NO: 48 | SEQ ID NO: 80 |
| 5 | SEQ ID NO: 49 | SEQ ID NO: 81 |
| 6 | SEQ ID NO: 50 | SEQ ID NO: 82 |
| 7 | SEQ ID NO: 51 | SEQ ID NO: 83 |
| 8 | SEQ ID NO: 52 | SEQ ID NO: 84 |
| 9 | SEQ ID NO: 53 | SEQ ID NO: 85 |
| 10 | SEQ ID NO: 54 | SEQ ID NO: 86 |
| 11 | SEQ ID NO: 55 | SEQ ID NO: 87 |
| 12 | SEQ ID NO: 56 | SEQ ID NO: 88 |
| 13 | SEQ ID NO: 57 | SEQ ID NO: 89 |
| 14 | SEQ ID NO: 58 | SEQ ID NO: 90 |
| 15 | SEQ ID NO: 59 | SEQ ID NO: 91 |
| 16 | SEQ ID NO: 60 | SEQ ID NO: 92 |
| 17 | SEQ ID NO: 61 | SEQ ID NO: 93 |
| 18 | SEQ ID NO: 62 | SEQ ID NO: 94 |
| 19 | SEQ ID NO: 63 | SEQ ID NO: 95 |
| 20 | SEQ ID NO: 64 | SEQ ID NO: 96 |
| 21 | SEQ ID NO: 65 | SEQ ID NO: 97 |
| 22 | SEQ ID NO: 66 | SEQ ID NO: 98 |
| 23 | SEQ ID NO: 67 | SEQ ID NO: 99 |
| 24 | SEQ ID NO: 68 | SEQ ID NO: 100 |
| 25 | SEQ ID NO: 69 | SEQ ID NO: 101 |
| 26 | SEQ ID NO: 70 | SEQ ID NO: 102 |
| 27 | SEQ ID NO: 71 | SEQ ID NO: 103 |
| 28 | SEQ ID NO: 72 | SEQ ID NO: 104 |
| 29 | SEQ ID NO: 73 | SEQ ID NO: 105 |
| 30 | SEQ ID NO: 74 | SEQ ID NO: 106 |
| 31 | SEQ ID NO: 75 | SEQ ID NO: 107 |
| 32 | SEQ ID NO: 76 | SEQ ID NO: 108 |
| 33 | SEQ ID NO: 58 | SEQ ID NO: 109 |
| 34 | SEQ ID NO: 58 | SEQ ID NO: 110 |
| 35 | SEQ ID NO: 58 | SEQ ID NO: 111 |
| 36 | SEQ ID NO: 65 | SEQ ID NO: 112 |
| 37 | SEQ ID NO: 65 | SEQ ID NO: 113 |
| 38 | SEQ ID NO: 65 | SEQ ID NO: 114 |

TABLE II

| | Column A | Column B |
|---|---|---|
| 1 | SEQ ID NO: 147 | SEQ ID NO: 179 |
| 2 | SEQ ID NO: 148 | SEQ ID NO: 180 |
| 3 | SEQ ID NO: 149 | SEQ ID NO: 181 |
| 4 | SEQ ID NO: 150 | SEQ ID NO: 182 |
| 5 | SEQ ID NO: 151 | SEQ ID NO: 183 |
| 6 | SEQ ID NO: 152 | SEQ ID NO: 184 |
| 7 | SEQ ID NO: 153 | SEQ ID NO: 185 |
| 8 | SEQ ID NO: 154 | SEQ ID NO: 186 |
| 9 | SEQ ID NO: 155 | SEQ ID NO: 187 |
| 10 | SEQ ID NO: 156 | SEQ ID NO: 188 |
| 11 | SEQ ID NO: 157 | SEQ ID NO: 189 |
| 12 | SEQ ID NO: 158 | SEQ ID NO: 190 |
| 13 | SEQ ID NO: 159 | SEQ ID NO: 191 |
| 14 | SEQ ID NO: 160 | SEQ ID NO: 192 |
| 15 | SEQ ID NO: 161 | SEQ ID NO: 193 |
| 16 | SEQ ID NO: 162 | SEQ ID NO: 194 |
| 17 | SEQ ID NO: 163 | SEQ ID NO: 195 |
| 18 | SEQ ID NO: 164 | SEQ ID NO: 196 |
| 19 | SEQ ID NO: 165 | SEQ ID NO: 197 |
| 20 | SEQ ID NO: 166 | SEQ ID NO: 198 |
| 21 | SEQ ID NO: 167 | SEQ ID NO: 199 |
| 22 | SEQ ID NO: 168 | SEQ ID NO: 200 |
| 23 | SEQ ID NO: 169 | SEQ ID NO: 201 |
| 24 | SEQ ID NO: 170 | SEQ ID NO: 202 |
| 25 | SEQ ID NO: 171 | SEQ ID NO: 203 |
| 26 | SEQ ID NO: 172 | SEQ ID NO: 204 |
| 27 | SEQ ID NO: 173 | SEQ ID NO: 205 |
| 28 | SEQ ID NO: 174 | SEQ ID NO: 206 |
| 29 | SEQ ID NO: 175 | SEQ ID NO: 207 |
| 30 | SEQ ID NO: 176 | SEQ ID NO: 208 |
| 31 | SEQ ID NO: 177 | SEQ ID NO: 209 |
| 32 | SEQ ID NO: 178 | SEQ ID NO: 210 |

TABLE III

| | Column A | Column B |
|---|---|---|
| 1 | SEQ ID NO: 211 | SEQ ID NO: 231 |
| 2 | SEQ ID NO: 212 | SEQ ID NO: 231 |
| 3 | SEQ ID NO: 213 | SEQ ID NO: 231 |
| 4 | SEQ ID NO: 214 | SEQ ID NO: 231 |
| 5 | SEQ ID NO: 215 | SEQ ID NO: 231 |
| 6 | SEQ ID NO: 216 | SEQ ID NO: 231 |
| 7 | SEQ ID NO: 217 | SEQ ID NO: 231 |
| 8 | SEQ ID NO: 218 | SEQ ID NO: 231 |
| 9 | SEQ ID NO: 219 | SEQ ID NO: 231 |
| 10 | SEQ ID NO: 220 | SEQ ID NO: 231 |
| 11 | SEQ ID NO: 211 | SEQ ID NO: 232 |
| 12 | SEQ ID NO: 212 | SEQ ID NO: 232 |
| 13 | SEQ ID NO: 213 | SEQ ID NO: 232 |
| 14 | SEQ ID NO: 214 | SEQ ID NO: 232 |
| 15 | SEQ ID NO: 215 | SEQ ID NO: 232 |
| 16 | SEQ ID NO: 216 | SEQ ID NO: 232 |
| 17 | SEQ ID NO: 217 | SEQ ID NO: 232 |
| 18 | SEQ ID NO: 218 | SEQ ID NO: 232 |
| 19 | SEQ ID NO: 219 | SEQ ID NO: 232 |
| 20 | SEQ ID NO: 220 | SEQ ID NO: 232 |
| 21 | SEQ ID NO: 211 | SEQ ID NO: 233 |
| 22 | SEQ ID NO: 212 | SEQ ID NO: 233 |
| 23 | SEQ ID NO: 213 | SEQ ID NO: 233 |
| 24 | SEQ ID NO: 214 | SEQ ID NO: 233 |
| 25 | SEQ ID NO: 215 | SEQ ID NO: 233 |
| 26 | SEQ ID NO: 216 | SEQ ID NO: 233 |
| 27 | SEQ ID NO: 217 | SEQ ID NO: 233 |
| 28 | SEQ ID NO: 218 | SEQ ID NO: 233 |
| 29 | SEQ ID NO: 219 | SEQ ID NO: 233 |
| 30 | SEQ ID NO: 220 | SEQ ID NO: 233 |
| 31 | SEQ ID NO: 211 | SEQ ID NO: 234 |
| 32 | SEQ ID NO: 212 | SEQ ID NO: 234 |
| 33 | SEQ ID NO: 213 | SEQ ID NO: 234 |
| 34 | SEQ ID NO: 214 | SEQ ID NO: 234 |
| 35 | SEQ ID NO: 215 | SEQ ID NO: 234 |
| 36 | SEQ ID NO: 216 | SEQ ID NO: 234 |
| 37 | SEQ ID NO: 217 | SEQ ID NO: 234 |
| 38 | SEQ ID NO: 218 | SEQ ID NO: 234 |
| 39 | SEQ ID NO: 219 | SEQ ID NO: 234 |
| 40 | SEQ ID NO: 220 | SEQ ID NO: 234 |
| 41 | SEQ ID NO: 221 | SEQ ID NO: 236 |
| 42 | SEQ ID NO: 222 | SEQ ID NO: 236 |
| 43 | SEQ ID NO: 223 | SEQ ID NO: 236 |
| 44 | SEQ ID NO: 224 | SEQ ID NO: 236 |
| 45 | SEQ ID NO: 225 | SEQ ID NO: 236 |
| 46 | SEQ ID NO: 226 | SEQ ID NO: 236 |
| 47 | SEQ ID NO: 227 | SEQ ID NO: 236 |
| 48 | SEQ ID NO: 228 | SEQ ID NO: 236 |
| 49 | SEQ ID NO: 229 | SEQ ID NO: 236 |
| 50 | SEQ ID NO: 230 | SEQ ID NO: 236 |
| 51 | SEQ ID NO: 221 | SEQ ID NO: 237 |
| 52 | SEQ ID NO: 222 | SEQ ID NO: 237 |
| 53 | SEQ ID NO: 223 | SEQ ID NO: 237 |
| 54 | SEQ ID NO: 224 | SEQ ID NO: 237 |
| 55 | SEQ ID NO: 225 | SEQ ID NO: 237 |
| 56 | SEQ ID NO: 226 | SEQ ID NO: 237 |
| 57 | SEQ ID NO: 227 | SEQ ID NO: 237 |
| 58 | SEQ ID NO: 228 | SEQ ID NO: 237 |
| 59 | SEQ ID NO: 229 | SEQ ID NO: 237 |
| 60 | SEQ ID NO: 230 | SEQ ID NO: 237 |
| 61 | SEQ ID NO: 221 | SEQ ID NO: 238 |
| 62 | SEQ ID NO: 222 | SEQ ID NO: 238 |
| 63 | SEQ ID NO: 223 | SEQ ID NO: 238 |
| 64 | SEQ ID NO: 224 | SEQ ID NO: 238 |
| 65 | SEQ ID NO: 225 | SEQ ID NO: 238 |
| 66 | SEQ ID NO: 226 | SEQ ID NO: 238 |
| 67 | SEQ ID NO: 227 | SEQ ID NO: 238 |
| 68 | SEQ ID NO: 228 | SEQ ID NO: 238 |
| 69 | SEQ ID NO: 229 | SEQ ID NO: 238 |
| 70 | SEQ ID NO: 230 | SEQ ID NO: 238 |
| 71 | SEQ ID NO: 221 | SEQ ID NO: 239 |
| 72 | SEQ ID NO: 222 | SEQ ID NO: 239 |
| 73 | SEQ ID NO: 223 | SEQ ID NO: 239 |
| 74 | SEQ ID NO: 224 | SEQ ID NO: 239 |
| 75 | SEQ ID NO: 225 | SEQ ID NO: 239 |
| 76 | SEQ ID NO: 226 | SEQ ID NO: 239 |
| 77 | SEQ ID NO: 227 | SEQ ID NO: 239 |
| 78 | SEQ ID NO: 228 | SEQ ID NO: 239 |
| 79 | SEQ ID NO: 229 | SEQ ID NO: 239 |
| 80 | SEQ ID NO: 230 | SEQ ID NO: 239 |
| 81 | SEQ ID NO: 215 | SEQ ID NO: 235 |
| 82 | SEQ ID NO: 216 | SEQ ID NO: 235 |
| 83 | SEQ ID NO: 226 | SEQ ID NO: 240 |
| 84 | SEQ ID NO: 211 | SEQ ID NO: 241 |
| 85 | SEQ ID NO: 212 | SEQ ID NO: 241 |
| 86 | SEQ ID NO: 213 | SEQ ID NO: 241 |
| 87 | SEQ ID NO: 214 | SEQ ID NO: 241 |
| 88 | SEQ ID NO: 215 | SEQ ID NO: 241 |
| 89 | SEQ ID NO: 216 | SEQ ID NO: 241 |
| 90 | SEQ ID NO: 217 | SEQ ID NO: 241 |
| 91 | SEQ ID NO: 218 | SEQ ID NO: 241 |
| 92 | SEQ ID NO: 219 | SEQ ID NO: 241 |
| 93 | SEQ ID NO: 220 | SEQ ID NO: 241 |
| 94 | SEQ ID NO: 211 | SEQ ID NO: 254 |
| 95 | SEQ ID NO: 212 | SEQ ID NO: 254 |
| 96 | SEQ ID NO: 213 | SEQ ID NO: 254 |
| 97 | SEQ ID NO: 214 | SEQ ID NO: 254 |
| 98 | SEQ ID NO: 215 | SEQ ID NO: 254 |
| 99 | SEQ ID NO: 216 | SEQ ID NO: 254 |
| 100 | SEQ ID NO: 217 | SEQ ID NO: 254 |
| 101 | SEQ ID NO: 218 | SEQ ID NO: 254 |
| 102 | SEQ ID NO: 219 | SEQ ID NO: 254 |
| 103 | SEQ ID NO: 220 | SEQ ID NO: 254 |
| 104 | SEQ ID NO: 211 | SEQ ID NO: 255 |
| 105 | SEQ ID NO: 212 | SEQ ID NO: 255 |
| 106 | SEQ ID NO: 213 | SEQ ID NO: 255 |
| 107 | SEQ ID NO: 214 | SEQ ID NO: 255 |
| 108 | SEQ ID NO: 215 | SEQ ID NO: 255 |
| 109 | SEQ ID NO: 216 | SEQ ID NO: 255 |
| 110 | SEQ ID NO: 217 | SEQ ID NO: 255 |
| 111 | SEQ ID NO: 218 | SEQ ID NO: 255 |
| 112 | SEQ ID NO: 219 | SEQ ID NO: 255 |
| 113 | SEQ ID NO: 220 | SEQ ID NO: 255 |
| 114 | SEQ ID NO: 211 | SEQ ID NO: 256 |
| 115 | SEQ ID NO: 212 | SEQ ID NO: 256 |
| 116 | SEQ ID NO: 213 | SEQ ID NO: 256 |
| 117 | SEQ ID NO: 214 | SEQ ID NO: 256 |
| 118 | SEQ ID NO: 215 | SEQ ID NO: 256 |
| 119 | SEQ ID NO: 216 | SEQ ID NO: 256 |
| 120 | SEQ ID NO: 217 | SEQ ID NO: 256 |
| 121 | SEQ ID NO: 218 | SEQ ID NO: 256 |
| 122 | SEQ ID NO: 219 | SEQ ID NO: 256 |
| 123 | SEQ ID NO: 220 | SEQ ID NO: 256 |
| 124 | SEQ ID NO: 211 | SEQ ID NO: 257 |
| 125 | SEQ ID NO: 212 | SEQ ID NO: 257 |
| 126 | SEQ ID NO: 213 | SEQ ID NO: 257 |
| 127 | SEQ ID NO: 214 | SEQ ID NO: 257 |
| 128 | SEQ ID NO: 215 | SEQ ID NO: 257 |
| 129 | SEQ ID NO: 216 | SEQ ID NO: 257 |
| 130 | SEQ ID NO: 217 | SEQ ID NO: 257 |
| 131 | SEQ ID NO: 218 | SEQ ID NO: 257 |
| 132 | SEQ ID NO: 219 | SEQ ID NO: 257 |
| 133 | SEQ ID NO: 220 | SEQ ID NO: 257 |

In some embodiments, an inhibitory nucleic acid according to the present disclosure comprises: (i) nucleic acid comprising the nucleotide sequence (including the modifications thereto) of SEQ ID NQ:212; and (ii) nucleic acid comprising the nucleotide sequence (including the modifications thereto) of SEQ ID NO:231.

In some embodiments, an inhibitory nucleic acid according to the present disclosure comprises: (i) nucleic acid comprising the nucleotide sequence (including the modifications thereto) of SEQ ID NO:213; and (ii) nucleic acid comprising the nucleotide sequence (including the modifications thereto) of SEQ ID NO:231.

In some embodiments, an inhibitory nucleic acid according to the present disclosure comprises: (i) nucleic acid comprising the nucleotide sequence (including the modifications thereto) of SEQ ID NO:214; and (ii) nucleic acid comprising the nucleotide sequence (including the modifications thereto) of SEQ ID NO:231.

In some embodiments, an inhibitory nucleic acid according to the present disclosure comprises: (i) nucleic acid comprising the nucleotide sequence (including the modifications thereto) of SEQ ID NO:215; and (ii) nucleic acid comprising the nucleotide sequence (including the modifications thereto) of SEQ ID NO:231.

In some embodiments, an inhibitory nucleic acid according to the present disclosure comprises: (i) nucleic acid comprising the nucleotide sequence (including the modifications thereto) of SEQ ID NO:216; and (ii) nucleic acid comprising the nucleotide sequence (including the modifications thereto) of SEQ ID NO:231.

In some embodiments, an inhibitory nucleic acid according to the present disclosure comprises: (i) nucleic acid comprising the nucleotide sequence (including the modifications thereto) of SEQ ID NO:217; and (ii) nucleic acid comprising the nucleotide sequence (including the modifications thereto) of SEQ ID NO:231.

In some embodiments, an inhibitory nucleic acid according to the present disclosure comprises: (i) nucleic acid comprising the nucleotide sequence (including the modifications thereto) of SEQ ID NO:218; and (ii) nucleic acid comprising the nucleotide sequence (including the modifications thereto) of SEQ ID NO:231.

In some embodiments, an inhibitory nucleic acid according to the present disclosure comprises: (i) nucleic acid comprising the nucleotide sequence (including the modifications thereto) of SEQ ID NO:219; and (ii) nucleic acid comprising the nucleotide sequence (including the modifications thereto) of SEQ ID NO:231.

In some embodiments, an inhibitory nucleic acid according to the present disclosure comprises: (i) nucleic acid comprising the nucleotide sequence (including the modifications thereto) of SEQ ID NO:220; and (ii) nucleic acid comprising the nucleotide sequence (including the modifications thereto) of SEQ ID NO:231.

In some embodiments, an inhibitory nucleic acid according to the present disclosure comprises: (i) nucleic acid comprising the nucleotide sequence (including the modifications thereto) of SEQ ID NO:215; and (ii) nucleic acid comprising the nucleotide sequence (including the modifications thereto) of SEQ ID NO:232.

In some embodiments, an inhibitory nucleic acid according to the present disclosure comprises: (i) nucleic acid comprising the nucleotide sequence (including the modifications thereto) of SEQ ID NO:215; and (ii) nucleic acid comprising the nucleotide sequence (including the modifications thereto) of SEQ ID NO:233.

In some embodiments, an inhibitory nucleic acid according to the present disclosure comprises: (i) nucleic acid comprising the nucleotide sequence (including the modifications thereto) of SEQ ID NO:215; and (ii) nucleic acid comprising the nucleotide sequence (including the modifications thereto) of SEQ ID NO:234.

In some embodiments, an inhibitory nucleic acid according to the present disclosure comprises: (i) nucleic acid comprising the nucleotide sequence (including the modifications thereto) of SEQ ID NO:213; and (ii) nucleic acid comprising the nucleotide sequence (including the modifications thereto) of SEQ ID NO:232.

In some embodiments, an inhibitory nucleic acid according to the present disclosure comprises: (i) nucleic acid comprising the nucleotide sequence (including the modifications thereto) of SEQ ID NO:213; and (ii) nucleic acid comprising the nucleotide sequence (including the modifications thereto) of SEQ ID NO:233.

In some embodiments, an inhibitory nucleic acid according to the present disclosure comprises: (i) nucleic acid comprising the nucleotide sequence (including the modifications thereto) of SEQ ID NQ:213; and (ii) nucleic acid comprising the nucleotide sequence (including the modifications thereto) of SEQ ID NO:234.

In some embodiments, an inhibitory nucleic acid according to the present disclosure comprises: (i) nucleic acid comprising the nucleotide sequence (including the modifications thereto) of SEQ ID NO:216; and (ii) nucleic acid comprising the nucleotide sequence (including the modifications thereto) of SEQ ID NO:232.

In some embodiments, an inhibitory nucleic acid according to the present disclosure comprises: (i) nucleic acid comprising the nucleotide sequence (including the modifications thereto) of SEQ ID NO:216; and (ii) nucleic acid comprising the nucleotide sequence (including the modifications thereto) of SEQ ID NO:233.

In some embodiments, an inhibitory nucleic acid according to the present disclosure comprises: (i) nucleic acid comprising the nucleotide sequence (including the modifications thereto) of SEQ ID NO:216; and (ii) nucleic acid comprising the nucleotide sequence (including the modifications thereto) of SEQ ID NO:234.

In some embodiments, an inhibitory nucleic acid according to the present disclosure comprises: (i) nucleic acid comprising the nucleotide sequence (including the modifications thereto) of SEQ ID NO:218; and (ii) nucleic acid comprising the nucleotide sequence (including the modifications thereto) of SEQ ID NO:232.

In some embodiments, an inhibitory nucleic acid according to the present disclosure comprises: (i) nucleic acid comprising the nucleotide sequence (including the modifications thereto) of SEQ ID NO:218; and (ii) nucleic acid comprising the nucleotide sequence (including the modifications thereto) of SEQ ID NO:233.

In some embodiments, an inhibitory nucleic acid according to the present disclosure comprises: (i) nucleic acid comprising the nucleotide sequence (including the modifications thereto) of SEQ ID NO:218; and (ii) nucleic acid comprising the nucleotide sequence (including the modifications thereto) of SEQ ID NO:234.

In some embodiments, an inhibitory nucleic acid according to the present disclosure comprises: (i) nucleic acid comprising the nucleotide sequence (including the modifications thereto) of SEQ ID NO:211; and (ii) nucleic acid comprising the nucleotide sequence (including the modifications thereto) of SEQ ID NO:241.

In some embodiments, an inhibitory nucleic acid according to the present disclosure comprises: (i) nucleic acid comprising the nucleotide sequence (including the modifications thereto) of SEQ ID NO:212; and (ii) nucleic acid comprising the nucleotide sequence (including the modifications thereto) of SEQ ID NO:241.

In some embodiments, an inhibitory nucleic acid according to the present disclosure comprises: (i) nucleic acid comprising the nucleotide sequence (including the modifications thereto) of SEQ ID NO:213; and (ii) nucleic acid comprising the nucleotide sequence (including the modifications thereto) of SEQ ID NO:241.

In some embodiments, an inhibitory nucleic acid according to the present disclosure comprises: (i) nucleic acid comprising the nucleotide sequence (including the modifications thereto) of SEQ ID NO:214; and (ii) nucleic acid comprising the nucleotide sequence (including the modifications thereto) of SEQ ID NO:241.

In some embodiments, an inhibitory nucleic acid according to the present disclosure comprises: (i) nucleic acid comprising the nucleotide sequence (including the modifications thereto) of SEQ ID NO:215; and (ii) nucleic acid comprising the nucleotide sequence (including the modifications thereto) of SEQ ID NO:241.

In some embodiments, an inhibitory nucleic acid according to the present disclosure comprises: (i) nucleic acid comprising the nucleotide sequence (including the modifications thereto) of SEQ ID NO:216; and (ii) nucleic acid comprising the nucleotide sequence (including the modifications thereto) of SEQ ID NO:241.

In some embodiments, an inhibitory nucleic acid according to the present disclosure comprises: (i) nucleic acid comprising the nucleotide sequence (including the modifications thereto) of SEQ ID NO:217; and (ii) nucleic acid comprising the nucleotide sequence (including the modifications thereto) of SEQ ID NO:241.

In some embodiments, an inhibitory nucleic acid according to the present disclosure comprises: (i) nucleic acid comprising the nucleotide sequence (including the modifications thereto) of SEQ ID NQ:218; and (ii) nucleic acid comprising the nucleotide sequence (including the modifications thereto) of SEQ ID NO:241.

In some embodiments, an inhibitory nucleic acid according to the present disclosure comprises: (i) nucleic acid comprising the nucleotide sequence (including the modifications thereto) of SEQ ID NO:219; and (ii) nucleic acid comprising the nucleotide sequence (including the modifications thereto) of SEQ ID NO:241.

In some embodiments, an inhibitory nucleic acid according to the present disclosure comprises: (i) nucleic acid comprising the nucleotide sequence (including the modifications thereto) of SEQ ID NO:220; and (ii) nucleic acid comprising the nucleotide sequence (including the modifications thereto) of SEQ ID NO:241.

In some embodiments, an inhibitory nucleic acid according to the present disclosure comprises: (i) nucleic acid comprising the nucleotide sequence (including the modifications thereto) of SEQ ID NO:228; and (ii) nucleic acid comprising the nucleotide sequence (including the modifications thereto) of SEQ ID NO:236.

In some embodiments, an inhibitory nucleic acid according to the present disclosure comprises: (i) nucleic acid comprising the nucleotide sequence (including the modifications thereto) of SEQ ID NO:226; and (ii) nucleic acid comprising the nucleotide sequence (including the modifications thereto) of SEQ ID NO:236.

In some embodiments, an inhibitory nucleic acid according to the present disclosure comprises: (i) nucleic acid comprising the nucleotide sequence (including the modifications thereto) of SEQ ID NO:230; and (ii) nucleic acid comprising the nucleotide sequence (including the modifications thereto) of SEQ ID NO:236.

In some embodiments, an inhibitory nucleic acid according to the present disclosure comprises: (i) nucleic acid comprising the nucleotide sequence (including the modifications thereto) of SEQ ID NO:230; and (ii) nucleic acid comprising the nucleotide sequence (including the modifications thereto) of SEQ ID NO:239.

In some embodiments, an inhibitory nucleic acid according to the present disclosure comprises: (i) nucleic acid comprising the nucleotide sequence (including the modifications thereto) of SEQ ID NO:228; and (ii) nucleic acid comprising the nucleotide sequence (including the modifications thereto) of SEQ ID NO:239.

In some embodiments, an inhibitory nucleic acid according to the present disclosure comprises: (i) nucleic acid comprising the nucleotide sequence (including the modifications thereto) of SEQ ID NO:230; and (ii) nucleic acid comprising the nucleotide sequence (including the modifications thereto) of SEQ ID NO:237.

In some embodiments, an inhibitory nucleic acid according to the present disclosure comprises: (i) nucleic acid comprising the nucleotide sequence (including the modifications thereto) of SEQ ID NO:228; and (ii) nucleic acid comprising the nucleotide sequence (including the modifications thereto) of SEQ ID NO:238.

In some embodiments, an inhibitory nucleic acid according to the present disclosure comprises: (i) nucleic acid comprising the nucleotide sequence (including the modifications thereto) of SEQ ID NO:226; and (ii) nucleic acid comprising the nucleotide sequence (including the modifications thereto) of SEQ ID NO:237.

In some embodiments, an inhibitory nucleic acid according to the present disclosure comprises: (i) nucleic acid comprising the nucleotide sequence (including the modifications thereto) of SEQ ID NO:226; and (ii) nucleic acid comprising the nucleotide sequence (including the modifications thereto) of SEQ ID NO:238.

In some embodiments, an inhibitory nucleic acid according to the present disclosure comprises: (i) nucleic acid comprising the nucleotide sequence (including the modifications thereto) of SEQ ID NO:230; and (ii) nucleic acid comprising the nucleotide sequence (including the modifications thereto) of SEQ ID NO:238.

In some embodiments, an inhibitory nucleic acid according to the present disclosure comprises: (i) nucleic acid comprising the nucleotide sequence (including the modifications thereto) of SEQ ID NO:226; and (ii) nucleic acid comprising the nucleotide sequence (including the modifications thereto) of SEQ ID NO:239.

In some embodiments, an inhibitory nucleic acid according to the present disclosure comprises: (i) nucleic acid comprising the nucleotide sequence (including the modifications thereto) of SEQ ID NO:215; and (ii) nucleic acid comprising the nucleotide sequence (including the modifications thereto) of SEQ ID NO:235.

In some embodiments, an inhibitory nucleic acid according to the present disclosure comprises: (i) nucleic acid comprising the nucleotide sequence (including the modifications thereto) of SEQ ID NO:216; and (ii) nucleic acid comprising the nucleotide sequence (including the modifications thereto) of SEQ ID NO:235.

In some embodiments, an inhibitory nucleic acid according to the present disclosure comprises: (i) nucleic acid comprising the nucleotide sequence (including the modifications thereto) of SEQ ID NO:226; and (ii) nucleic acid comprising the nucleotide sequence (including the modifications thereto) of SEQ ID NO:240.

In some embodiments, an inhibitory nucleic acid according to the present disclosure comprises: (i) nucleic acid comprising the nucleotide sequence (including the modifications thereto) of SEQ ID NO:215; and (ii) nucleic acid comprising the nucleotide sequence (including the modifications thereto) of SEQ ID NO:254.

Inhibitory nucleic acids described herein may comprise a GalNAc targeting moiety, such as any GalNAc moiety or monomer as described herein. In some embodiments, an inhibitory nucleic acid according to the present disclosure comprises: (i) nucleic acid comprising the nucleotide sequence (including the modifications thereto) of SEQ ID NO:215; and/or (ii) nucleic acid comprising the nucleotide sequence (including the modifications thereto) of SEQ ID NO:235 or 234, wherein said nucleic acid further comprises a GalNAc moiety (preferably a triantennary GalNAc moiety, or at least one GalNAc monomer as described using a Formula provided below, such as ‘GalNAc monomer 1’, ‘GalNAc monomer 2’, ‘GalNAc monomer 3’, ‘GalNAc monomer 4’ or ‘GalNAc monomer 5’). In some embodiments, an inhibitory nucleic acid according to the present disclosure comprises: (i) nucleic acid comprising the nucleotide sequence (including the modifications thereto) of SEQ ID NO:215; and/or (ii) nucleic acid comprising the nucleotide sequence (including the modifications thereto) of SEQ ID NO:254, 241 or 231, wherein said nucleic acid further comprises a GalNAc moiety (preferably a triantennary GalNAc moiety, or at least one GalNAc monomer as described using a Formula provided below, such as ‘GalNAc monomer 1’, ‘GalNAc monomer 2’, ‘GalNAc monomer 3’, ‘GalNAc monomer 4’, or ‘GalNAc monomer 5’). In some embodiments an inhibitory nucleic acid comprises three GalNAc moieties, e.g. in tandem. For example, three ‘GalNAc monomer 1’, three ‘GalNAc monomer 2’, three ‘GalNAc monomer 3’, three ‘GalNAc monomer 4’, or three ‘GalNAc monomer 5’. In some embodiments an inhibitory nucleic acid comprises a combination of GalNAc moieties selected from two or more of ‘GalNAc monomer 1’, ‘GalNAc monomer 2’, ‘GalNAc monomer 3’, ‘GalNAc monomer 4’, and ‘GalNAc monomer 5’.

In some embodiments, an inhibitory nucleic acid according to the present disclosure comprises: (i) nucleic acid comprising the nucleotide sequence (including the modifications thereto) of SEQ ID NO:215; and/or (ii) nucleic acid comprising the nucleotide sequence (including the modifications thereto) of SEQ ID NO:254, 235 or 234, wherein said nucleic acid further comprises a triantennary GalNAc moiety. In some embodiments, an inhibitory nucleic acid according to the present disclosure comprises: (i) nucleic acid comprising the nucleotide sequence (including the modifications thereto) of SEQ ID NO:215; and/or (ii) nucleic acid comprising the nucleotide sequence (including the modifications thereto) of SEQ ID NO:235, wherein said nucleic acid further comprises at least one (preferably 3) ‘GalNAc monomer 3’ as described below.

In some embodiments, an inhibitory nucleic acid according to the present disclosure comprises: (i) nucleic acid comprising the nucleotide sequence (including the modifications thereto) of SEQ ID NO:215; and/or (ii) nucleic acid comprising the nucleotide sequence (including the modifications thereto) of SEQ ID NO:254, 241 or 231, wherein said nucleic acid further comprises at least one ‘GalNAc monomer 3’ as described below. In some embodiments, an inhibitory nucleic acid according to the present disclosure comprises: (i) nucleic acid comprising the nucleotide sequence (including the modifications thereto) of SEQ ID NO:215; and/or (ii) nucleic acid comprising the nucleotide sequence (including the modifications thereto) of SEQ ID NO:254, 241 or 231, wherein said nucleic acid further comprises three (or at least three) ‘GalNAc monomer 3’ as described below.

In some embodiments, an inhibitory nucleic acid according to the present disclosure comprises: (i) nucleic acid comprising the nucleotide sequence (including the modifications thereto) of SEQ ID NO:215; and/or (ii) nucleic acid comprising the nucleotide sequence (including the modifications thereto) of SEQ ID NO:254, 255 or 231, wherein said nucleic acid further comprises at least one ‘GalNAc monomer 5’ as described below. In some embodiments, an inhibitory nucleic acid according to the present disclosure comprises: (i) nucleic acid comprising the nucleotide sequence (including the modifications thereto) of SEQ ID NO:215; and/or (ii) nucleic acid comprising the nucleotide sequence (including the modifications thereto) of SEQ ID NO:254, 255 or 231, wherein said nucleic acid further comprises three (or at least three) ‘GalNAc monomer 5’ as described below.

In some embodiments, an inhibitory nucleic acid according to the present disclosure comprises: (i) nucleic acid comprising the nucleotide sequence (including the modifications thereto) of SEQ ID NO:215; and/or (ii) nucleic acid comprising the nucleotide sequence (including the modifications thereto) of SEQ ID NO:254, 256 or 231, wherein said nucleic acid further comprises at least one ‘GalNAc monomer 4’ as described below. In some embodiments, an inhibitory nucleic acid according to the present disclosure comprises: (i) nucleic acid comprising the nucleotide sequence (including the modifications thereto) of SEQ ID NO:215; and/or (ii) nucleic acid comprising the nucleotide sequence (including the modifications thereto) of SEQ ID NO:254, 256 or 231, wherein said nucleic acid further comprises three (or at least three) ‘GalNAc monomer 4’ as described below.

In some embodiments, an inhibitory nucleic acid according to the present disclosure comprises: (i) nucleic acid comprising the nucleotide sequence (including the modifications thereto) of SEQ ID NO:215; and/or (ii) nucleic acid comprising the nucleotide sequence (including the modifications thereto) of SEQ ID NO:254, 257 or 231, wherein said nucleic acid further comprises at least one ‘GalNAc monomer 1’ as described below. In some embodiments, an inhibitory nucleic acid according to the present disclosure comprises: (i) nucleic acid comprising the nucleotide sequence (including the modifications thereto) of SEQ ID NO:215; and/or (ii) nucleic acid comprising the nucleotide sequence (including the modifications thereto) of SEQ ID NO:254, 257 or 231, wherein said nucleic acid further comprises three (or at least three) ‘GalNAc monomer 1’ as described below.

In some embodiments, an inhibitory nucleic acid according to the present disclosure comprises: (i) nucleic acid comprising the nucleotide sequence (including the modifications thereto) of SEQ ID NO:215; and/or (ii) nucleic acid comprising the nucleotide sequence (including the modifications and targeting moieties thereto) of SEQ ID NO:235, 241, 255, 256 or 257.

Two or more GalNAc monomers may be linked by one or more phosphodiester and/or phosphorothioate bonds. At least two of the monomers may be linked by a phosphodiester bond. At least two of the monomers may be linked by a phosphorothioate bond. In some cases, all GalNAc monomers are linked by phosphorothioate bonds.

Inhibitory nucleic acids according to the present disclosure may be produced in accordance with techniques well known to the skilled person.

For example, inhibitory nucleic acids may be produced recombinantly by transcription of a nucleic acid sequence encoding the inhibitory nucleic acid. A nucleic acid encoding an inhibitory nucleic acid according to the present disclosure may e.g. be contained within an expression vector for expression of the inhibitory nucleic acid.

Transcription may be performed in cell-free transcription reactions using recombinant enzymes (e.g. RNA polymerase) for transcription of the inhibitory nucleic acids. Alternatively, production of an inhibitory nucleic acid according to the present disclosure may be performed in a cell comprising nucleic acid encoding the inhibitory nucleic acid, and may employ cellular enzymes (e.g. RNA polymerase) for transcription. Production of an inhibitory nucleic acid according to the present disclosure by expression within a cell may comprise transcription from a vector. Introduction of nucleic acid/vectors for the purposes of production of inhibitory nucleic acids according to the present disclosure may be performed in any of the ways known in the art (e.g. transfection, transduction, electroporation, etc.). Expression of an inhibitory nucleic acid can be regulated using a cell-specific promoter (e.g. a liver cell-specific promoter).

For example, an shRNA molecule according to the present disclosure may be produced within a cell by transcription from a vector encoding the shRNA. shRNAs may be produced within a cell by transfecting the cell with a vector encoding the shRNA sequence under control of an RNA polymerase promoter.

An siRNA molecule according to the present disclosure may be produced within a cell by transcription from a vector encoding shRNA encoding/comprising the siRNA, and subsequent processing of the shRNA molecule by cellular DICER to form the siRNA molecule.

Inhibitory nucleic acids may also be synthesised using standard solid or solution phase synthesis techniques which are well known in the art. Solid phase synthesis may use phosphoramidite chemistry. Briefly, a solid supported nucleotide may be detritylated, then coupled with a suitably activated nucleoside phosphoramidite to form a phosphite triester linkage. Capping may then occur, followed by oxidation of the phosphite triester with an oxidant, typically iodine. The cycle may then be repeated to yield a polynucleotide.

The present disclosure provides nucleic acid comprising or encoding an inhibitory nucleic acid according to the present disclosure. In some embodiments, nucleic acid comprising or encoding an inhibitory nucleic acid comprises, or consists of, DNA and/or RNA.

The present disclosure also provides a vector comprising the nucleic acid comprising or encoding an inhibitory nucleic acid according to the present disclosure.

Nucleic acids and vectors according to the present disclosure may be provided in purified or isolated form, i.e. from other nucleic acid, or naturally-occurring biological material.

The nucleotide sequence of a nucleic acid comprising or encoding an inhibitory nucleic acid according to the present disclosure may be contained in a vector, e.g. an expression vector. A ‘vector’ as used herein is a nucleic acid molecule used as a vehicle to transfer exogenous nucleic acid into a cell. The vector may be a vector for expression of the nucleic acid in the cell. Such vectors may include a promoter sequence operably linked to the nucleotide sequence encoding the sequence to be expressed. A vector may also include a termination codon and expression enhancers. Any suitable vectors, promoters, enhancers and termination codons known in the art may be used to express nucleic acid from a vector according to the present disclosure.

The term ‘operably linked’ may include the situation where a selected nucleic acid sequence and regulatory nucleic acid sequence (e.g. promoter and/or enhancer) are covalently linked in such a way as to place the expression of nucleic acid sequence under the influence or control of the regulatory sequence (thereby forming an expression cassette). Thus, a regulatory sequence is operably linked to the selected nucleic acid sequence if the regulatory sequence is capable of affecting transcription of the nucleic acid sequence.

Suitable vectors include plasmids, binary vectors, DNA vectors, mRNA vectors, viral vectors (e.g. gammaretroviral vectors (e.g. murine Leukemia virus (MLV)-derived vectors), lentiviral vectors, adenovirus vectors, adeno-associated virus vectors, vaccinia virus vectors and herpesvirus vectors), transposon-based vectors, and artificial chromosomes (e.g. yeast artificial chromosomes).

In some embodiments, the vector may be a eukaryotic vector, e.g. a vector comprising the elements necessary for expression of nucleic acid from the vector in a eukaryotic cell. In some embodiments, the vector may be a mammalian vector, e.g. comprising a cytomegalovirus (CMV) or SV40 promoter to drive expression. In some embodiments, the vector comprises a cell- or tissue-specific promoter. In some embodiments, the vector comprises a liver cell-specific promoter.

The present disclosure also provides a plurality of inhibitory nucleic acids according to the present disclosure. The present disclosure also provides nucleic acids and vectors comprising or encoding a plurality of inhibitory nucleic acids according to the present disclosure.

Individual inhibitory nucleic acids of a plurality of inhibitory nucleic acids according to the present disclosure may be identical or non-identical. Similarly, in embodiments wherein a nucleic acid/vector comprising or encoding an inhibitory nucleic acid according to the present disclosure comprises/encodes more than one inhibitory nucleic acid according to the present disclosure, the inhibitory nucleic acids comprised/encoded by the nucleic acid/vector may be identical or non-identical.

In some embodiments, nucleic acids/vectors may encode one of 1, 2, 3, 4, 5, 6, 7, 8, 9, 10, 11, 12, 13, 14, 15, 16, 17, 18, 19 or 20 inhibitory nucleic acids according to the present disclosure. In some embodiments, nucleic acids/vectors may encode multiple (e.g. 2, 3, 4, 5, 6, 7, 8, 9, 10, etc.) copies of a given inhibitory nucleic acid according to the present disclosure.

In some embodiments, a plurality of inhibitory nucleic acids according to the present disclosure may be a plurality of non-identical inhibitory nucleic acids. In some embodiments, a plurality of inhibitory nucleic acids may comprise one of 1, 2, 3, 4, 5, 6, 7, 8, 9, 10, 11, 12, 13, 14, 15, 16, 17, 18, 19 or 20 non-identical inhibitory nucleic acids. In some embodiments, nucleic acids/vectors may comprise/encode a plurality of non-identical inhibitory nucleic acids according to the present disclosure.

The following two paragraphs further define pluralities of non-identical inhibitory nucleic acids in accordance with embodiments of pluralities of inhibitory nucleic acids according to the present disclosure, and also in accordance with embodiments of nucleic acids/vectors comprising/encoding a plurality of non-identical inhibitory nucleic acids according to the present disclosure.

In some embodiments, the non-identical inhibitory nucleic acids comprise or encode non-identical antisense nucleic acids. In such embodiments, the non-identical antisense nucleic acids may each independently conform to any embodiment of an antisense nucleic acid as described hereinabove.

In some embodiments, the non-identical inhibitory nucleic acids may comprise or encode antisense nucleic acids targeting non-identical target nucleotide sequences. In such embodiments, the non-identical target nucleotide sequences may each independently conform to any embodiment of a target nucleotide sequence for an antisense nucleic acid as described hereinabove.

The present disclosure also provides a cell comprising or expressing (i) an inhibitory nucleic acid according to the present disclosure, (ii) nucleic acid comprising or encoding an inhibitory nucleic acid according to the present disclosure, and/or (iii) a vector comprising nucleic acid comprising or encoding an inhibitory nucleic acid according to the present disclosure.

The cell may be a eukaryotic cell, e.g. a mammalian cell. The mammal may be a primate (rhesus, cynomolgous, non-human primate or human) or a non-human mammal (e.g. rabbit, guinea pig, rat, mouse or other rodent (including any animal in the order Rodentia), cat, dog, pig, sheep, goat, cattle (including cows, e.g. dairy cows, or any animal in the order Bos), horse (including any animal in the order Equidae), donkey, and non-human primate). In preferred embodiments, the cell may be a human cell. In some embodiments, the cell may be a liver cell.

The present disclosure also provides a method for producing a cell comprising a nucleic acid or vector according to the present disclosure, comprising introducing a nucleic acid or vector according to the present disclosure into a cell. In some embodiments, introducing a nucleic acid or vector according to the present disclosure into a cell comprises transformation, transfection, electroporation or transduction (e.g. retroviral transduction).

The present disclosure also provides a method for producing an inhibitory nucleic acid according to the present disclosure or a nucleic acid comprising or encoding an inhibitory nucleic acid according to the present disclosure, comprising culturing a cell comprising nucleic acid comprising or encoding an inhibitory nucleic acid according to the present disclosure or a vector according to the present disclosure under conditions suitable for expression of the nucleic acid or vector by the cell. In some embodiments, the methods are performed in vitro.

The present disclosure also provides compositions comprising nucleic acids (including inhibitory nucleic acids, nucleic acids comprising/encoding an inhibitory nucleic acid, expression vectors comprising/encoding such nucleic acids) or cells according to the present disclosure.

In therapeutic and prophylactic applications, the compositions of the present disclosure are preferably formulated as a medicament or pharmaceutical composition (suitable for clinical use). Such compositions may comprise the nucleic acid or cell together with one or more other pharmaceutically-acceptable ingredients well known to those skilled in the art.

Compositions of the present disclosure may comprise one or more pharmaceutically-acceptable carriers (e.g. liposomes, micelles, microspheres, nanoparticles), diluents/excipients (e.g. starch, cellulose, a cellulose derivative, a polyol, dextrose, maltodextrin, magnesium stearate), adjuvants, fillers, buffers, preservatives (e.g. vitamin A, vitamin E, vitamin C, retinyl palmitate, selenium, cysteine, methionine, citric acid, sodium citrate, methyl paraben, propyl paraben), anti-oxidants (e.g. vitamin A, vitamin E, vitamin C, retinyl palmitate, selenium), lubricants (e.g. magnesium stearate, talc, silica, stearic acid, vegetable stearin), binders (e.g. sucrose, lactose, starch, cellulose, gelatin, polyethylene glycol (PEG), polyvinylpyrrolidone (PVP), xylitol, sorbitol, mannitol), stabilisers, solubilisers, surfactants (e.g., wetting agents), masking agents or colouring agents (e.g. titanium oxide).

The term ‘pharmaceutically-acceptable’ as used herein pertains to compounds, ingredients, materials, compositions, dosage forms, etc., which are, within the scope of sound medical judgment, suitable for use in contact with the tissues of the subject in question (e.g. a human subject) without excessive toxicity, irritation, allergic response, or other problem or complication, commensurate with a reasonable benefit/risk ratio. Each carrier, diluent, excipient, adjuvant, filler, buffer, preservative, anti-oxidant, lubricant, binder, stabiliser, solubiliser, surfactant, masking agent, colouring agent, flavouring agent or sweetening agent of a composition according to the present disclosure must also be ‘acceptable’ in the sense of being compatible with the other ingredients of the formulation. Suitable carriers, diluents, excipients, adjuvants, fillers, buffers, preservatives, anti-oxidants, lubricants, binders, stabilisers, solubilisers, surfactants, masking agents, colouring agents, flavouring agents or sweetening agents can be found in standard pharmaceutical texts, for example, Remington's ‘The Science and Practice of Pharmacy’ (Ed. A. Adejare), 23^{rd} Edition (2020), Academic Press.

Compositions according to the present disclosure may be prepared by any methods well known in the art of pharmacy. Such methods include the step of bringing into association the active compound with a carrier which constitutes one or more accessory ingredients. In general, the formulations are prepared by uniformly and intimately bringing into association the active compound with carriers (e.g., liquid carriers, finely divided solid carrier, etc.), and then shaping the product, if necessary.

The compositions may be prepared for topical, parenteral, systemic, intracavitary, intravenous, intra-arterial, intramuscular, intrathecal, intraocular, intravitreal, intraconjunctival, subretinal, suprachoroidal, subcutaneous, intradermal, intrathecal, oral, nasal or transdermal routes of administration which may include injection or infusion. Suitable formulations may comprise the selected agent in a sterile or isotonic medium. The formulation and mode of administration may be selected according to the agent to be administered, and disease to be treated/prevented.

The compositions of the present disclosure may be formulated in fluid, including gel, form. Fluid formulations may be formulated for administration by injection or infusion (e.g. via catheter) to a selected organ or region of the human or animal body. A further aspect of the present disclosure relates to a method of formulating or producing a medicament or pharmaceutical composition according to the present disclosure, the method comprising formulating a pharmaceutical composition or medicament by mixing an agent with a pharmaceutically acceptable carrier, adjuvant, excipient or diluent.

Nucleic acids (including inhibitory nucleic acids, expression vectors), cells and compositions according to the present disclosure may be modified and/or be formulated to facilitate delivery to, and/or uptake by, a cell/tissue of interest, e.g. a liver cell (hepatocyte) or hepatic tissue.

Strategies for targeted delivery of such species are reviewed e.g. in Li et al., Int. J. Mol. Sci. (2015) 16: 19518-19536 and Fu et al., Bioconjug Chem. (2014) 25(9): 1602-1608, which are hereby incorporated by reference in their entirety. In particular, nucleic acids according to the present disclosure may employ a delivery platform described in Hu et al., Sig. Transduc. Tar. Ther. (2020) 5(101) (incorporated by reference hereinabove), or Tatiparti et al. ‘siRNA Delivery Strategies: A Comprehensive Review of Recent Developments.’ Ed. Thomas Nann. Nanomaterials 7.4 (2017): 77, and Lehto T et al., Adv Drug Deliv Rev. 2016, 106(Pt A):172-182, which are hereby incorporated by reference in their entirety.

In some embodiments, articles of the present disclosure may be encapsulated in a nanoparticle or a liposome. In some embodiments, articles of the present disclosure may be (covalently or non-covalently) associated with a cell-penetrating peptide (e.g. a protein transduction domain, trojan peptide, arginine-rich peptide, vectocell peptide), a cationic polymer, a cationic lipid or a viral carrier.

Nanoparticles may be organic, e.g. micelles, liposomes, proteins, solid-lipid particles, solid polymer particles, dendrimers, and polymer therapeutics. Nanoparticles may be inorganic, e.g. such as nanotubes or metal particles, optionally with organic molecules added. In some embodiments, a nanoparticle is a nanoparticle described in Chen et al., Mol Ther Methods Clin Dev. (2016) 3:16023, which is hereby incorporated by reference in its entirety. In some embodiments, a nanoparticle is a PLGA, polypeptide, poly(O-amino ester), DOPE, β-cyclodextrin-containing polycation, linear PEI, PAMAM dendrimer, branched PEI, chitosan or polyphosophoester nanoparticle.

In some embodiments, a nucleic acid according to the present disclosure (e.g. an inhibitory nucleic acid, a nucleic acid comprising/encoding an inhibitory nucleic acid, or an expression vector) comprises modification to incorporate one or more moieties facilitating delivery to, and/or uptake by, a cell type or tissue of interest. In some embodiments, a nucleic acid according to the present disclosure is linked (e.g. chemically conjugated to) one or more moieties facilitating delivery to, and/or uptake by, a cell type or tissue of interest.

Modification to, and formulation of, nucleic acids to facilitate targeted delivery to cell types and/or tissues of interest is described e.g. in Lorenzer et al., J Control Release (2015) 203:1-15, which is hereby incorporated by reference in its entirety. The moiety facilitating delivery to, and/or uptake by, a cell type or tissue of interest may bind selectively to the target cell type/tissue of interest. The moiety may facilitate traversal of the cell membrane of the target cell type and/or of cells of the tissue of interest. The moiety may bind to a molecule expressed at the cell surface of the target cell type/tissue of interest. The moiety may facilitate internalisation of the nucleic acid by the target cell type/tissue of interest (e.g. by endocytosis).

Moieties facilitating delivery to, and/or uptake by, cell types or tissues of interest are described e.g. in Benizri et al., Bioconjug Chem. (2019) 30(2): 366-383, which is hereby incorporated by reference in its entirety. Such moieties include e.g. N-acetylgalactosamine (GalNAc), α-tocopherol, cell-penetrating peptide, nucleic acid aptamer, antibody and antigen-binding fragments/derivatives thereof, cholesterol, squalene, polyethylene glycol (PEG), fatty acid (e.g. palmitic acid) and nucleolipid moieties.

In some embodiments, the moiety may e.g. be a peptide/polypeptide (e.g. an antibody, fragment or derivative thereof, peptide aptamer or cell-penetrating peptide) or nucleic acid (e.g. a nucleic acid aptamer) which binds to the target cell type/tissue of interest, e.g. via interaction with a molecule expressed at the cell surface of the target cell type/tissue of interest.

In some embodiments, a nucleic acid according to the present disclosure comprises a moiety facilitating delivery to, and/or uptake by, a liver cell (e.g. a hepatocyte) and/or hepatic tissue. In such embodiments, the moiety may facilitate traversal of the hepatocyte cell membrane. The moiety may bind to a molecule expressed at the cell surface of hepatocytes. In some embodiments, a molecule expressed at the cell surface of hepatocytes is an asialoglycoprotein receptor, e.g. ASGR1 or ASGR2. The moiety may facilitate internalisation of a nucleic acid by hepatocytes (e.g. by endocytosis).

In some embodiments, the moiety may e.g. be a peptide/polypeptide (e.g. an antibody, fragment or derivative thereof, peptide aptamer or cell-penetrating peptide) or nucleic acid (e.g. a nucleic acid aptamer) which binds to a hepatocyte and/or hepatic tissue, e.g. via interaction with a molecule expressed at the cell surface of a hepatocyte (e.g. an asialoglycoprotein receptor, e.g. ASGR1 or ASGR2).

In some embodiments, the moiety is, or comprises, GalNAc. In some embodiments, a nucleic acid is conjugated to GalNAc. GalNAc interacts with asialoglycoprotein receptors expressed by hepatocytes. Nucleic acids conjugated to GalNAc are efficiently internalised by hepatic cells via receptor-mediated endocytosis following binding of GalNAc to ASGPR (see e.g. Nair et al., J. Am. Chem. Soc. (2014) 136(49): 16958-16961). In some embodiments, a nucleic acid is conjugated to one or more (e.g. 1, 2, 3, 4 or more) GalNAc moieties. In some embodiments, one or more GalNAc moieties may be covalently associated to the 5′ or 3′ end of one or more strands of a nucleic acid. In some embodiments, a nucleic acid is conjugated to a triantennary GalNAc carbohydrate moiety (such moieties are described e.g. in Nair et al., supra).

In some embodiments, the moiety is, or comprises, α-tocopherol (i.e. vitamin E). In some embodiments, a nucleic acid is conjugated to α-tocopherol. Nucleic acid-α-tocopherol conjugates have been employed for targeted delivery of nucleic acids to the liver (see e.g. Nishina et al., Mol Ther. (2008) 16(4):734-740). In some embodiments, a nucleic acid is conjugated to one or more (e.g. 1, 2, 3, 4 or more) α-tocopherol moieties. In some embodiments, one or more α-tocopherol moieties may be covalently associated to the 5′ or 3′ end of one or more strands of a nucleic acid.

Nucleic acids according to the present disclosure (e.g. inhibitory nucleic acids according to the present disclosure) may comprise a GalNAc monomer as described below and in PCT/EP2022/072271, which is incorporated herein by reference in its entirety. It will be appreciated that, dependent on the nature of R₁, the description below relates to both monomers suitable for oligonucleotide synthesis and GalNAc-oligonucleotide conjugates made using said GalNAc monomers, for example, GalNAc-bearing nucleic acids, e.g. siRNAs for reducing gene and/or protein expression of ITFG1.

The GalNAc monomer may be a compound of Formula 1, Formula 2, or Formula 3

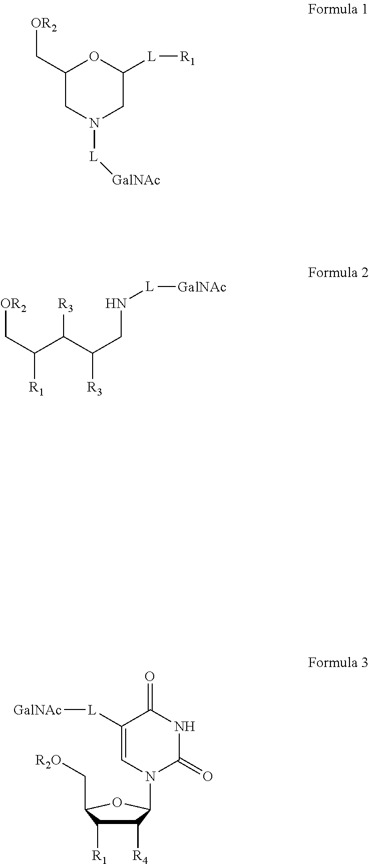

or a pharmaceutically acceptable salt thereof;
wherein
R₁ is O—PN(C₁₋₄alkyl)₂OCH₂CH₂CN, OH, a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage;
R₂ is H or a protecting group;
each R₃ is independently C₁₋₄alkyl, OC₁₋₄alkyl, C₁₋₄haloalkyl, OC₁₋₄haloalkyl or H; R₄ is H, OH, OC₁₋₄alkyl or halogen;
L is —(W—Y)ₖ—W—X—;
k is 0 to 5;
each W is independently L1 or L2;
each L1 is (CH₂)n, where n is independently 1 to 25;
each L2 is CH₂CH₂(HetCH₂CH₂)ₘ, where m independently is 1 to 24, and Het is independently a heteroatom;
X is a bond, Het, —CH₂—, —CO—, *O—CH₂—CO, *-(Het)CH₂C≡C—, or *—CH₂C≡C—, where * if present denotes the point of attachment to W; and
each Y is independently CONZ, O—CH₂—CONZ, NZCO, SO₂NZ, O—CH₂SO₂NZ or NZSO₂, where Z is H, C₁₋₄alkyl or a protecting group; and wherein GalNAc may be protected may be deprotected.

In some embodiments, X is a bond, Het, —CH₂—, —CO—, *-(Het)CH₂C≡C—, or *—CH₂C≡C—, where * if present denotes the point of attachment to W; and
each Y is independently CONZ, NZCO, SO₂NZ or NZSO₂, where Z is H, C₁₄alkyl or a protecting group In some embodiments where the compound is a compound of Formula 1, Formula 2, or Formula 3, L is a linker selected from -L1-X—, -L1-Y-L1-X—, -L1-Y-L2-X—, -L2-X—, -L2-Y-L1-X—, or -L2-Y-L2-X—;
L1 is (CH₂)n, where n is 2 to 25;
L2 is CH₂CH₂(HetCH₂CH₂)ₘ, where m is 1 to 12, and Het is a heteroatom;
X is a bond, Het, —CH₂—, —CO—, *-(Het)CH₂C≡C—, or *—CH₂C≡C—; and
Y is CONZ, NZCO, SO₂NZ or NZSO₂, where Z is H, C₁₋₄alkyl or a protecting group.

In some embodiments where the compound is a compound of Formula 1, Formula 2, or Formula 3, L is a linker selected from -L1-X—, -L1-Y-L1-X—, -L1-Y-L2-X—, -L2-X—, -L2-Y-L1-X—, or -L2-Y-L2-X—;
L1 is (CH₂)n, where n is 2 to 25;
L2 is CH₂CH₂(HetCH₂CH₂)ₘ, where m is 1 to 12, and Het is a heteroatom;
X is a bond, Het, —CH₂—, —CO—, *O—CH₂—CO, *-(Het)CH₂C≡C—, or *—CH₂C≡C—; and
Y is CONZ, O—CH₂—CONZ, NZCO, SO₂NZ, O—CH₂SO₂NZ or NZSO₂, where Z is H, C₁₄alkyl or a protecting group.

In some embodiments, the compound is a compound of Formula 1

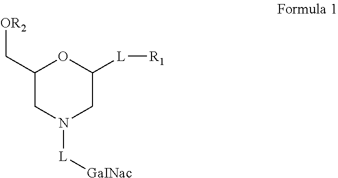

or a pharmaceutically acceptable salt thereof;
wherein
R₁ is O—PN(C₁₋₄alkyl)₂OCH₂CH₂CN, OH, a phosphoramidite linkage to an oligonucleotide or a polystyrene bead or long chain alkylamine controlled-pore glass (LCAA-CPG) which is connected through a succinic, diglycolic or hydroquinone-O,O′diacetic acid linkage;
R₂ is H or a protecting group;
each R₃ is independently C₁₋₄alkyl, OC₁₋₄alkyl, C₁₋₄haloalkyl, OC₁₋₄haloalkyl or H;
R₄ is H, OH, OC₁₋₄alkyl or halogen;
L is —(W—Y)ₖ—W—X—;
k is 0 to 5;
each W is independently L1 or L2;
each L1 is (CH₂)n, where n is independently 1 to 25; each L2 is CH₂CH₂(HetCH₂CH₂)ₘ, where m independently is 1 to 24, and Het is independently a heteroatom;
X is a bond, Het, —CH₂— —CO— or *O—CH₂—CO, for example a bond, Het, —CH₂—, or —CO—; and
each Y is independently CONZ, O—CH₂—CONZ, NZCO, SO₂NZ, O—CH₂SO₂NZ or NZSO₂, for example, CONZ, NZCO, SO₂NZ or NZSO₂, where Z is H, C₁₋₄alkyl or a protecting group; and wherein GalNAc may be protected may be deprotected.

In some embodiments, the compound is a compound of Formula 2

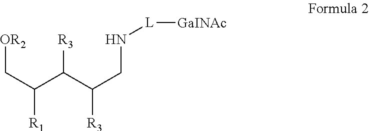

or a pharmaceutically acceptable salt thereof;
wherein
R₁ is O—PN(C₁₋₄alkyl)₂OCH₂CH₂CN, OH, a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage;
R₂ is H or a protecting group;
each R₂ is independently C₁₋₄alkyl, OC₁₋₄alkyl, C₁₋₄haloalkyl, OC₁₋₄haloalkyl or H;
R₄ is H, OH, OC₁₋₄alkyl or halogen;
L is —(W—Y)ₖ—W—X—;
k is 0 to 5;
each W is independently L1 or L2;
each L1 is (CH₂)ₙ, where n is independently 1 to 25;
each L2 is CH₂CH₂(HetCH₂CH₂)ₘ, where m independently is 1 to 24, and Het is independently a heteroatom;
X is a bond, Het, —CH₂— —CO— or *O—CH₂—CO, for example a bond, Het, —CH₂—, or —CO—; and
each Y is independently CONZ, O—CH₂—CONZ, NZCO, SO₂NZ, O—CH₂SO₂NZ or NZSO₂, for example, CONZ, NZCO, SO₂NZ or NZSO₂, where Z is H, C₁₋₄alkyl or a protecting group; and wherein GalNAc may be protected may be deprotected.

In some embodiments, the compound is a compound of Formula 3a

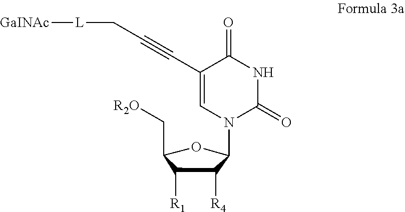

or a pharmaceutically acceptable salt thereof;
wherein
R₁ is O—PN(C₁₋₄alkyl)₂OCH₂CH₂CN, OH, a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage;
R₂ is H or a protecting group;
each R₂ is independently C₁₋₄alkyl, OC₁₋₄alkyl, C₁₋₄haloalkyl, OC₁₋₄haloalkyl or H;
R₄ is H, OH, OC₁₋₄alkyl or halogen;
L is —(W—Y)ₖ—W—X—;
k is 0 to 5;
each W is independently L1 or L2;
each L1 is (CH₂)ₙ, where n is independently 1 to 25;
each L2 is CH₂CH₂(HetCH₂CH₂)ₘ, where m independently is 1 to 24, and Het is independently a heteroatom;
X is a bond, Het, —CH₂— —CO— or *O—CH₂—CO, for example a bond, Het, —CH₂—, or —CO—; and
each Y is independently CONZ, O—CH₂—CONZ, NZCO, SO₂NZ, O—CH₂SO₂NZ or NZSO₂, for example, CONZ, NZCO, SO₂NZ or NZSO₂, where Z is H, C₁₋₄alkyl or a protecting group; and wherein GalNAc may be protected may be deprotected.

In some embodiments where the compound is a compound of Formula 1, Formula 2, or Formula 3a, L is a linker selected from -L1-X—, -L1-Y-L1-X—, -L1-Y-L2-X—, -L2-X—, -L2-Y-L1-X—, or -L2-Y-L2-X—;
L1 is (CH₂)ₙ, where n is 2 to 25;
L2 is CH₂CH₂(HetCH₂CH₂)ₘ, where m is 1 to 12, and Het is a heteroatom;
X is a bond, Het, —CH₂— —CO— or *O—CH₂—CO, for example a bond, Het, —CH₂—, or —CO—; and
each Y is independently CONZ, O—CH₂—CONZ, NZCO, SO₂NZ, O—CH₂SO₂NZ or NZSO₂, for example, CONZ, NZCO, SO₂NZ or NZSO₂, where Z is H, C₁₋₄alkyl or a protecting group.

It will be understood that references to compounds include, where appropriate, pharmaceutically acceptable salts, hydrates and solvates thereof.

### GalNAc

GalNAc (IUPAC name N-acetylgalactosamine) is an amino sugar derivative of galactose. It is used as a targeting ligand in antisense and saRNA and siRNA hepatic therapies.

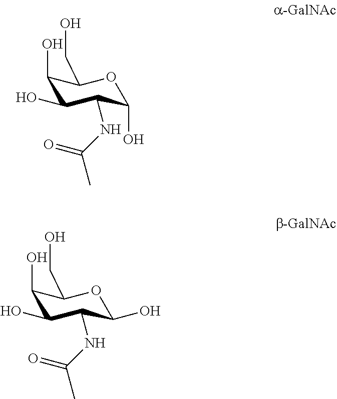

In the monomers and conjugates described herein, the GalNAc is attached via C₁. The skilled person will appreciate that, as is conventional in sugar chemistry, α- and β-stereochemistries are possible. Both stereochemistries are envisaged. On some embodiments, the a-stereochemistry is used. In some embodiments, and as exemplified herein, the β-stereochemistry is used.

During the synthesis of oligonucleotides and oligonucleotides conjugates, the GalNAc may be protected, for example with acetyl groups. Accordingly, it will be appreciated that the term GalNAc as used herein refers both to the structure having free hydroxyls as shown and the structure in which these hydroxyls are protected with suitable protecting groups.

That is, GalNAc as written in the formulae described herein may, unless otherwise specified, be a moiety as shown below, where P is hydrogen or a protecting group (for example, acetyl).

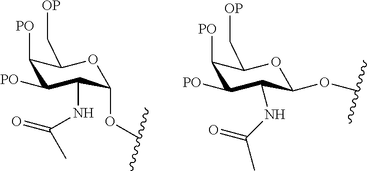

Generally, in structures in which R₁ is O—PN(C₁₋₄alkyl)₂OCH₂CH₂CN, OH, or a polystyrene bead or LCAA-CPG, GalNAc is protected, such that the GalNAc is protected for the reaction to form an oligonucleotide or an oligonucleotide conjugate. For example, the GalNAc may be fully protected with acetyl groups. That is, each P may be a protecting group, for example acetyl.

In structures in which R₁ is a phosphoramidite linkage to an oligonucleotide, the GalNAc may be protected (for example, immediately after synthesis) as described above or may be unprotected, as is normal for final products of this type. That is, each P may be hydrogen.

### The Group R₁

R₁ represents a chemical moiety for attaching the monomer to an oligonucleotide chain, a precursor group, or a point of attachment to an oligonucleotide chain. Phosphoramidite chemistry, as discussed herein, is preferred. Accordingly, in monomer units, R₁ is suitably O—PN(C₁₋₄alkyl)₂OCH₂CH₂CN, wherein said alkyl may be linear or branched, preferably iso-propyl.

For example, R₁ may be

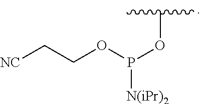

In some embodiments, R₁ is OH, said free hydroxyl group being suitable for reaction with, for example, a chlorophosphoramidite such as 2-cyanoethyl N,N-diisopropylchlorophosphoramidite.

In some embodiments, R₁ is a phosphoramidite linkage to an oligonucleotide.

In some embodiments, R₁ is a long chain alkylamine controlled-pore glass (LCAA-CPG) which is connected through a succinic, diglycolic or hydroquinone-O,O′diacetic acid linkage.

### Group R₂

Monomers disclosed herein may include hydroxyl groups which are protected during synthesis steps. It will be appreciated that the invention extends to both these free hydroxyls and protected forms thereof. Accordingly, R₂ may be a protecting group or H.

In some cases, R₂ is selected from Tr, MMTr, DMTr or TMTr protecting groups. Tr is trityl. MMTr is 4′-methoxytrityl. DMTr is dimethoxytrityl (IUPAC name bis-(4-methoxyphenyl)-phenylmethyl). TMTr is 4′, 4′, 4′-trimethoxytrityl. They are protecting groups widely used for protection of the 5′-hydroxy group in nucleosides, particularly in oligonucleotide synthesis. The skilled person will appreciate that other suitable protecting groups may be used.

Group R₃ Where present, each R₃ may be H or a suitable substituent. Suitably, R₃ is C₁₋₄alkyl, OC₁₋₄alkyl, C₁₋₄haloalkyl, OC₁₄haloalkyl or H.

In some embodiments, R₃ is a methyl group, a methoxyl group or H. In some embodiments, R₃ is H.

Group R₄ Monomers disclosed herein may include a substituent group which is commonly used in oligonucleotide monomers. This is denoted R₄. Accordingly R₄ may be a substituent or H.

Suitable R₄ substituents include hydroxyl, OC₁₋₄alkyl (preferably Ome), and halogen (preferably F).

### Linkers

The general formulae include linkers. Collectively, there are referred to herein as L. Linkers may be the same or different. For example, different linkers may be preferred for different formulae described herein, and where more than one linker is present in a formula, those linkers may be the same or different.

Suitably when the compound is a compound of Formula 1, Formula 2, or Formula 3,
L is —(W—Y)ₖ—W—X—;
wherein
k is 0 to 5;
each W is independently L1 or L2;
each L1 is (CH₂)ₙ, where n is 1 to 25;
each L2 is CH₂CH₂(HetCH₂CH₂)ₘ, where m is 1 to 24, and Het is a heteroatom;
X is a bond, a heteroatom (Het), —CH₂—, —CO—, *O—CH₂—CO, *-(Het)CH₂C≡C—, or *—CH₂C≡C—, where * if present denotes the point of attachment to W; and
each Y is independently CONZ, O—CH₂—CONZ, NZCO, SO₂NZ, O—CH₂SO₂NZ or NZSO₂, where Z is H, C₁₋₄alkyl or a protecting group.

In some embodiments,
k is 0 to 5;
each W is independently L1 or L2;
each L1 is (CH₂)ₙ, where n is 1 to 25;
each L2 is CH₂CH₂(HetCH₂CH₂)ₘ, where m is 1 to 24, and Het is a heteroatom;
X is a bond, a heteroatom (Het), —CH₂—, —CO—, *-(Het)CH₂C≡C—, or *—CH₂C≡C—, where * if present denotes the point of attachment to W; and
each Y is independently CONZ, NZCO, SO₂NZ or NZSO₂, where Z is H, C₁₄alkyl or a protecting group.

In some embodiments, k is 0 to 2. In some embodiments, k is 0 or 1.

In some embodiments, L is -L1-X—, -L1-Y-L1-X—, -L1-Y-L2-X—, -L2-X—, -L2-Y-L1-X—, -L2-Y-L2-X—, or -L1-Y-L2-Y-L1-X—.

In some embodiments, L is -L1-X—, -L1-Y-L1-X—, -L1-Y-L2-X—, -L2-X—, -L2-Y-L1-X—, or -L2-Y-L2-X—; wherein
each L1 is (CH₂)ₙ, where n is 2 to 25;
each L2 is CH₂CH₂(HetCH₂CH₂)ₘ, where m is 1 to 12, and Het is a heteroatom;
X is a bond, a heteroatom (Het), —CH₂—, —CO—, *O—CH₂—CO, *-(Het)CH₂C≡C—, or *—CH₂C≡C—; for example, a bond, a heteroatom (Het), —CH₂—, —CO—, *-(Het)CH₂C≡C—, or *—CH₂C≡C—; and
each Y is CONZ, O—CH₂—CONZ, NZCO, SO₂NZ, O—CH₂SO₂NZ or NZSO₂, where Z is H, C₁₋₄alkyl or a protecting group; for example Y is CONZ, NZCO, SO₂NZ or NZSO₂.

In some embodiments, L is -L1-X—, -L1-Y-L1-X—, -L1-Y-L2-X—, -L2-X—, -L2-Y-L1-X—, or -L2-Y-L2-X—;
each L1 is (CH₂)ₙ, where n is 2 to 10;
each L2 is CH₂CH₂(OCH₂CH₂)ₘ, where m is 1 to 5;
X is a bond, —CH₂—, —CO—, *O—CH₂—CO, —O—, *—OCH₂C≡C—, or *—CH₂C≡C—, for example, a bond, —CH₂—, —CO—, —O—, *—OCH₂C≡C—, or *—CH₂C≡C—; and
Y is CONZ or O—CH₂—CONZ, for example O—CH₂—CONZ, where Z is H, C₁₋₄alkyl or a protecting group.

In some embodiments, L is -L1-X—, -L1-Y-L1-X—, -L1-Y-L2-X—, -L2-X—, -L2-Y-L1-X—, or -L2-Y-L2-X—;
each L1 is (CH₂)ₙ, where n is 2 to 10;
each L2 is CH₂CH₂(OCH₂CH₂)ₘ where m is 1 to 5; X is a bond, —CO—, *O—CH₂—CO, —O—, *—OCH₂C≡C—, or *—CH₂C≡C—, for example, a bond, —CO—, —O—, *—OCH₂C≡C—, or *—CH₂C≡C—; and
Y is CONZ or O—CH₂—CONZ, for example O—CH₂—CONZ, where Z is H, C₁₋₄alkyl or a protecting group.

Suitably when the compound is a compound of Formula 1, Formula 2, or Formula 3a,
L is —(W—Y)ₖ—W—X—;
wherein
k is 0 to 5;
each W is independently L1 or L2;
each L1 is (CH₂)ₙ, where n is 1 to 25;
each L2 is CH₂CH₂(HetCH₂CH₂)ₘ, where m is 1 to 24, and Het is a heteroatom;
X is a bond, a heteroatom (Het), —CH₂—, *O—CH₂—CO, or —CO—, for example, a bond, a heteroatom (Het), —CH₂—, or —CO—; and
each Y is independently CONZ, O—CH₂—CONZ, NZCO, SO₂NZ, O—CH₂SO₂NZ or NZSO₂, for example, CONZ, NZCO, SO₂NZ or NZSO₂, where Z is H, C₁₋₄alkyl or a protecting group.

In some embodiments, k is 0 to 2. In some embodiments, k is 0 or 1.

In some embodiments, L is -L1-X—, -L1-Y-L1-X—, -L1-Y-L2-X—, -L2-X—, -L2-Y-L1-X—, -L2-Y-L2-X—, or -L1-Y-L2-Y-L1-X—.

In some embodiments, L is -L1-X—, -L1-Y-L1-X—, -L1-Y-L2-X—, -L2-X—, -L2-Y-L1-X—, or -L2-Y-L2-X—;
wherein
each L1 is (CH₂)ₙ, where n is 2 to 25;
each L2 is CH₂CH₂(HetCH₂CH₂)ₘ, where m is 1 to 12, and Het is a heteroatom;
X is a bond, Het, —CH₂—, *O—CH₂—CO, or —CO—, for example, a bond, Het, —CH₂—, or —CO—; and
each Y is CONZ, O—CH₂—CONZ, NZCO, SO₂NZ, O—CH₂SO₂NZ or NZSO₂, for example, CONZ, NZCO, SO₂NZ or NZSO₂, where Z is H, C₁₋₄alkyl or a protecting group.

In some embodiments, L is -L1-X—, -L1-Y-L1-X—, -L1-Y-L2-X—, -L2-X—, -L2-Y-L1-X—, or -L2-Y-L2-X—;
each L1 is (CH₂)ₙ, where n is 2 to 10;
each L2 is CH₂CH₂(OCH₂CH₂)ₘ, where m is 1 to 5;
X is a bond, —CO—,*O—CH₂—CO, or —O—, for example, a bond, —CO—, or —O—; and
Y is CONZ or O—CH₂—CONZ, for example O—CH₂—CONZ, where Z is H, C₁₋₄alkyl or a protecting group.

It will be understood that where the linker is attached to GalNAc, the arrangement is GalNAc-L-. It will be understood that where the linker is attached to R₁, the arrangement is Ri-L-. That is, the directionality of the linker should be read GalNAc-L1-X—, GalNAc-L1-Y-L1-X—, GalNAc-L1-Y-L2-X—, GalNAc-L2-X—, GalNAc-L2-Y-L1-X—, GalNAc-L2-Y-L2-X—, Ri-L1-X—, R₁-L1-Y-L1-X—, R₁-L1-Y-L2-X—, R₁-L2-X—, Ri-L2-Y-L1-X—, R₁-L2-Y-L2-X—, etc.

In some embodiments, a linker L is selected from -L1-X—, -L1-Y-L1-X—, and -L1-Y-L2-X—.

### L1

L1 is (CH₂)ₙ. That is, where present L1 is an alkylene chain. Optionally, the alkylene chain may be optionally substituted with one or more substituents selected from halogen (for example, F or CI), C₁₋₄alkyl, C₁₋₄haloalkyl, OC₁₋₄alkyl and OC₁₋₄haloalkyl.

N is 1 to 25. That is, the alkylene chain comprises 1 to 25 carbon atoms. In some embodiments, n is 2 to 25. That is, the alkylene chain comprises 2 to 25 carbon atoms. In some embodiments, n is 2 to 10. That is, the alkylene chain comprises 2 to 10 carbon atoms. In some embodiments, n is 2; that is the alkylene chain is ethylene. In some embodiments, n is 3 to 10. In some embodiments, n is 8 to 10. In some embodiments, n is 3 to 6.

In some embodiments, n is 4 to 9. In some embodiments n is 4; that is, the alkylene chain is butylene. In some embodiments n is 5; that is, the alkylene chain is pentylene. In some embodiments n is 6; that is, the alkylene chain is hexylene. In some embodiments n is 7; that is, the alkylene chain is heptylene. In some embodiments n is 8; that is, the alkylene chain is octylene. In some embodiments n is 9; that is, the alkylene chain is nonylene.

### L2

L2 is CH₂CH₂(HetCH₂CH₂)ₘ; that is, where present L2 is a (HetCH₂CH₂) chain, m is 1 to 24, and Het is a heteroatom, such that the linker comprises 1 to 24 repeating (HetCH₂CH₂) units, in addition to leading ethylene. Additional valencies on a heteroatom, where present, may be occupied by H or C₁₋₄alkyl, preferably H. Exemplary heteroatoms include O, S and N (for example, NH).

In some embodiments, m is 1 to 12. In some embodiments, Het is an oxygen atom.

Where m is 1 and Het is an oxygen atom, L2 is —CH₂CH₂OCH₂CH₂-; where m is 2 and Het is an oxygen atom, L2 is —CH₂CH₂OCH₂CH₂OCH₂CH₂—, and so on. In some embodiments, m is 1, 2, 3 or 4. In some embodiments, m is 1, 2 or 3. In some embodiments, m is 1 or 2. In some embodiments, m is 2.

### The X Group

In some embodiments, X is —CO—. In other words, the linker is attached via an acyl group. In some embodiments the linker is -L1-CO—, for example pentanoyl (—CO(CH₂)₄—) or decanoyl (—CO(CH₂)₉—).

In some embodiments, X is a bond. In other words, the linker may be attached to the leading CH₂ of L1 or L2, as appropriate. For example, the linker may be -L1-, -L1-Y-L1-, -L1-Y-L2-, -L2-, -L2-Y-L1-, or -L2-Y-L2-. In some embodiments the linker is -L1-; for example ethylene.

In some embodiments, X is a heteroatom (Het). Additional valencies on a heteroatom, where present, may be occupied by H or C₁₋₄alkyl, preferably H. Exemplary heteroatoms include O, S and N (for example, NH). In some preferred embodiments, X is —O—.

In some embodiments, X is *-(Het)CH₂C≡C—, where * is the point of attachment to W. Additional valencies on a heteroatom, where present, may be occupied by H or C₁₋₄alkyl, preferably H. Exemplary heteroatoms include O, S and N (for example, NH). In some preferred embodiments, X is *—OCH₂C≡C—.

In some embodiments, X is *—CH₂C≡C—, where * is the point of attachment to W. In other words, the linker is attached via a propargylene group. In some embodiments the linker is -L1-CH₂C≡C—.

In some embodiments, X is —CH₂—.

In some embodiments, X is *O—CH₂—CO. It will be appreciated that in units of formula L2-X, X is *O—CH₂—CO may be appropriate as the synthetic route may include oxidising the terminal alcohol of a PEG chain.

### The Y Group

Each Y is independently CONZ, O—CH₂—CONZ, NZCO, SO₂NZ, O—CH₂SO₂NZ or NZSO₂, where Z is H, C₁₋₄alkyl or a protecting group. In some embodiments, Y is CONZ or SO₂NZ, where Z is H, C₁₋₄alkyl (for example, methyl) or a protecting group.

Similarly, it will be appreciated that in units of formula L2-Y, Y is O—CH₂—CONZ or O—CH₂SO₂NZ may be suitable as the synthetic route may include oxidising the terminal alcohol of a PEG chain.

In some embodiments where more than one Y is present, each Y is the same. In some embodiments, Y is CONZ; in other words, Y provides an amide linkage which may be optionally protected. Suitable nitrogen protecting groups are known in the art and include benzyl (Bn). In some embodiments, Z is H, Bn or C₁₄alkyl.

In some embodiments, Y is CONH. That is, the linker is selected from -L1-CONH-L1-X—, -L1-CONH-L2-X—, -L2-CONH-L1-X—, and -L2-CONH-L2-X—; preferably -L1-CONH-L1-X— and -L1-CONH-L2-X—; more preferably -L1-CONH-L2-X—.

In one preferred embodiment, the linker is —(CH₂)₄—CONH—CH₂CH₂OCH₂CH₂—; that is the linker is -L1-Y-L2-X—, L1 is present and n is 4, Y is CONH, L2 is present and m is 1, and X is a bond.

In one preferred embodiment, the linker is —(CH₂)₅—CONH—(CH₂)₄—CO—; that is the linker is -L1-Y-L1-X—, where X is —CO—, Y is CONH, both L1 chains are present, and one n is 4 and the other n is 5.

In one preferred embodiment, the linker is —(CH₂)₄—CONH—CH₂CH₂—(OCH₂CH₂)₂—CONH—(CH₂)₅—; that is the linker is -L1-Y-L2-Y-L1-X—, where X is a bond, each Y is CONH, the first L1 has n is 4, the second L1 has n is 5, and m is 2.

In one preferred embodiment, the linker is —(CH₂)₄—CONH—CH₂CH₂(OCH₂CH₂)—O—(CH₂)C≡C—; that is the linker is -L1-Y-L2-X—, L1 is present and n is 4, Y is CONH, L2 is present and m is 1, and the X is *—O(CH₂)C≡C—.

### Some Preferred Linkers

In some embodiments, the linker L is -L1-Y-L1-X— (such that k is 1 and both W are L1), where X is —CO—, Y is CONH, one n is 4 and the other n is 5. That is, the linker is —(CH₂)₄—CONH—(CH₂)₅—CO—. [Linker 1]

In some embodiments, the linker L is -L1-Y-L2-X— (such that k is 1, one W is L1 and the other W is L2), where n is 4, Y is CONH, m is 4, Het is 0, and X is —CO—. That is, the linker is —(CH₂)₄—CONH—CH₂CH₂(OCH₂CH₂)₄—CO—. [Linker 2]

In some embodiments, the linker L is -L1-X— (such that k is 0, and W is L1), where n is 9, and X is —CO—. That is, the linker is —(CH₂)₉—CO—. [Linker 3]

In some embodiments, the linker L is -L1-Y-L2-X— (such that k is 1, one W is L1 and the other W is L2), where n is 4, Y is CONH, m is 1, Het is 0, and X is —O—CH₂—CO. That is, the linker is —(CH₂)₄—CONH—CH₂CH₂(OCH₂CH₂)—O—CH₂—CO—. [Linker 4]

In some embodiments, the linker L is -L2-X— (such that k is 0, and W is L2), where m is 3, Het is O, and X is —O—CH₂—CO—. That is, the linker is —CH₂CH₂(OCH₂CH₂)₃—O—CH₂—CO—. [Linker 5]

In some embodiments, the linker L is -L2-Y-L1-X— (such that k is 1, and one W is L2 and the other W is L1), where m is 1, Het is 0, Y is O—CH₂—CONH (that is, Z is H), n is 5, and X is —CO—. That is, the linker is —CH₂CH₂(OCH₂CH₂)—O—CH₂—CONH—(CH₂)₅—CO—. [Linker 6]

In some embodiments, the linker L is -(L1-Y)₂-L1-X— (such that k is 2, and all three W are L1), where one n is 4, another n is 3 and the final n is 1, one Y is CONH (that is, Z is H), and the other Y is NHCO (that is, Z is H), and X is —CH₂C≡C—. That is, the linker is —(CH₂)₄—CONH—(CH₂)₃—NHCO—CH₂—CH₂C≡C—. [Linker 7]

In some embodiments, the linker L is -(L1-Y)₂-L1-X— (such that k is 2, and all three W are L1), where one n is 4, another n is 3 and the final n is 4, one Y is CONH (that is, Z is H), and the other Y is NHCO (that is, Z is H), and X is a bond. That is, the linker is —(CH₂)₄—CONH—(CH₂)₃—NHCO—(CH₂)₄—. [Linker 7—hydrogenated].

In some embodiments, the linker L is -L2-Y-L2-X— (such that k is 1, and both W are L2), where one m is 1, and the other m is 1, both Het are O, Y is O—CH₂—CONH (that is, Z is H), and X is —OCH₂C≡C— (that is, Het is O). That is, the linker is —CH₂CH₂(OCH₂CH₂)—O—CH₂—CONH—CH₂CH₂(OCH₂CH₂)—OCH₂C≡C—. [Linker 8].

In some embodiments, the linker L is -L2-Y-L2-X— (such that k is 1, and both W are L2), where one m is 1, and the other m is 2, both Het are O, Y is O—CH₂—CONH (that is, Z is H), and X is —CH₂—. That is, the linker is —CH₂CH₂(OCH₂CH₂)—O—CH₂—CONH—CH₂CH₂(OCH₂CH₂)₂—CH₂—. [Linker 8—hydrogenated]

In some embodiments, the linker L is -L2-Y-L1-Y-L1-X— (such that k is 2, and one W is L2 and the other two W are L1), where m is 1, one Y is O—CH₂—CONH (that is, Z is H), and one Y is NHCO (that is, Z is H), one n is 3, and the other n is 1, and X is —CH₂C≡C—. That is, the linker is —CH₂CH₂(OCH₂CH₂)—O—CH₂—CONH—(CH₂)₃—NHCO—CH₂—CH₂C≡C—. [Linker 9]

In some embodiments, the linker L is -L2-Y-L1-Y-L1-X— (such that k is 2, and one W is L2 and the other two W are L1), where m is 1, one Y is O—CH₂—CONH (that is, Z is H), and one Y is NHCO (that is, Z is H), one n is 3, and the other n is 4, and X is a bond. That is, the linker is —CH₂CH₂(OCH₂CH₂)—O—CH₂—CONH—(CH₂)₃—NHCO—(CH₂)₄—. [Linker 9—hydrogenated]

In some embodiments, the linker L is -L1-Y-L2-X— (such that k is 1, and one W is L1 and the other W is L2), where n is 4, Y is CONH (that is, Z is H), m is 1, and X is —OCH₂C≡C— (that is, Het is 0). That is, the linker is —(CH₂)₄—CONH—CH₂CH₂(OCH₂CH₂)—OCH₂C≡C—. [Linker 10]

In some embodiments, the linker L is -L1-Y-L2-X— (such that k is 1, and one W is L1 and the other W is L2), where n is 4, Y is CONH (that is, Z is H), m is 2, and X is —CH₂—. That is, the linker is —(CH₂)₄—CONH—CH₂CH₂(OCH₂CH₂)₂—CH₂—. [Linker 10—hydrogenated]

### Formula 1

In some embodiments, the compound is a compound of Formula 1. Suitably, the two linkers are different and may be distinguished as L^{a} and L^{b}, as shown in Formula 1a. In some embodiments, the stereochemistry is as shown in Formula 1b.

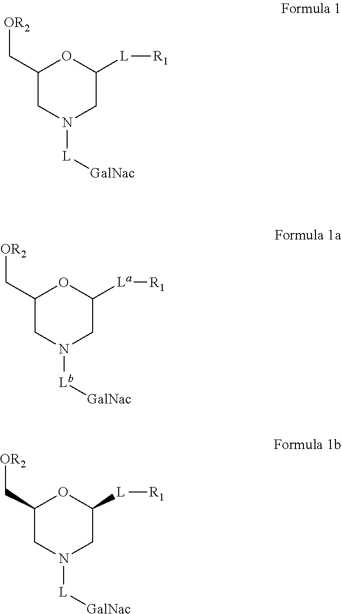

Preferably, L^{a} is a linker of formula -L1-X—, optionally wherein n is 2 and X is an oxygen atom. That is, L^{a} is -(ethylene)O—, and the substituent is R₁-(ethylene)O—.

That is, the compound may be a compound of the following formula:

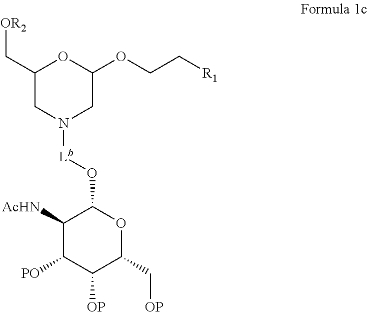

In some alternative embodiments, L^{a} is L2, optionally where m is 1 or 2, preferably 1.

Preferably, L^{b} is a linker of formula -L1-X—, optionally wherein n is 4 and X is —CO—. That is, L^{b} is -(butylene)CO—.

That is, the compound may be a compound of the following formula:

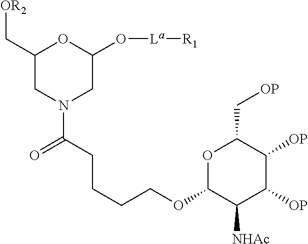

In some embodiments, the invention provides a monomer of the following formula (GalNAc Monomer 1):

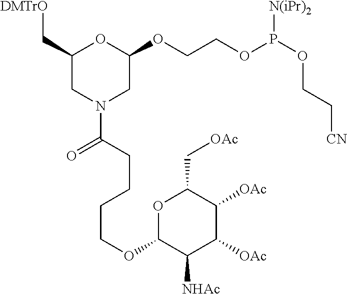

That is, R₁ is O—PN(iPr)₂OCH₂CH₂CN; R₂ is DMTr; a first linker (L^{a}) is -L1-X—, where n=2 and X is an oxygen atom; and the second linker (L^{b}) is -L1-X—, where X is —CO— and n is 4; and GalNAc is protected with acetyl groups (that is, P is Ac).

### Formula 2

In some embodiments, the compound is a compound of Formula 2.

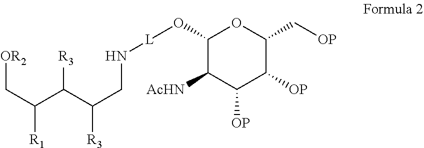

Preferably, each R₃ is independently a methyl group, a methoxyl group or H. More preferably, R₃ is H.

In some embodiments, the compound is a compound of Formula 2a or 2b:

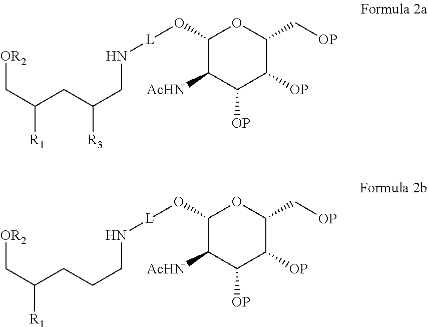

Preferably, the linker is -L1-X—, where X is —CO—. That is, the linker is attached to the amine of the core motif by an amide bond. In some embodiments, n is 8 to 10. In some embodiments n is 9; that is, the linker is decanoyl.

In some embodiments, the linker is -L1-Y-L1-X—, where X is —CO— and Y is CONZ. That is, the linker is attached to the amine of the core motif by an amide bond. In some embodiments, the L1 groups may have different values for n. In some embodiments, each n is independently 4 or 5. In some embodiments, the linker is —(CH₂)₅—Y—(CH₂)₄—X—, for example, —(CH₂)₅—CONH—(CH₂)₄—CO—.

In some embodiments, the invention provides a monomer of the following formula (GalNAc Monomer 2):

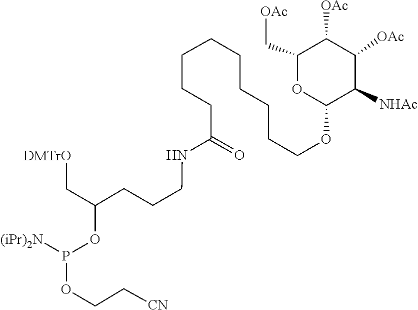

That is, R₁ is O—PN(iPr)₂OCH₂CH₂CN; R₂ is DMTr; both R₃ are H; the linker is -L1-X—, where X is —CO— and n is 9, and GalNAc is protected with acetyl groups (that is, P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage; R₂ is OH, DMTr); both R₃ are H; the linker L is -L1-Y-L1-X— (such that k is 1 and both W are L1), where X is —CO—, Y is CONH, one n is 4 and the other n is 5 (that is, the linker is —(CH₂)₄—CONH—(CH₂)₅—CO—) [Linker 1], and GalNAc is protected with acetyl groups (that is P is Ac).

In some embodiments, the invention provides a monomer of the following formula (GalNAc Monomer 4):

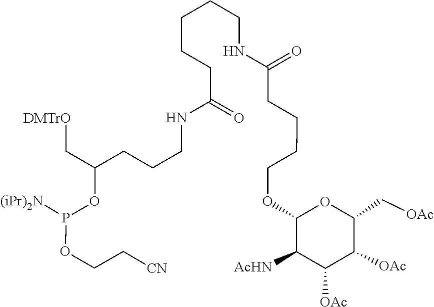

That is, R₁ is O—PN(iPr)₂OCH₂CH₂CN; R₂ is DMTr; both R₃ are H; the linker is -L1-Y-L1-X—, where X is —CO—, Y is CONH, one n is 4 and the other n is 5 (that is, the linker is —(CH₂)₅—CONH—(CH₂)₄—CO—), and GalNAc is protected with acetyl groups (that is, P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage; R₂ is OH or DMTr; both R₃ are H; the linker L is -L1-Y-L2-X— (such that k is 1, one W is L1 and the other W is L2), where n is 4, Y is CONH, m is 4, Het is 0, X is —CO— (that is, the linker is —(CH₂)₄—CONH—CH₂CH₂(OCH₂CH₂)₄—CO— [Linker 2]), and GalNAc is protected with acetyl groups (that is P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage; R₂ is OH or DMTr; both R₃ are H; the linker L is -L1-X— (such that k is 0, and W is L1), where n is 9, X is —CO— (that is, the linker is —(CH₂)₉—CO— [Linker 3]), and GalNAc is protected with acetyl groups (that is P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage;
R₂ is OH or DMTr; both R₃ are H; the linker L is -L1-Y-L2-X— (such that k is 1, one W is L1 and the other W is L2), where n is 4, Y is CONH, m is 1, Het is 0, X is —O—CH₂—CO (that is, the linker is —(CH₂)₄—CONH—CH₂CH₂(OCH₂CH₂)—O—CH₂—CO— [Linker 4]), and GalNAc is protected with acetyl groups (that is P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage; R₂ is OH or DMTr; both Ra are H; the linker L is -L2-X— (such that k is 0, and W is L2), where m is 3, Het is 0, X is O—CH₂—CO (that is, the linker is —CH₂CH₂(OCH₂CH₂)₃—O—CH₂—CO— [Linker 5]), and GalNAc is protected with acetyl groups (that is P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage; R₂ is OH or DMTr; both R₃ are H; the linker L is -L2-Y-L1-X— (such that k is 1, and one W is L2 and the other W is L1), where m is 1, Het is O, Y is O—CH₂—CONH (that is, Z is H), n is 5, X is —CO— (that is, the linker is —CH₂CH₂(OCH₂CH₂)—O—CH₂—CONH—(CH₂)e-CO— [Linker 6]), and GalNAc is protected with acetyl groups (that is P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage; R₂ is OH or DMTr; both R₃ are H; the linker L is -(L1-Y)₂-L1-X— (such that k is 2, and all three W is L1), where one n is 4, another n is 3 and the final n is 1, one Y is CONH (that is, Z is H), and the other Y is NHCO (that is, Z is H), and X is —CH₂C≡C— (that is, the linker is —(CH₂)₄—CONH—(CH₂)₃—NHCO—CH₂—CH₂C≡C— [Linker 7]), and GalNAc is protected with acetyl groups (that is P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage; R₂ is OH or DMTr; both Ra are H; the linker L is -(L1-Y)₂-L1-X— (such that k is 2, and all three W are L1), where one n is 4, another n is 3 and the final n is 4, one Y is CONH (that is, Z is H), and the other Y is NHCO (that is, Z is H), and X is a bond (that is, the linker is —(CH₂)₄—CONH—(CH₂)₃—NHCO—(CH₂)₄— [Linker 7—hydrogenated]), and GalNAc is protected with acetyl groups (that is P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage; R₂ is OH or DMTr; both R₃ are H; the linker L is -L2-Y-L2-X— (such that k is 1, and both W are L2), where one m is 1, and the other m is 1, both Het are O, Y is O—CH₂—CONH (that is, Z is H), and X is —OCH₂C≡C— (that is, Het is O) (that is, the linker is —CH₂CH₂(OCH₂CH₂)—O—CH₂—CONH—CH₂CH₂(OCH₂CH₂)—OCH₂C≡C—[Linker 8]), and GalNAc is protected with acetyl groups (that is P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage; R₂ is OH or DMTr; both R₃ are H; the linker L is -L2-Y-L2-X— (such that k is 1, and both W are L2), where one m is 1, and the other m is 2, both Het are O, Y is O—CH₂—CONH (that is, Z is H), and X is —CH₂— (that is, the linker is —CH₂CH₂(OCH₂CH₂)—O—CH₂—CONH—CH₂CH₂(OCH₂CH₂)₂—CH₂— [Linker 8—hydrogenated), and GalNAc is protected with acetyl groups (that is P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage; R₂ is OH or DMTr; both R₃ are H; the linker L is -L2-Y-L1-Y-L1-X— (such that k is 2, and one W is L2 and the other two W are L1), where m is 1, one Y is O—CH₂—CONH (that is, Z is H), and one Y is NHCO (that is, Z is H), one n is 3, and the other n is 1, and X is —CH₂C≡C— (that is, the linker is —CH₂CH₂(OCH₂CH₂)—O—CH₂—CONH—(CH₂)₃—NHCO—CH₂—CH₂C≡C— [Linker 9]), and GalNAc is protected with acetyl groups (that is P is Ac).In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage;
R₂ is OH or DMTr; both Ra are H; the linker L is -L2-Y-L1-Y-L1-X— (such that k is 2, and one W is L2 and the other two W are L1), where m is 1, one Y is O—CH₂—CONH (that is, Z is H), and one Y is NHCO (that is, Z is H), one n is 3, and the other n is 4, and X is a bond (that is, the linker is —CH₂CH₂(OCH₂CH₂)—O—CH₂—CONH—(CH₂)₃—NHCO—(CH₂)₄— [Linker 9—hydrogenated), and GalNAc is protected with acetyl groups (that is P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage;
R₂ is OH or DMTr; both R₃ are H; the linker L is -L1-Y-L2-X— (such that k is 1, and one W is L1 and the other W is L2), where n is 4, Y is CONH (that is, Z is H), m is 1, and X is —OCH₂C≡C— (that is, Het is O) (that is, the linker is —(CH₂)₄—CONH—CH₂CH₂(OCH₂CH₂)—OCH₂C≡C— [Linker 10]), and GalNAc is protected with acetyl groups (that is P is Ac).

In some embodiments, the invention provides a compound of Formula 2 in which R₁ is a phosphoramidite linkage to an oligonucleotide, or a polystyrene bead or long chain alkylamine controlled-pore glass (LCAA-CPG) which is connected through a succinic, diglycolic or hydroquinone-O,O′diacetic acid linkage;
R₂ is OH or DMTr; both Ra are H; the linker L is -L1-Y-L2-X— (such that k is 1, and one W is L1 and the other W is L2), where n is 4, Y is CONH (that is, Z is H), m is 2, and X is —CH₂— (that is, the linker is —(CH₂)₄—CONH—CH₂CH₂(OCH₂CH₂)₂—CH₂—), and GalNAc is protected with acetyl groups (that is P is Ac).

### Formula 3

In some embodiments, the compound is a compound of Formula 3.

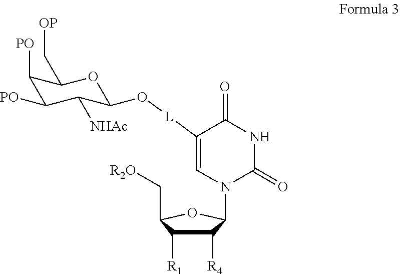

Preferably, R₄ is H, OH, OC₁₋₄alkyl or halogen. More preferably, R₄ is H.

Preferably, X is *—OCH₂C≡C—, where * denotes the point of attachment to W. In some embodiments, the linker is -L1-Y-L2-X—, such as -L1-Y-L2—OCH₂C≡C— (that is, X is *—OCH₂C≡C—). In some embodiments, Y is CONH. In some embodiments, n is 4, Y is CONH, and m is 1.

In some embodiments, the compound is a compound of Formula 3a

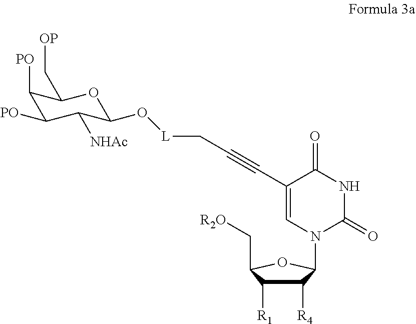

Preferably, X is an oxygen atom. In some embodiments, the linker is -L1-Y-L2-X—, such as -L1-Y-L2-O-(that is, X is an oxygen atom). In some embodiments, Y is CONH. In some embodiments, n is 4, Y is CONH, and m is 1.

In some embodiments, the linker is —(CH₂)₄—CONH—(CH₂)₅—CO— [Linker 1]

In some embodiments, the linker is —(CH₂)₄—CONH—CH₂CH₂(OCH₂CH₂)₄—CO— [Linker 2]

In some embodiments, the linker is —(CH₂)₉—CO— [Linker 3]

In some embodiments, the linker is —(CH₂)₄—CONH—CH₂CH₂(OCH₂CH₂)—O—CH₂—CO— [Linker 4]

In some embodiments, the linker is —CH₂CH₂(OCH₂CH₂)₃—O—CH₂—CO— [Linker 5]

In some embodiments, the linker is —CH₂CH₂(OCH₂CH₂)—O—CH₂—CONH—(CH₂)₅—CO— [Linker 6]

In some embodiments, the linker is —(CH₂)₄—CONH—(CH₂)₃—NHCO—CH₂—CH₂C≡C— [Linker 7]

In some embodiments, the linker is —(CH₂)₄—CONH—(CH₂)₃—NHCO—(CH₂)₄— [Linker 7—hydrogenated]

In some embodiments, the linker is —CH₂CH₂(OCH₂CH₂)—O—CH₂—CONH—CH₂CH₂(OCH₂CH₂)—OCH₂C≡C— [Linker 8]

In some embodiments, the linker is —CH₂CH₂(OCH₂CH₂)—O—CH₂—CONH—CH₂CH₂(OCH₂CH₂)₂—CH₂— [Linker 6—hydrogenated]

In some embodiments, the linker is —CH₂CH₂(OCH₂CH₂)—O—CH₂—CONH—(CH₂)₃—NHCO—CH₂—CH₂C≡C— [Linker 9]

In some embodiments, the linker is —CH₂CH₂(OCH₂CH₂)—O—CH₂—CONH—(CH₂)₃—NHCO—(CH₂)₄— [Linker 9—hydrogenated]

In some embodiments, the linker is —(CH₂)₄—CONH—CH₂CH₂(OCH₂CH₂)—OCH₂C≡C— [Linker 10]

In some embodiments, the linker is —(CH₂)₄—CONH—CH₂CH₂(OCH₂CH₂)₂—CH₂— [Linker 10—hydrogenated]

In some embodiments, the invention provides a monomer of the following formula (GalNAc Monomer 3):

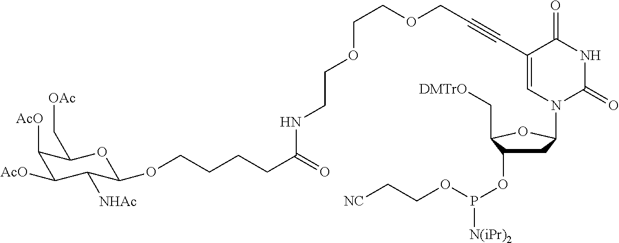

That is in Formula 3, R₁ is O—PN(iPr)₂OCH₂CH₂CN; R₂ is DMTr; R₄ is H; the linker L is -L1-Y-L2-X—, where n is 4, Y is CONH, m is 1 and X is *—OCH₂C≡C—, and GalNAc is protected with acetyl groups (that is, P is Ac). That is in Formula 3a, R₁ is O—PN(iPr)₂OCH₂CH₂CN; R₂ is DMTr; R₄ is H; the linker L is -L1-Y-L2-X—, where n is 4, Y is CONH, m is 1 and X is an oxygen atom, and GalNAc is protected with acetyl groups (that is, P is Ac).

In some embodiments, the invention provides a monomer of the following formula (GalNAc Monomer 5):

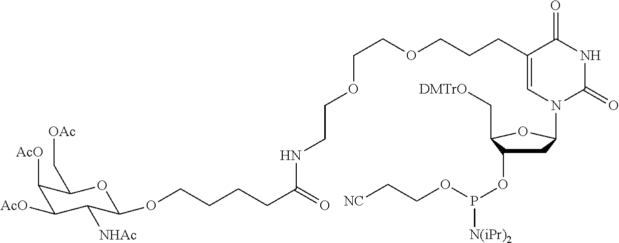

That is in Formula 3, R₁ is O—PN(iPr)₂OCH₂CH₂CN; R₂ is DMTr; R₄ is H; the linker L is -L1-Y-L2-X—, where n is 4, Y is CONH, m is 2 and X is —CH₂—, and GalNAc is protected with acetyl groups (that is, P is Ac). That is, L2 is —CH₂CH₂OCH₂CH₂OCH₂CH₂—.

The term ‘monomer residue’ refers to a monomer unit bound within the oligonucleotide chain at one or more positions. In the structure below, the wavy lines denote points of attachment within an oligonucleotide chain or a terminus of an oligonucleotide chain if appropriate.

Accordingly, an inhibitory nucleic acid according to the present disclosure may comprise at least one GalNAc monomer residue as described herein.

Suitably, an inhibitory nucleic acid (oligonucleotide) according to the present disclosure may comprise adjacent GalNAc monomer residues as described herein. In some embodiments, the inhibitory nucleic acid comprises preferably two, or more preferably three, adjacent monomer residues. It will be appreciated that said monomer residue(s) may be located at any point within the nucleotide chain. In some preferred embodiments, said monomer residue(s) are located at or near the 3′ end of the oligonucleotide. In some embodiments, said monomer residue(s) are located at or near the 5′ end of the oligonucleotide.

In some embodiments, the disclosure provides an oligonucleotide (e.g. inhibitory nucleic acid) comprising at least one monomer residue of formula:

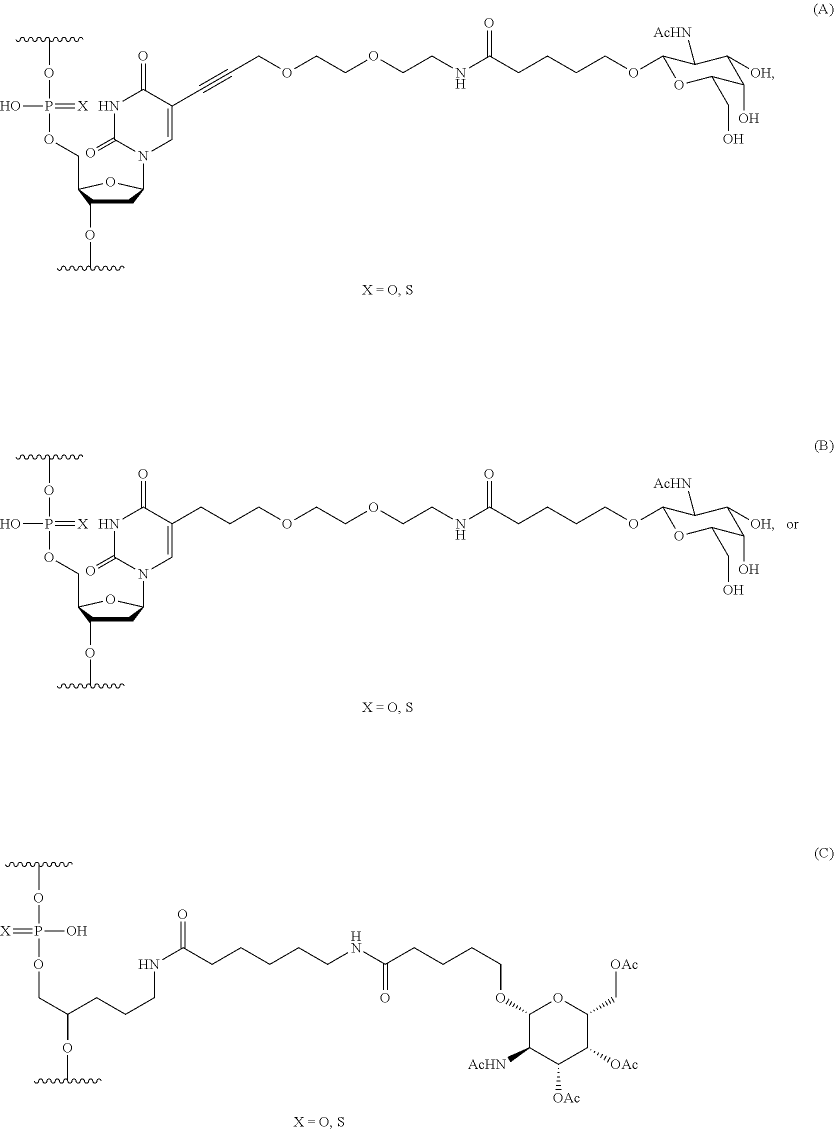

optionally comprising three copies of said monomer residue; that is, three copies of a monomer of formula (A), (B) or (C). Suitably, said copies are successive. In other words, the oligonucleotide comprises three adjacent monomer residues, said monomer residues being selected from one of formula (A), (B) or (C). In some embodiments, X is O. In some embodiments, X is S. Oligonucleotides and siRNAs exemplified herein comprise three monomer residues of formula (A) wherein X is O, X is S and X is S, respectively. Other oligonucleotides and siRNAs exemplified herein comprise three monomer residues of formula (B) wherein X is O, X is S and X is S, respectively. Yet other oligonucleotides and siRNAs exemplified herein comprise three monomer residues of formula (C) wherein X is O, X is S and X is S, respectively. Said monomer residue(s) may be located at the 3′ end of the oligonucleotide. Alternatively, said monomer residue(s) may be located at the 5′ end of the oligonucleotide or at another point in the chain.

In some embodiments, an inhibitory nucleic acid according to the present disclosure is selected from those drawn in FIG. 18A, 18B or 18C. The waved line indicates a nucleotide chain. In some embodiments, an inhibitory nucleic acid (e.g. siRNA) according to the invention is selected from those drawn in FIG. 19A, 19B or 19C. The waved lines indicate RNA strands. That is, an inhibitory nucleic acid according to the invention may comprise three successive monomers as shown in FIGS. 18A, B and C, or FIGS. 19A, B and C.

In some embodiments, the monomer is selected from:

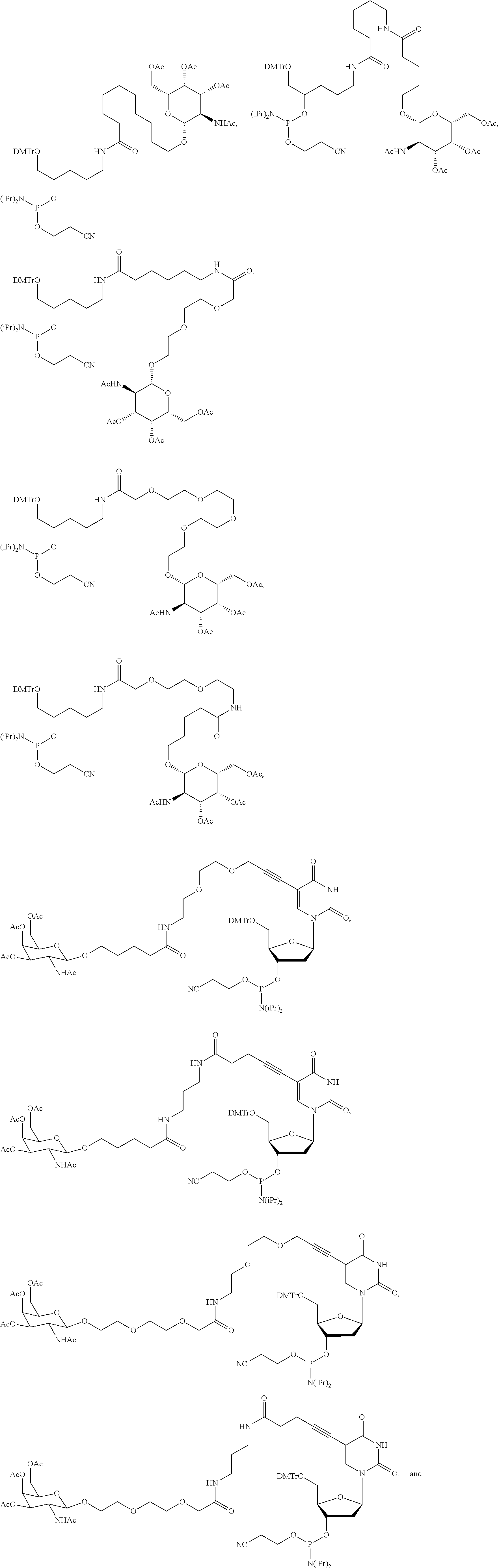

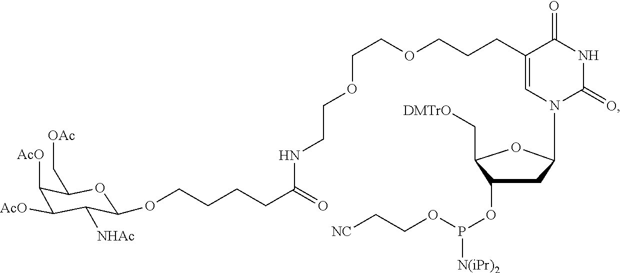

and, where the monomer contains an alkyne bond, the corresponding monomer in which that bond is fully hydrogenated.

Conjugates of biomolecules may be produced utilising ‘click chemistry’, as described e.g. in I and Brechbiel Cancer Biother Radiopharm. (2009) 24(3):289-302 and Astakhova et al., Mol Pharm. (2018) 15(8): 2892-2899, both of which are hereby incorporated by reference in their entirety. In some embodiments, conjugation may employ akyne-azide or thio-maleimide approaches. In some embodiments, a nucleic acid may be conjugated to a moiety facilitating delivery to, and/or uptake by, a cell type or tissue of interest e.g. at the 3′ and/or 5′ end of one or more strands of the nucleic acid.

Nucleic acids may be conjugated to one or more moieties facilitating delivery to, and/or uptake by, cell types or tissues of interest via a linker. In some embodiments, a linker may be or comprise a nucleotide sequence. The nucleotide sequence of a linker may comprise one or more modified nucleotides as described herein.

### Therapeutic and Prophylactic Applications

The inhibitory nucleic acids, nucleic acids, expression vectors, cells and compositions described herein find use in therapeutic and prophylactic methods.

The present disclosure provides an inhibitory nucleic acid, nucleic acid, expression vector, or composition described herein for use in a method of medical treatment or prophylaxis. Also provided is the use of an inhibitory nucleic acid, nucleic acid, expression vector, or composition described herein in the manufacture of a medicament for treating or preventing a disease or condition. Also provided is a method of treating or preventing a disease or condition, comprising administering to a subject a therapeutically or prophylactically effective amount of an inhibitory nucleic acid, nucleic acid, expression vector, or composition described herein.

The terms ‘disorder’, ‘disease’ and ‘condition’ may be used interchangeably and refer to a pathological issue of a body part, organ or system which may be characterised by an identifiable group of signs or symptoms.

Therapeutic or prophylactic intervention in accordance with the present disclosure may be effective to reduce the development or progression of a disease/condition, alleviate the symptoms of a disease/condition or reduce the pathology of a disease/condition. The intervention may be effective to prevent progression of the disease/condition, e.g. to prevent worsening of, or to slow the rate of development of, the disease/condition. In some embodiments the intervention may lead to an improvement in the disease/condition, e.g. a reduction in the symptoms of the disease/condition or reduction in some other correlate of the severity/activity of the disease/condition. In some embodiments the intervention may prevent development of the disease/condition to a later stage (e.g. a more severe stage, or a chronic stage).

The terms ‘develop’, ‘developing’, and ‘development’, e.g. of a disorder, as used herein refer both to the onset of a disease as well as the progression, exacerbation or worsening of a disease state/correlate thereof.

It will be appreciated that the articles of the present disclosure (inhibitory nucleic acids, nucleic acids, expression vectors, cells and compositions described herein) may be used for the treatment/prevention of any disease/condition that would derive therapeutic or prophylactic benefit from a reduction in the level of gene and/or protein expression of ITFG1, and/or a reduction in the level of a function of ITFG1.

The disease/condition to be treated/prevented in accordance with the present disclosure may be a disease/condition in which gene and/or protein expression of ITFG1 and/or a function of ITFG1 is pathologically-implicated. For example, the disease/condition may be a disease/condition in which elevated gene and/or protein expression of ITFG1, and/or an increased level of the function of ITFG1 is implicated in the pathology of the disease/condition.

The disease/condition may be characterised by an increased level of gene and/or protein expression of ITFG1, or an increased level of a function of ITFG1 (e.g. as compared to the level of expression or the relevant function in the absence of the disease/condition). The disease/condition may be a disease/condition in which an increased level of gene and/or protein expression of ITFG1, and/or an increased level of a function of ITFG1, is positively associated with the onset, development or progression of the disease/condition. The disease/condition may be a disease/condition in which an increased level of gene and/or protein expression of ITFG1, and/or an increased level of a function of ITFG1, is positively associated with the severity of one or more symptoms of the disease/condition. The disease/condition may be a disease/condition for which an increased level of gene and/or protein expression of ITFG1, and/or an increased level of a function of ITFG1, is a risk factor for the onset, development or progression of the disease/condition.

The increased level of gene and/or protein expression of ITFG1, and/or the increased level of a function of ITFG1, in accordance with the preceding paragraph may be in cells, tissue and/or an organ in which one or more symptoms of the disease/condition manifest. In some embodiments, the increased level of gene and/or protein expression of ITFG1, and/or the increased level of a function of ITFG1, may be in cells of the liver (e.g. hepatocytes), hepatic tissue and/or in the liver.

Therapeutic or prophylactic intervention in accordance with the present disclosure may achieve a reduction in the level of gene and/or protein expression of ITFG1, and/or a reduction in the level of a function of ITFG1 (i.e. in the treated subject). In some embodiments, the therapeutic/prophylactic intervention may achieve a reduction in the level of gene and/or protein expression of ITFG1, and/or a reduction in the level of a function of ITFG1, in cells, tissue and/or an organ in which one or more symptoms of the disease/condition manifest. In some embodiments, the therapeutic/prophylactic intervention may achieve a reduction in the level of gene and/or protein expression of ITFG1, and/or a reduction in the level of a function of ITFG1, in cells of the liver (e.g. hepatocytes), hepatic tissue and/or in the liver.

The articles of the present disclosure find use in therapeutic or prophylactic intervention for the treatment/prevention of fibrosis. WO 2022/025827 A1 (incorporated by reference hereinabove) demonstrates that RNAi-mediated knockdown of expression of ITFG1 attenuates the development of fibrosis, and in particular fibrosis of the liver in a murine model of NAFLD (see Example 2 of WO 2022/025827 A1).

Accordingly, in some embodiments a disease/condition to be treated/prevented in accordance with the present disclosure is fibrosis, or a disease/condition characterised by fibrosis. As used herein, a disease/condition which is ‘characterised by fibrosis’ is a disease in which fibrosis is a symptom of the disease/condition.

Fibrosis can be triggered by pathological conditions, e.g. conditions, infections or disease states that lead to production of pro-fibrotic factors (e.g. as TGFβ1). Fibrosis may be caused by physical injury/stimuli, chemical injury/stimuli or environmental injury/stimuli. Physical injury/stimuli may occur during surgery, e.g. iatrogenic causes. Chemical injury/stimuli may include drug-induced fibrosis, e.g. following chronic administration of drugs such as bleomycin, cyclophosphamide, amiodarone, procainamide, penicillamine, gold and nitrofurantoin (Daba et al., Saudi Med J. (2004) 25(6): 700-706). Environmental injury/stimuli may include exposure to asbestos fibres or silica.

Fibrosis can be of any tissue/organ of the body. In some embodiments, fibrosis is of the lung (e.g. bronchioles, alveoli), airways (e.g. nasal cavity, oral cavity, pharynx, larynx, trachea, bronchi), heart, kidney, liver, skeletal muscle, blood vessels, eye, skin, pancreas, bowel, small intestine, large intestine, colon, joints, brain, or bone marrow. Fibrosis may also occur in multiple tissues/organs at once.

In some embodiments, fibrosis may be of an organ or tissue of the respiratory system, e.g. the lung (e.g. bronchioles, alveoli), or airways (e.g. nasal cavity, oral cavity, pharynx, larynx, trachea, bronchi). In some embodiments, fibrosis may be of an organ or tissue of the cardiovascular system, e.g. the heart or blood vessels. In some embodiments, fibrosis may be of an organ or tissue of the gastrointestinal system, e.g. of the liver, bowel, small intestine, large intestine, colon, or pancreas. In some embodiments, fibrosis may be of the eye. In some embodiments, fibrosis may be of the skin. In some embodiments, fibrosis may be of an organ or tissue of the nervous system, e.g. the brain. In some embodiments, fibrosis may be of the bone marrow. In some embodiments, fibrosis may be of the joints. In some embodiments, fibrosis may be of an organ or tissue of the urinary system, e.g. the kidneys. In some embodiments, fibrosis may be of an organ or tissue of the musculoskeletal system, e.g. muscle tissue. In some embodiments, fibrosis may be of an organ or tissue of one or more organ systems.

### Diseases and Conditions Characterised by Fibrosis Include, but are not Limited to:

Diseases/conditions affecting the liver such as chronic liver disease, liver fibrosis, cirrhosis, non-alcoholic fatty liver disease (NAFLD), hepatitis, steatohepatitis, non-alcoholic steatohepatitis (NASH), alcoholic liver disease (ALD), alcoholic fatty liver (AFL), alcoholic hepatitis, alcoholic steatohepatitis (ASH), primary biliary cholangitis (PBC), schistosomal liver disease, liver cancer and hepatocellular carcinoma (HCC);
Diseases/conditions affecting the respiratory system such as pulmonary fibrosis, interstitial lung disease (ILD), idiopathic interstitial pneumonia (IIP), idiopathic pulmonary fibrosis (IPF), cystic fibrosis, progressive massive fibrosis, scleroderma, obliterative bronchiolitis, Hermansky-Pudlak syndrome, asbestosis, silicosis, sarcoidosis, tumor stroma in lung disease, chronic obstructive pulmonary disease (COPD), emphysema, pneumonia, pulmonary edema, chronic bronchitis and asthma;
Diseases/conditions affecting the cardiovascular system such as hypertrophic cardiomyopathy (HCM), dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertension, hypertensive heart disease, arrhythmogenic right ventricular cardiomyopathy (ARVC), atherosclerosis, chronic pulmonary hypertension, AIDS-associated pulmonary hypertension, varicose veins and cerebral infarcts;
Diseases/conditions affecting the kidneys such as tubulointerstitial fibrosis, glomerular fibrosis, renal fibrosis, nephritic syndrome, Alport's syndrome, HIV-associated nephropathy, polycystic kidney disease, Fabry's disease, diabetic nephropathy, chronic glomerulonephritis and nephritis associated with systemic lupus;
Diseases/conditions affecting the pancreas such as pancreatic fibrosis, cystic fibrosis and chronic pancreatitis;
Diseases/conditions affecting the nervous system such as gliosis, Alzheimer's disease and multiple sclerosis;
Diseases/conditions affecting the musculoskeletal system such as muscular dystrophy, Duchenne muscular dystrophy (DMD), Becker's muscular dystrophy (BMD) and fibrotic myopathy;
Diseases/conditions affecting the gastrointestinal system such as inflammatory bowel disease (IBD), Crohn's disease, microscopic colitis and primary sclerosing cholangitis (PSC);
Diseases/conditions affecting the skin such as scleroderma, nephrogenic systemic fibrosis, Dupuytren's contracture and cutis keloid;
Diseases/conditions affecting the eye such as Grave's ophthalmopathy, epiretinal fibrosis, retinal fibrosis, subretinal fibrosis, subretinal fibrosis associated with macular degeneration (e.g. wet age-related macular degeneration (AMD)), diabetic retinopathy, glaucoma, corneal fibrosis, post-surgical fibrosis (e.g. of the posterior capsule following cataract surgery, or of the bleb following trabeculectomy for glaucoma), conjunctival fibrosis and subconjunctival fibrosis;
Diseases/conditions affecting the joints such as arthrofibrosis, arthritis and adhesive capsulitis;
Diseases/conditions affecting multiple tissues/organ systems, including progressive systemic sclerosis (PSS), chronic graft versus host disease (GVHD); fibrotic pre-neoplastic and fibrotic neoplastic disease, and fibrosis induced by chemical or environmental insult (e.g., cancer chemotherapy, pesticides, radiation/cancer radiotherapy);
Cancers, such as liver cancer, hepatocellular carcinoma, gastric cancer, esophageal cancer, lung cancer, head and neck cancer, colorectal cancer, pancreatic cancer, cervical cancer, and vulvar cancer;
Mediastinal fibrosis, retroperitoneal fibrosis, myelofibrosis and Peyronie's disease.

In some embodiments, fibrosis is fibrosis of the liver. In some embodiments, the disease/condition to be treated is selected from: chronic liver disease, liver fibrosis, cirrhosis, non-alcoholic fatty liver disease (NAFLD), hepatitis, steatohepatitis, non-alcoholic steatohepatitis (NASH), alcoholic liver disease (ALD), alcoholic fatty liver (AFL), alcoholic hepatitis, alcoholic steatohepatitis (ASH), primary biliary cholangitis (PBC), schistosomal liver disease, congenital liver disease, liver cancer and hepatocellular carcinoma (HCC).

In aspects and embodiments of the present disclosure, an inhibitory nucleic acid according to the present disclosure is provided for use in the treatment or prevention of a disease/condition characterised by fibrosis described herein. Also provided is the use of an inhibitory nucleic acid according to the present disclosure in the manufacture of a medicament for use in treating or preventing a disease/condition characterised by fibrosis described herein. Also provided is a method of treating or preventing a disease/condition characterised by fibrosis described herein, comprising administering to a subject a therapeutically- or prophylactically-effective amount of an inhibitory nucleic acid according to the present disclosure.

The articles of the present disclosure also find use in the treatment/prevention of diseases/conditions that would derive therapeutic or prophylactic benefit from an increase in the number/proportion of ITFG1-expressing cells (e.g. hepatocytes, lung cells, myoblasts), and/or increased cell proliferation/population expansion of ITFG1-expressing cells (e.g. hepatocytes, lung cells, myoblasts). WO 2022/025827 A1 (incorporated by reference hereinabove) demonstrates that RNAi-mediated knockdown of expression of ITFG1 promotes proliferation of hepatocytes, lung cells and myoblasts.

In some embodiments a disease/condition to be treated/prevented in accordance with the present disclosure is a disease/condition characterised by damage to and/or death of cells of the liver (e.g. hepatocytes)/liver tissue/the liver. Diseases/conditions characterised by damage to and/or death of cells of the liver (e.g. hepatocytes)/liver tissue/the liver include chronic liver disease, liver fibrosis, cirrhosis, non-alcoholic fatty liver disease (NAFLD), hepatitis, steatohepatitis, non-alcoholic steatohepatitis (NASH), alcoholic liver disease (ALD), alcoholic fatty liver (AFL), alcoholic hepatitis, alcoholic steatohepatitis (ASH), primary biliary cholangitis (PBC), primary sclerosing cholangitis (PSC), schistosomal liver disease, congenital liver disease, liver cancer, hepatocellular carcinoma (HCC), acute liver injury (ALI), acute liver failure, acute liver disease, viral hepatitis, liver ischemia-reperfusion injury (IRI; e.g. ‘warm’ ischemia-reperfusion (WIR)), radiation-induced liver disease (RILD), drug-induced liver injury (DILI; e.g. acetaminophen-induced liver injury), autoimmune liver injury, liver transplantation, extended hepatectomy, small-for-size syndrome, split liver grafts, and cholestatic liver disease.

In some embodiments, the disease/condition to be treated/prevented in accordance with the present disclosure is a non-hepatic disease/condition, i.e. it is not associated with the liver. Such diseases/conditions may still be characterised as described hereinabove, e.g. characterised by an increased level of gene and/or protein expression of ITFG1, or an increased level of a function of ITFG1.

In some embodiments a disease/condition to be treated/prevented in accordance with the present disclosure is a disease/condition characterised by damage to and/or death of cells of the respiratory system (e.g. cells of the lung, e.g. epithelial cells of the lung)/a tissue of the respiratory system (e.g. lung tissue)/an organ of the respiratory system (e.g. the lung). Diseases/conditions characterised by damage to and/or death of respiratory system (e.g. cells of the lung, e.g. epithelial cells of the lung)/a tissue of the respiratory system (e.g. lung tissue)/an organ of the respiratory system (e.g. the lung) include pulmonary fibrosis, interstitial lung disease (ILD), idiopathic interstitial pneumonia (IIP), idiopathic pulmonary fibrosis (IPF), cystic fibrosis, progressive massive fibrosis, scleroderma, obliterative bronchiolitis, Hermansky-Pudlak syndrome, asbestosis, silicosis, sarcoidosis, tumor stroma in lung disease, chronic obstructive pulmonary disease (COPD), emphysema, pneumonia, pulmonary edema, chronic bronchitis and asthma.

In some embodiments a disease/condition to be treated/prevented in accordance with the present disclosure is a disease/condition characterised by damage to and/or death of muscle cells/muscle tissue. Diseases/conditions characterised by muscle cells/muscle tissue include muscular dystrophy, Duchenne muscular dystrophy (DMD), Becker's muscular dystrophy (BMD), fibrotic myopathy, hypertrophic cardiomyopathy (HCM), dilated cardiomyopathy (DCM), myocardial fibrosis, myocarditis, endomyocardial fibrosis, myocardial infarction and arrhythmogenic right ventricular cardiomyopathy (ARVC).

In some embodiments, a disease/condition to be treated/prevented in accordance with the present disclosure is a disease/condition that would derive therapeutic and/or prophylactic benefit from an increase in the proliferation/survival and/or number/proportion of renal cells, e.g. tubular cells and/or ductal cells. In some embodiments, the disease/condition is a renal tubular disorder, renal tubular acidosis, hypokalemia, hyperkalemia, acute tubular necrosis, Fanconi syndrome, Liddle syndrome, nephrogenic diabetes insipidus, pseudohypoaldosteronism type I, chronic kidney disease or acute kidney injury.

In some embodiments, a disease/condition to be treated/prevented in accordance with the present disclosure is a disease/condition that would derive therapeutic and/or prophylactic benefit from an increase in the proliferation/survival and/or number/proportion of glial cells and/or neuronal cells. In some embodiments, the disease/condition is a neurodegenerative disease, amyotrophic lateral sclerosis, epilepsy, multiple sclerosis, frontotemporal dementia, Parkinson's disease, Alzheimer's disease, Huntington's disease, multiple system atrophy, or a prion disease.

In some embodiments, a disease/condition to be treated/prevented in accordance with the present disclosure is a disease/condition that would derive therapeutic and/or prophylactic benefit from an increase in the proliferation/survival and/or number/proportion of intestinal enterocytes and/or enteroendocrine cells. In some embodiments, the disease/condition is an inflammatory bowel disease, ulcerative colitis, Crohn's disease, inflammatory bowel syndrome, a chronic inflammatory condition of the gastrointestinal tract or coeliac disease.

The articles of the present disclosure also find use in methods for forming and/or regenerating a tissue, e.g. liver tissue, lung tissue or muscle tissue. The articles of the present disclosure find use in methods for healing (i.e. repairing damage to) a tissue, e.g. liver tissue, lung tissue or muscle tissue. WO 2022/025827 A1 (incorporated by reference hereinabove) demonstrates that RNAi-mediated knockdown of expression of ITFG1 promotes wound healing and liver regeneration in vitro and in vivo (Example 2 of WO 2022/025827 A1), through promoting proliferation of hepatocytes. As noted above, WO 2022/025827 A1 also shows that RNAi-mediated knockdown of expression of ITFG1 promoted proliferation of lung cells and myoblasts.

In particular embodiments, the present disclosure contemplates the use of the articles of the disclosure to promote the repair of and/or reverse damage to liver cells/tissue. The present disclosure contemplates to employ the articles of the disclosure to enhance/improve hepatic function in subjects having impaired hepatic function (e.g. as a consequence of a disease/condition or other hepatic insult (e.g. injury, e.g. DILI)).

The articles of the present disclosure are useful for promoting the proliferation of hepatocytes, for the generation of functional, hepatic tissue. Thus, the articles of the present disclosure are provided herein for promoting the proliferation, survival and/or function of hepatocytes; promoting the growth, maintenance and/or function of hepatic tissue; promoting the regeneration of hepatocytes and/or hepatic tissue; and/or preserving/improving hepatic function.

In addition to other properties described herein, in some embodiments the articles of the present disclosure possess one or more of the following properties, e.g. after administration to a subject, and optionally when compared to the subject before administration:
Reduce/prevent fibrosis;
Reduce/prevent steatosis;
Reduce/prevent cirrhosis;
Reduce/prevent inflammation;
Reduce/prevent NASH;
Delay the onset or progression of liver failure;
Reduce fibrosis score, e.g. as measurable by the Fibrosis-4 (FIB-4) Index for Liver Fibrosis;
Reduce steatosis score, e.g. as measurable by the steatosis-associated fibrosis estimator (SAFE) score or the CAP score;
Reduce/prevent hepatic stellate cell activity, measurable e.g. by Acta2 mRNA expression;
Increase/promote liver function, measurable e.g. by increased albumin protein expression;
Reduce/prevent levels of circulating liver markers, e.g. ALT, AST and/or bilirubin; and/or
Reduce/prevent liver toxicity, measurable e.g. by reduced levels of ALT, AST and/or bilirubin.

Administration of the articles of the present disclosure is preferably in a ‘therapeutically-effective’ or ‘prophylactically-effective’ amount, this being sufficient to show therapeutic or prophylactic benefit to the subject. The actual amount administered, and rate and time-course of administration, will depend on the nature and severity of the disease/condition and the particular article administered. Prescription of treatment, e.g. decisions on dosage etc., is within the responsibility of general practitioners and other medical doctors, and typically takes account of the disease/disorder to be treated, the condition of the individual subject, the site of delivery, the method of administration and other factors known to practitioners. Examples of the techniques and protocols mentioned above can be found in Remington's ‘The Science and Practice of Pharmacy’ (ed. A. Adejare), 23^{rd} Edition (2020), Academic Press.

Administration of the articles of the present disclosure may be topical, parenteral, systemic, intracavitary, intravenous, intra-arterial, intramuscular, intrathecal, intraocular, intravitreal, intraconjunctival, subretinal, suprachoroidal, subcutaneous, intradermal, intrathecal, oral, nasal or transdermal. Administration may be by injection or infusion.

In some aspects and embodiments in accordance with the present disclosure, inhibitory nucleic acids, nucleic acids, expression vectors, cells and compositions described herein may be administered to the liver, e.g. to one or more hepatocytes. In some cases, inhibitory nucleic acids, nucleic acids, expression vectors, cells and compositions described herein may be administered to the blood (i.e. intravenous/intra-arterial administration), subcutaneously or orally.

In some aspects and embodiments in accordance with the present disclosure there may be targeted delivery of articles of the present disclosure, i.e. wherein the concentration of the relevant agent in the subject is increased in some parts of the body relative to other parts of the body. In some embodiments, the methods comprise intravenous, intra-arterial, intramuscular or subcutaneous administration and wherein the relevant article is formulated in a targeted agent delivery system. Suitable targeted delivery systems include, for example, nanoparticles, liposomes, micelles, beads, polymers, metal particles, dendrimers, antibodies, aptamers, nanotubes or micro-sized silica rods. Such systems may comprise a magnetic element to direct the agent to the desired organ or tissue. Suitable nanocarriers and delivery systems will be apparent to one skilled in the art.

In some cases, the relevant agent is formulated for targeted delivery to specific cells, tissue(s) and/or organ(s). In some cases, the relevant agent is formulated for targeted delivery to cells of the liver (e.g. hepatocytes), hepatic tissue and/or the liver.

The particular mode and/or site of administration may be selected in accordance with the location where reduction of gene and/or protein expression of ITFG1 is required, e.g. cells of the liver (e.g. hepatocytes), hepatic tissue and/or the liver.

In some embodiments, therapeutic or prophylactic intervention according to the present disclosure may further comprise administering another agent for the treatment/prevention of the disease/condition.

Administration of an article of the present disclosure may be alone or in combination with other treatments, either simultaneously or sequentially dependent upon the condition to be treated. Simultaneous administration refers to administration with another therapeutic agent together, for example as a pharmaceutical composition containing both agents (combined preparation), or immediately after each other and optionally via the same route of administration (e.g. to the same tissue, artery, vein or other blood vessel). Sequential administration refers to administration of one agent followed after a given time interval by separate administration of another agent. It is not required that the two agents are administered by the same route, although this is the case in some embodiments. The time interval may be any time interval.

Multiple doses of the articles of the present disclosure may be provided. One or more, or each, of the doses may be accompanied by simultaneous or sequential administration of another therapeutic agent.

Multiple doses may be separated by a predetermined time interval, which may be selected to be one of 1, 2,3, 4, 5, 6, 7, 8, 9,10,11,12,13,14,15,16,17,18,19, 20, 21, 22, 23, 24, 25, 26, 27, 28, 29, 30, or 31 days, or 1, 2, 3, 4, 5, or 6 months. By way of example, doses may be given once every 7, 14, 21 or 28 days (plus or minus 3, 2, or 1 days).

Articles of the present disclosure may be formulated in a sustained release delivery system, in order to release the inhibitory nucleic acid, nucleic acid, expression vector or composition at a predetermined rate.

Sustained release delivery systems may maintain a constant drug/therapeutic/prophylactic concentration for a specified period of time. In some embodiments, an inhibitory nucleic acid, nucleic acid, expression vector or composition described herein is formulated in a liposome, gel, implant, device, or drug-polymer conjugate e.g. hydrogel.

In accordance with various aspects of the present invention, methods are provided which are for, or which comprise (e.g. in the context of treatment/prevention of a disease/condition described herein) reducing ITFG1 gene and/or protein expression, and/or increasing cell proliferation/population expansion of ITFG1-expressing cells (e.g. hepatocytes). Also provided are agents according to the present disclosure for use in such methods, and the use of agents according to the present disclosure in manufacture of compositions (e.g. medicaments) for use in such methods. It will be appreciated that the methods typically comprise administering an agent according to the present disclosure to a subject.

Similarly, reduced ITFG1 gene and/or protein expression, and/or increased cell proliferation/population expansion of ITFG1-expressing cells (e.g. hepatocytes) may be observed in a subject following therapeutic or prophylactic intervention in accordance with the present disclosure (e.g. compared to the level prior to intervention).

In some embodiments, therapeutic/prophylactic intervention in accordance with the present disclosure may be described as being ‘associated with’ one or more of the effects described in the preceding paragraph. The skilled person is readily able to evaluate such properties using techniques that are routinely practiced in the art.

### Subjects

A subject in accordance with the various aspects of the present disclosure may be any animal or human. Therapeutic and prophylactic applications may be in human or animals (veterinary use).

The subject to be administered with an article of the present disclosure (e.g. in accordance with therapeutic or prophylactic intervention) may be a subject in need of such intervention. The subject is preferably mammalian, more preferably human. The subject may be a non-human mammal, but is more preferably human. The subject may be male or female. The subject may be a patient.

A subject may have (e.g. may have been diagnosed with) a disease or condition described herein, may be suspected of having such a disease/condition, or may be at risk of developing/contracting such a disease/condition. In embodiments according to the present disclosure, a subject may be selected for treatment according to the methods based on characterisation for one or more markers of such a disease/condition.

### Kits

In some aspects of the present disclosure a kit of parts is provided. In some embodiments the kit may have at least one container having a predetermined quantity of an inhibitory nucleic acid, nucleic acid, expression vector, cell or composition described herein.

In some embodiments, the kit may comprise materials for producing an inhibitory nucleic acid, nucleic acid, expression vector, cell or composition described herein.

The kit may provide the inhibitory nucleic acid, nucleic acid, expression vector, cell or composition together with instructions for administration to a patient in order to treat a specified disease/condition.

In some embodiments the kit may further comprise at least one container having a predetermined quantity of another therapeutic agent (e.g. as described herein). In such embodiments, the kit may also comprise a second medicament or pharmaceutical composition such that the two medicaments or pharmaceutical compositions may be administered simultaneously or separately such that they provide a combined treatment for the specific disease or condition.

Kits according to the present disclosure may include instructions for use, e.g. in the form of an instruction booklet or leaflet. The instructions may include a protocol for performing any one or more of the methods described herein.

### Sequence Identity

Pairwise and multiple sequence alignment for the purposes of determining percent identity between two or more amino acid or nucleic acid sequences can be achieved in various ways known to a person of skill in the art, for instance, using publicly available computer software such as ClustalOmega (Söding, J. 2005, Bioinformatics 21, 951-960), T-coffee (Notredame et al. 2000, J. Mol. Biol. (2000) 302, 205-217), Kalign (Lassmann and Sonnhammer 2005, BMC Bioinformatics, 6(298)) and MAFFT (Katoh and Standley 2013, Molecular Biology and Evolution, 30(4) 772-780) software. When using such software, the default parameters, e.g. for gap penalty and extension penalty, are preferably used.

Sequences

| SEQ | | |
|---|---|---|
| ID | | |
| NO: | DESCRIPTION | SEQUENCE |
| 1 | Human ITFG1 isoform | MAAAGRLPSSWALFSPLLAGLALLGVGPVPARALHNVTAELFGAE |
| | 1 (NCBI: | AWGTLAAFGDLNSDKQTDLFVLRERNDLIVFLADQNAPYFKPKVK |
| | NP_110417.2) | VSFKNHSALITSVVPGDYDGDSQMDVLLTYLPKNYAKSELGAVIF |
| | | WGQNQTLDPNNMTILNRTFQDEPLIMDFNGDLIPDIFGITNESNQ |
| | | PQILLGGNLSWHPALTTTSKMRIPHSHAFIDLTEDFTADLFLTTL |
| | | NATTSTFQFEIWENLDGNFSVSTILEKPQNMMVVGQSAFADFDGD |
| | | GHMDHLLPGCEDKNCQKSTIYLVRSGMKQWVPVLQDFSNKGTLWG |
| | | FVPFVDEQQPTEIPIPITLHIGDYNMDGYPDALVILKNTSGSNQQ |
| | | AFLLENVPCNNASCEEARRMFKVYWELTDLNQIKDAMVATFFDIY |
| | | EDGILDIVVLSKGYTKNDFAIHTLKNNFEADAYFVKVIVLSGLCS |
| | | NDCPRKITPFGVNQPGPYIMYTTVDANGYLKNGSAGQLSQSAHLA |
| | | LQLPYNVLGLGRSANFLDHLYVGIPRPSGEKSIRKQEWTAIIPNS |
| | | QLIVIPYPHNVPRSWSAKLYLTPSNIVLLTAIALIGVCVFILAII |
| | | GILHWQEKKADDREKRQEAHRFHFDAM |
| 2 | FG-GAP repeat | VVGQSAFADFDGDGHMDHLLPGCEDKNCQKSTIYLV |
| 3 | Human ITFG1 signal | MAAAGRLPSSWALFSPLLAGLALLGVGPVPARA |
| | peptide | |
| 4 | Human ITFG1 | VLLTAIALIGVCVFILAIIGI |
| | transmembrane | |
| | region | |
| 5 | Human ITFG1 isoform | LHNVTAELFGAEAWGTLAAFGDLNSDKQTDLFVLRERNDLIVFLA |
| | 1 mature | DQNAPYFKPKVKVSFKNHSALITSVVPGDYDGDSQMDVLLTYLPK |
| | | NYAKSELGAVIFWGQNQTLDPNNMTILNRTFQDEPLIMDFNGDLI |
| | | PDIFGITNESNQPQILLGGNLSWHPALTTTSKMRIPHSHAFIDLT |
| | | EDFTADLFLTTLNATTSTFQFEIWENLDGNFSVSTILEKPQNMMV |
| | | VGQSAFADFDGDGHMDHLLPGCEDKNCQKSTIYLVRSGMKQWVPV |
| | | LQDFSNKGTLWGFVPFVDEQQPTEIPIPITLHIGDYNMDGYPDAL |
| | | VILKNTSGSNQQAFLLENVPCNNASCEEARRMFKVYWELTDLNQI |
| | | KDAMVATFFDIYEDGILDIVVLSKGYTKNDFAIHTLKNNFEADAY |
| | | FVKVIVLSGLCSNDCPRKITPFGVNQPGPYIMYTTVDANGYLKNG |
| | | SAGQLSQSAHLALQLPYNVLGLGRSANFLDHLYVGIPRPSGEKSI |
| | | RKQEWTAIIPNSQLIVIPYPHNVPRSWSAKLYLTPSNIVLLTAIA |
| | | LIGVCVFILAIIGILHWQEKKADDREKRQEAHRFHFDAM |
| 6 | Human ITFG1 isoform | MDVLLTYLPKNYAKSELGAVIFWGQNQTLDPNNMTILNRTFQDEP |
| | 2 (NCBI: | LIMDFNGDLIPDIFGITNESNQPQILLGGNLSWHPALTTTSKMRI |
| | NP_001291931.1) | PHSHAFIDLTEDFTADLFLTTLNATTSTFQFEIWENLDGNFSVST |
| | | ILEKPQNMMVVGQSAFADFDGDGHMDHLLPGCEDKNCQKSTIYLV |
| | | RSGMKQWVPVLQDFSNKGTLWGFVPFVDEQQPTEIPIPITLHIGD |
| | | YNMDGYPDALVILKNTSGSNQQAFLLENVPCNNASCEEARRMFKV |
| | | YWELTDLNQIKDAMVATFFDIYEDGILDIVVLSKGYTKNDFAIHT |
| | | LKNNFEADAYFVKVIVLSGLCSNDCPRKITPFGVNQPGPYIMYTT |
| | | VDANGYLKNGSAGQLSQSAHLALQLPYNVLGLGRSANFLDHLYVG |
| | | IPRPSGEKSIRKQEWTAIIPNSQLIVIPYPHNVPRSSHRCLCFHL |
| | | GNNWHFTLAGKESR |
| 7 | Macaca fascicularis | MAAAGRLPSSWALLSPLLTGLALLGVGPVPARALHNVTAELFGAE |
| | ITFG1 (NCBI | AWGTLAAFGDLNSDKQTDLFVLRERNDLIVFLADQNAPYFKPKVK |
| | XP_005591903.2) | VSFKNHSALITSVVPGDYDGDSQMDVLLTYLPKNYAKSELGAVIF |
| | | WGQNQTLDPNNMTILNRTFQDEPLIMDFNGDLIPDIFGITNESNQ |
| | | PQILLGGNLSWHPALTTTSKMRIPHSHAFIDLTEDFTADLFLTTL |
| | | NATSSTFQFEIWENLDGNFSVSTILEKPQNMMVVGQSAFADFDGD |
| | | GHMDHLLPGCEDKNCQKSTIYLVRSGTKQWVPVLQDFSNKGTLWG |
| | | FVPFVDEQQPTEIPIPITLHIGDYNMDGYPDALVILKNTSGSNQQ |
| | | AFLLENVPCNNASCEEARRMFKVYWELTDLNQIKDAMVATFFDIY |
| | | EDGILDIVVLSKGYTKNDFAIHTLKNNFEADAYFVKVIVLSGLCS |
| | | NDCPRKITPFGVNQPGPYIMYTTVDANGYLKNGSAGQLSQSAHLA |
| | | LQLPYNVLGLGRSANFLDHLYVGIPRPSGEKSIRKQEWTAIIPNS |
| | | QLIVIPYPHNVPRSWSAKLYLTPSNIVLLTAIALIGVCVFILAII |
| | | GILHWQEKKADDREKRQEAHRFHFDAM |
| 8 | Mus musculus ITFG1 | MAAGRLPSARAVLAPLFLGLALLSVGPAPARALHNVTAELFGAEA |
| | (NCBI: NP_082283.2) | WGTLAAFGDLNSDKQTDLFVLRERNDLIVFLADQSAPYFKPKVKV |
| | | SLKTLSALVTSVVPGDYDGDSQMDVLLTYFPQNHTNSELGAVIFW |
| | | GQNQTLDPKNMTILNRTFHDQPLIMDFNGDLIPDVFGITNESSQP |
| | | QILLGGDLSWHPALTTKSKMRDPHSHAFIDLTEDFTADLFLTTLT |
| | | ASNAFQFEIWENLGGNFSIHSVFEKPKNLVVVGQSAFADFDGDGH |
| | | MDHLLPGCEDKDCQKSAIYLMRSGTGQWVPVLQDFSNKGTLWGFV |
| | | PFVHEEQPTTIPIPLTLHIGDYNMDGYPDALAILKNTSGSNQQAF |
| | | LLENVPCNNASCEEVHRMFKVYWDLAGLNLIKDAIVATFFDIYED |
| | | GILDIIVLSKGYTKNDVAIHTLKNNFEADAYFVKVIVLSGLCSND |
| | | CPRKITPFGVNQPGPYIMYTTVDANGYLKNGSAGQLSQSAHLALQ |
| | | LPYNVLGLGRSANFLDHLFVGIPRPSGEKSIRKQEWTAIIPNSQL |
| | | IVIPYPHNVPRSWSAKLYLTPSNIVLLTAVALIGVCIFILAIIAI |
| | | LHWQEKKADDREKRQEAHRFHFDAM |
| 9 | RNA encoding human | GGGGGCUGAGGGGCUGCCAUGGCGGCGGCGGGCCGGCUCCCGAGC |
| | ITFG1 isoform 1 | UCCUGGGCCCUCUUCUCGCCGCUCCUCGCAGGGCUUGCACUACUG |
| | (NCBI: NM_030790.5) | GGAGUCGGGCCGGUCCCAGCGCGGGCGCUGCACAACGUCACGGCC |
| | | GAGCUCUUUGGGGCCGAGGCCUGGGGCACCCUUGCGGCUUUCGGG |
| | | GACCUCAACUCCGACAAGCAGACGGAUCUCUUCGUGCUGCGGGAA |
| | | AGAAAUGACUUAAUCGUCUUUUUGGCAGACCAGAAUGCACCCUAU |
| | | UUUAAACCCAAAGUAAAGGUAUCUUUCAAGAAUCACAGUGCAUUG |
| | | AUAACAAGUGUAGUCCCUGGGGAUUAUGAUGGAGAUUCUCAAAUG |
| | | GAUGUCCUUCUGACAUAUCUUCCCAAAAAUUAUGCCAAGAGUGAA |
| | | UUAGGAGCUGUUAUCUUCUGGGGACAAAAUCAAACAUUAGAUCCU |
| | | AACAAUAUGACCAUACUCAAUAGGACUUUUCAAGAUGAGCCACUA |
| | | AUUAUGGAUUUCAAUGGUGAUCUAAUUCCUGAUAUUUUUGGUAUC |
| | | ACAAAUGAAUCCAACCAGCCACAGAUACUAUUAGGAGGGAAUUUA |
| | | UCAUGGCAUCCAGCAUUGACCACUACAAGUAAAAUGCGAAUUCCA |
| | | CAUUCUCAUGCAUUUAUUGAUCUGACUGAAGAUUUUACAGCAGAU |
| | | UUAUUCCUGACGACAUUGAAUGCCACCACUAGUACCUUCCAGUUU |
| | | GAAAUAUGGGAAAAUUUGGAUGGAAACUUCUCUGUCAGUACUAUA |
| | | UUGGAAAAACCUCAAAAUAUGAUGGUGGUUGGACAGUCAGCAUUU |
| | | GCAGACUUUGAUGGAGAUGGACACAUGGAUCAUUUACUGCCAGGC |
| | | UGUGAAGAUAAAAAUUGCCAAAAGAGUACCAUCUACUUAGUGAGA |
| | | UCUGGGAUGAAGCAGUGGGUUCCAGUCCUACAAGAUUUCAGCAAU |
| | | AAGGGCACACUCUGGGGCUUUGUGCCAUUUGUGGAUGAACAGCAA |
| | | CCAACUGAAAUACCAAUUCCAAUUACCCUUCAUAUUGGAGACUAC |
| | | AAUAUGGAUGGCUAUCCAGACGCUCUGGUCAUACUAAAGAACACA |
| | | UCUGGAAGCAACCAGCAGGCCUUUUUACUGGAGAACGUCCCUUGU |
| | | AAUAAUGCAAGCUGUGAAGAGGCGCGUCGAAUGUUUAAAGUCUAC |
| | | UGGGAGCUGACAGACCUAAAUCAAAUUAAGGAUGCCAUGGUUGCC |
| | | ACCUUCUUUGACAUUUACGAAGAUGGAAUCUUGGACAUUGUAGUG |
| | | CUAAGUAAAGGAUAUACAAAGAAUGAUUUUGCCAUUCAUACACUA |
| | | AAAAAUAACUUUGAAGCAGAUGCUUAUUUUGUUAAAGUUAUUGUU |
| | | CUUAGUGGUCUGUGUUCUAAUGACUGUCCUCGUAAGAUAACACCC |
| | | UUUGGAGUGAAUCAACCUGGACCUUAUAUCAUGUAUACAACUGUA |
| | | GAUGCAAAUGGGUAUCUGAAAAAUGGAUCAGCUGGCCAACUCAGC |
| | | CAAUCCGCACAUUUAGCUCUCCAACUACCAUACAACGUGCUUGGU |
| | | UUAGGUCGGAGCGCAAAUUUUCUUGACCAUCUCUACGUUGGUAUU |
| | | CCCCGUCCAUCUGGAGAAAAAUCUAUACGAAAACAAGAGUGGACU |
| | | GCAAUCAUUCCAAAUUCCCAGCUAAUUGUCAUUCCAUACCCUCAC |
| | | AAUGUCCCUCGAAGUUGGAGUGCCAAACUGUAUCUUACACCAAGU |
| | | AAUAUUGUUCUGCUUACUGCUAUAGCUCUCAUCGGUGUCUGUGUU |
| | | UUCAUCUUGGCAAUAAUUGGCAUUUUACAUUGGCAGGAAAAGAAA |
| | | GCAGAUGAUAGAGAAAAACGACAAGAAGCCCACCGGUUUCAUUUU |
| | | GAUGCUAUGUGACUUGCCUUUAAUAUUACAUAAUGGAAUGGCUGU |
| | | UCACUUGAUUAGUUGAAACACAAAUUCUGGCUUGAAAAAAUAGGG |
| | | GAGAUUAAAUAUUAUUUAUAAAUGAUGUAUCCCAUGGUAAUUAUU |
| | | GGAAAGUAUUCAAAUAAAUAUGGUUUGAAUAUGUCACAAGGUCUU |
| | | UUUUUUUAAAGCACUUUGUAUAUAAAAAUUUGGGUUCUCUAUUCU |
| | | GUAGUGCUGUACAUUUUUGUUCCUUUGUGGAAUGUGUUGCAUGUA |
| | | CUCCAGUGUUUGUGUAUUUAUAAUCUUAUUUGCAUCAUGAUGAUG |
| | | GAAAAAGUUGUGUAAAUAAAAAUAAUUAAAUGAGCAGGAAUUUUU |
| | | GUGUCCACUUGACUUGGUCUUGCUUCUUAUUCUAAUGAUGCAAAU |
| | | UAUACUUUUGUGAAUAUAUCACGGAGUCAUUAGGCAUUCAGCUUC |
| | | AUCACAGCAGGUCAGGGGUCUCACUGAUGGCAUACAAUAUAGUGA |
| | | UCGGGUACUCUGACUUGGUAGCACAGUAAGACAGACUUGCCUUAA |
| | | ACUCCUAAUUCAACCACUUACAAAGUCAUUGUUUGAACUUGGCUC |
| | | UUGUUUAACCUCUGUAAACCUCAGUUUUCUUGUUUAUUCAGUGGG |
| | | GCUAAUACUUGAGUUACUGUAAACAUUAAAUGGGAUGAUGUAUGU |
| | | GAAGUGCUUAGCUUGGUGCCUAGCACAGAGUAAGUGGUCAAUAUG |
| | | UGGUAGUUGUCAUUAUUAAUAUUUUAGAUGAUCUUAUUAGACUUA |
| | | UACAUCUAAUUAUAGAAAUACAUAGACUUGAUAGAAUUUUAUUUU |
| | | CAGGCAUGAAGAAAUAUUCUUUGGAAAAGCUAAAUUUUUGGUGAU |
| | | UGACAUAAAGAUUUACUUGCUCAUAUUAACUAAAAAUUAUAGUAC |
| | | UCUCCAAGAAUUAAUGUGCCCUAAAAAUUUUCCUCCAAAAACUUA |
| | | UCCUUAUCAUGUGAUAAUGAAGAACAUUUGAUUUCUUGAAAGGAA |
| | | ACUGCUGUAGGCAGCAUCUGGGAAUGCAAAUCUUCAAUCACAUUU |
| | | CUAUUCUCAAACACUUGGAGAAGUCUAUAAUUUACAUUCAGACUU |
| | | CAAUGCAAAUUUUGUAUUGUGAACUUCACAUUUCCAAAAAGUUAC |
| | | UUUAAAAAGACUUUAAGACUGAAAAAAAAAAGUUUAUCAAUGCUA |
| | | AUAAUUUUCUAGUAUGCAAAUGGACAUGUGAUGCCUAUAAAACAC |
| | | AAAAAUUUCUCUGAAAACAAUUUUGUUCUUAUUUUUUUCUUUAUA |
| | | GUUCACUGAGAUUGGCAUGUGUUUUUACUUUGUAUCUAAGCAUGU |
| | | UAACAUGUCUUCUUAAUAAAUAUUCCUUAUUGAAA |
| 10 | RNA encoding human | GAGCGUGAGGGCGGCUUUCACCCUAGCUCCACUGCUUUUUUAGCG |
| | ITFG1 isoform 2 | UCGGAAGGGCAGCCCUAGUGGGAGAGAGAAAAAGGAGCCGGCAGC |
| | (NCBI: | GGCUCUUACGCGUCCCGGGGCUGCGCGCCACUCUCUCGGCCGGAA |
| | NM_001305002.2) | AUGACUUAAUCGUCUUUUUGGCAGACCAGAAUGCACCCUAUUUUA |
| | | AACCCAAAGUAAAGGUAUCUUUCAAGAAUCACAGUGCAUUGAUAA |
| | | CAAGUGUAGUCCCUGGGGAUUAUGAUGGAGAUUCUCAAAUGGAUG |
| | | UCCUUCUGACAUAUCUUCCCAAAAAUUAUGCCAAGAGUGAAUUAG |
| | | GAGCUGUUAUCUUCUGGGGACAAAAUCAAACAUUAGAUCCUAACA |
| | | AUAUGACCAUACUCAAUAGGACUUUUCAAGAUGAGCCACUAAUUA |
| | | UGGAUUUCAAUGGUGAUCUAAUUCCUGAUAUUUUUGGUAUCACAA |
| | | AUGAAUCCAACCAGCCACAGAUACUAUUAGGAGGGAAUUUAUCAU |
| | | GGCAUCCAGCAUUGACCACUACAAGUAAAAUGCGAAUUCCACAUU |
| | | CUCAUGCAUUUAUUGAUCUGACUGAAGAUUUUACAGCAGAUUUAU |
| | | UCCUGACGACAUUGAAUGCCACCACUAGUACCUUCCAGUUUGAAA |
| | | UAUGGGAAAAUUUGGAUGGAAACUUCUCUGUCAGUACUAUAUUGG |
| | | AAAAACCUCAAAAUAUGAUGGUGGUUGGACAGUCAGCAUUUGCAG |
| | | ACUUUGAUGGAGAUGGACACAUGGAUCAUUUACUGCCAGGCUGUG |
| | | AAGAUAAAAAUUGCCAAAAGAGUACCAUCUACUUAGUGAGAUCUG |
| | | GGAUGAAGCAGUGGGUUCCAGUCCUACAAGAUUUCAGCAAUAAGG |
| | | GCACACUCUGGGGCUUUGUGCCAUUUGUGGAUGAACAGCAACCAA |
| | | CUGAAAUACCAAUUCCAAUUACCCUUCAUAUUGGAGACUACAAUA |
| | | UGGAUGGCUAUCCAGACGCUCUGGUCAUACUAAAGAACACAUCUG |
| | | GAAGCAACCAGCAGGCCUUUUUACUGGAGAACGUCCCUUGUAAUA |
| | | AUGCAAGCUGUGAAGAGGCGCGUCGAAUGUUUAAAGUCUACUGGG |
| | | AGCUGACAGACCUAAAUCAAAUUAAGGAUGCCAUGGUUGCCACCU |
| | | UCUUUGACAUUUACGAAGAUGGAAUCUUGGACAUUGUAGUGCUAA |
| | | GUAAAGGAUAUACAAAGAAUGAUUUUGCCAUUCAUACACUAAAAA |
| | | AUAACUUUGAAGCAGAUGCUUAUUUUGUUAAAGUUAUUGUUCUUA |
| | | GUGGUCUGUGUUCUAAUGACUGUCCUCGUAAGAUAACACCCUUUG |
| | | GAGUGAAUCAACCUGGACCUUAUAUCAUGUAUACAACUGUAGAUG |
| | | CAAAUGGGUAUCUGAAAAAUGGAUCAGCUGGCCAACUCAGCCAAU |
| | | CCGCACAUUUAGCUCUCCAACUACCAUACAACGUGCUUGGUUUAG |
| | | GUCGGAGCGCAAAUUUUCUUGACCAUCUCUACGUUGGUAUUCCCC |
| | | GUCCAUCUGGAGAAAAAUCUAUACGAAAACAAGAGUGGACUGCAA |
| | | UCAUUCCAAAUUCCCAGCUAAUUGUCAUUCCAUACCCUCACAAUG |
| | | UCCCUCGAAGCUCUCAUCGGUGUCUGUGUUUUCAUCUUGGCAAUA |
| | | AUUGGCAUUUUACAUUGGCAGGAAAAGAAAGCAGAUGAUAGAGAA |
| | | AAACGACAAGAAGCCCACCGGUUUCAUUUUGAUGCUAUGUGACUU |
| | | GCCUUUAAUAUUACAUAAUGGAAUGGCUGUUCACUUGAUUAGUUG |
| | | AAACACAAAUUCUGGCUUGAAAAAAUAGGGGAGAUUAAAUAUUAU |
| | | UUAUAAAUGAUGUAUCCCAUGGUAAUUAUUGGAAAGUAUUCAAAU |
| | | AAAUAUGGUUUGAAUAUGUCACAAGGUCUUUUUUUUUAAAGCACU |
| | | UUGUAUAUAAAAAUUUGGGUUCUCUAUUCUGUAGUGCUGUACAUU |
| | | UUUGUUCCUUUGUGGAAUGUGUUGCAUGUACUCCAGUGUUUGUGU |
| | | AUUUAUAAUCUUAUUUGCAUCAUGAUGAUGGAAAAAGUUGUGUAA |
| | | AUAAAAAUAAUUAAAUGAGCAGGAAUUUUUGUGUCCACUUGACUU |
| | | GGUCUUGCUUCUUAUUCUAAUGAUGCAAAUUAUACUUUUGUGAAU |
| | | AUAUCACGGAGUCAUUAGGCAUUCAGCUUCAUCACAGCAGGUCAG |
| | | GGGUCUCACUGAUGGCAUACAAUAUAGUGAUCGGGUACUCUGACU |
| | | UGGUAGCACAGUAAGACAGACUUGCCUUAAACUCCUAAUUCAACC |
| | | ACUUACAAAGUCAUUGUUUGAACUUGGCUCUUGUUUAACCUCUGU |
| | | AAACCUCAGUUUUCUUGUUUAUUCAGUGGGGCUAAUACUUGAGUU |
| | | ACUGUAAACAUUAAAUGGGAUGAUGUAUGUGAAGUGCUUAGCUUG |
| | | GUGCCUAGCACAGAGUAAGUGGUCAAUAUGUGGUAGUUGUCAUUA |
| | | UUAAUAUUUUAGAUGAUCUUAUUAGACUUAUACAUCUAAUUAUAG |
| | | AAAUACAUAGACUUGAUAGAAUUUUAUUUUCAGGCAUGAAGAAAU |
| | | AUUCUUUGGAAAAGCUAAAUUUUUGGUGAUUGACAUAAAGAUUUA |
| | | CUUGCUCAUAUUAACUAAAAAUUAUAGUACUCUCCAAGAAUUAAU |
| | | GUGCCCUAAAAAUUUUCCUCCAAAAACUUAUCCUUAUCAUGUGAU |
| | | AAUGAAGAACAUUUGAUUUCUUGAAAGGAAACUGCUGUAGGCAGC |
| | | AUCUGGGAAUGCAAAUCUUCAAUCACAUUUCUAUUCUCAAACACU |
| | | UGGAGAAGUCUAUAAUUUACAUUCAGACUUCAAUGCAAAUUUUGU |
| | | AUUGUGAACUUCACAUUUCCAAAAAGUUACUUUAAAAAGACUUUA |
| | | AGACUGAAAAAAAAAAGUUUAUCAAUGCUAAUAAUUUUCUAGUAU |
| | | GCAAAUGGACAUGUGAUGCCUAUAAAACACAAAAAUUUCUCUGAA |
| | | AACAAUUUUGUUCUUAUUUUUUUCUUUAUAGUUCACUGAGAUUGG |
| | | CAUGUGUUUUUACUUUGUAUCUAAGCAUGUUAACAUGUCUUCUUA |
| | | AUAAAUAUUCCUUAUUGAAA |
| 11 | RNA encoding | GGGGGCCGAGGGGCUGCCAUGGCGGCGGCGGGCCGGCUGCCGAGC |
| | Macaca fascicularis | UCCUGGGCGCUCCUGUCGCCGCUCCUCACGGGGCUUGCACUACUG |
| | ITFG1 (NCBI: | GGAGUCGGGCCGGUCCCAGCGCGGGCGCUGCACAACGUCACGGCC |
| | XM_005591846.3) | GAGCUCUUUGGGGCCGAGGCCUGGGGCACCCUUGCGGCUUUCGGG |
| | | GACCUCAACUCCGACAAGCAGACGGACCUCUUCGUGCUGCGGGAA |
| | | AGAAACGACUUAAUCGUCUUUUUGGCAGACCAGAAUGCACCCUAU |
| | | UUUAAACCUAAAGUAAAGGUAUCUUUCAAGAAUCACAGUGCAUUG |
| | | AUAACAAGUGUAGUCCCUGGGGAUUAUGAUGGAGAUUCUCAAAUG |
| | | GAUGUCCUUCUGACAUAUCUUCCCAAAAAUUAUGCCAAGAGUGAA |
| | | UUAGGAGCUGUUAUCUUCUGGGGACAAAAUCAAACGUUAGAUCCU |
| | | AACAAUAUGACCAUACUCAAUAGGACUUUUCAAGAUGAGCCACUA |
| | | AUUAUGGAUUUCAAUGGUGAUCUAAUUCCUGAUAUUUUUGGUAUC |
| | | ACAAAUGAAUCCAACCAGCCACAGAUACUAUUAGGAGGGAAUUUA |
| | | UCAUGGCAUCCUGCAUUGACCACUACAAGUAAAAUGCGAAUUCCA |
| | | CAUUCUCAUGCAUUUAUUGAUCUGACUGAAGAUUUUACAGCAGAU |
| | | UUAUUUCUGACGACAUUGAAUGCCACCUCUAGUACCUUCCAGUUU |
| | | GAGAUAUGGGAAAAUUUGGAUGGAAACUUCUCUGUCAGUACUAUA |
| | | UUGGAAAAACCUCAAAAUAUGAUGGUGGUUGGACAGUCAGCAUUU |
| | | GCAGACUUUGAUGGAGAUGGACACAUGGAUCAUUUACUGCCAGGC |
| | | UGUGAAGAUAAAAAUUGCCAAAAGAGUACCAUCUACUUAGUGAGA |
| | | UCUGGGACAAAGCAGUGGGUUCCAGUCCUACAAGAUUUCAGUAAU |
| | | AAGGGCACACUCUGGGGCUUUGUGCCGUUUGUGGAUGAACAGCAA |
| | | CCAACUGAAAUACCAAUUCCAAUUACCCUUCAUAUUGGAGACUAC |
| | | AAUAUGGAUGGCUAUCCAGAUGCUCUGGUCAUACUAAAGAACACA |
| | | UCUGGAAGCAACCAGCAGGCCUUUUUACUGGAGAACGUCCCUUGU |
| | | AAUAAUGCAAGCUGUGAAGAGGCGCGUCGAAUGUUCAAAGUCUAC |
| | | UGGGAGCUGACAGACCUAAAUCAAAUUAAGGAUGCCAUGGUUGCC |
| | | ACCUUCUUUGACAUUUACGAAGAUGGAAUCUUGGACAUUGUAGUG |
| | | CUAAGUAAAGGAUAUACAAAGAAUGAUUUUGCCAUCCAUACACUA |
| | | AAAAAUAACUUUGAAGCAGAUGCUUAUUUUGUUAAAGUUAUUGUU |
| | | CUUAGUGGUCUGUGUUCUAAUGACUGUCCUCGUAAGAUAACACCC |
| | | UUUGGAGUGAAUCAACCUGGACCUUAUAUCAUGUAUACAACUGUU |
| | | GAUGCAAAUGGGUAUCUGAAAAAUGGAUCAGCUGGUCAACUCAGC |
| | | CAAUCCGCACAUUUAGCUCUCCAACUACCAUACAACGUGCUUGGU |
| | | UUAGGUCGGAGCGCAAAUUUUCUUGACCAUCUGUAUGUUGGUAUU |
| | | CCCCGUCCAUCUGGAGAAAAAUCUAUACGAAAACAAGAGUGGACU |
| | | GCAAUCAUUCCAAAUUCCCAGCUAAUUGUCAUUCCAUACCCUCAC |
| | | AAUGUCCCUCGAAGUUGGAGUGCCAAACUAUAUCUUACACCAAGU |
| | | AACAUUGUUCUGCUUACUGCCAUAGCUCUCAUCGGUGUCUGUGUU |
| | | UUCAUCUUGGCAAUAAUUGGCAUUUUACAUUGGCAGGAAAAGAAA |
| | | GCAGAUGAUAGAGAAAAACGACAAGAAGCCCACCGGUUUCAUUUU |
| | | GAUGCUAUGUGACUUGCCUUUAAUGUUACAUAAUGGAAUGGCUGU |
| | | UCACUUGAUUAGUUGAAACACAAAUUCUGGCUUAAAAAAUAGGAG |
| | | AGAUUAAAUAUUAUUUGUAAAUGAUGUAUCCCAUGGUAAUUAUUG |
| | | GAAAGUAUUCAAAUAAAUAUGGUUUGAAUAUGUCACAAGGUCCUU |
| | | UUUUUUUUUAAAAGCACUUUGUAUAUAAAAAGUUUGGGUUCUUUA |
| | | UUCCGUAGUGCUGUACAUUUCUGUUCCUUUGUGGAAUGUGUUGCA |
| | | UGUACUCCCGUGUGUGUGUAUUUAUAAUCUUAUUAGUGUCAUGAU |
| | | GAUGGAAAAAGAUGUGUGUAAAUAAAAAUAAUUAAAUGAGCAGGA |
| | | A |
| 12 | RNA encoding Mus | CACGACAGCCGGAAGUGAGUUCCCGGGCAGACGCCGCCCUAGCUC |
| | musculus ITFG1 | CUCGGCGUCUUGGUCGUCGGAGAAACAGCCCCAGGGCAGCGUGAG |
| | (NCBI: NM_028007.3) | AGUGCAGCGAGGGGCUGCCCGUGAGGCCGCACCCCGAGUCUUUUC |
| | | UCGGUCGGCCGGUCAGUCGCACGGUGCUUUGCGGCUGCCGUCAAG |
| | | CGCGGCGGAGGCCGGCCGGGCCGAAGGCGCCAUGGCGGCAGGCCG |
| | | GCUUCCUAGCGCCAGGGCGGUGCUGGCCCCGCUGUUCUUGGGGCU |
| | | CGCGCUGCUGAGCGUUGGGCCUGCCCCGGCGCGGGCUCUGCACAA |
| | | CGUCACGGCCGAGCUCUUUGGGGCUGAGGCCUGGGGCACCCUGGC |
| | | GGCCUUCGGUGACCUCAACUCCGACAAACAGACGGACCUCUUCGU |
| | | GCUGCGGGAAAGAAAUGACUUAAUUGUCUUCUUGGCAGAUCAGAG |
| | | UGCACCCUAUUUUAAACCCAAGGUAAAGGUAUCCCUGAAGACUUU |
| | | GAGUGCGUUGGUAACAAGUGUGGUCCCUGGUGAUUAUGAUGGGGA |
| | | UUCACAAAUGGAUGUCCUGCUCACAUACUUUCCCCAAAAUCAUAC |
| | | CAACAGUGAACUAGGAGCUGUUAUCUUUUGGGGACAAAAUCAAAC |
| | | AUUAGAUCCUAAAAACAUGACCAUACUCAAUAGGACUUUUCAUGA |
| | | CCAACCACUGAUUAUGGACUUCAAUGGUGAUCUAAUUCCUGAUGU |
| | | GUUUGGUAUCACAAAUGAAUCCAGCCAACCGCAGAUACUGCUAGG |
| | | AGGAGAUUUAUCAUGGCAUCCUGCCUUGACCACUAAGAGUAAAAU |
| | | GCGAGAUCCACAUUCCCAUGCAUUUAUUGAUCUGACUGAAGAUUU |
| | | UACAGCAGAUUUAUUCCUCACGACAUUGACUGCCUCUAAUGCCUU |
| | | CCAGUUUGAGAUAUGGGAAAAUUUGGGUGGAAACUUCUCUAUCCA |
| | | UAGUGUCUUUGAAAAACCUAAAAAUCUGGUGGUGGUUGGACAGUC |
| | | AGCAUUUGCAGACUUUGAUGGAGAUGGACACAUGGAUCACUUGCU |
| | | GCCAGGCUGUGAAGAUAAGGACUGUCAGAAAAGUGCCAUAUAUUU |
| | | AAUGAGAUCUGGAACAGGACAGUGGGUUCCUGUGCUUCAGGAUUU |
| | | UAGCAAUAAGGGUACACUCUGGGGCUUUGUGCCAUUUGUGCAUGA |
| | | AGAACAACCAACCACAAUACCAAUCCCACUCACACUUCACAUUGG |
| | | AGACUACAACAUGGACGGCUACCCAGAUGCUCUGGCUAUAUUGAA |
| | | GAAUACAUCUGGAAGCAAUCAGCAAGCCUUUUUACUGGAGAACGU |
| | | CCCAUGCAAUAAUGCGAGCUGCGAAGAAGUGCAUCGGAUGUUUAA |
| | | AGUCUACUGGGACCUAGCAGGCCUAAAUCUAAUCAAAGACGCCAU |
| | | AGUUGCCACCUUUUUUGAUAUUUAUGAAGAUGGAAUCUUGGACAU |
| | | UAUAGUCCUCAGUAAAGGAUACACCAAGAAUGAUGUUGCCAUCCA |
| | | UACACUCAAAAAUAACUUUGAGGCAGAUGCUUACUUUGUUAAAGU |
| | | CAUUGUUCUUAGUGGUCUAUGUUCUAAUGACUGUCCUCGUAAGAU |
| | | AACACCUUUUGGAGUAAAUCAGCCUGGACCUUAUAUCAUGUACAC |
| | | AACAGUAGAUGCAAAUGGAUAUCUGAAAAACGGCUCAGCUGGACA |
| | | GCUCAGCCAGUCAGCACACUUAGCUCUUCAGCUACCUUACAAUGU |
| | | ACUUGGCUUAGGCCGAAGUGCCAAUUUCCUGGAUCAUCUUUUUGU |
| | | UGGUAUUCCCCGUCCAUCUGGAGAGAAGUCUAUAAGAAAACAAGA |
| | | GUGGACAGCAAUCAUUCCAAAUUCCCAGCUGAUUGUCAUUCCAUA |
| | | UCCUCACAAUGUCCCUCGAAGCUGGAGUGCCAAAUUGUACCUGAC |
| | | ACCAAGUAACAUUGUCCUGCUGACUGCGGUAGCCCUCAUUGGAGU |
| | | CUGCAUUUUUAUCCUGGCAAUAAUCGCCAUUUUACAUUGGCAGGA |
| | | AAAGAAAGCCGAUGACAGAGAAAAACGACAGGAAGCACAUCGGUU |
| | | UCAUUUUGAUGCUAUGUGACUUUGCUGAAGUAGUACAUCCUGGAA |
| | | AUCACUUGAUUAGUGAAACAUUAAAUUUUGACUCCUAGUUUAAAA |
| | | AAAAUGAUGGAUCUGAGAUAUUUAUAAAUUAUGCCUUCCUUGUCA |
| | | AUUAUUGGAAAAUACUGAAAUAAAUAUGGCCUGAAUAUCUCAUAA |
| | | AGUCUGUUUUAAGCACUUUGUAUAUAAUAAUUUAGGUUCUUAAUU |
| | | UAGCAGCACUGAGCAUUUGUGUUCCUUUGUGGAAUGUGUUGCAUG |
| | | UACUUGGUGUAUCUGUGUUUACAAUGUUGUUAGACUGAGGAUGGU |
| | | GAAAGGACGUGUAAAUAAAAACUAUUUAAAUGACCUGUGGCCAGG |
| | | GCGUUGUUCUGCCUAAAUUGAUCUUGUUUUUAUCUUUAAAAUGCA |
| | | UACUUUCACAAAUAUGUGCCAGAUAAAGUUAUUCAAGUAACAUUC |
| | | UUCACUGCAGAUCAGACUGCUCAUGCGUGGCACAGAAUGAAACUA |
| | | AAUAGCCAGGCUUUGGAAUCAUAGGAGGACAGCUUGCCUUAGAUG |
| | | CCUGGUUCUACAACUUAGAGUUAUCUGAAUAUGGCUGCUGCUUAG |
| | | CGUCUCCGUAAGCCUCAGUUUUGUUUGUAUGAUGAGGCUAAUAUG |
| | | CUUGUUCACUUGAGUUGUCAUGAGCAUACAUUGUAUGAUGUACAU |
| | | GAUGUACUUAGCUCAGUGUCUGAGCAUACAGAAAGCACUCGUCAA |
| | | GCAGCUGCUGUCAUGAUUGCUACUGUAGGUGAUCUUAUUCUACUA |
| | | CUGUAUACAAUGGAUUGUCACCGAAGACAUAAGGACUCUUUGGAA |
| | | AACUUAAAUUAUUGGUGGUACAUAUCUUACAUAGUUAACUUGUUU |
| | | AACUAGCUCUAAUACUAUCCAAGAGUAAAUGUAUCCUGAAAGCUG |
| | | GUAUUUGAAUACUAAUCCUUGCACAGUAACAUAAAUGAUUUGCUC |
| | | UCUUGAAAGAAAGCUGAUAGAGGCAAACUCUUCAGGUGCCAGCAU |
| | | GCCAUACAGUUCUGUCUCAAAAGACGGGAAAGGUCUGUCCCAUUU |
| | | CUACAGACCUCAGGUUUCAUGUUAGAAACUUCACAUUGCCAACAU |
| | | UUACUUUAAAAUUAUUUAAAUCUAGAUUUCAGGAGAGAGGUGAUA |
| | | AAAUUAUUAUCAAAGCUAACAAUAUGCUGAUUGUUAAGACAUGCU |
| | | GUGGCUGUAAAAUAUAAAAUUCCUCUGGAACAGUUUUGUUUGUGU |
| | | UAUUUUCCUCACUGCGAUUCACUGGAGGGCAUGUGCUCCUUUUUA |
| | | UUUCUAUCUAAGCAUGUUAAUAUACCUUCUUAAUAAAUACUCCUU |
| | | ACUGAAUAAAAACGCUGUGGCAAGC |
| 13 | ITFG1-001 Target | CUGACUGAAGAUUUUACAGCA |
| 14 | ITFG1-002 Target | AAUGACUGUCCUCGUAAGAUA |
| 15 | ITFG1-003 Target | AUGACUGUCCUCGUAAGAUAA |
| 16 | ITFG1-004 Target | UUACAUUGGCAGGAAAAGAAA |
| 17 | ITFG1-005 Target | CUUCGUGCUGCGGGAAAGAAA |
| 18 | ITFG1-006 Target | UUCUAAUGACUGUCCUCGUAA |
| 19 | ITFG1-007 Target | GAAGAUUUUACAGCAGAUUUA |
| 20 | ITFG1-008 Target | GUGGUUGGACAGUCAGCAUUU |
| 21 | ITFG1-009 Target | ACUGAAGAUUUUACAGCAGAU |
| 22 | ITFG1-010 Target | UUGUGGAAUGUGUUGCAUGUA |
| 23 | ITFG1-011 Target | CAGUCAGCAUUUGCAGACUUU |
| 24 | ITFG1-012 Target | UUGAUCUGACUGAAGAUUUUA |
| 25 | ITFG1-013 Target | CUGAAGAUUUUACAGCAGAUU |
| 26 | ITFG1-014 Target | AAUGGUGAUCUAAUUCCUGAU |
| 27 | ITFG1-015 Target | GACUGAAGAUUUUACAGCAGA |
| 28 | ITFG1-016 Target | GUGGAAUGUGUUGCAUGUACU |
| 29 | ITFG1-017 Target | UUGGACAGUCAGCAUUUGCAG |
| 30 | ITFG1-018 Target | UUUACAUUGGCAGGAAAAGAA |
| 31 | ITFG1-019 Target | UUUCAUUUUGAUGCUAUGUGA |
| 32 | ITFG1-020 Target | AUGGAGAUGGACACAUGGAUC |
| 33 | ITFG1-021 Target | AAGAUUUUACAGCAGAUUUAU |
| 34 | ITFG1-022 Target | UGCAUUUAUUGAUCUGACUGA |
| 35 | ITFG1-023 Target | UGGUGGUUGGACAGUCAGCAU |
| 36 | ITFG1-024 Target | GCAGACUUUGAUGGAGAUGGA |
| 37 | ITFG1-025 Target | AUUGAUCUGACUGAAGAUUUU |
| 38 | ITFG1-026 Target | CAAUGGUGAUCUAAUUCCUGA |
| 39 | ITFG1-027 Target | UUCAAUGGUGAUCUAAUUCCU |
| 40 | ITFG1-028 Target | CAUUUUACAUUGGCAGGAAAA |
| 41 | ITFG1-029 Target | UUGGUAUCACAAAUGAAUCCA |
| 42 | ITFG1-030 Target | CAGCAUUUGCAGACUUUGAUG |
| 43 | ITFG1-031 Target | UGGAGAUGGACACAUGGAUCA |
| 44 | ITFG1-032 Target | UCCUUUGUGGAAUGUGUUGCA |
| 45 | ITFG1-001 Antisense | UGCUGUAAAAUCUUCAGUCAGUU |
| 46 | ITFG1-002 Antisense | UAUCUUACGAGGACAGUCAUUUU |
| 47 | ITFG1-003 Antisense | UUAUCUUACGAGGACAGUCAUUU |
| 48 | ITFG1-004 Antisense | UUUCUUUUCCUGCCAAUGUAAUU |
| 49 | ITFG1-005 Antisense | UUUCUUUCCCGCAGCACGAAGUU |
| 50 | ITFG1-006 Antisense | UUACGAGGACAGUCAUUAGAAUU |
| 51 | ITFG1-007 Antisense | UAAAUCUGCUGUAAAAUCUUCUU |
| 52 | ITFG1-008 Antisense | AAAUGCUGACUGUCCAACCACUU |
| 53 | ITFG1-009 Antisense | AUCUGCUGUAAAAUCUUCAGUUU |
| 54 | ITFG1-010 Antisense | UACAUGCAACACAUUCCACAAUU |
| 55 | ITFG1-011 Antisense | AAAGUCUGCAAAUGCUGACUGUU |
| 56 | ITFG1-012 Antisense | UAAAAUCUUCAGUCAGAUCAAUU |
| 57 | ITFG1-013 Antisense | AAUCUGCUGUAAAAUCUUCAGUU |
| 58 | ITFG1-014 Antisense | AUCAGGAAUUAGAUCACCAUUUU |
| 59 | ITFG1-015 Antisense | UCUGCUGUAAAAUCUUCAGUCUU |
| 60 | ITFG1-016 Antisense | AGUACAUGCAACACAUUCCACUU |
| 61 | ITFG1-017 Antisense | CUGCAAAUGCUGACUGUCCAAUU |
| 62 | ITFG1-018 Antisense | UUCUUUUCCUGCCAAUGUAAAUU |
| 63 | ITFG1-019 Antisense | UCACAUAGCAUCAAAAUGAAAUU |
| 64 | ITFG1-020 Antisense | GAUCCAUGUGUCCAUCUCCAUUU |
| 65 | ITFG1-021 Antisense | AUAAAUCUGCUGUAAAAUCUUUU |
| 66 | ITFG1-022 Antisense | UCAGUCAGAUCAAUAAAUGCAUU |
| 67 | ITFG1-023 Antisense | AUGCUGACUGUCCAACCACCAUU |
| 68 | ITFG1-024 Antisense | UCCAUCUCCAUCAAAGUCUGCUU |
| 69 | ITFG1-025 Antisense | AAAAUCUUCAGUCAGAUCAAUUU |
| 70 | ITFG1-026 Antisense | UCAGGAAUUAGAUCACCAUUGUU |
| 71 | ITFG1-027 Antisense | AGGAAUUAGAUCACCAUUGAAUU |
| 72 | ITFG1-028 Antisense | UUUUCCUGCCAAUGUAAAAUGUU |
| 73 | ITFG1-029 Antisense | UGGAUUCAUUUGUGAUACCAAUU |
| 74 | ITFG1-030 Antisense | CAUCAAAGUCUGCAAAUGCUGUU |
| 75 | ITFG1-031 Antisense | UGAUCCAUGUGUCCAUCUCCAUU |
| 76 | ITFG1-032 Antisense | UGCAACACAUUCCACAAAGGAUU |
| 77 | ITFG1-001 Sense | CUGACUGAAGAUUUUACAGCA |
| 78 | ITFG1-002 Sense | AAUGACUGUCCUCGUAAGAUA |
| 79 | ITFG1-003 Sense | AUGACUGUCCUCGUAAGAUAA |
| 80 | ITFG1-004 Sense | UUACAUUGGCAGGAAAAGAAA |
| 81 | ITFG1-005 Sense | CUUCGUGCUGCGGGAAAGAAA |
| 82 | ITFG1-006 Sense | UUCUAAUGACUGUCCUCGUAA |
| 83 | ITFG1-007 Sense | GAAGAUUUUACAGCAGAUUUA |
| 84 | ITFG1-008 Sense | GUGGUUGGACAGUCAGCAUUU |
| 85 | ITFG1-009 Sense | ACUGAAGAUUUUACAGCAGAU |
| 86 | ITFG1-010 Sense | UUGUGGAAUGUGUUGCAUGUA |
| 87 | ITFG1-011 Sense | CAGUCAGCAUUUGCAGACUUU |
| 88 | ITFG1-012 Sense | UUGAUCUGACUGAAGAUUUUA |
| 89 | ITFG1-013 Sense | CUGAAGAUUUUACAGCAGAUU |
| 90 | ITFG1-014 Sense | AAUGGUGAUCUAAUUCCUGAU |
| 91 | ITFG1-015 Sense | GACUGAAGAUUUUACAGCAGA |
| 92 | ITFG1-016 Sense | GUGGAAUGUGUUGCAUGUACU |
| 93 | ITFG1-017 Sense | UUGGACAGUCAGCAUUUGCAG |
| 94 | ITFG1-018 Sense | UUUACAUUGGCAGGAAAAGAA |
| 95 | ITFG1-019 Sense | UUUCAUUUUGAUGCUAUGUGA |
| 96 | ITFG1-020 Sense | AUGGAGAUGGACACAUGGAUC |
| 97 | ITFG1-021 Sense | AAGAUUUUACAGCAGAUUUAU |
| 98 | ITFG1-022 Sense | UGCAUUUAUUGAUCUGACUGA |
| 99 | ITFG1-023 Sense | UGGUGGUUGGACAGUCAGCAU |
| 100 | ITFG1-024 Sense | GCAGACUUUGAUGGAGAUGGA |
| 101 | ITFG1-025 Sense | AUUGAUCUGACUGAAGAUUUU |
| 102 | ITFG1-026 Sense | CAAUGGUGAUCUAAUUCCUGA |
| 103 | ITFG1-027 Sense | UUCAAUGGUGAUCUAAUUCCU |
| 104 | ITFG1-028 Sense | CAUUUUACAUUGGCAGGAAAA |
| 105 | ITFG1-029 Sense | UUGGUAUCACAAAUGAAUCCA |
| 106 | ITFG1-030 Sense | CAGCAUUUGCAGACUUUGAUG |
| 107 | ITFG1-031 Sense | UGGAGAUGGACACAUGGAUCA |
| 108 | ITFG1-032 Sense | UCCUUUGUGGAAUGUGUUGCA |
| 109 | ITFG1-014 Sense M04 | AAUGGUGAUGUAAUUCCUGAU |
| 110 | ITFG1-014 Sense M05 | AAUGGUGAUAUAAUUCCUGAU |
| 111 | ITFG1-014 Sense M06 | AAUGGUGAUUUAAUUCCUGAU |
| 112 | ITFG1-021 Sense M04 | AAGAUUUUAGAGCAGAUUUAU |
| 113 | ITFG1-021 Sense M05 | AAGAUUUUAAAGCAGAUUUAU |
| 114 | ITFG1-021 Sense M06 | AAGAUUUUAUAGCAGAUUUAU |
| 115 | ITFG1-001 dsiRNA | AUCUGACUGAAGAUUUUACAGCAGAUU |
| | Target | |
| 116 | ITFG1-002 dsiRNA | CUAAUGACUGUCCUCGUAAGAUAACAC |
| | Target | |
| 117 | ITFG1-003 dsiRNA | UAAUGACUGUCCUCGUAAGAUAACACC |
| | Target | |
| 118 | ITFG1-004 dsiRNA | UUUUACAUUGGCAGGAAAAGAAAGCAG |
| | Target | |
| 119 | ITFG1-005 dsiRNA | CUCUUCGUGCUGCGGGAAAGAAAUGAC |
| | Target | |
| 120 | ITFG1-006 dsiRNA | UGUUCUAAUGACUGUCCUCGUAAGAUA |
| | Target | |
| 121 | ITFG1-007 dsiRNA | CUGAAGAUUUUACAGCAGAUUUAUUCC |
| | Target | |
| 122 | ITFG1-008 dsiRNA | UGGUGGUUGGACAGUCAGCAUUUGCAG |
| | Target | |
| 123 | ITFG1-009 dsiRNA | UGACUGAAGAUUUUACAGCAGAUUUAU |
| | Target | |
| 124 | ITFG1-010 dsiRNA | CUUUGUGGAAUGUGUUGCAUGUACUCC |
| | Target | |
| 125 | ITFG1-011 dsiRNA | GACAGUCAGCAUUUGCAGACUUUGAUG |
| | Target | |
| 126 | ITFG1-012 dsiRNA | UAUUGAUCUGACUGAAGAUUUUACAGC |
| | Target | |
| 127 | ITFG1-013 dsiRNA | GACUGAAGAUUUUACAGCAGAUUUAUU |
| | Target | |
| 128 | ITFG1-014 dsiRNA | UCAAUGGUGAUCUAAUUCCUGAUAUUU |
| | Target | |
| 129 | ITFG1-015 dsiRNA | CUGACUGAAGAUUUUACAGCAGAUUUA |
| | Target | |
| 130 | ITFG1-016 dsiRNA | UUGUGGAAUGUGUUGCAUGUACUCCAG |
| | Target | |
| 131 | ITFG1-017 dsiRNA | GGUUGGACAGUCAGCAUUUGCAGACUU |
| | Target | |
| 132 | ITFG1-018 dsiRNA | AUUUUACAUUGGCAGGAAAAGAAAGCA |
| | Target | |
| 133 | ITFG1-019 dsiRNA | GGUUUCAUUUUGAUGCUAUGUGACUUG |
| | Target | |
| 134 | ITFG1-020 dsiRNA | UGAUGGAGAUGGACACAUGGAUCAUUU |
| | Target | |
| 135 | ITFG1-021 dsiRNA | UGAAGAUUUUACAGCAGAUUUAUUCCU |
| | Target | |
| 136 | ITFG1-022 dsiRNA | CAUGCAUUUAUUGAUCUGACUGAAGAU |
| | Target | |
| 137 | ITFG1-023 dsiRNA | GAUGGUGGUUGGACAGUCAGCAUUUGC |
| | Target | |
| 138 | ITFG1-024 dsiRNA | UUGCAGACUUUGAUGGAGAUGGACACA |
| | Target | |
| 139 | ITFG1-025 dsiRNA | UUAUUGAUCUGACUGAAGAUUUUACAG |
| | Target | |
| 140 | ITFG1-026 dsiRNA | UUCAAUGGUGAUCUAAUUCCUGAUAUU |
| | Target | |
| 141 | ITFG1-027 dsiRNA | AUUUCAAUGGUGAUCUAAUUCCUGAUA |
| | Target | |
| 142 | ITFG1-028 dsiRNA | GGCAUUUUACAUUGGCAGGAAAAGAAA |
| | Target | |
| 143 | ITFG1-029 dsiRNA | UUUUGGUAUCACAAAUGAAUCCAACCA |
| | Target | |
| 144 | ITFG1-030 dsiRNA | GUCAGCAUUUGCAGACUUUGAUGGAGA |
| | Target | |
| 145 | ITFG1-031 dsiRNA | GAUGGAGAUGGACACAUGGAUCAUUUA |
| | Target | |
| 146 | ITFG1-032 dsiRNA | GUUCCUUUGUGGAAUGUGUUGCAUGUA |
| | Target | |
| 147 | ITFG1-001 dsiRNA | AAUCUGCUGUAAAAUCUUCAGUCAGAU |
| | Antisense | |
| 148 | ITFG1-002 dsiRNA | GUGUUAUCUUACGAGGACAGUCAUUAG |
| | Antisense | |
| 149 | ITFG1-003 dsiRNA | GGUGUUAUCUUACGAGGACAGUCAUUA |
| | Antisense | |
| 150 | ITFG1-004 dsiRNA | CUGCUUUCUUUUCCUGCCAAUGUAAAA |
| | Antisense | |
| 151 | ITFG1-005 dsiRNA | GUCAUUUCUUUCCCGCAGCACGAAGAG |
| | Antisense | |
| 152 | ITFG1-006 dsiRNA | UAUCUUACGAGGACAGUCAUUAGAACA |
| | Antisense | |
| 153 | ITFG1-007 dsiRNA | GGAAUAAAUCUGCUGUAAAAUCUUCAG |
| | Antisense | |
| 154 | ITFG1-008 dsiRNA | CUGCAAAUGCUGACUGUCCAACCACCA |
| | Antisense | |
| 155 | ITFG1-009 dsiRNA | AUAAAUCUGCUGUAAAAUCUUCAGUCA |
| | Antisense | |
| 156 | ITFG1-010 dsiRNA | GGAGUACAUGCAACACAUUCCACAAAG |
| | Antisense | |
| 157 | ITFG1-011 dsiRNA | CAUCAAAGUCUGCAAAUGCUGACUGUC |
| | Antisense | |
| 158 | ITFG1-012 dsiRNA | GCUGUAAAAUCUUCAGUCAGAUCAAUA |
| | Antisense | |
| 159 | ITFG1-013 dsiRNA | AAUAAAUCUGCUGUAAAAUCUUCAGUC |
| | Antisense | |
| 160 | ITFG1-014 dsiRNA | AAAUAUCAGGAAUUAGAUCACCAUUGA |
| | Antisense | |
| 161 | ITFG1-015 dsiRNA | UAAAUCUGCUGUAAAAUCUUCAGUCAG |
| | Antisense | |
| 162 | ITFG1-016 dsiRNA | CUGGAGUACAUGCAACACAUUCCACAA |
| | Antisense | |
| 163 | ITFG1-017 dsiRNA | AAGUCUGCAAAUGCUGACUGUCCAACC |
| | Antisense | |
| 164 | ITFG1-018 dsiRNA | UGCUUUCUUUUCCUGCCAAUGUAAAAU |
| | Antisense | |
| 165 | ITFG1-019 dsiRNA | CAAGUCACAUAGCAUCAAAAUGAAACC |
| | Antisense | |
| 166 | ITFG1-020 dsiRNA | AAAUGAUCCAUGUGUCCAUCUCCAUCA |
| | Antisense | |
| 167 | ITFG1-021 dsiRNA | AGGAAUAAAUCUGCUGUAAAAUCUUCA |
| | Antisense | |
| 168 | ITFG1-022 dsiRNA | AUCUUCAGUCAGAUCAAUAAAUGCAUG |
| | Antisense | |
| 169 | ITFG1-023 dsiRNA | GCAAAUGCUGACUGUCCAACCACCAUC |
| | Antisense | |
| 170 | ITFG1-024 dsiRNA | UGUGUCCAUCUCCAUCAAAGUCUGCAA |
| | Antisense | |
| 171 | ITFG1-025 dsiRNA | CUGUAAAAUCUUCAGUCAGAUCAAUAA |
| | Antisense | |
| 172 | ITFG1-026 dsiRNA | AAUAUCAGGAAUUAGAUCACCAUUGAA |
| | Antisense | |
| 173 | ITFG1-027 dsiRNA | UAUCAGGAAUUAGAUCACCAUUGAAAU |
| | Antisense | |
| 174 | ITFG1-028 dsiRNA | UUUCUUUUCCUGCCAAUGUAAAAUGCC |
| | Antisense | |
| 175 | ITFG1-029 dsiRNA | UGGUUGGAUUCAUUUGUGAUACCAAAA |
| | Antisense | |
| 176 | ITFG1-030 dsiRNA | UCUCCAUCAAAGUCUGCAAAUGCUGAC |
| | Antisense | |
| 177 | ITFG1-031 dsiRNA | UAAAUGAUCCAUGUGUCCAUCUCCAUC |
| | Antisense | |
| 178 | ITFG1-032 dsiRNA | UACAUGCAACACAUUCCACAAAGGAAC |
| | Antisense | |
| 179 | ITFG1-001 dsiRNA | CUGACUGAAGAUUUUACAGCAGATT |
| | Sense | |
| 180 | ITFG1-002 dsiRNA | AAUGACUGUCCUCGUAAGAUAACAC |
| | Sense | |
| 181 | ITFG1-003 dsiRNA | AUGACUGUCCUCGUAAGAUAACACC |
| | Sense | |
| 182 | ITFG1-004 dsiRNA | UUACAUUGGCAGGAAAAGAAAGCAG |
| | Sense | |
| 183 | ITFG1-005 dsiRNA | CUUCGUGCUGCGGGAAAGAAAUGAC |
| | Sense | |
| 184 | ITFG1-006 dsiRNA | UUCUAAUGACUGUCCUCGUAAGATA |
| | Sense | |
| 185 | ITFG1-007 dsiRNA | GAAGAUUUUACAGCAGAUUUAUUCC |
| | Sense | |
| 186 | ITFG1-008 dsiRNA | GUGGUUGGACAGUCAGCAUUUGCAG |
| | Sense | |
| 187 | ITFG1-009 dsiRNA | ACUGAAGAUUUUACAGCAGAUUUAT |
| | Sense | |
| 188 | ITFG1-010 dsiRNA | UUGUGGAAUGUGUUGCAUGUACUCC |
| | Sense | |
| 189 | ITFG1-011 dsiRNA | CAGUCAGCAUUUGCAGACUUUGATG |
| | Sense | |
| 190 | ITFG1-012 dsiRNA | UUGAUCUGACUGAAGAUUUUACAGC |
| | Sense | |
| 191 | ITFG1-013 dsiRNA | CUGAAGAUUUUACAGCAGAUUUATT |
| | Sense | |
| 192 | ITFG1-014 dsiRNA | AAUGGUGAUCUAAUUCCUGAUAUTT |
| | Sense | |
| 193 | ITFG1-015 dsiRNA | GACUGAAGAUUUUACAGCAGAUUTA |
| | Sense | |
| 194 | ITFG1-016 dsiRNA | GUGGAAUGUGUUGCAUGUACUCCAG |
| | Sense | |
| 195 | ITFG1-017 dsiRNA | UUGGACAGUCAGCAUUUGCAGACTT |
| | Sense | |
| 196 | ITFG1-018 dsiRNA | UUUACAUUGGCAGGAAAAGAAAGCA |
| | Sense | |
| 197 | ITFG1-019 dsiRNA | UUUCAUUUUGAUGCUAUGUGACUTG |
| | Sense | |
| 198 | ITFG1-020 dsiRNA | AUGGAGAUGGACACAUGGAUCAUTT |
| | Sense | |
| 199 | ITFG1-021 dsiRNA | AAGAUUUUACAGCAGAUUUAUUCCT |
| | Sense | |
| 200 | ITFG1-022 dsiRNA | UGCAUUUAUUGAUCUGACUGAAGAT |
| | Sense | |
| 201 | ITFG1-023 dsiRNA | UGGUGGUUGGACAGUCAGCAUUUGC |
| | Sense | |
| 202 | ITFG1-024 dsiRNA | GCAGACUUUGAUGGAGAUGGACACA |
| | Sense | |
| 203 | ITFG1-025 dsiRNA | AUUGAUCUGACUGAAGAUUUUACAG |
| | Sense | |
| 204 | ITFG1-026 dsiRNA | CAAUGGUGAUCUAAUUCCUGAUATT |
| | Sense | |
| 205 | ITFG1-027 dsiRNA | UUCAAUGGUGAUCUAAUUCCUGATA |
| | Sense | |
| 206 | ITFG1-028 dsiRNA | CAUUUUACAUUGGCAGGAAAAGAAA |
| | Sense | |
| 207 | ITFG1-029 dsiRNA | UUGGUAUCACAAAUGAAUCCAACCA |
| | Sense | |
| 208 | ITFG1-030 dsiRNA | CAGCAUUUGCAGACUUUGAUGGAGA |
| | Sense | |
| 209 | ITFG1-031 dsiRNA | UGGAGAUGGACACAUGGAUCAUUTA |
| | Sense | |
| 210 | ITFG1-032 dsiRNA | UCCUUUGUGGAAUGUGUUGCAUGTA |
| | Sense | |
| 211 | CG_200167 | (smA)(sfU)(mC)(fA)(mG)(fG)(mA)(fA)(mU)(fU)(mA |
| | | )(fG)(mA)(fU)(mC)(fA)(mC)(mC)(mA)(mU)(smU)(sm |
| | | U)(mU) |
| 212 | CG_200168 | (smA)(sfU)(mC)(fA)(mG)(fG)(mA)(fA)(mU)(mU)(mA |
| | | )(fG)(mA)(fU)(mC)(fA)(mC)(mC)(mA)(mU)(smU)(sm |
| | | U)(mU) |
| 213 | CG_200169 | (smA)(sfU)(mC)(fA)(mG)(fG)(mA)(fA)(mU)(fU)(mA |
| | | )(mG)(mA)(fU)(mC)(fA)(mC)(mC)(mA)(mU)(smU)(sm |
| | | U)(mU) |
| 214 | CG_200170 | (smA)(sfU)(mC)(fA)(mG)(fG)(mA)(fA)(mU)(mU)(mA |
| | | )(mG)(mA)(fU)(mC)(fA)(mC)(mC)(mA)(mU)(smU)(sm |
| | | U)(mU) |
| 215 | CG_200171 | (smA)(sfU)(mC)(mA)(mG)(fG)(mA)(fA)(mU)(fU)(mA |
| | | )(fG)(mA)(fU)(mC)(fA)(mC)(mC)(mA)(mU)(smU)(sm |
| | | U)(mU) |
| 216 | CG_200172 | (smA)(sfU)(mC)(fA)(mG)(fG)(mA)(mA)(mU)(fU)(mA |
| | | )(fG)(mA)(fU)(mC)(fA)(mC)(mC)(mA)(mU)(smU)(sm |
| | | U)(mU) |
| 217 | CG_200173 | (smA)(sfU)(mC)(mA)(mG)(mG)(mA)(fA)(mU)(fU)(mA |
| | | )(fG)(mA)(fU)(mC)(fA)(mC)(mC)(mA)(mU)(smU)(sm |
| | | U)(mU) |
| 218 | CG_200174 | (smA)(sfU)(mC)(mA)(mG)(fG)(mA)(mA)(mU)(fU)(mA |
| | | )(fG)(mA)(fU)(mC)(fA)(mC)(mC)(mA)(mU)(smU)(sm |
| | | U)(mU) |
| 219 | CG_200175 | (smA)(smU)(mC)(mA)(fG)(mG)(fA)(mA)(mU)(fU)(mA |
| | | )(fG)(mA)(fU)(mC)(fA)(mC)(mC)(mA)(mU)(smU)(sm |
| | | U)(mU) |
| 220 | CG_200176 | (smA)(sfU)(mC)(mA)(mG)(mG)(mA)(mA)(mU)(fU)(mA |
| | | )(fG)(mA)(fU)(mC)(fA)(mC)(mC)(mA)(mU)(smU)(sm |
| | | U)(mU) |
| 221 | CG_200177 | (smA)(sfU)(mA)(fA)(mA)(fU)(mC)(fU)(mG)(fC)(mU |
| | | )(fG)(mU)(fA)(mA)(fA)(mA)(mU)(mC)(mU)(smU)(sm |
| | | U)(mU) |
| 222 | CG_200178 | (smA)(sfU)(mA)(fA)(mA)(fU)(mC)(fU)(mG)(mC)(mU |
| | | )(fG)(mU)(fA)(mA)(fA)(mA)(mU)(mC)(mU)(smU)(sm |
| | | U)(mU) |
| 223 | CG_200179 | (smA)(sfU)(mA)(fA)(mA)(fU)(mC)(fU)(mG)(fC)(mU |
| | | )(mG)(mU)(fA)(mA)(fA)(mA)(mU)(mC)(mU)(smU)(sm |
| | | U)(mU) |
| 224 | CG_200180 | (smA)(sfU)(mA)(fA)(mA)(fU)(mC)(fU)(mG)(mC)(mU |
| | | )(mG)(mU)(fA)(mA)(fA)(mA)(mU)(mC)(mU)(smU)(sm |
| | | U)(mU) |
| 225 | CG_200181 | (smA)(sfU)(mA)(mA)(mA)(fU)(mC)(fU)(mG)(fC)(mU |
| | | )(fG)(mU)(fA)(mA)(fA)(mA)(mU)(mC)(mU)(smU)(sm |
| | | U)(mU) |
| 226 | CG_200182 | (smA)(sfU)(mA)(fA)(mA)(fU)(mC)(mU)(mG)(fC)(mU |
| | | )(fG)(mU)(fA)(mA)(fA)(mA)(mU)(mC)(mU)(smU)(sm |
| | | U)(mU) |
| 227 | CG_200183 | (smA)(sfU)(mA)(mA)(mA)(mU)(mC)(fU)(mG)(fC)(mU |
| | | )(fG)(mU)(fA)(mA)(fA)(mA)(mU)(mC)(mU)(smU)(sm |
| | | U)(mU) |
| 228 | CG_200184 | (smA)(sfU)(mA)(mA)(mA)(fU)(mC)(mU)(mG)(fC)(mU |
| | | )(fG)(mU)(fA)(mA)(fA)(mA)(mU)(mC)(mU)(smU)(sm |
| | | U)(mU) |
| 229 | CG_200185 | (smA)(smU)(mA)(mA)(fA)(mU)(fC)(mU)(mG)(fC)(mU |
| | | )(fG)(mU)(fA)(mA)(fA)(mA)(mU)(mC)(mU)(smU)(sm |
| | | U)(mU) |
| 230 | CG_200186 | (smA)(sfU)(mA)(mA)(mA)(mU)(mC)(mU)(mG)(fC)(mU |
| | | )(fG)(mU)(fA)(mA)(fA)(mA)(mU)(mC)(mU)(smU)(sm |
| | | U)(mU) |
| 231 | CG_200187 | (smA)(smA)(mU)(mG)(mG)(mU)(fG)(mA)(fU)(fC)(fU |
| | | )(mA)(mA)(mU)(mU)(mC)(mC)(mU)(smG)(smA)(mU) |
| 232 | CG_200188 | (mAps)(mAps)(mU)(mG)(mG)(mU)(fG)(mA)(fU)(fG)( |
| | | fU)(mA)(mA)(mU)(mU)(mC)(mC)(mU)(mGps)(mAps)(m |
| | | U) |
| 233 | CG_200189 | (mAps)(mAps)(mU)(mG)(mG)(mU)(fG)(mA)(fU)(fA)( |
| | | fU)(mA)(mA)(mU)(mU)(mC)(mC)(mU)(mGps)(mAps)(m |
| | | U) |
| 234 | CG_200190 | (mAps)(mAps)(mU)(mG)(mG)(mU)(fG)(mA)(fU)(fU)( |
| | | fU)(mA)(mA)(mU)(mU)(mC)(mC)(mU)(mGps)(mAps)(m |
| | | U) |
| 235 | CG_200191 | (mAps)(mAps)(mU)(mG)(mG)(mU)(fG)(mA)(fU)(fU)( |
| | | fU)(mA)(mA)(mU)(mU)(mC)(mC)(mU)(mG)(mA)(mU)(t |
| | | riGal) |
| 236 | CG_200192 | (smA)(smA)(mG)(mA)(mU)(mU)(fU)(mU)(fA)(fC)(fA |
| | | )(mG)(mC)(mA)(mG)(mA)(mU)(mU)(smU)(smA)(mU) |
| 237 | CG_200193 | (mAps)(mAps)(mG)(mA)(mU)(mU)(fU)(mU)(fA)(fG)( |
| | | fA)(mG)(mC)(mA)(mU)(mA)(mU)(mU)(mUps)(mAps)(m |
| | | U) |
| 238 | CG_200194 | (mAps)(mAps)(mG)(mA)(mU)(mU)(fU)(mU)(fA)(fA)( |
| | | fA)(mG)(mC)(mA)(mU)(mA)(mU)(mU)(mUps)(mAps)(m |
| | | U) |
| 239 | CG_200195 | (mAps)(mAps)(mG)(mA)(mU)(mU)(fU)(mU)(fA)(fU)( |
| | | fA)(mG)(mC)(mA)(mU)(mA)(mU)(mU)(mUps)(mAps)(m |
| | | U) |
| 240 | CG_200196 | (mAps)(mAps)(mG)(mA)(mU)(mU)(fU)(mU)(fA)(fC)( |
| | | fA)(mG)(mC)(mA)(mG)(mA)(mU)(mU)(mU)(mA)(mU)(t |
| | | riGal) |
| 241 | CG_200197 | (mAps)(mAps)(mU)(mG)(mG)(mU)(fG)(mA)(fU)(fU)( |
| | | fU)(mA)(mA)(mU)(mU)(mC)(mC)(mU)(mG)(mA)(mU)(p |
| | | tGalps)(ptGalps)(ptGal) |
| 242 | hslTFG1 qPCR F | TGATGGAGATGGACACATGG |
| | primer | |
| 243 | hslTFG1 qPCR R | CATCCACAAATGGCACAAAG |
| | primer | |
| 244 | mmltfg1 qPCR F | CAATGTCCCTCGAAGCTGGAG |
| | primer | |
| 245 | mmltfg1 qPCR R | TTCTCTGTCATCGGCTTTCTTTTC |
| | primer | |
| 246 | Ki67 qPCR F primer | AGAAACGCCAACCAAGAGGA |
| 247 | Ki67 qPCR R primer | GACTGGGGCCGTTCCTTGAT |
| 248 | Acta2 qPCR F primer | CTAAGGCCAACCGGGAGAAA |
| 249 | Acta2 qPCR R primer | GAGTCCAGCACAATACCAGT |
| 250 | rnltfg1 qPCR F primer | GCCGCAGATACTGTTAGGAGG |
| 251 | rnltfg1 qPCR R primer | GGATAGAGAAGTTTCCACCCAAA |
| 252 | mflTFG1 qPCR F | GAGCGCAAATTTTCTTGACC |
| | primer | |
| 253 | hmflTFG1 qPCR R | GGGACATTGTGAGGGTATGG |
| | primer | |
| 254 | CG_200191/ | (mAps)(mAps)(mU)(mG)(mG)(mU)(fG)(mA)(fU)(fU) |
| | CG_200197 [no | (fU)(mA)(mA)(mU)(mU)(mC)(mC)(mU)(mG)(mA)(mU) |
| | targeting moiety] | |
| 255 | SEQ ID NO: 254, tGal | (mAps)(mAps)(mU)(mG)(mG)(mU)(fG)(mA)(fU)(fU) |
| | | (fU)(mA)(mA)(mU)(mU)(mC)(mC)(mU)(mG)(mA)(mU) |
| | | (tGalps)(tGalps)(tGal) |
| 256 | SEQ ID NO: 254, gGal | (mAps)(mAps)(mU)(mG)(mG)(mU)(fG)(mA)(fU)(fU) |
| | | (fU)(mA)(mA)(mU)(mU)(mC)(mC)(mU)(mG)(mA)(mU) |
| | | (gGalps)(gGalps)(gGal) |
| 257 | SEQ ID NO: 254, mGal | (mAps)(mAps)(mU)(mG)(mG)(mU)(fG)(mA)(fU)(fU) |
| | | (fU)(mA)(mA)(mU)(mU)(mC)(mC)(mU)(mG)(mA)(mU) |
| | | (mGalps)(mGalps)(mGal) |

In the sequences of the table above, G=guanosine-3′-phosphate, C=cytidine-3′-phosphate, U=uridine-3′-phosphate, T=thymidine-3′-phosphate, A=adenosine-3′-phosphate, mU=2′-O-methyluridine-3′-phosphate, mA=2′-O-methyladenosine-3′-phosphate, mG=2′-O-methylguanosine-3′-phosphate, mC=2′-O-methylcytidine-3′-phosphate, smU=2′-O-methyluridine-3′-phosphorothioate, smA=2′-O-methyladenosine-3′-phosphorothioate, smG=2′-O-methylguanosine-3′-phosphorothioate, smC=2′-O-methylcytidine-3′-phosphorothioate, fU=2′-fluorouridine-3′-phosphate, fA=2′-fluoroadenosine-3′-phosphate, fG=2′-fluoroguanosine-3′-phosphate, fC=2′-fluorocytidine-3′-phosphate, sfC=2′-fluorocytidine-3′-phosphorothioate, sfG=2′-fluoroguanosine-3′-phosphorothioate, sfA=2′-fluoroadenosine-3′-phosphorothioate, sfU=2′-fluorouridine-3′-phosphorothioate; ps=phosphorothioate linkage, triGal=triantennary GalNAc moiety; ptGal=monomer of formula 3/3a, such as ‘GalNAc monomer 3’; tGal=monomer of formula 3/3a, such as ‘GalNAc monomer 5’; gGal=monomer of formula 2/2a/2b, such as ‘GalNAc monomer 4’; and mGal=monomer of formula 111 a/1b/1c, such as ‘GalNAc monomer 1’.

The invention includes the combination of the aspects and preferred features described except where such a combination is clearly impermissible or expressly avoided.

The section headings used herein are for organisational purposes only and are not to be construed as limiting the subject matter described.

Aspects and embodiments of the present disclosure will now be illustrated, by way of example, with reference to the accompanying figures. Further aspects and embodiments will be apparent to those skilled in the art. All documents mentioned in this text are incorporated herein by reference.

Throughout this specification, including the claims which follow, unless the context requires otherwise, the word ‘comprise,’ and variations such as ‘comprises’ and ‘comprising,’ will be understood to imply the inclusion of a stated integer or step or group of integers or steps but not the exclusion of any other integer or step or group of integers or steps.

It must be noted that, as used in the specification and the appended claims, the singular forms ‘a,’ ‘an,’ and ‘the’ include plural referents unless the context clearly dictates otherwise. Ranges may be expressed herein as from ‘about’ one particular value, and/or to ‘about’ another particular value. When such a range is expressed, another embodiment includes from the one particular value and/or to the other particular value. Similarly, when values are expressed as approximations, by the use of the antecedent ‘about,’ it will be understood that the particular value forms another embodiment.

Where a nucleic acid sequence is disclosed herein, the reverse complement thereof is also expressly contemplated.

Methods described herein may be performed in vitro or in vivo. In some embodiments, methods described herein are performed in vitro. The term ‘in vitro’ is intended to encompass experiments with cells in culture whereas the term ‘in vivo’ is intended to encompass experiments with intact multi-cellular organisms.

### BRIEF DESCRIPTION OF THE FIGURES

Embodiments and experiments illustrating the principles of the invention will now be discussed with reference to the accompanying figures.

FIG. 1. Bar chart showing knockdown of gene expression of ITFG1 in HuH7 cells by different dsiRNAs. HuH7 cells were transfected with the indicated dsiRNAs and analysed by qPCR for gene expression of ITFG1 as described in Example 2.

FIG. 2. Bar chart showing knockdown of gene expression of ITFG1 in HuH7 cells by the indicated siRNA and dsiRNA molecules. HuH7 cells were transfected with the indicated siRNAs and analysed by qPCR for gene expression of ITFG1 as described in Example 4.

FIG. 3. Bar chart showing knockdown of gene expression of ITFG1 in HuH7 cells by the indicated siRNA and dsiRNA molecules. HuH7 cells were transfected with the indicated siRNAs and analysed by qPCR for gene expression of ITFG1 as described in Example 4.

FIG. 4. Bar chart showing knockdown of gene expression of ITFG1 in HuH7 cells by the indicated siRNA and dsiRNA molecules. HuH7 cells were transfected with the indicated siRNAs and analysed by qPCR for gene expression of ITFG1 as described in Example 4.

FIG. 5. Photograph of a western blot showing knockdown of expression of ITFG1 at the protein level. HuH7 cells were transfected with the indicated siRNAs at the indicated concentrations and analysed by western blot using an anti-ITFG1 antibody in order to determine the level of expression at the protein level, as described in Example 6.

FIGS. 6A and 6B. Bar charts showing the effect of knockdown of expression of ITFG1 by the indicated siRNAs on proliferation of HuH7 cells. HuH7 cells were transfected with the indicated dsiRNAs and analysed by (6A) CellTiter-Glo Luminescent Cell Viability Assay, or (6B) CCK8 Assay in order to determine the effect on cell proliferation, as described in Example 7.

FIG. 7. Graph showing knockdown of expression of Itfg1 in vivo in mice. Mice were administered a single dose of the indicated siRNA at 2 mg/kg, and the level of expression of Itfg1 was analysed in samples obtained after the indicated number of days, as described in Example 10.

FIG. 8. Graph showing knockdown of expression of Itfg1 in vivo in mice. Mice were administered a single dose of the indicated siRNA at 2 mg/kg, 6 mg/kg or 10 mg/kg, and the level of expression of Itfg1 was subsequently analysed as described in Example 10.

FIGS. 9A and 9B. Graphs showing the results of evaluation of liver toxicity following administration of the indicated siRNAs to mice. Mice were administered a single dose of the indicated siRNA at 10 mg/kg, and the serum levels of (9A) aspartate transaminase (AST) and (9B) alanine aminotransferase (ALT) were subsequently analysed, as described in Example 11. Thresholds for toxicity are indicated.

FIG. 10. Graph showing the results of evaluation of knockdown of expression of Itfg1 in vivo in mice over time, following administration of a single, 6 mg/kg bodyweight dose of CG_200277 as described in Example 12.

FIG. 11. Graph showing knockdown of expression of Itfg1 in vivo in mice following administration of different doses of CG_200277 and CG_200280, as described in Example 12.

FIGS. 12A and 12B. Graphs showing the results of evaluation of liver toxicity following administration of multiple doses of CG_200277 to mice. Mice were administered multiple doses of CG_200277 at 20 mg/kg, and the serum levels of (12A) aspartate transaminase (AST) and (12B) alanine aminotransferase (ALT) were subsequently analysed, as described in Example 12. Circles=PBS, squares=CG_200277.

FIGS. 13A and 13B. Graph showing the results of Ki67-expressing (Ki67+) cells 42 hours following partial hepatectomy of CG_200277 or PBS-treated mice (13A). The livers were processed for immunostaining and the proportion of Ki67+ cells were analyzed as described in Example 15. Graphs showing the liver weights of CG_200280 or PBS-treated mice as absolute weights or as a ratio to the respective mouse's body weight (13B).

FIGS. 14A to 14I. Graphs showing various in vivo results in WFD-treated mice at baseline and after 12 additional weeks of CG_200277 or PBS treatment, following monthly administrations of 6 mg/kg bodyweight dose of CG_200277 or PBS as described in Example 16.2. (14A) Knockdown of expression of Itfg1 mRNA, (14B) Knockdown of expression of ITFG1 protein, (14C) quantification of ITFG1 protein, (14D) fibrosis score, (14E) steatosis score, (14F) expression of Ki67, (14G) expression of Acta2 mRNA, (14H) expression of Smooth-Muscle Actin protein, (14I) Albumin expression levels were evaluated as described in Example 16.2.

FIG. 15. Graph showing quantification of Picro Sirius Red-stained area in livers of mice fed CDHF Diet and additional 4 weeks of 6 mg/kg bodyweight dose of CG_200280 or PBS treatment. Livers were processed as described in Example 16.3.

FIGS. 16A to 16C. Graphs showing various in vivo results in mice that underwent bile duct ligation (BDL) surgery as described in Example 16.4. (16A) Graph showing body weights of the mice that received PBS or a single, 6 mg/kg bodyweight dose of CG_200277 for up to 13 days after bile duct ligation surgery. Circles=PBS, squares=CG_200277. (16B) Graphs showing sera correlates of liver toxicity PBS and CG_200277-treated groups (aspartate transaminase (AST) and alanine aminotransferase (ALT) and Total Bilirubin). (16C) Graph showing fibrosis scores of the liver of mice from PBS and CG_200277-treated groups.

FIGS. 17A to 17D. Graphs showing in vivo results of mice that received 6 and 12 repeated monthly injections of 6 mg/kg body weight of CG_200277 or PBS as described in Example 17. (17A) Graphs showing correlates of liver toxicity AST, ALT and albumin from the sera from mice treated for 6 months. (17B) Graphs showing wet liver weights and liver:body weight ratios of the mice treated for 6 months. (17C) Graphs showing correlates of liver toxicity ALT and AST from the sera from mice treated for 12 months. (17D) Graphs showing wet liver weights and liver:body weight ratios of the mice treated for 12 months.

FIGS. 18A to 18C. Schematic of an inhibitory nucleic acid according to the present disclosure conjugated with GalNAc monomers described herein. The waved line indicates a nucleotide chain.

FIGS. 19A to 19C. Schematic of a double-stranded inhibitory nucleic acid, e.g. siRNA, according to the present disclosure conjugated with GalNAc monomers described herein. The waved lines indicate nucleotide strands.

FIGS. 20A to 20C. Graphs showing knockdown of expression of Itfg1 in vivo in rats and cynomolgus monkeys following administration of PBS, 2 mg/kg or 6 mg/kg CG_200280, as described in Example 18. (20A) Graph showing relative Iftg1 expression in rat livers, harvested 7 days post-injection. (20B) Graph showing relative Iftg1 expression in rat kidneys, harvested 7 days post-injection. (20C) Graph showing relative Iftg1 expression in cynomolgus monkey livers, quantified from liver biopsies taken at 0, 14, 28, 56 and 84 days post-injection.

FIG. 21. Graphs showing the results of evaluation of liver toxicity in vivo in rats immediately prior to, and 48 hours after, administration of a single dose of CG_200280 (30 mg/kg, 100 mg/kg, or 400 mg/kg body weight).

### EXAMPLES

In the following Examples, the inventors describe the identification and characterisation of siRNA molecules for inhibiting gene and protein expression of ITFG1.

### Example 1: Inhibitory Nucleic Acids for RNAi-Mediated Knockdown of ITFG1 Expression

siRNAs targeting different regions of mRNA encoding human ITFG1 were designed using a bioinformatics platform with an algorithmic score predicting the knockdown efficacy of the siRNA. The platform was also able to filter out stretches of the mRNA with minor allele frequency (MAF)<0.01, 0 mismatch with Macaca fascicularis, off-target score ≤0.1 in both human and Macaca fascicularis. The off-target score is a composite score which sums up the following: ‘1’ is scored when the siRNA binds with 100% identity to an off-target gene, ‘0.1’ is scored when the siRNA binds with 1 mismatch to an off-target gene, ‘0.01’ is scored when the siRNA binds with 2 mismatches to an off-target gene.

The mRNA sequences of ITFG1 targeted by the siRNAs are shown in SEQ ID NOs:13 to 44. The nucleotide sequences of the antisense nucleic acids of the siRNAs (i.e. the guide strands) targeting SEQ ID NOs:13 to 44 are shown in SEQ ID NOs:45 to 76. The siRNAs were also converted to 27-mer dsiRNA molecules. The mRNA sequences of ITFG1 targeted by the dsiRNAs are shown in SEQ ID NOs:115 to 146. The nucleotide sequences of the antisense nucleic acids of the dsiRNAs targeting SEQ ID NOs: 115 to 146 are shown in SEQ ID NOs:147 to 178.

The following inhibitory nucleic acids were produced:

TABLE 1
dsiRNA targeting ITFG1 mRNA

| Identifier | Antisense (guide) | Sense (passenger) |
|---|---|---|
| CG_200135 | SEQ ID NO: 147 | SEQ ID NO: 179 |
| CG_200136 | SEQ ID NO: 148 | SEQ ID NO: 180 |
| CG_200137 | SEQ ID NO: 149 | SEQ ID NO: 181 |
| CG_200138 | SEQ ID NO: 150 | SEQ ID NO: 182 |
| CG_200139 | SEQ ID NO: 151 | SEQ ID NO: 183 |
| CG_200140 | SEQ ID NO: 152 | SEQ ID NO: 184 |
| CG_200141 | SEQ ID NO: 153 | SEQ ID NO: 185 |
| CG_200142 | SEQ ID NO: 154 | SEQ ID NO: 186 |
| CG_200143 | SEQ ID NO: 155 | SEQ ID NO: 187 |
| CG_200144 | SEQ ID NO: 156 | SEQ ID NO: 188 |
| CG_200145 | SEQ ID NO: 157 | SEQ ID NO: 189 |
| CG_200146 | SEQ ID NO: 158 | SEQ ID NO: 190 |
| CG_200147 | SEQ ID NO: 159 | SEQ ID NO: 191 |
| CG_200148 | SEQ ID NO: 160 | SEQ ID NO: 192 |
| CG_200149 | SEQ ID NO: 161 | SEQ ID NO: 193 |
| CG_200150 | SEQ ID NO: 162 | SEQ ID NO: 194 |
| CG_200151 | SEQ ID NO: 163 | SEQ ID NO: 195 |
| CG_200152 | SEQ ID NO: 164 | SEQ ID NO: 196 |
| CG_200153 | SEQ ID NO: 165 | SEQ ID NO: 197 |
| CG_200154 | SEQ ID NO: 166 | SEQ ID NO: 198 |
| CG_200155 | SEQ ID NO: 167 | SEQ ID NO: 199 |
| CG_200156 | SEQ ID NO: 168 | SEQ ID NO: 200 |
| CG_200157 | SEQ ID NO: 169 | SEQ ID NO: 201 |
| CG_200158 | SEQ ID NO: 170 | SEQ ID NO: 202 |
| CG_200159 | SEQ ID NO: 171 | SEQ ID NO: 203 |
| CG_200160 | SEQ ID NO: 172 | SEQ ID NO: 204 |
| CG_200161 | SEQ ID NO: 173 | SEQ ID NO: 205 |
| CG_200162 | SEQ ID NO: 174 | SEQ ID NO: 206 |
| CG_200163 | SEQ ID NO: 175 | SEQ ID NO: 207 |
| CG_200164 | SEQ ID NO: 176 | SEQ ID NO: 208 |
| CG_200165 | SEQ ID NO: 177 | SEQ ID NO: 209 |
| CG_200166 | SEQ ID NO: 178 | SEQ ID NO: 210 |

TABLE 2
siRNAs comprising nucleotide modifications
targeting ITFG1 mRNA

| Identifier | Antisense (guide) | Sense (passenger) |
|---|---|---|
| CG_200197 | SEQ ID NO: 211 | SEQ ID NO: 231 |
| CG_200198 | SEQ ID NO: 212 | SEQ ID NO: 231 |
| CG_200199 | SEQ ID NO: 213 | SEQ ID NO: 231 |
| CG_200200 | SEQ ID NO: 214 | SEQ ID NO: 231 |
| CG_200201 | SEQ ID NO: 215 | SEQ ID NO: 231 |
| CG_200202 | SEQ ID NO: 216 | SEQ ID NO: 231 |
| CG_200203 | SEQ ID NO: 217 | SEQ ID NO: 231 |
| CG_200204 | SEQ ID NO: 218 | SEQ ID NO: 231 |
| CG_200205 | SEQ ID NO: 219 | SEQ ID NO: 231 |
| CG_200206 | SEQ ID NO: 220 | SEQ ID NO: 231 |
| CG_200207 | SEQ ID NO: 211 | SEQ ID NO: 232 |
| CG_200208 | SEQ ID NO: 212 | SEQ ID NO: 232 |
| CG_200209 | SEQ ID NO: 213 | SEQ ID NO: 232 |
| CG_200210 | SEQ ID NO: 214 | SEQ ID NO: 232 |
| CG_200211 | SEQ ID NO: 215 | SEQ ID NO: 232 |
| CG_200212 | SEQ ID NO: 216 | SEQ ID NO: 232 |
| CG_200213 | SEQ ID NO: 217 | SEQ ID NO: 232 |
| CG_200214 | SEQ ID NO: 218 | SEQ ID NO: 232 |
| CG_200215 | SEQ ID NO: 219 | SEQ ID NO: 232 |
| CG_200216 | SEQ ID NO: 220 | SEQ ID NO: 232 |
| CG_200217 | SEQ ID NO: 211 | SEQ ID NO: 233 |
| CG_200218 | SEQ ID NO: 212 | SEQ ID NO: 233 |
| CG_200219 | SEQ ID NO: 213 | SEQ ID NO: 233 |
| CG_200220 | SEQ ID NO: 214 | SEQ ID NO: 233 |
| CG_200221 | SEQ ID NO: 215 | SEQ ID NO: 233 |
| CG_200222 | SEQ ID NO: 216 | SEQ ID NO: 233 |
| CG_200223 | SEQ ID NO: 217 | SEQ ID NO: 233 |
| CG_200224 | SEQ ID NO: 218 | SEQ ID NO: 233 |
| CG_200225 | SEQ ID NO: 219 | SEQ ID NO: 233 |
| CG_200226 | SEQ ID NO: 220 | SEQ ID NO: 233 |
| CG_200227 | SEQ ID NO: 211 | SEQ ID NO: 234 |
| CG_200228 | SEQ ID NO: 212 | SEQ ID NO: 234 |
| CG_200229 | SEQ ID NO: 213 | SEQ ID NO: 234 |
| CG_200230 | SEQ ID NO: 214 | SEQ ID NO: 234 |
| CG_200231 | SEQ ID NO: 215 | SEQ ID NO: 234 |
| CG_200232 | SEQ ID NO: 216 | SEQ ID NO: 234 |
| CG_200233 | SEQ ID NO: 217 | SEQ ID NO: 234 |
| CG_200234 | SEQ ID NO: 218 | SEQ ID NO: 234 |
| CG_200235 | SEQ ID NO: 219 | SEQ ID NO: 234 |
| CG_200236 | SEQ ID NO: 220 | SEQ ID NO: 234 |
| CG_200237 | SEQ ID NO: 221 | SEQ ID NO: 236 |
| CG_200238 | SEQ ID NO: 222 | SEQ ID NO: 236 |
| CG_200239 | SEQ ID NO: 223 | SEQ ID NO: 236 |
| CG_200240 | SEQ ID NO: 224 | SEQ ID NO: 236 |
| CG_200241 | SEQ ID NO: 225 | SEQ ID NO: 236 |
| CG_200242 | SEQ ID NO: 226 | SEQ ID NO: 236 |
| CG_200243 | SEQ ID NO: 227 | SEQ ID NO: 236 |
| CG_200244 | SEQ ID NO: 228 | SEQ ID NO: 236 |
| CG_200245 | SEQ ID NO: 229 | SEQ ID NO: 236 |
| CG_200246 | SEQ ID NO: 230 | SEQ ID NO: 236 |
| CG_200247 | SEQ ID NO: 221 | SEQ ID NO: 237 |
| CG_200248 | SEQ ID NO: 222 | SEQ ID NO: 237 |
| CG_200249 | SEQ ID NO: 223 | SEQ ID NO: 237 |
| CG_200250 | SEQ ID NO: 224 | SEQ ID NO: 237 |
| CG_200251 | SEQ ID NO: 225 | SEQ ID NO: 237 |
| CG_200252 | SEQ ID NO: 226 | SEQ ID NO: 237 |
| CG_200253 | SEQ ID NO: 227 | SEQ ID NO: 237 |
| CG_200254 | SEQ ID NO: 228 | SEQ ID NO: 237 |
| CG_200255 | SEQ ID NO: 229 | SEQ ID NO: 237 |
| CG_200256 | SEQ ID NO: 230 | SEQ ID NO: 237 |
| CG_200257 | SEQ ID NO: 221 | SEQ ID NO: 238 |
| CG_200258 | SEQ ID NO: 222 | SEQ ID NO: 238 |
| CG_200259 | SEQ ID NO: 223 | SEQ ID NO: 238 |
| CG_200260 | SEQ ID NO: 224 | SEQ ID NO: 238 |
| CG_200261 | SEQ ID NO: 225 | SEQ ID NO: 238 |
| CG_200262 | SEQ ID NO: 226 | SEQ ID NO: 238 |
| CG_200263 | SEQ ID NO: 227 | SEQ ID NO: 238 |
| CG_200264 | SEQ ID NO: 228 | SEQ ID NO: 238 |
| CG_200265 | SEQ ID NO: 229 | SEQ ID NO: 238 |
| CG_200266 | SEQ ID NO: 230 | SEQ ID NO: 238 |
| CG_200267 | SEQ ID NO: 221 | SEQ ID NO: 239 |
| CG_200268 | SEQ ID NO: 222 | SEQ ID NO: 239 |
| CG_200269 | SEQ ID NO: 223 | SEQ ID NO: 239 |
| CG_200270 | SEQ ID NO: 224 | SEQ ID NO: 239 |
| CG_200271 | SEQ ID NO: 225 | SEQ ID NO: 239 |
| CG_200272 | SEQ ID NO: 226 | SEQ ID NO: 239 |
| CG_200273 | SEQ ID NO: 227 | SEQ ID NO: 239 |
| CG_200274 | SEQ ID NO: 228 | SEQ ID NO: 239 |
| CG_200275 | SEQ ID NO: 229 | SEQ ID NO: 239 |
| CG_200276 | SEQ ID NO: 230 | SEQ ID NO: 239 |
| CG_200277 | SEQ ID NO: 215 | SEQ ID NO: 235 |
| CG_200278 | SEQ ID NO: 216 | SEQ ID NO: 235 |
| CG_200279 | SEQ ID NO: 226 | SEQ ID NO: 240 |
| CG_200280 | SEQ ID NO: 215 | SEQ ID NO: 241 |

### Example 2: Evaluation of Knockdown of ITFG1 Expression Using dsiRNAs

The dsiRNAs shown in Table 1 of Example 1 were evaluated for their ability to inhibit gene expression of ITFG1, as follows.

Cells of the human hepatocyte carcinoma HuH7 cell line (Cellosaurus Accession: CVCL_0336) were maintained in culture in vitro. HuH7 cells were cultured with DMEM high glucose supplemented with 10% FBS, 1% sodium pyruvate and 1% PenStrep in a humidified incubator at 37° C. and 5% CO₂.

5,000 HuH7 cells per well were seeded in wells of 96-well plates, and transfected with the different ITFG1-targeting dsiRNAs or non-targeting siRNAs (NT1 to NT5) at a final concentration of 20 nM (5 replicates for each condition), using the transfection agent DharmaFECT3 (Horizon), in accordance with the manufacturer's instructions.

The transfected cells were maintained in culture as described above. The cells were then harvested for analysis of the level of mRNA encoding ITFG1 at 3 days post-transfection.

The cells were washed with PBS, and followed with the process of Cell-to-Ct assay for the quantification of the level of mRNA encoding ITFG1 using SingleShot Cell Lysis RT-qPCR kit (Bio-Rad), in accordance with the manufacturer's instructions. The primers used for qPCR are shown in SEQ ID NOs:242 and 243.

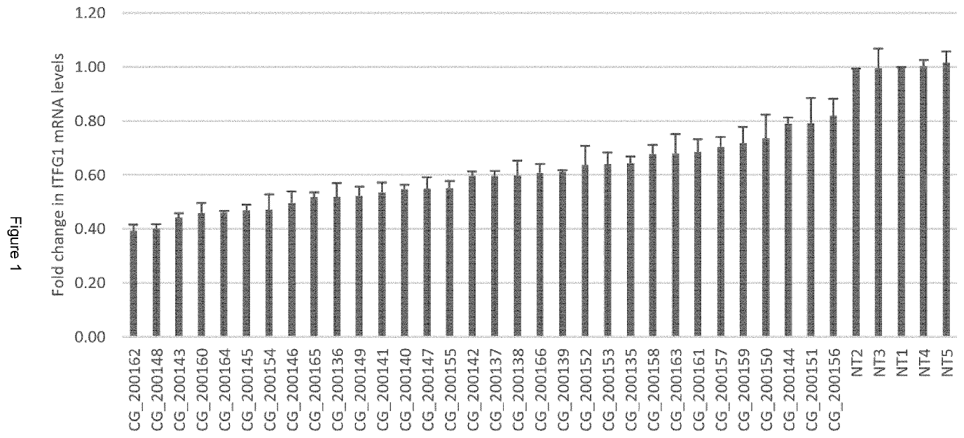

The level of ITFG1 knockdown was calculated by 2\hat{ }-ddct, normalizing to housekeeping gene PPIB, and further normalizing to non-targeting siRNA 1 (NT1) control. Fold change in the level of ITFG1 mRNA was calculated as: fold change=2^{-(sample (ITFG1 Ct-PPIB Ct)-NT1 (ITFG1 Ct-PPIBCt)}. The results are shown in FIG. 1.

### Example 3: Evaluation of Functional Consequences of Knockdown of ITFG1 Expression Using dsiRNAs

The effect of ITFG1 knockdown by the dsiRNAs shown in Table 1 of Example 1 on cell proliferation was investigated, as follows.

2,500 HuH7 cells were transfected with the dsiRNAs of Table 1 or non-targeting siRNAs (NT1 or NT2), and maintained in culture for 3 days, as described in Example 2. Cells were subsequently harvested, and viable cells were detected using (i) the CellTiter-Glo Luminescent Cell Viability Assay (Promega), (ii) the CCK8 Assay Kit (Dojindo), or (iii) Ki67 immunostaining, in accordance with the manufacturer's instructions.

For the CellTiter-Glo Luminescent Cell Viability Assay, cells were incubated in lysis buffer and CellTiter-Glo for 20 min. The resulting luminescence was then read in a plate reader. For the CCK8 Assay, cells were incubated with CCK8 reagent for 1 h, and then absorbance at 450 nm was measured using a plate reader in order to determine the concentration of the coloured product. For Ki67 immunostaining, cells were fixed in 4% paraformaldehyde, washed with PBS, permeabilized in PBS with 0.2% TritonX-100, blocked with 2% BSA, incubated with anti-Ki67 antibody overnight, washed with PBS, incubated with anti-rabbit-AF568 secondary antibody and DAPI (nuclear stain), washed with PBS, and 25 images of both Ki67 and DAPI staining were obtained per replicate by confocal microscopy. The numbers of Ki67+ cells and DAPI-stained nuclei were analysed by ImageJ, and the proportion of Ki67+ cells was obtained by dividing the number of Ki67+ cells by total number of DAPI-stained nuclei. Fold changes in luminescence, absorbance, or Ki67+ cell proportions were determined relative to values obtained from cells transfected with non-targeting siRNA control.

The results are shown in the table below. Several dsiRNAs were found to upregulate proliferation of Huh7 cells by >10% compared to cells transfected with NT1.

| | Huh7 | | | HepG2 | | |
|---|---|---|---|---|---|---|
| CG ID | CTG | CCK8 | Ki67 | CTG | CCK8 | Ki67 |
| CG_200135 | 0.83 | 1.19 | | 0.72 | 0.94 | |
| CG_200136 | 0.91 | 1.04 | | 0.74 | 0.89 | |
| CG_200137 | 0.92 | 0.88 | | 0.84 | 0.78 | |
| CG_200138 | 0.94 | 1.09 | | 0.81 | 0.82 | |
| CG_200139 | 0.82 | 1.10 | | 0.67 | 0.70 | |
| CG_200140 | 0.98 | 1.05 | | 1.04 | 1.06 | |
| CG_200141 | 0.97 | 1.00 | | 0.74 | 1.14 | |
| CG_200142 | 0.97 | 0.89 | | 0.91 | 1.04 | |
| CG_200143 | 1.05 | 0.96 | | 1.09 | 1.01 | |
| CG_200144 | 1.12 | 0.99 | 1.13 | 0.96 | 0.97 | 0.94 |
| CG_200145 | 1.07 | 1.14 | 0.99 | 0.84 | 1.03 | 0.79 |
| CG_200146 | 1.08 | 1.27 | 1.09 | 0.93 | 0.95 | 1.12 |
| CG_200147 | 1.10 | 1.12 | 0.98 | 0.86 | 1.02 | 0.43 |
| CG_200148 | 1.21 | 1.09 | 1.10 | 1.23 | 1.00 | 0.85 |
| CG_200149 | 1.13 | 1.07 | 1.05 | 1.14 | 1.06 | 1.00 |
| CG_200150 | 0.90 | 0.83 | | 0.67 | 1.07 | |
| CG_200151 | 0.84 | 0.83 | | 0.63 | 1.18 | |
| CG_200152 | 0.99 | 0.94 | | 0.85 | 0.85 | |
| CG_200153 | 1.01 | 1.08 | | 0.96 | 0.91 | |
| CG_200154 | 0.80 | 1.07 | 0.89 | 0.93 | 1.07 | 0.59 |
| CG_200155 | 1.18 | 1.20 | 1.14 | 1.27 | 1.03 | 1.09 |
| CG_200156 | 0.97 | 1.23 | 1.09 | 0.91 | 1.06 | 0.73 |
| CG_200157 | 0.86 | 1.14 | 0.98 | 0.79 | 0.86 | 0.51 |
| CG_200158 | 0.86 | 0.98 | 1.18 | 0.86 | 0.82 | 0.84 |
| CG_200159 | 1.02 | 1.15 | 1.05 | 0.95 | 0.93 | 1.00 |
| CG_200160 | 0.89 | 1.13 | | 0.83 | 0.96 | |
| CG_200161 | 0.92 | 1.03 | | 0.88 | 1.09 | |
| CG_200162 | 0.96 | 0.93 | | 0.89 | 0.95 | |
| CG_200163 | 0.85 | 0.85 | | 0.63 | 0.83 | |
| CG_200164 | 1.06 | 1.10 | | 0.98 | 1.09 | |
| CG_200165 | 0.87 | 0.97 | | 1.09 | 1.14 | |
| CG_200166 | 1.07 | 0.92 | | 0.85 | 1.04 | |
| NT1 | 0.94 | 1.00 | 1.03 | 0.98 | 0.99 | 1.01 |
| NT2 | 1.06 | | 0.97 | 1.02 | 1.01 | 0.99 |

### Example 4: Evaluation of Knockdown of ITFG1 Expression Using siRNAs Comprising Nucleotide Modifications

ITFG1 knockdown by the siRNAs comprising nucleotide modifications shown in Table 2 of Example 1 on cell proliferation was investigated, as follows.

HuH7 cells were transfected with CG_200198, CG_200199, CG_200200, CG_200201, CG_200202, CG_200203, CG_200204, CG_200205, CG_200206 (siRNAs having the antisense sequence of SEQ ID NO:58, and comprising modified nucleotides), CG_200148 (dsiRNA targeting the same region of ITFG1 mRNA) or non-targeting siRNA (NT1 or NT2) using RNAiMAX (Life Technologies), and maintained in culture for 3 days, as described in Example 2. Fold change in the level of mRNA encoding ITFG1 was determined as described in Example 2.

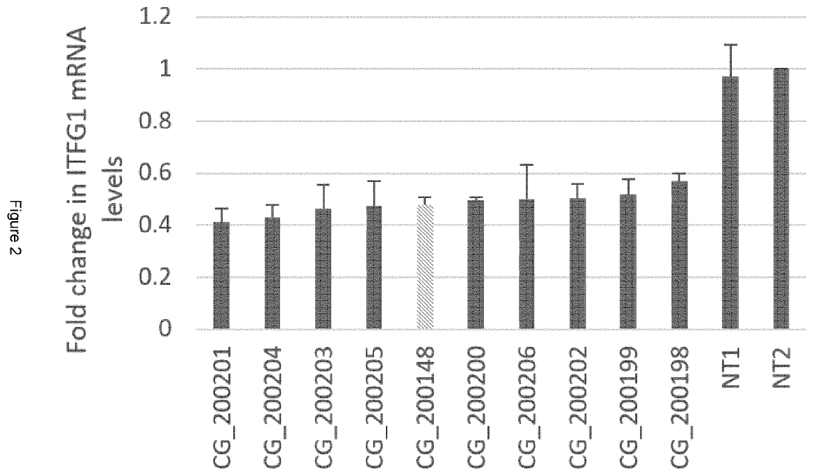

The results are shown in FIG. 2. Certain of the siRNAs comprising nucleotide modifications were found to more potently inhibit ITFG1 expression relative to dsiRNA targeting the same region of ITFG1 mRNA.

In further experiments, HuH7 cells were transfected with CG_200244, CG_200246, CG_200242, CG_200243, CG_200241, CG_200237, CG_200238, CG_200239, CG_200240, CG_200245 (siRNAs having the antisense sequence of SEQ ID NO:65, and comprising modified nucleotides), CG_200155 (dsiRNA targeting the same region of ITFG1 mRNA), CG_200199 or non-targeting siRNA (NT), and maintained in culture for 3 days, as described in Example 4 above. Fold change in the level of mRNA encoding ITFG1 was determined as described in Example 2.

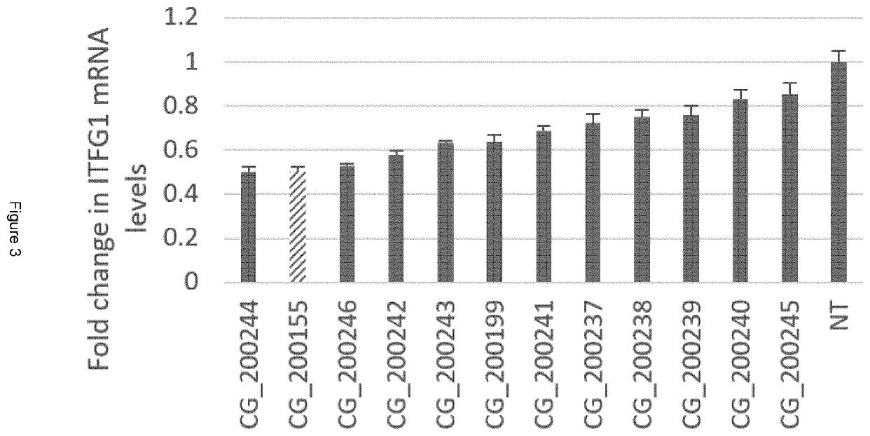

The results are shown in FIG. 3. Certain of the siRNAs comprising nucleotide modifications were found to more potently inhibit ITFG1 expression relative to dsiRNA targeting the same region of ITFG1 mRNA.

In further experiments, ITFG1 knockdown by siRNAs comprising nucleotide modifications and passenger strand mismatches were investigated. HuH7 cells were transfected with CG_200211, CG_200221, CG_200231, CG_200209, CG_200219, CG_200229, CG_200212, CG_200222, CG_200232, CG_200214, CG_200224, CG_200234, CG_200252, CG_200262, CG_200272, CG_200254, CG_200264, CG_200274, CG_200256, CG_200266, CG_200276 (i.e. siRNAs having the antisense sequence of SEQ ID NO:58, comprising modified nucleotides and further comprising mismatched nucleotides in the passenger strand), CG_200201, CG_200199, CG_200202, CG_200204, CG_200242, CG_200244, CG_200246 (the corresponding siRNAs comprising modified nucleotides and lacking mismatches in the passenger strand) or non-targeting siRNA (NT1), and maintained in culture for 3 days, as described in Example 4 above. Fold change in the level of mRNA encoding ITFG1 was determined as described in Example 2.

The results are shown in FIG. 4. Certain of the siRNAs comprising mismatched nucleotides in the passenger strand were found to more potently inhibit ITFG1 expression relative to their corresponding siRNA lacking mismatches in the passenger strand.

### Example 5: Evaluation of IC50 Values for Knockdown of ITFG1 Expression

CG_200231, CG_200232 and CG_200242 (see Table 2 of Example 1) were evaluated in order to determine their IC50 for knockdown of expression of ITFG1 in HuH7 cells, and of Itfg1 in cells of the mouse hepatocyte cell line AML12 (Cellosaurus Accession: CVCL_0140).

In further experiments, ITFG1 knockdown by siRNAs comprising nucleotide modifications and passenger strand mismatches was evaluated in order to determine IC50 values. HuH7 cells were transfected with CG_200202, CG_200204, CG_200231, CG_200232 (i.e. siRNAs having the antisense sequence of SEQ ID NO:58, comprising modified nucleotides and further comprising mismatched nucleotides in the passenger strand), CG_200242 (i.e. siRNA having the antisense sequence of SEQ ID NO:65, comprising modified nucleotides and lacking mismatches in the passenger strand) or non-targeting siRNA (NT1) at concentrations ranging from 1.6 pM to 50 nM, and maintained in culture for 3 days, as described in Example 4. The fold change in the level of mRNA encoding ITFG1 was determined as described in Example 2. The experiment in AML12 cells was performed in the exact same manner as for HuH7 cells, except that the primers used for qPCR are shown in SEQ ID NOs:244 and 245.

The results were as follows:

| siRNA molecule | IC50 in Huh7 cells (pM) | IC50 in AML12 cells (pM) |
|---|---|---|
| CG_200202 | 13.6 | 180.4 |
| CG_200204 | 21.09 | Not Determined |
| CG_200231 | 32.35 | 114 |
| CG_200232 | 27.31 | 134.9 |
| CG_200242 | 98.87 | 184 |

The siRNAs were found to inhibit expression of ITFG1 in human cells and Itfg1 in mouse cells with high potency.

In further experiments, the IC50 for ITFG1 knockdown in HuH7 and AML12 cells was investigated again for CG_200231, CG_200232 and CG_200242. Briefly, cells were transfected with the siRNAs at concentrations ranging from 80 fM to 5 nM, and maintained in culture for 3 days, as described above. Fold change in the level of mRNA encoding ITFG1 was determined as described in Example 2.

The results were as follows:

| SIRNA | IC50 in Huh7 (pM) | IC50 in AML12 (pM) |
|---|---|---|
| CG_200231 | 2.552 | 18.18 |
| CG_200232 | 1.485 | 27.24 |
| CG_200242 | 17.8 | 94.65 |

### Example 6 Evaluation of Inhibition of ITFG1 Protein Expression

CG_200231, CG_200232 and CG_200242 (see Table 2 of Example 1) were evaluated to confirm their ability to knockdown expression of ITFG1 at the protein level.

Briefly, HuH7 cells were transfected with 30 nM or 1 nM of siRNA and maintained in culture for 3 days, as described in Example 4 above. The cells were then harvested in standard RIPA buffer supplemented with a cocktail of protease inhibitors, and subsequently sonicated. Protein concentrations were determined by BCA assay according to manufacturer's protocol. A reducing buffer was added to each of the protein samples, which were then boiled to denature the proteins. The protein samples were then resolved by SDS-PAGE, transferred to a PVDF membrane, blocked, and incubated with primary anti-ITFG1 antibody, washed, incubated with secondary detection antibody, washed and then developed using ECL substrate. The western blots were imaged on the ChemiDoc Imaging System.

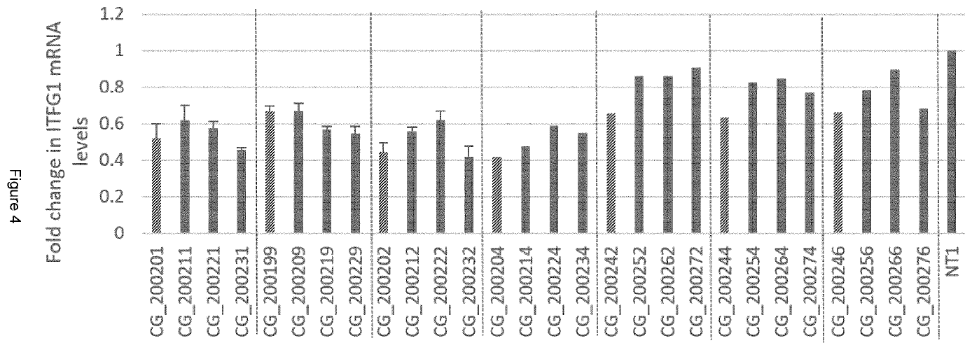

The results are shown in FIG. 5. The siRNAs evaluated were found to knockdown expression of ITFG1 at the protein level in a dose-dependent fashion.

### Example 7: Evaluation of Functional Consequences of Knockdown of ITFG1 Expression Using siRNAs Comprising Nucleotide Modifications

The effect of ITFG1 knockdown by CG_200231, CG_200232 and CG_200242 (see Table 2 of Example 1) on cell proliferation was investigated, as follows.

HuH7 cells were transfected with CG_200231, CG_200232 and CG_200242 or non-targeting siRNA (NT) with RNAiMAX (Life Technologies), and maintained in culture for 3 days, as described in Example 3. Cells were subsequently harvested, and viable cells were detected using the CellTiter-Glo Luminescent Cell Viability Assay (Promega), or (ii) the CCK8 Assay Kit (Dojindo), as described in Example 3.

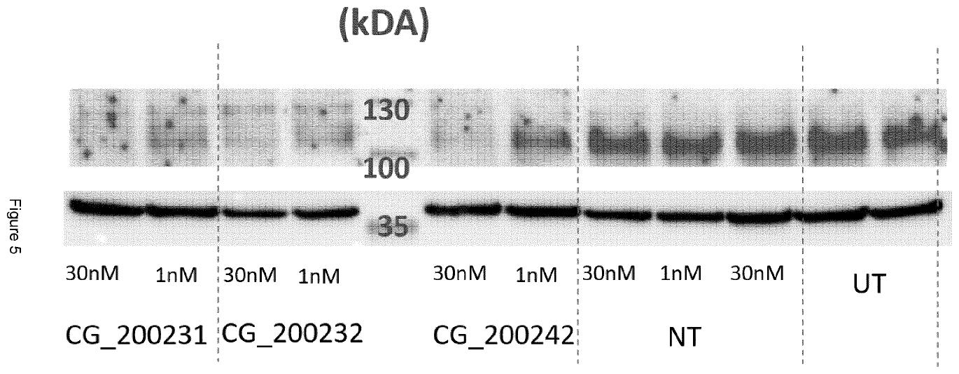

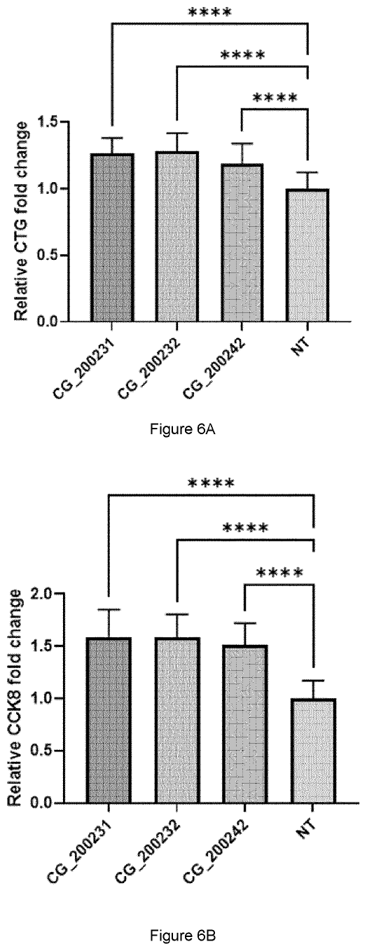

The results are shown in FIGS. 6A and 6B, and are summarised in the table below:

| | Fold change in CCK8 | Fold change in CellTiter- |
|---|---|---|
| siRNA molecule | relative to NT | Glo relative to NT |
| CG_200231 | 1.58 | 1.26 |
| CG_200232 | 1.58 | 1.28 |
| CG_200242 | 1.50 | 1.18 |
| NT | 1.00 | 1.00 |

The siRNAs were found to potently promote cell proliferation.

### Example 8: Evaluation of Cytotoxicity

CG_200202, CG_200231, CG_200232 and CG_200242 were investigated in order to determine their cytotoxicity in human Huh7 cells and mouse AML12 cells.

Briefly, siRNAs were transfected into Huh7 and HepG2 cells at concentrations between 1 to 200 nM and harvested for CCK8 analysis 3 days after transfection, as described in Example 7. Therapeutic index was calculated by IC50 divided by the half-maximal cytotoxic concentration (CC50).

The results were as follows:

| | Huh7 | | AML12 | |
|---|---|---|---|---|
| siRNA | | Therapeutic | | Therapeutic |
| molecule | CC50 (nM) | index | CC50 (nM) | index |
| CG_200202 | >200 | >1000 | 60 | 631 |
| CG_200231 | >200 | >1000 | >200 | >1000 |
| CG_200232 | >200 | >1000 | >200 | >1000 |
| CG_200242 | >200 | >1000 | >200 | >1000 |

CG_200202 lacking passenger strand mismatches was determined to be cytotoxic to mouse AML12 cells, whereas the equivalent molecules comprising passenger strand mismatches (CG_200231, CG_200232) were not.

### Example 9: Evaluation of Off-Target Knockdown of ITFG1 Expression In Vivo

CG_200202, CG_200231, CG_200232 and CG_200242 were evaluated in order to determine whether they cause off-target knockdown of the expression of genes other than ITFG1 in human Huh7 cells, or of genes other than Itfg1 in mouse AML12 cells.

In further experiments, HuH7 or AML12 cells were transfected with CG_200201, CG_200202, CG_200231, CG_200232, CG_200242, as described in Example 4, and maintained in culture for 16 to 24 hours. RNA was purified by column purification (Favorgen) and used for library preparation, then sequenced on the Illumina sequencer. Differential expression analysis was performed with the NT samples as the reference. Off-target genes were defined as the genes in siRNA-treated samples that were differentially expressed from NT samples by fold-change ≥−2.

The results were as follows:

| | | # off-target genes |
|---|---|---|
| | | downregulated below |
| siRNA molecule | Cell line | threshold |
| CG_200201 | Huh7 | 14 |
| CG_200231 | Huh7 | 8 |
| CG_200202 | Huh7 | 15 |
| CG_200232 | Huh7 | 11 |
| CG_200242 | Huh7 | 0 |
| CG_200231 | AML12 | 2 |
| CG_200232 | AML12 | 8 |
| CG_200242 | AML12 | 2 |

CG_200201 and CG_200202 lacking passenger strand mismatches were determined to cause knockdown of a much greater number of off-target genes than the equivalent molecules comprising passenger strand mismatches (CG_200231, CG_200232).

### Example 10: Evaluation of Knockdown of ITFG1 Expression In Vivo

GalNAc-conjugated CG_200277, CG_200278 and CG_200279 were evaluated for their ability to knockdown expression of ITGF1 in vivo.

Mice were injected subcutaneously single 2 mg/kg bodyweight dose of CG_200277, CG_200278, CG_200279 conjugated to GalNAc or PBS at Day 0. The livers of the mice were harvested at Days 4, 7, or 14 after injection into Trizol, and processed for column-based RNA extraction as per manufacturer's protocol (Favorgen). cDNA synthesis was performed with Maxima RT H-system, and qPCR was performed with SYBER Green (Merck). The primers used for qPCR are shown in SEQ ID NOs:244 and 245. The level of Itfg1 knockdown was calculated by 2\hat{ }-ddct, normalizing to housekeeping gene Ppib, and further normalizing to PBS control. Fold change in the level of Itfg1 mRNA was calculated as: fold change=2^{-(sample (Itfg1 Ct-Ppib Ct)-PBS (Itfg1 Ct-Ppib Ct)}. The results are shown in FIG. 7. Mice administered with a single, 2 mg/kg dose of CG_200277, CG_200278 or CG_200279 conjugated to GalNAc displayed robust knockdown of Itfg1 expression, which is sustained for at least 14 days.

In further experiments, in vivo knockdown of Itfg1 expression by CG_200277, CG_200278 and CG_200279 was investigated following administration of a single dose of the GalNAc-conjugated siRNA, at a concentration of 2 mg/kg, 6 mg/kg or 10 mg/kg. Mice were injected subcutaneously with a single dose of CG_200277, CG_200278, or CG_200279 conjugated to GalNAc or PBS at Day 0. The livers of the mice were harvested at Day 7 and processed for qPCR as described above.

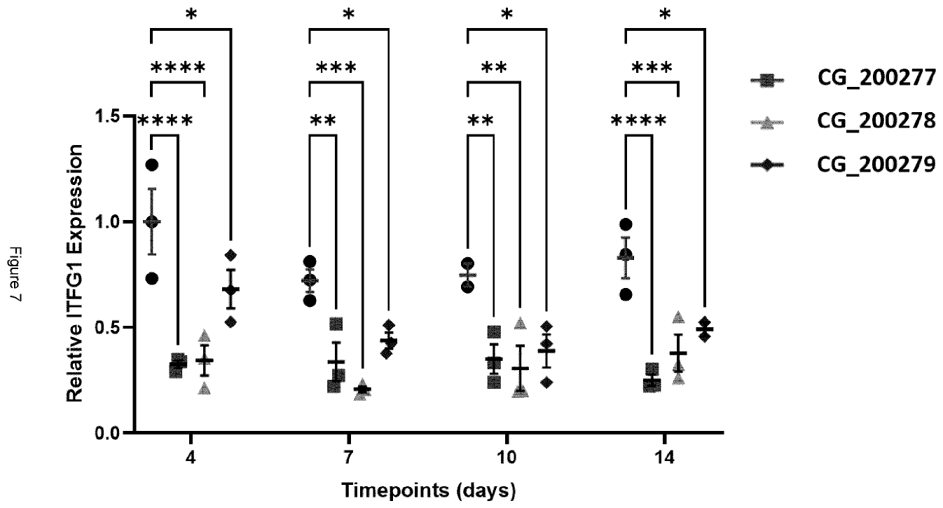

The results are shown in FIG. 8. CG_200277, CG_200278 or CG_200279 conjugated to GalNAc displayed dose-dependent knockdown of Itfg1 expression in vivo.

### Example 11: Evaluation of Toxicity In Vivo

GalNAc-conjugated CG_200277, CG_200278 and CG_200279 were evaluated for their toxicity in vivo, as determined by analysis of serum AST and ALT levels.

Sera were collected from mice via the submandibular vein at 11 and 4 days before siRNA administration. Mice were injected subcutaneously with a single 10 mg/kg bodyweight dose of CG_200277, CG_200278, CG_200279 conjugated to GalNAc or PBS at Day 0, and sera were collected on Days 2 and 4 after the administration. The serum AST and ALT levels were determined using commercially-available kits (Abcam) according to the manufacturer's protocols.

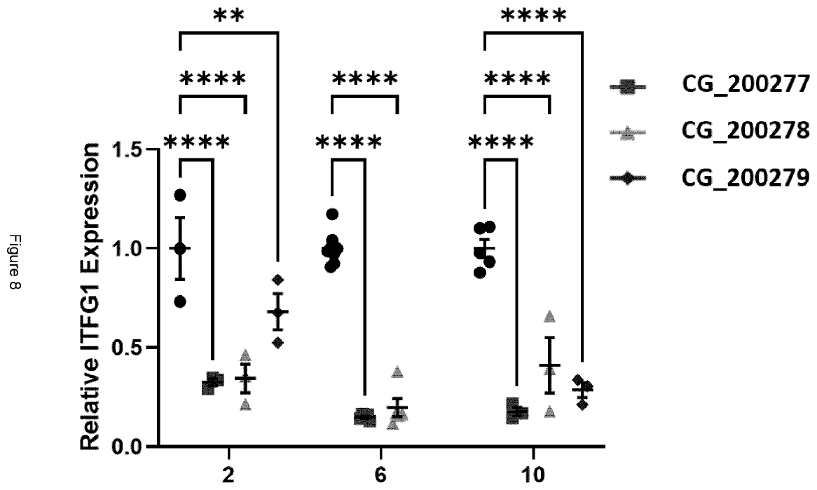

The results are shown in FIGS. 9A and 9B. Administration of CG_200277, CG_200278 or CG_200279 conjugated to GalNAc at a single dose of 10 mg/kg was found not to result in elevated AST or ALT levels, and thus no significant toxicity of the molecules was detected.

### Example 12: Further In Vivo Characterisation of siRNA Molecules Targeting ITFG1

The duration of knockdown of Iftg1 expression following treatment with CG_200277 was investigated.

Briefly, mice were injected with a single dose of CG_200277 at 6 mg/kg bodyweight, and livers were harvested at various timepoints from 1 to 6 weeks post-injection. RNA was extracted from the livers, and processed for qPCR analysis of the level of Itfg1 mRNA as described in Example 10.

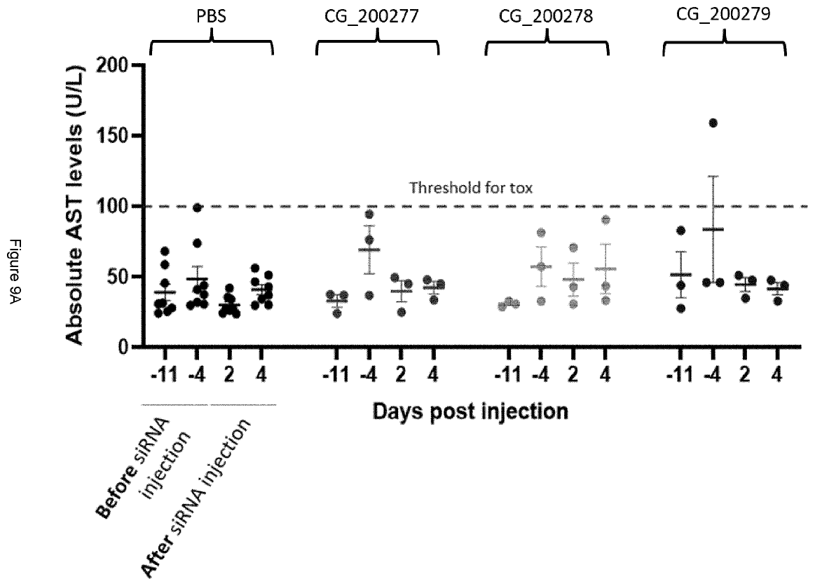

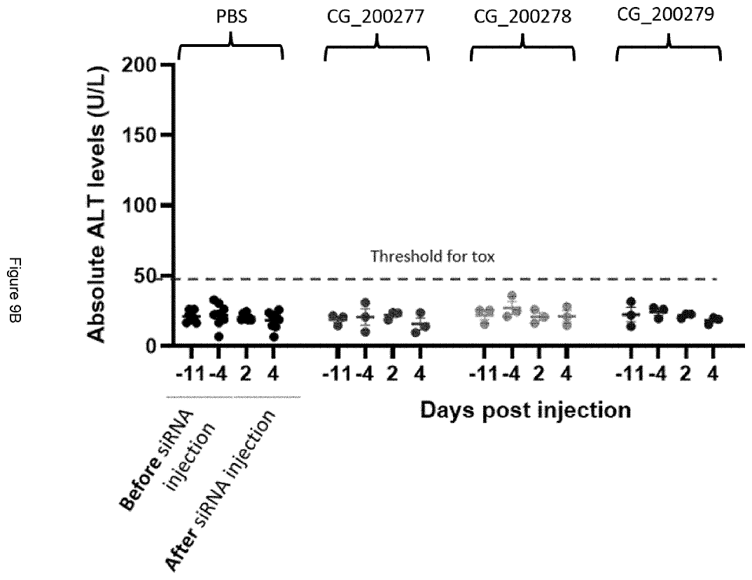

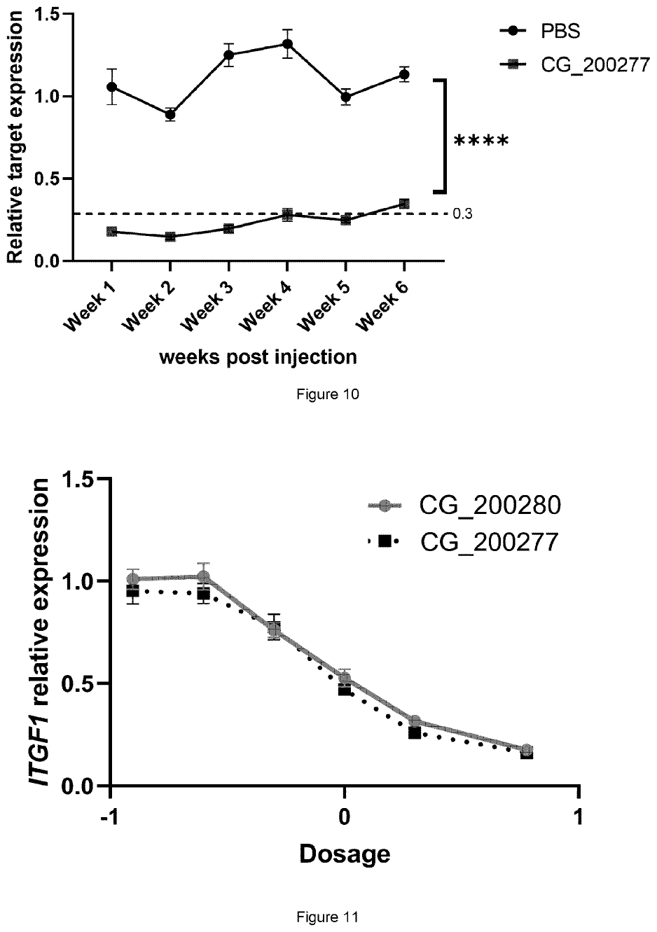

The results are shown in FIG. 10. A single dose of CG_200277 at 6 mg/kg bodyweight was found to induce at least ˜70% knockdown of Iftg1 expression for a period of at least 6 weeks.

In further experiments, mice are injected with siRNA targeting ITFG1 (e.g. CG_200277, CG_200278, CG_200279 or CG_200280) at doses ranging from 2 to 10 mg/kg bodyweight, and livers are harvested at various timepoints from 1 to 20 weeks post-injection. RNA is extracted from the livers, and processed for qPCR analysis of the level of Itfg1 mRNA as described in Example 10. The relevant siRNA is found to induce >80% knockdown of Itfg1 expression for >2 weeks, and to sustain 250% knockdown of Itfg1 expression for up to 4 weeks, indicating a favourable pharmacodynamic profile enabling lower dosing frequency.

In further experiments, the inventors investigated the dose of CG_200277 required to achieve 50% (ED50) and 80% (ED80) knockdown of Itfg1 expression 1 week after injection. Briefly, mice were injected with a single dose of CG_200277, or CG_200280 at 0.125, 0.25, 0.5, 1, 2, or 6 mg/kg bodyweight, and livers were harvested 1 week post-injection. RNA was extracted from the livers, and processed for qPCR analysis of the level of Itfg1 mRNA as described in Example 10.

The results are shown in FIG. 11. CG_200277 and CG_200280 were found to be highly potent at inducing knockdown of Itfg1 expression, with both compounds having an ED50 of 0.8 mg/kg and an ED80 of 3.4 mg/kg or 3.6 mg/kg respectively.

In further experiments, the inventors investigated the multidose toxicity of CG_200277. Briefly, mice were injected subcutaneously with three 20 mg/kg bodyweight doses of CG_200277 over the course of 1 week, on Days 0, 3, and 7. Sera were collected from the mice via the submandibular vein on Days 1, 4, 8, 10 and 14, and analysed for correlates of liver toxicity as described in Example 11. The livers were harvested at Day 14, and processed for fixed-formalin paraffin embedding (FFPE), then sectioned and stained with hematoxylin and eosin (H&E) for subsequent pathological evaluation.

The results are shown in FIGS. 12A and 12B. No obvious increase in serum levels of ALT or AST were detected in CG_200277-treated mice as compared to PBS-treated control mice. Histological analysis of the liver, kidney and spleen by the pathologist verified that no abnormalities were observed in the CG_200277-treated group.

In further experiments, the inventors investigate the acute toxicity of CG_200277. Briefly, mice are given weekly subcutaneous injections of CG_200277 at dosages up to 300 mg/kg bodyweight starting on Day 0, for up to 6 weeks. Sera are collected from the mice via the submandibular vein from 7 days prior to the first dose, and at multiple times over the duration of the injection and 4 weeks following the last injection. The sera are analysed for correlates of liver toxicity as described in Example 11. No obvious increase in serum levels of ALT or AST are detected in CG_200277-treated mice as compared to PBS-treated control mice.

### Example 13: Evaluation of In Vitro Stability of siRNA Targeting ITFG1 Comprising Nucleotide Modifications

In order to evaluate stability of siRNAs comprising nucleotide modifications, CG_200280 is incubated in human serum or liver lysosome extract for various time points up to 7 days at 37° C. Subsequently, the siRNAs and their degradation products are purified from the reaction mixture using silica gel columns, and analysed by LC-MS. The siRNAs are found to be highly stable and nuclease-resistant, with >70% of the full length nucleotide sequence of the siRNA remaining after incubation in the presence of human serum or liver lysosome extract for 3 days, and 250% of the full length nucleotide sequence of the siRNA remaining after incubation in the presence of human serum or liver lysosome extract for 7 days. Specifically, after 24 hours incubation of CG_200280 in human serum, 93% of SEQ ID NO:215 and 91% of SEQ ID NO:241 remained. The results indicate that the siRNAs will be stable under physiological conditions for prolonged pharmacodynamics.

### Example 14: In Vitro Evaluation of Immunogenicity of siRNAs Targeting ITFG1 Comprising Nucleotide Modifications

In order to evaluate the immunogenicity of siRNAs comprising nucleotide modifications, siRNAs of Table 2 of Example 1 are incubated with human peripheral blood mononuclear cells (PBMCs) for up to 24 hours in culture in vitro, and the cell culture supernatant is collected analysed for the expression of proinflammatory cytokines including IL-6, IL-10 and TNFα. The siRNAs are found not to significantly upregulate expression of these cytokines, relative to expression by control PBMCs incubated with PBS.

### Example 15: Evaluation of the Ability of siRNAs Targeting ITFG1 to Induce Hepatocyte Proliferation In Vivo

Mice were administered with siRNA targeting ITFG1 (e.g. CG_200277 or CG_200280) at 6 mg/kg bodyweight, or PBS (control). 1 week after injection of the siRNA or PBS, mice were subjected to two-thirds partial hepatectomy (see e.g. Mitchell and Willenbring Nat. Protoc. (2008) 3(7):1167-70) which induced hepatocyte proliferation. Livers were harvested from mouse 42 hours after partial hepatectomy, and evaluated for hepatocyte proliferation via immunostaining analysis of the expression of Ki67, a classical marker of proliferation. Briefly, livers were fixed in 4% paraformaldehyde and equilibrated in 30% sucrose solution before being embedded in Optimal Cutting Temperature embedding medium. The livers were sectioned and underwent routine immunostaining with antibody against Ki67 (Abcam), a secondary antibody that recognized the Ki67 antibody (Life Technologies), and counterstained with DAPI to label the nuclei. Images of the stained sections were acquired on an automated imaging system, and an ImageJ macro was employed to automatically count the proportion of cells that expressed Ki67.

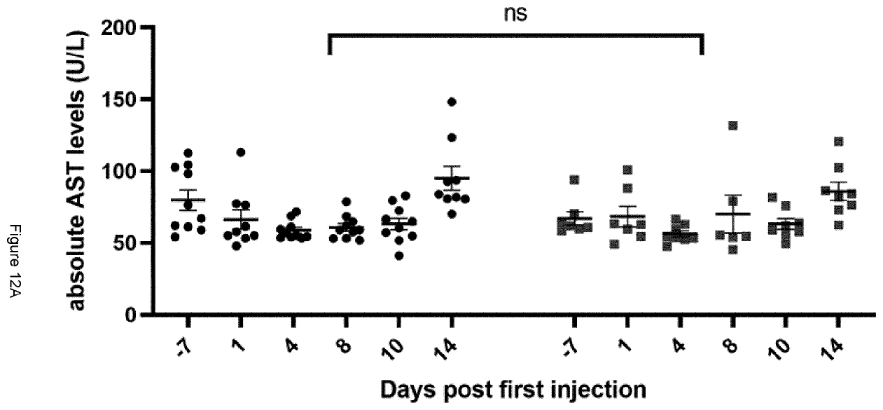

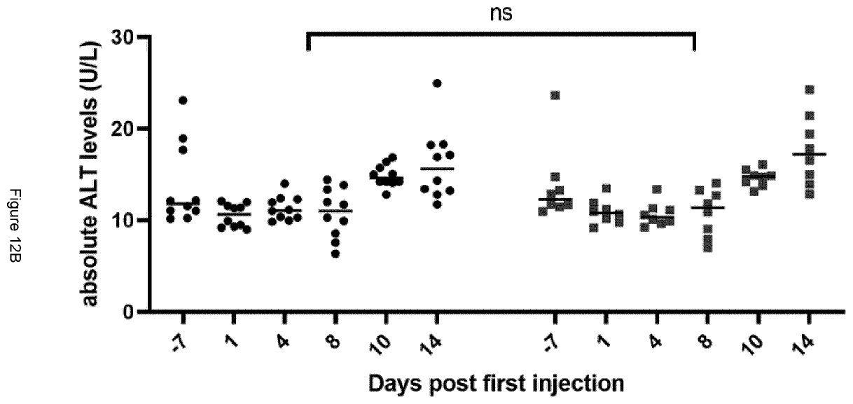

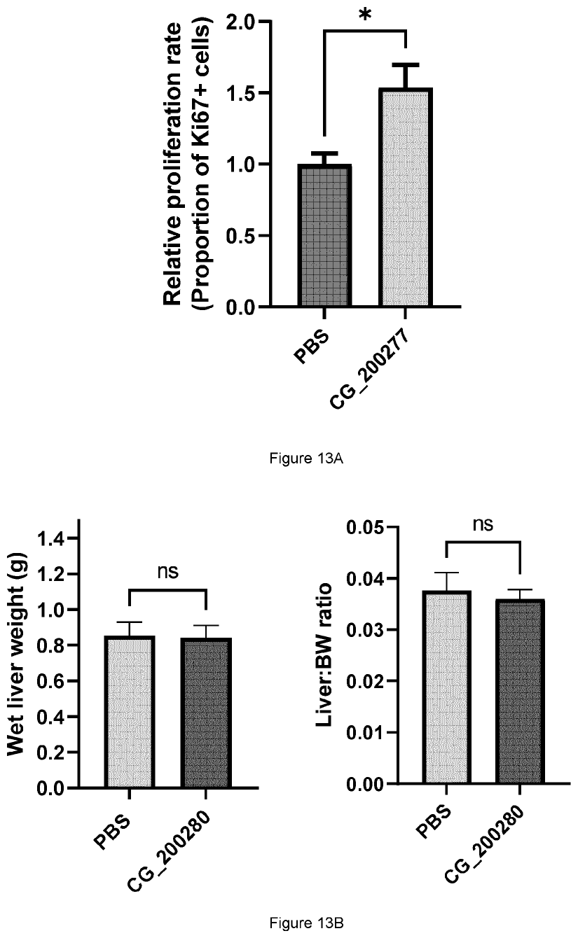

Mice administered siRNA targeting ITFG1 (e.g. CG_200277 or CG_200280) was found to have more than 50% increase in proportion of proliferating hepatocytes after partial hepatectomy as compared to mice administered PBS, as shown in FIG. 13A.

To evaluate the cessation of accelerated proliferation induced by siRNA targeting ITFG1 (e.g. CG_200280), mice were administered with siRNA targeting ITFG1 (e.g. CG_200280) at 6 mg/kg bodyweight, or PBS (control). 10 days after injection of the siRNA or PBS, mice were subjected to two-thirds partial hepatectomy as described above. Fourteen days after partial hepatectomy, the livers were harvested from the mice and weighed. FIG. 13B shows that the weights of regrown livers from PBS- or CG_200280-treated mice are not significantly different, indicating that Itfg1 knockdown induced by CG_200280 does not in result in uncontrolled proliferation and hepatomegaly.

### Example 16: Evaluation of the Ability of CG 200277 to Ameliorate Liver Disease In Vivo

### 16.1 Chemically-Induced Liver Fibrosis

The ability of siRNA targeting ITFG1 (e.g. CG_200277, CG_200278, CG_200279 or CG_200280) to inhibit liver fibrosis or slow the progression of fibrosis in a model of chemically-induced liver fibrosis is evaluated.

Briefly, mice are administered thioacetamide thrice weekly for 4 to 12 weeks in order to induce liver fibrosis (see e.g. Delire et al. J. Clin. Transl. Hepatol. (2015) 3(1): 53-66), and subsequently subcutaneously injected with siRNA targeting ITFG1 (e.g. CG_200277, CG_200278, CG_200279 or CG_200280) at doses ranging from 2 to 10 mg/kg bodyweight, or PBS (control), once every 2 to 4 weeks, for various periods of time up to 12 weeks. Livers are harvested from mice at various timepoints, and evaluated histologically for the extent of liver fibrosis. Mice treated with siRNA targeting ITFG1 display significantly less liver fibrosis as compared to control mice administered PBS.

In further experiments, mice are first subcutaneously injected with siRNA targeting ITFG1 (e.g. CG_200277, CG_200278, CG_200279 or CG_200280) at doses ranging from 2 to 10 mg/kg bodyweight. The mice are then administered thioacetamide thrice weekly for 4 to 12 weeks in order to induce liver fibrosis, from one week after the first siRNA injection. The siRNA is administered once every 2 to 4 weeks, at doses ranging from 2 to 10 mg/kg bodyweight, or PBS (control) during the entire period of thioacetamide treatment. Livers are harvested from mice at various timepoints, and evaluated histologically for the extent of liver fibrosis. Mice treated with siRNA targeting ITFG1 display significantly less liver fibrosis as compared to control mice administered PBS.

### 16.2 WDF Model of Liver Fibrosis and Steatosis

The ability of siRNA targeting ITFG1 (e.g. CG_200277 or CG_200280) to inhibit liver fibrosis and steatosis, or slow their progression, in a mouse model of non-alcoholic fatty liver disease (NAFLD) is evaluated.

Briefly, mice receive a Western Diet with fructose (WDF) for a period of 6 months to establish NAFLD (see e.g. Baena et al., Sci Rep (2016) 6: 26149, Machado et al., PloS One (2015) 10:e0127991). Livers were harvested from mice after 6 months of WDF to establish baseline characteristics. The remaining mice were subsequently subcutaneously injected with CG_200277 at 6 mg/kg bodyweight, or PBS (control), once every week, for 12 weeks. Livers were harvested from mice, and evaluated for the extent of ITFG1 knockdown potency, liver fibrosis, steatosis, proliferation and hepatic stellate cell activity. Part of the livers was processed for fixed-formalin paraffin embedding (FFPE), then sectioned and stained with hematoxylin and eosin (H&E) and Picro Sirius Red for subsequent pathological evaluation as described in Example 12. RNA was extracted from the livers, and processed for qPCR analysis as described in Example 10. The markers evaluated by qPCR are Itfg1, Ki67 and Acta2. The primers used for qPCR are shown in SEQ ID Nos: 246 to 249. Sera were collected from the mice via the submandibular vein at 4, 8 and 12 weeks of CG_200277 treatment and analysed for Albumin levels with a commercial kit (Abcam). Part of the livers were processed for protein analysis by Western Blot as described in Example 6.

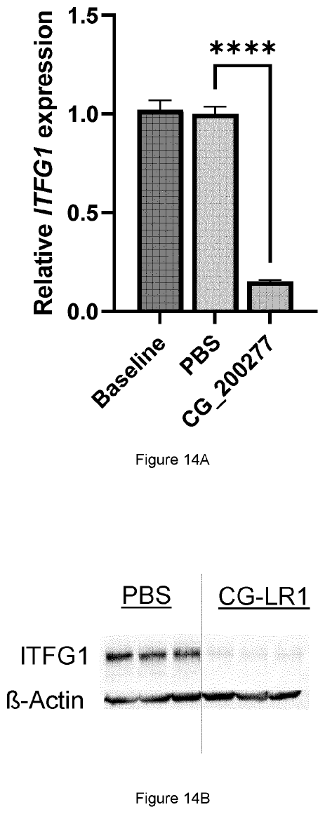

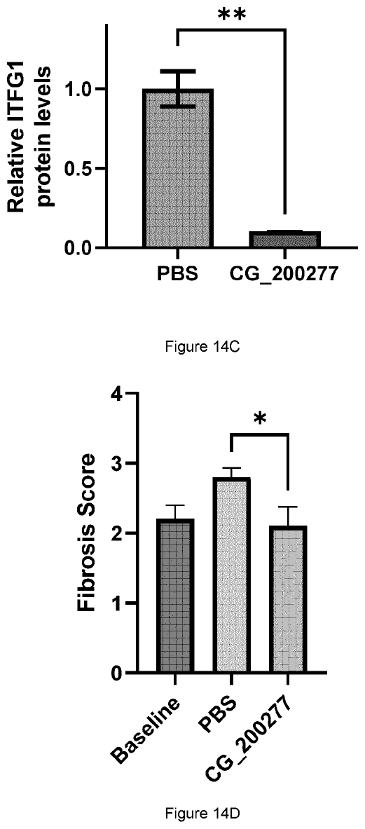

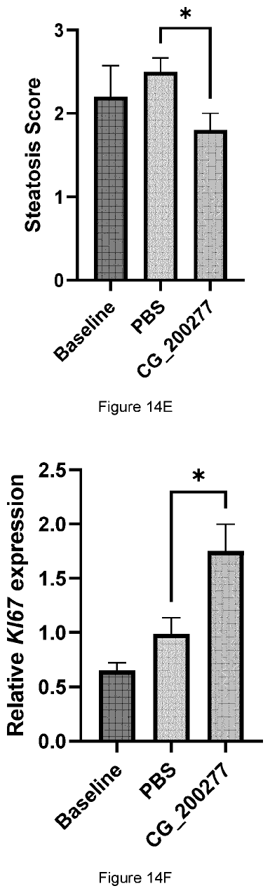

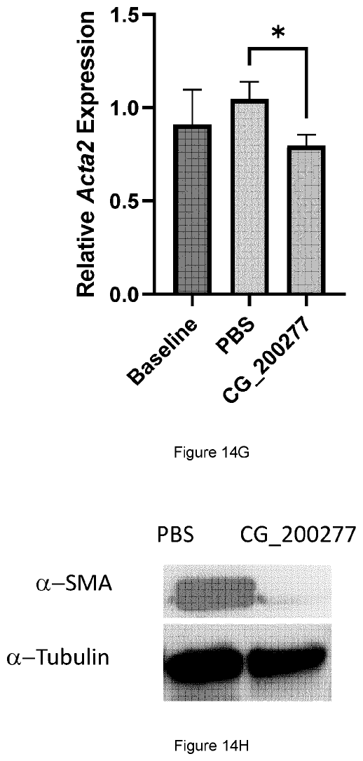

Compared to PBS controls, mice treated with CG_200277 had 85% ITFG1 knockdown at the mRNA level, and 90% knockdown at the protein level, reduced fibrosis (some mice in CG_200277 group had fibrosis scores lower than those of baseline group), reduced steatosis (some mice in CG_200277 group had steatosis scores lower than those of baseline group), increased proliferation (increased Ki67 mRNA expression), reduced hepatic stellate cell activity (lower Acta2 mRNA and smooth-muscle actin protein expression) and comparable liver function (similar Albumin protein levels) as shown in FIGS. 14A to 14I.

### 16.3 CDHFD Model of Liver Fibrosis and Steatosis

The ability of siRNA targeting ITFG1 (CG_200277 or CG_200280) to inhibit liver fibrosis and steatosis in a mouse model of acute liver injury and fat accumulation was evaluated.

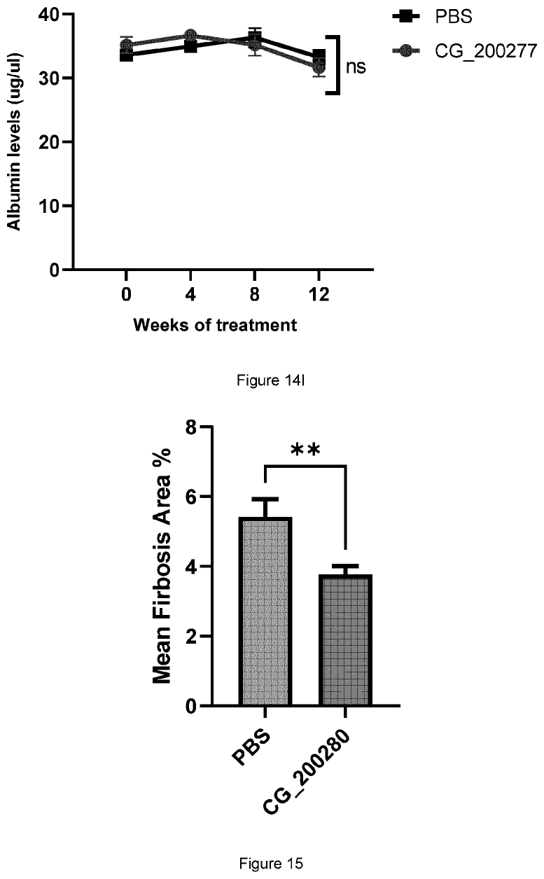

Briefly, mice received a choline-deficient high fat diet (CDHFD) for 8 weeks to establish acute steatosis and fibrosis (see e.g. Raubenheimer et al., Diabetes (2006) 55(7):2015-0-2020, Suzuki-Kemuriyama et al., FEBS Open Bio (2021) 11: 2950-2965). Mice were subsequently subcutaneously injected with siRNA targeting ITFG1 (CG_200280) at 6 mg/kg bodyweight, or PBS (control), once every week, for 4 weeks. Livers were processed for H&E and PSR staining as described in Example 16.2 for image quantification and pathological evaluated. The sections stained with Picro Sirius Red were imaged and the positively stained areas were quantified by ImageJ. Mice treated with siRNA targeting ITFG1 displayed significantly less liver fibrosis and/or steatosis as compared to control mice administered PBS (FIG. 15).

### 16.4 Primary Bile Duct Ligation Model of Liver Fibrosis and Cholestasis

The ability of siRNA targeting ITFG1 (CG_200277) to inhibit liver fibrosis in a mouse model of acute cholestasis-induced liver injury is evaluated.

Briefly, mice received a single 6 mg/kg body weight dose of CG_200277 or PBS. 7 days after the administration, they were subjected to primary bile duct ligation surgery (e.g. Tag et al., J Vis Exp (2015) 96:52438) to induce acute obstructive cholestasis. Additional doses of CG_200277 or PBS were administered to the mice at 6 mg/kg body weight 1, 3, 5, and 7 days after the surgery. The mice were weighed on the day of and 3, 5, 7, 10 and 13 days after surgery. Sera and livers were harvested from the mice 14 days after the surgery. Sera were evaluated for correlates of liver toxicity like AST, ALT and Total Bilirubin on Roche Cedex Bio Analyzer. The livers were processed for FFPE and histopathological evaluation as described in Example 12.

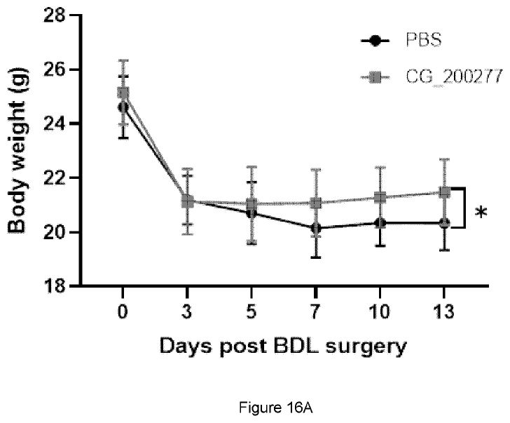

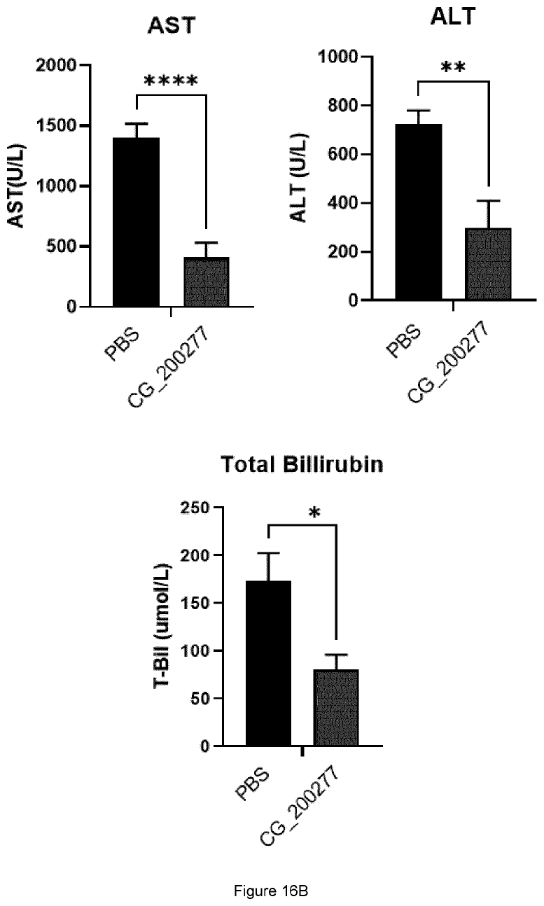

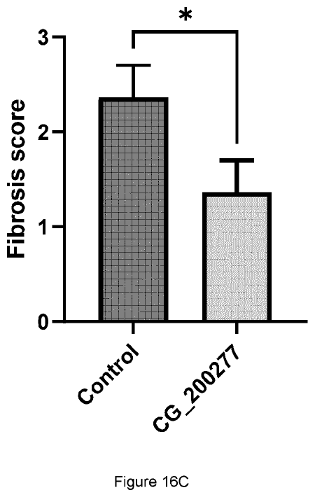

Compared to the PBS controls, the mice that received CG_200277 had higher body weights throughout the 2 weeks after the surgery (FIG. 16A), lower AST, ALT and Total Bilirubin (FIG. 16B), and lower fibrosis score (FIG. 16C).

### Example 17: Evaluation of Long-Term In Vivo Toxicity

The long-term in vivo toxicity of siRNA targeting ITFG1 was evaluated.

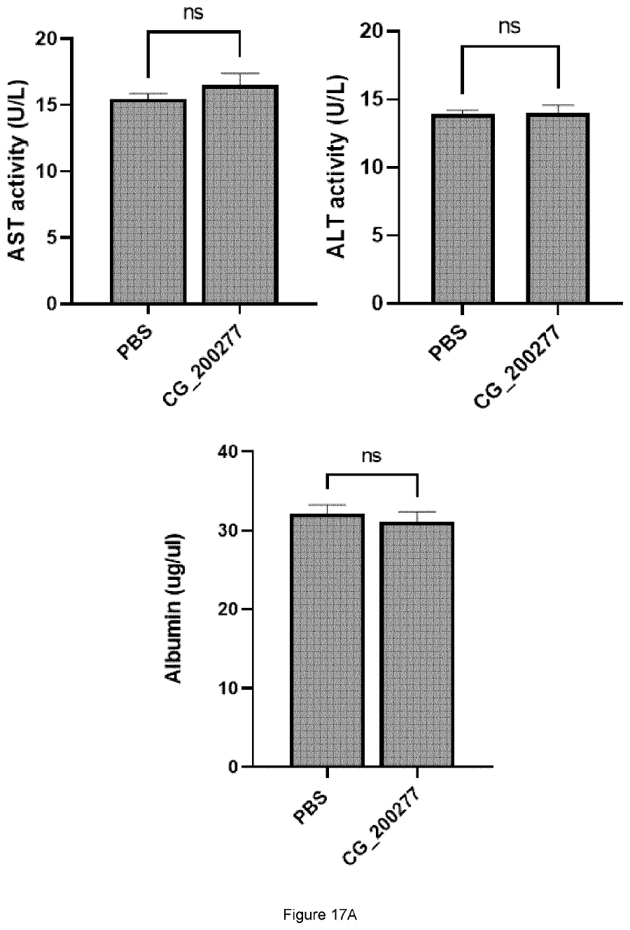

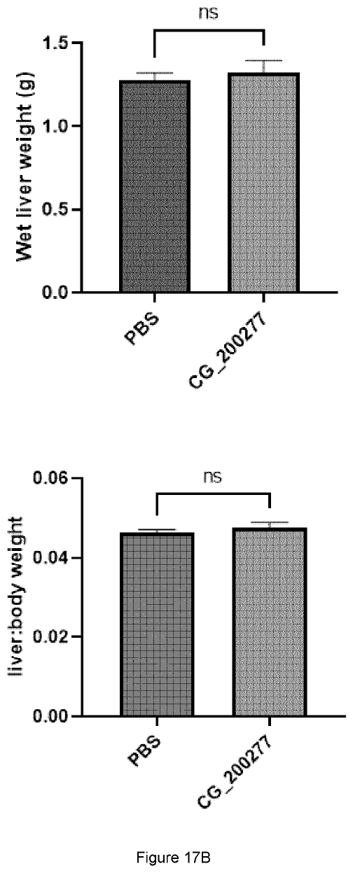

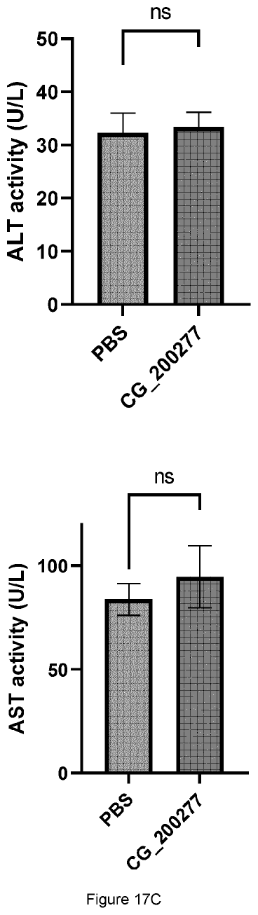

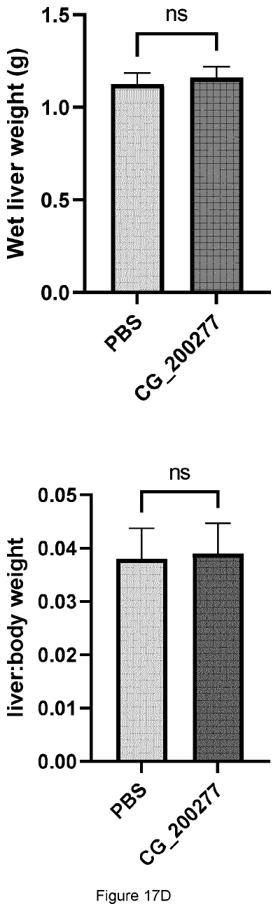

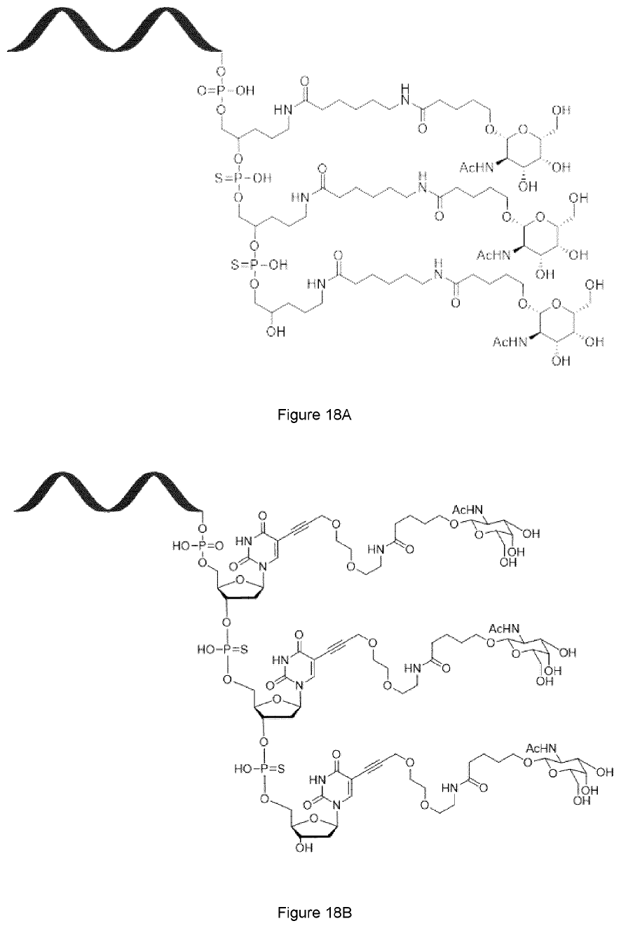

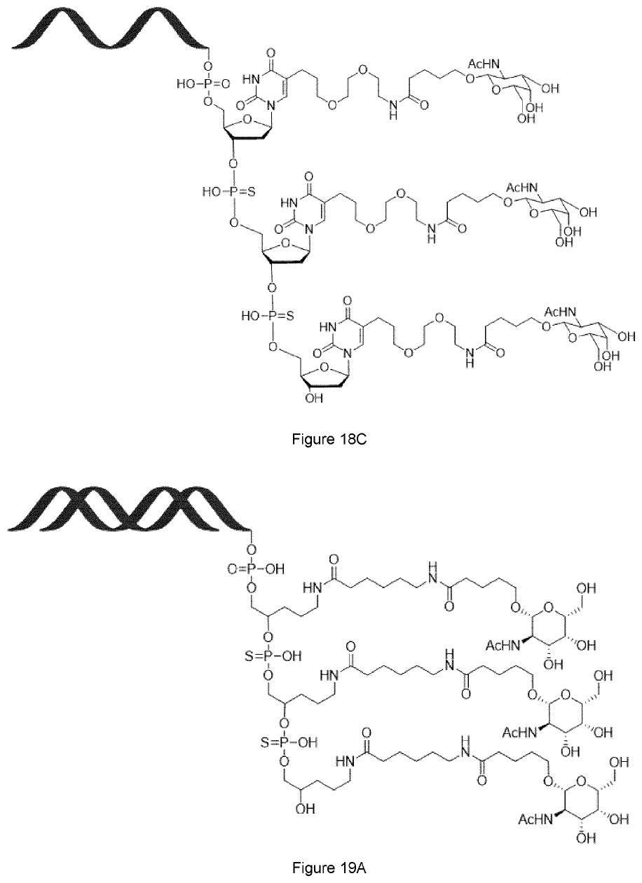

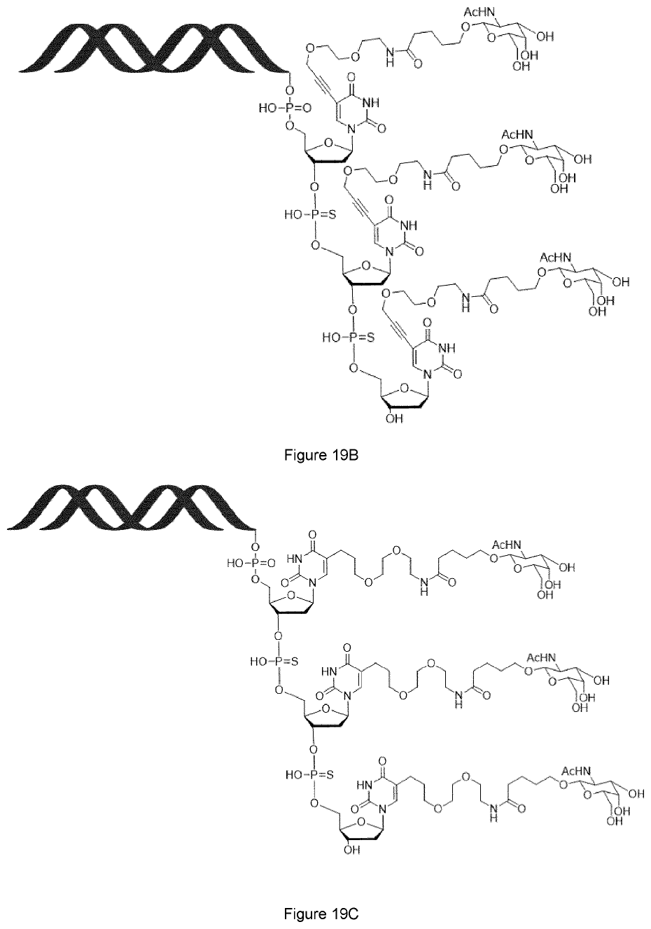

Mice were administered with a monthly dose of siRNA targeting ITFG1 (CG_200277) over 1 year to maintain various levels of IHfg1 knockdown. The body and wet liver weights of the mice were documented. Sera were collected from the mice via the submandibular vein, while livers, kidneys, spleen, bone marrow and other organs were harvested from mice 6 and 12 months from the first injection and evaluated as described in Example 12. Mice that received 6 monthly doses of CG_200277 did not exhibit differential AST, ALT and ALB to the PBS controls (FIG. 17A), and there was no histopathological difference between the tissues from CG_200277-treated and control groups (livers, kidneys, spleens and lymph nodes). The wet liver weights and liver:body weight ratio of the mice in the CG_200277 and control groups were also found to be similar (FIG. 17B). Long-term administration of siRNA targeting ITFG1 did not cause significant toxicity. Similar findings were observed for the mice treated with PBS or CG_200277 for 12 months (FIGS. 17C and 17D).

### Example 18: Evaluation of CG 200280-Induced Knockdown of ITFG1 Expression in Rat and Non-Human Primate (Cynomolgus Monkey) Livers

The knockdown potency of siRNA targeting ITFG1 (CG_200280) in rats and cynomolgus monkeys was evaluated.

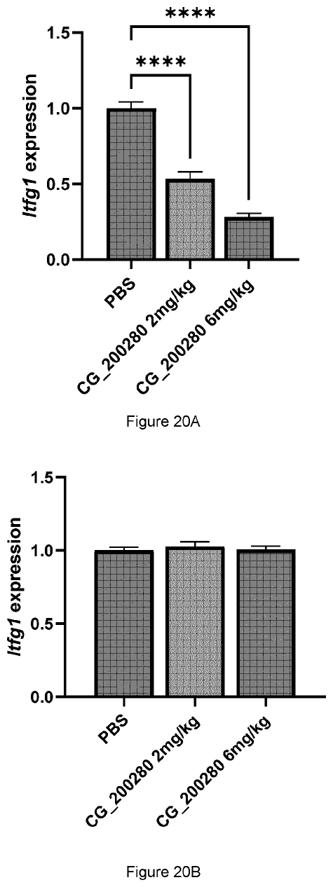

Rats were administered with PBS, or a single dose of siRNA targeting ITFG1 (CG_200280) subcutaneously at one of the two dosages (2 mg/kg or 6 mg/kg body weight). The livers and kidneys were harvested 7 days post-injection and processed for quantification of ITFG1 expression by qPCR as described in Example 10, using primers of SEQ IDs 250 and 251: Despite a single mismatch outside of the seed region between the siRNA and Rat ortholog of Itfg1, dose-dependent knockdown of ITFG1 expression was observed in rat livers (FIG. 20A). No change was observed in ITFG1 expression of rat kidneys, underscoring the specificity of the GalNAc conjugate to liver (FIG. 20B).

Cynomolgus monkeys were administered with PBS, or a single dose of siRNA targeting ITFG1 (CG_200280) subcutaneously at either 2 mg/kg or 6 mg/kg body weight on Day 0. On Days 14, 28, 56 and 84, 2 biopsies were taken from each cynomolgus monkey's liver, and processed for quantification of ITFG1 expression by qPCR as described in Example 10, using primers of SEQ IDs: 252 and 253. Dose-dependant knockdown of ITFG1 expression was observed in cynomolgus monkey livers, with 6 mg/kg producing 80% knockdown on Day 14. Both dosages maintained approximately 50% knockdown 84 days after siRNA administration (FIG. 20C).

### Example 19: Evaluation of Serum Correlates of Toxicity in Rats Administered with Single High Dosages of siRNAs

The toxicity of the ITFG1-targeting siRNAs in rats was evaluated.

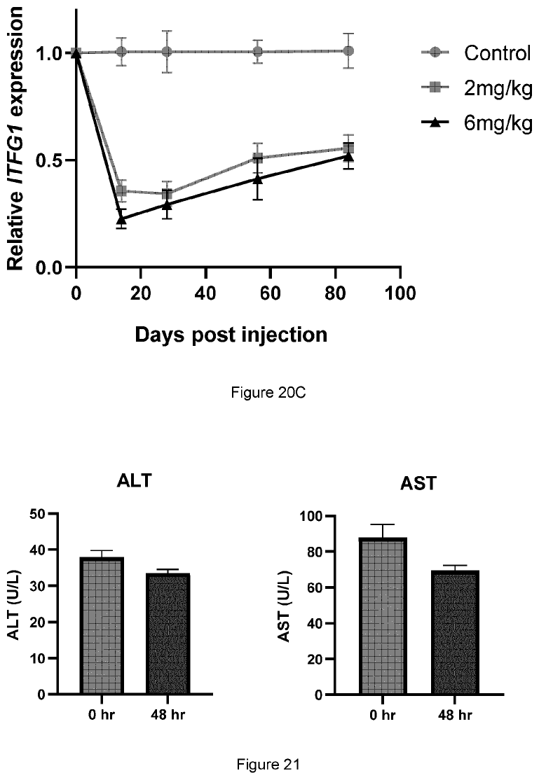

Rats were administered with PBS, or a single dose of siRNA targeting ITFG1 (CG_200280) of various dosages (30 mg/kg, 100 mg/kg or 400 mg/kg body weight). Sera were collected from the rats immediately before injection, and 48 hours after injection. The sera were analyzed for serum correlates of toxicity like AST and ALT. No significant difference in AST and ALT levels between the two timepoints was observed for all treatment groups (400 mg/kg, FIG. 21).

### Example 20: Evaluation of Pharmacokinetics of CG 200280 in Rat and Cynomolgus Monkey Plasma

Pharmacokinetics of CG_200280 was evaluated in the plasma of rats and cynomolgus monkeys.

At 0 hours, rats were subcutaneously injected with a single dose of 0, 30, 100 or 400 mg/kg of CG_200280, while cynomolgus monkeys were subcutaneously injected with a single dose of CG_200280 at 0, 2 or 6 mg/kg bodyweight. Plasma was collected from the animals right before injection (0 minute), 15 minutes, 30 minutes, 1 hour, 2 hours, 4 hours, 8 hours, 12 hours, 24 hours and 48 hours after injection. The amount of full length SEQ ID Nos: 215 and 241 present in each plasma sample was analyzed by LC-MS to calculate half-life (t_{1/2}), time taken to reach maximum plasma concentration (Tₘₐₓ), maximum concentration of each sequence detected in the plasma across all timepoints (Cₘₐₓ), and the amount of body exposure to the sequences (AUC). The results are summarized in the tables below.

Rats

| | Dosage group | t_{1/2} | Tₘₐₓ | Cₘₐₓ | AUC |
|---|---|---|---|---|---|
| Strand | (mg/kg) | (hour) | (hour) | (ng/mL) | (h · μg/mL) |
| SEQ ID | 30 | — | 1.17 | 7.71 | 19.1 |
| NO: 215 | 100 | 1.4 | 10.79 | 35.7 | 155 |
| | 400 | 4.3 | 1.17 | 121 | 676 |
| SEQ ID | 30 | — | 1.3 | 7.65 | 18.5 |
| NO: 241 | 100 | 1.4 | 0.9 | 37.2 | 167 |
| | 400 | 4.2 | 1.2 | 124 | 714 |

Cynomolgus monkeys

| | Dosage group | t_{1/2} | Tₘₐₓ | Cₘₐₓ | AUC |
|---|---|---|---|---|---|
| Strand | (mg/kg) | (hour) | (hour) | (ng/mL) | (h · μg/mL) |
| SEQ ID | 2 | 1.9 | 1.7 | 646 | 2.65 |
| NO: 215 | 6 | 1.7 | 2 | 2030 | 10.8 |
| SEQ ID | 2 | 1.9 | 1.7 | 588 | 2.62 |
| NO: 241 | 6 | 1.7 | 2 | 2380 | 13.1 |

### Example 21: Evaluation of Knockdown of ITFG1 Expression and Toxicity in Primary Human Hepatocytes by Free Uptake

21.1 Potency of siRNA Knockdown of ITFG1 Expression

Freshly-thawed primary human hepatocytes are incubated in vitro with siRNA targeting ITFG1 (e.g. CG_200277, CG_200278 or CG_200279) at concentrations ranging from 0.1 nM to 10 μM for 24 hours, and RNA is subsequently harvested from the cells using a column-based kit according to manufacturer's instructions (Favorgen). ITFG1 mRNA expression is evaluated by qPCR as described in Example 2 above. siRNA targeting ITFG1 is found to inhibit ITFG1 expression in primary human hepatocytes by free uptake with an IC50 value of between 1 nM and 500 nM.

### 21.2 Toxicity

Freshly-thawed primary human hepatocytes are incubated in vitro with siRNA targeting ITFG1 (e.g. CG_200277, CG_200278 or CG_200279) at concentrations ranging from 1 nM to 10 μM for 72 hours. Cell viability is subsequently evaluated using the CellTiter-Glo Luminescent Cell Viability Assay (Promega) according to manufacturer's instructions. siRNA targeting ITFG1 is found not to display significant toxicity to primary human hepatocytes at concentrations ≤10 μM.

### Example 22: Evaluation of siRNA-Mediated Knockdown of ITFG1 Expression and Functional Consequences Thereof in Non-Hepatic Cell Lines

22.1 Potency of siRNA Knockdown of ITFG1 Expression

Non-hepatic cell lines of human and/or mouse origin such as CCL206 (mouse lung) and CRL1722 (mouse myoblast) are transfected with siRNA targeting ITFG1 (e.g. CG_200277, CG_200278, CG_200279 or CG_200280) at concentrations ranging from 80 fM to 100 nM, and maintained in culture for 3 days, as described above. Fold change in the level of mRNA encoding ITFG1 is determined as described in Example 2 (for human cell lines) or Example 5 (for mouse cell lines). siRNAs targeting ITFG1 are found to inhibit ITFG1 expression in non-hepatic cell lines with IC50 values of no more than 1 nM.

22.2 Potency of Induction of Proliferation by siRNA Knockdown of ITFG1

Non-hepatic cell lines of human and/or mouse origin such as CCL206 (mouse lung) and CRL1722 (mouse myoblast) are transfected with siRNA targeting ITFG1 (e.g. CG_200277, CG_200278, CG_200279, or CG_200280), and maintained in culture for 3 days, as described above. Cell proliferation is measured by a viability assay such as CellTiter-Glo or CCK-8 as described in Example 7. Cell proliferation is also measured by a proliferation assay such as Ki67 immunostaining or wound healing. Fold change in the level of mRNA encoding ITFG1 is determined as described in Example 2 (for human cell lines) or Example 5 (for moue cell lines). siRNAs targeting ITFG1 are found to potently promote cell proliferation compared to NT control.

1. An inhibitory nucleic acid for reducing gene and/or protein expression of ITFG1, wherein the inhibitory nucleic acid comprises or encodes antisense nucleic acid targeting a nucleotide sequence comprising, or consisting of, one of SEQ ID NOs:26, 33, 13 to 25, 27 to 32, 33 to 44, 128, 135, 115 to 127, 129 to 134, or 136 to 146.

2. The inhibitory nucleic acid according to claim 1, wherein the inhibitory nucleic acid comprises or encodes antisense nucleic acid targeting a nucleotide sequence comprising, or consisting of, SEQ ID NO:26, 33, 128 or 135.

3. The inhibitory nucleic acid according to claim 1 or claim 2, wherein the inhibitory nucleic acid comprises or encodes antisense nucleic acid comprising or consisting of a nucleotide sequence having at least 75% sequence identity to one of SEQ ID NOs:58, 65, 45 to 57, 59 to 64, 66 to 76, 160, 167, 147 to 159, 161 to 166, or 168 to 178.

4. The inhibitory nucleic acid according to any one of claims 1 to 3, wherein the inhibitory nucleic acid comprises or encodes antisense nucleic acid comprising or consisting of a nucleotide sequence having at least 75% sequence identity to SEQ ID NO:58, 65, 160 or 167.

5. The inhibitory nucleic acid according to any one of claims 1 to 4, wherein the inhibitory nucleic acid comprises: (i) nucleic acid comprising the nucleotide sequence of one of SEQ ID NOs:58, 65, 45 to 57, 59 to 64, 66 to 76, 160, 167, 147 to 159, 161 to 166, or 168 to 178, or a nucleotide sequence having at least 75% sequence identity to one of SEQ ID NOs:58, 65, 45 to 57, 59 to 64, 66 to 76, 160, 167, 147 to 159, 161 to 166, or 168 to 178; and (ii) nucleic acid comprising a nucleotide sequence having the reverse complement of the nucleotide sequence of (i), or having at least 75% sequence identity to the reverse complement of the nucleotide sequence of (i).

6. The inhibitory nucleic acid according to any one of claims 1 to 5, wherein the inhibitory nucleic acid comprises, or consists of: (i) nucleic acid comprising a nucleotide sequence indicated in column A of Table I, and (ii) nucleic acid comprising a nucleotide sequence indicated in column B of Table I, wherein the sequences of columns A and B are selected from the same row of Table I.

7. The inhibitory nucleic acid according to any one of claims 1 to 5, wherein the inhibitory nucleic acid comprises, or consists of: (i) nucleic acid comprising a nucleotide sequence indicated in column A of Table II, and (ii) nucleic acid comprising a nucleotide sequence indicated in column B of Table II, wherein the sequences of columns A and B are selected from the same row of Table II.

8. The inhibitory nucleic acid according to any one of claims 1 to 7, wherein the inhibitory nucleic acid comprises one or more modified nucleotides selected from: 2′-O-methyluridine-3′-phosphate, 2′-O-methyladenosine-3′-phosphate, 2′-O-methylguanosine-3′-phosphate, 2′-O-methylcytidine-3′-phosphate, 2′-O-methyluridine-3′-phosphorothioate, 2′-O-methyladenosine-3′-phosphorothioate, 2′-O-methylguanosine-3′-phosphorothioate, 2′-O-methylcytidine-3′-phosphorothioate, 2′-fluorouridine-3′-phosphate, 2′-fluoroadenosine-3′-phosphate, 2′-fluoroguanosine-3′-phosphate, 2′-fluorocytidine-3′-phosphate, 2′-fluorocytidine-3′-phosphorothioate, 2′-fluoroguanosine-3′-phosphorothioate, 2′-fluoroadenosine-3′-phosphorothioate, and 2′-fluorouridine-3′-phosphorothioate.

9. The inhibitory nucleic acid according to any one of claims 1 to 8, wherein the inhibitory nucleic acid comprises, or consists of: (i) nucleic acid comprising a nucleotide sequence (including the modifications thereto) indicated in column A of Table III, and (ii) nucleic acid comprising a nucleotide sequence (including the modifications thereto) indicated in column B of Table III, wherein the sequences of columns A and B are selected from the same row of Table III;
optionally wherein the inhibitory nucleic acid comprises, or consists of:
(a) (i) SEQ ID NO:215, and (ii) SEQ ID NO:235;
(b) (i) SEQ ID NO:215, and (ii) SEQ ID NO:241;
(c) (i) SEQ ID NO:215, and (ii) SEQ ID NO:254;
(d) (i) SEQ ID NO:215, and (ii) SEQ ID NO:255;
(e) (i) SEQ ID NO:215, and (ii) SEQ ID NO:256;
(f) (i) SEQ ID NO:215, and (ii) SEQ ID NO:257;
(g) (i) SEQ ID NO:216, and (ii) SEQ ID NO:235; or
(h) (i) SEQ ID NO:226, and (ii) SEQ ID NO:240.

10. The inhibitory nucleic acid according to any one of claims 1 to 9, wherein the inhibitory nucleic acid comprises a moiety facilitating uptake of the inhibitory nucleic acid by hepatocytes, optionally wherein the moiety facilitating uptake of the inhibitory nucleic acid by hepatocytes is or comprises a N-acetylgalactosamine (GalNAc) moiety.

11. The inhibitory nucleic acid according to any one of claims 1 to 10, wherein the inhibitory nucleic acid is an siRNA.

12. A nucleic acid, optionally isolated, encoding an inhibitory nucleic acid according to any one of claims 1 to 11.

13. An expression vector, comprising a nucleic acid according to claim 12.

14. A composition comprising an inhibitory nucleic acid according to any one of claims 1 to 11, a nucleic acid according to claim 12, or an expression vector according to claim 13, and a pharmaceutically acceptable carrier, diluent, excipient or adjuvant.

15. A cell comprising an inhibitory nucleic acid according to any one of claims 1 to 11, a nucleic acid according to claim 12, or an expression vector according to claim 13.

16. An in vitro or in vivo method for reducing gene and/or protein expression of ITFG1 in a cell, comprising introducing an inhibitory nucleic acid according to any one of claims 1 to 11, a nucleic acid according to claim 12, or an expression vector according to claim 13 into a cell.

17. Use of an inhibitory nucleic acid according to any one of claims 1 to 11, a nucleic acid according to claim 12, an expression vector according to claim 13, or a composition according to claim 14, to reduce gene and/or protein expression of ITFG1 in vitro or in vivo.

18. An inhibitory nucleic acid according to any one of claims 1 to 11, a nucleic acid according to claim 12, an expression vector according to claim 13, or a composition according to claim 14, for use in a method of medical treatment or prophylaxis.

19. An inhibitory nucleic acid according to any one of claims 1 to 11, a nucleic acid according to claim 12, an expression vector according to claim 13, or a composition according to claim 14, for use in a method of treating or preventing a disease/condition selected from: a disease/condition characterised by fibrosis, a disease/condition characterised by damage to and/or death of cells of the liver, chronic liver disease, liver fibrosis, cirrhosis, non-alcoholic fatty liver disease (NAFLD), hepatitis, steatohepatitis, non-alcoholic steatohepatitis (NASH), alcoholic liver disease (ALD), alcoholic fatty liver (AFL), alcoholic hepatitis, alcoholic steatohepatitis (ASH), primary biliary cholangitis (PBC), schistosomal liver disease, congenital liver disease, liver cancer, hepatocellular carcinoma (HCC), acute liver injury (ALI), acute liver failure, acute liver disease, viral hepatitis, liver ischemia-reperfusion injury (IRI), warm ischemia-reperfusion (WIR), radiation-induced liver disease (RILD), drug-induced liver injury (DILI), acetaminophen-induced liver injury, autoimmune liver injury, liver transplantation, extended hepatectomy, small-for-size syndrome, split liver grafts, cholestatic liver disease, pulmonary fibrosis, interstitial lung disease (ILD), idiopathic interstitial pneumonia (IIP), idiopathic pulmonary fibrosis (IPF), cystic fibrosis, progressive massive fibrosis, scleroderma, obliterative bronchiolitis, Hermansky-Pudlak syndrome, asbestosis, silicosis, sarcoidosis, tumor stroma in lung disease, chronic obstructive pulmonary disease (COPD), emphysema, pneumonia, pulmonary edema, chronic bronchitis, asthma, hypertrophic cardiomyopathy (HCM), dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertension, hypertensive heart disease, arrhythmogenic right ventricular cardiomyopathy (ARVC), atherosclerosis, chronic pulmonary hypertension, AIDS-associated pulmonary hypertension, varicose veins, cerebral infarcts, tubulointerstitial fibrosis, glomerular fibrosis, renal fibrosis, nephritic syndrome, Alport's syndrome, HIV-associated nephropathy, polycystic kidney disease, Fabry's disease, diabetic nephropathy, chronic glomerulonephritis, nephritis associated with systemic lupus, pancreatic fibrosis, cystic fibrosis, chronic pancreatitis, gliosis, Alzheimer's disease, multiple sclerosis, muscular dystrophy, Duchenne muscular dystrophy (DMD), Becker's muscular dystrophy (BMD), fibrotic myopathy, inflammatory bowel disease (IBD), Crohn's disease, microscopic colitis, primary sclerosing cholangitis (PSC), scleroderma, nephrogenic systemic fibrosis, Dupuytren's contracture, cutis keloid, Grave's ophthalmopathy, epiretinal fibrosis, retinal fibrosis, subretinal fibrosis, subretinal fibrosis associated with macular degeneration (e.g. wet age-related macular degeneration (AMD)), diabetic retinopathy, glaucoma, corneal fibrosis, post-surgical fibrosis (e.g. of the posterior capsule following cataract surgery, or of the bleb following trabeculectomy for glaucoma), conjunctival fibrosis, subconjunctival fibrosis, arthrofibrosis, arthritis, adhesive capsulitis, progressive systemic sclerosis (PSS), chronic graft versus host disease (GVHD), fibrotic pre-neoplastic, fibrotic neoplastic disease, fibrosis induced by chemical or environmental insult, gastric cancer, esophageal cancer, lung cancer, head and neck cancer, colorectal cancer, pancreatic cancer, cervical cancer, vulvar cancer, mediastinal fibrosis, retroperitoneal fibrosis, myelofibrosis, Peyronie's disease, renal tubular disorder, renal tubular acidosis, hypokalemia, hyperkalemia, acute tubular necrosis, Fanconi syndrome, Liddle syndrome, nephrogenic diabetes insipidus,
pseudohypoaldosteronism type I, chronic kidney disease, acute kidney injury, a neurodegenerative disease, amyotrophic lateral sclerosis, epilepsy, frontotemporal dementia, Parkinson's disease, Huntington's disease, multiple system atrophy, a prion disease, ulcerative colitis, inflammatory bowel syndrome, a chronic inflammatory condition of the gastrointestinal tract or coeliac disease.

20. Use of an inhibitory nucleic acid according to any one of claims 1 to 11, a nucleic acid according to claim 12, an expression vector according to claim 13, or a composition according to claim 14, in the manufacture of a medicament for treating or preventing a disease/condition selected from: a disease/condition characterised by fibrosis, a disease/condition characterised by damage to and/or death of cells of the liver, chronic liver disease, liver fibrosis, cirrhosis, non-alcoholic fatty liver disease (NAFLD), hepatitis, steatohepatitis, non-alcoholic steatohepatitis (NASH), alcoholic liver disease (ALD), alcoholic fatty liver (AFL), alcoholic hepatitis, alcoholic steatohepatitis (ASH), primary biliary cholangitis (PBC), schistosomal liver disease, congenital liver disease, liver cancer, hepatocellular carcinoma (HCC), acute liver injury (ALI), acute liver failure, acute liver disease, viral hepatitis, liver ischemia-reperfusion injury (IRI), warm ischemia-reperfusion (WIR), radiation-induced liver disease (RILD), drug-induced liver injury (DILI), acetaminophen-induced liver injury, autoimmune liver injury, liver transplantation, extended hepatectomy, small-for-size syndrome, split liver grafts, cholestatic liver disease, pulmonary fibrosis, interstitial lung disease (ILD), idiopathic interstitial pneumonia (IIP), idiopathic pulmonary fibrosis (IPF), cystic fibrosis, progressive massive fibrosis, scleroderma, obliterative bronchiolitis, Hermansky-Pudlak syndrome, asbestosis, silicosis, sarcoidosis, tumor stroma in lung disease, chronic obstructive pulmonary disease (COPD), emphysema, pneumonia, pulmonary edema, chronic bronchitis, asthma, hypertrophic cardiomyopathy (HCM), dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertension, hypertensive heart disease, arrhythmogenic right ventricular cardiomyopathy (ARVC), atherosclerosis, chronic pulmonary hypertension, AIDS-associated pulmonary hypertension, varicose veins, cerebral infarcts, tubulointerstitial fibrosis, glomerular fibrosis, renal fibrosis, nephritic syndrome, Alport's syndrome, HIV-associated nephropathy, polycystic kidney disease, Fabry's disease, diabetic nephropathy, chronic glomerulonephritis, nephritis associated with systemic lupus, pancreatic fibrosis, cystic fibrosis, chronic pancreatitis, gliosis, Alzheimer's disease, multiple sclerosis, muscular dystrophy, Duchenne muscular dystrophy (DMD), Becker's muscular dystrophy (BMD), fibrotic myopathy, inflammatory bowel disease (IBD), Crohn's disease, microscopic colitis, primary sclerosing cholangitis (PSC), scleroderma, nephrogenic systemic fibrosis, Dupuytren's contracture, cutis keloid, Grave's ophthalmopathy, epiretinal fibrosis, retinal fibrosis, subretinal fibrosis, subretinal fibrosis associated with macular degeneration (e.g. wet age-related macular degeneration (AMD)), diabetic retinopathy, glaucoma, corneal fibrosis, post-surgical fibrosis (e.g. of the posterior capsule following cataract surgery, or of the bleb following trabeculectomy for glaucoma), conjunctival fibrosis, subconjunctival fibrosis, arthrofibrosis, arthritis, adhesive capsulitis, progressive systemic sclerosis (PSS), chronic graft versus host disease (GVHD), fibrotic pre-neoplastic, fibrotic neoplastic disease, fibrosis induced by chemical or environmental insult, gastric cancer, esophageal cancer, lung cancer, head and neck cancer, colorectal cancer, pancreatic cancer, cervical cancer, vulvar cancer, mediastinal fibrosis, retroperitoneal fibrosis, myelofibrosis, Peyronie's disease, renal tubular disorder, renal tubular acidosis, hypokalemia, hyperkalemia, acute tubular necrosis, Fanconi syndrome, Liddle syndrome, nephrogenic diabetes insipidus,
pseudohypoaldosteronism type I, chronic kidney disease, acute kidney injury, a neurodegenerative disease, amyotrophic lateral sclerosis, epilepsy, frontotemporal dementia, Parkinson's disease, Huntington's disease, multiple system atrophy, a pion disease, ulcerative colitis, inflammatory bowel syndrome, a chronic inflammatory condition of the gastrointestinal tract or coeliac disease.

21. A method of treating or preventing a disease or condition in a subject, comprising administering to a subject a therapeutically- or prophylactically-effective amount of an inhibitory nucleic acid according to any one of claims 1 to 11, a nucleic acid according to claim 12, an expression vector according to claim 13, or a composition according to claim 14, wherein the disease or condition is selected from: a disease/condition characterised by fibrosis, a disease/condition characterised by damage to and/or death of cells of the liver, chronic liver disease, liver fibrosis, cirrhosis, non-alcoholic fatty liver disease (NAFLD), hepatitis, steatohepatitis, non-alcoholic steatohepatitis (NASH), alcoholic liver disease (ALD), alcoholic fatty liver (AFL), alcoholic hepatitis, alcoholic steatohepatitis (ASH), primary biliary cholangitis (PBC), schistosomal liver disease, congenital liver disease, liver cancer, hepatocellular carcinoma (HCC), acute liver injury (ALI), acute liver failure, acute liver disease, viral hepatitis, liver ischemia-reperfusion injury (IRI), warm ischemia-reperfusion (WIR), radiation-induced liver disease (RILD), drug-induced liver injury (DILI), acetaminophen-induced liver injury, autoimmune liver injury, liver transplantation, extended hepatectomy, small-for-size syndrome, split liver grafts, cholestatic liver disease, pulmonary fibrosis, interstitial lung disease (ILD), idiopathic interstitial pneumonia (IIP), idiopathic pulmonary fibrosis (IPF), cystic fibrosis, progressive massive fibrosis, scleroderma, obliterative bronchiolitis, Hermansky-Pudlak syndrome, asbestosis, silicosis, sarcoidosis, tumor stroma in lung disease, chronic obstructive pulmonary disease (COPD), emphysema, pneumonia, pulmonary edema, chronic bronchitis, asthma, hypertrophic cardiomyopathy (HCM), dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertension, hypertensive heart disease, arrhythmogenic right ventricular cardiomyopathy (ARVC), atherosclerosis, chronic pulmonary hypertension, AIDS-associated pulmonary hypertension, varicose veins, cerebral infarcts, tubulointerstitial fibrosis, glomerular fibrosis, renal fibrosis, nephritic syndrome, Alport's syndrome, HIV-associated nephropathy, polycystic kidney disease, Fabry's disease, diabetic nephropathy, chronic glomerulonephritis, nephritis associated with systemic lupus, pancreatic fibrosis, cystic fibrosis, chronic pancreatitis, gliosis, Alzheimer's disease, multiple sclerosis, muscular dystrophy, Duchenne muscular dystrophy (DMD), Becker's muscular dystrophy (BMD), fibrotic myopathy, inflammatory bowel disease (IBD), Crohn's disease, microscopic colitis, primary sclerosing cholangitis (PSC), scleroderma, nephrogenic systemic fibrosis, Dupuytren's contracture, cutis keloid, Grave's ophthalmopathy, epiretinal fibrosis, retinal fibrosis, subretinal fibrosis, subretinal fibrosis associated with macular degeneration (e.g. wet age-related macular degeneration (AMD)), diabetic retinopathy, glaucoma, corneal fibrosis, post-surgical fibrosis (e.g. of the posterior capsule following cataract surgery, or of the bleb following trabeculectomy for glaucoma), conjunctival fibrosis, subconjunctival fibrosis, arthrofibrosis, arthritis, adhesive capsulitis, progressive systemic sclerosis (PSS), chronic graft versus host disease (GVHD), fibrotic pre-neoplastic, fibrotic neoplastic disease, fibrosis induced by chemical or environmental insult, gastric cancer, esophageal cancer, lung cancer, head and neck cancer, colorectal cancer, pancreatic cancer, cervical cancer, vulvar cancer, mediastinal fibrosis, retroperitoneal fibrosis, myelofibrosis, Peyronie's disease, renal tubular disorder, renal tubular acidosis, hypokalemia, hyperkalemia, acute tubular necrosis, Fanconi syndrome, Liddle syndrome, nephrogenic diabetes insipidus,
pseudohypoaldosteronism type I, chronic kidney disease, acute kidney injury, a neurodegenerative disease, amyotrophic lateral sclerosis, epilepsy, frontotemporal dementia, Parkinson's disease, Huntington's disease, multiple system atrophy, a prion disease, ulcerative colitis, inflammatory bowel syndrome, a chronic inflammatory condition of the gastrointestinal tract or coeliac disease.

22. The inhibitory nucleic acid for use according to claim 19, the use according to claim 20, or the method according to claim 21, wherein the disease/condition is a disease/condition characterised by fibrosis, or a disease/condition characterised by damage to and/or death of cells of the liver.

23. The inhibitory nucleic acid for use, the use, or the method according to claim 22, wherein the disease/condition characterised by fibrosis is selected from: chronic liver disease, liver fibrosis, cirrhosis, non-alcoholic fatty liver disease (NAFLD), hepatitis, steatohepatitis, non-alcoholic steatohepatitis (NASH), alcoholic liver disease (ALD), alcoholic fatty liver (AFL), alcoholic hepatitis, alcoholic steatohepatitis (ASH), primary biliary cholangitis (PBC), schistosomal liver disease, congenital liver disease, liver cancer, hepatocellular carcinoma (HCC), acute liver injury (ALI), acute liver failure, acute liver disease, viral hepatitis, liver ischemia-reperfusion injury (IRI), warm ischemia-reperfusion (WIR), radiation-induced liver disease (RILD), drug-induced liver injury (DILI), acetaminophen-induced liver injury, autoimmune liver injury, liver transplantation, extended hepatectomy, small-for-size syndrome, split liver grafts, cholestatic liver disease, pulmonary fibrosis, interstitial lung disease (ILD), idiopathic interstitial pneumonia (IIP), idiopathic pulmonary fibrosis (IPF), cystic fibrosis, progressive massive fibrosis, scleroderma, obliterative bronchiolitis, Hermansky-Pudlak syndrome, asbestosis, silicosis, sarcoidosis, tumor stroma in lung disease, chronic obstructive pulmonary disease (COPD), emphysema, pneumonia, pulmonary edema, chronic bronchitis, asthma, hypertrophic cardiomyopathy (HCM), dilated cardiomyopathy (DCM), fibrosis of the atrium, atrial fibrillation, fibrosis of the ventricle, ventricular fibrillation, myocardial fibrosis, Brugada syndrome, myocarditis, endomyocardial fibrosis, myocardial infarction, fibrotic vascular disease, hypertension, hypertensive heart disease, arrhythmogenic right ventricular cardiomyopathy (ARVC), atherosclerosis, chronic pulmonary hypertension, AIDS-associated pulmonary hypertension, varicose veins, cerebral infarcts, tubulointerstitial fibrosis, glomerular fibrosis, renal fibrosis, nephritic syndrome, Alport's syndrome, HIV-associated nephropathy, polycystic kidney disease, Fabry's disease, diabetic nephropathy, chronic glomerulonephritis, nephritis associated with systemic lupus, pancreatic fibrosis, cystic fibrosis, chronic pancreatitis, gliosis, Alzheimer's disease, multiple sclerosis, muscular dystrophy, Duchenne muscular dystrophy (DMD), Becker's muscular dystrophy (BMD), fibrotic myopathy,
inflammatory bowel disease (IBD), Crohn's disease, microscopic colitis, primary sclerosing cholangitis (PSC), scleroderma, nephrogenic systemic fibrosis, Dupuytren's contracture, cutis keloid, Grave's ophthalmopathy, epiretinal fibrosis, retinal fibrosis, subretinal fibrosis, subretinal fibrosis associated with macular degeneration (e.g. wet age-related macular degeneration (AMD)), diabetic retinopathy, glaucoma, corneal fibrosis, post-surgical fibrosis (e.g. of the posterior capsule following cataract surgery, or of the bleb following trabeculectomy for glaucoma), conjunctival fibrosis, subconjunctival fibrosis, arthrofibrosis, arthritis, adhesive capsulitis, progressive systemic sclerosis (PSS), chronic graft versus host disease (GVHD), fibrotic pre-neoplastic, fibrotic neoplastic disease, fibrosis induced by chemical or environmental insult, gastric cancer, esophageal cancer, lung cancer, head and neck cancer, colorectal cancer, pancreatic cancer, cervical cancer, vulvar cancer, mediastinal fibrosis, retroperitoneal fibrosis, myelofibrosis and Peyronie's disease.

24. The inhibitory nucleic acid for use, the use, or the method according to claim 23, wherein the disease/condition characterised by damage to and/or death of cells of the liver is selected from: chronic liver disease, liver fibrosis, cirrhosis, non-alcoholic fatty liver disease (NAFLD), hepatitis, steatohepatitis, non-alcoholic steatohepatitis (NASH), alcoholic liver disease (ALD), alcoholic fatty liver (AFL), alcoholic hepatitis, alcoholic steatohepatitis (ASH), primary biliary cholangitis (PBC), primary sclerosing cholangitis (PSC), schistosomal liver disease, congenital liver disease, liver cancer, hepatocellular carcinoma (HCC), acute liver injury (ALI), acute liver failure, acute liver disease, viral hepatitis, liver ischemia-reperfusion injury (IRI), warm ischemia-reperfusion (WIR), radiation-induced liver disease (RILD), drug-induced liver injury (DILI), acetaminophen-induced liver injury, autoimmune liver injury, liver transplantation, extended hepatectomy, small-for-size syndrome, split liver grafts, and cholestatic liver disease.


SEQUENCE LISTING (257 sequences)

SEQ ID NO: 1 - protein, 612 aa, Homo sapiens
MAAAGRLPSSWALFSPLLAGLALLGVGPVPARALHNVTAELFGAEAWGTLAAFGDLNSDKQTDLFVLRERNDLIVFLADQNAPYFKPKVKVSFKNHSALITSVVPGDYDGDSQMDVLLTYLPKNYAKSELGAVIFWGQNQTLDPNNMTILNRTFQDEPLIMDFNGDLIPDIFGITNESNQPQILLGGNLSWHPALTTTSKMRIPHSHAFIDLTEDFTADLFLTTLNATTSTFQFEIWENLDGNFSVSTILEKPQNMMVVGQSAFADFDGDGHMDHLLPGCEDKNCQKSTIYLVRSGMKQWVPVLQDFSNKGTLWGFVPFVDEQQPTEIPIPITLHIGDYNMDGYPDALVILKNTSGSNQQAFLLENVPCNNASCEEARRMFKVYWELTDLNQIKDAMVATFFDIYEDGILDIVVLSKGYTKNDFAIHTLKNNFEADAYFVKVIVLSGLCSNDCPRKITPFGVNQPGPYIMYTTVDANGYLKNGSAGQLSQSAHLALQLPYNVLGLGRSANFLDHLYVGIPRPSGEKSIRKQEWTAIIPNSQLIVIPYPHNVPRSWSAKLYLTPSNIVLLTAIALIGVCVFILAIIGILHWQEKKADDREKRQEAHRFHFDAM

SEQ ID NO: 2 - protein, 36 aa, synthetic construct
VVGQSAFADFDGDGHMDHLLPGCEDKNCQKSTIYLV

SEQ ID NO: 3 - protein, 33 aa, Homo sapiens
MAAAGRLPSSWALFSPLLAGLALLGVGPVPARA

SEQ ID NO: 4 - protein, 21 aa, synthetic construct
VLLTAIALIGVCVFILAIIGI

SEQ ID NO: 5 - protein, 579 aa, synthetic construct
LHNVTAELFGAEAWGTLAAFGDLNSDKQTDLFVLRERNDLIVFLADQNAPYFKPKVKVSFKNHSALITSVVPGDYDGDSQMDVLLTYLPKNYAKSELGAVIFWGQNQTLDPNNMTILNRTFQDEPLIMDFNGDLIPDIFGITNESNQPQILLGGNLSWHPALTTTSKMRIPHSHAFIDLTEDFTADLFLTTLNATTSTFQFEIWENLDGNFSVSTILEKPQNMMVVGQSAFADFDGDGHMDHLLPGCEDKNCQKSTIYLVRSGMKQWVPVLQDFSNKGTLWGFVPFVDEQQPTEIPIPITLHIGDYNMDGYPDALVILKNTSGSNQQAFLLENVPCNNASCEEARRMFKVYWELTDLNQIKDAMVATFFDIYEDGILDIVVLSKGYTKNDFAIHTLKNNFEADAYFVKVIVLSGLCSNDCPRKITPFGVNQPGPYIMYTTVDANGYLKNGSAGQLSQSAHLALQLPYNVLGLGRSANFLDHLYVGIPRPSGEKSIRKQEWTAIIPNSQLIVIPYPHNVPRSWSAKLYLTPSNIVLLTAIALIGVCVFILAIIGILHWQEKKADDREKRQEAHRFHFDAM

SEQ ID NO: 6 - protein, 464 aa, Homo sapiens
MDVLLTYLPKNYAKSELGAVIFWGQNQTLDPNNMTILNRTFQDEPLIMDFNGDLIPDIFGITNESNQPQILLGGNLSWHPALTTTSKMRIPHSHAFIDLTEDFTADLFLTTLNATTSTFQFEIWENLDGNFSVSTILEKPQNMMVVGQSAFADFDGDGHMDHLLPGCEDKNCQKSTIYLVRSGMKQWVPVLQDFSNKGTLWGFVPFVDEQQPTEIPIPITLHIGDYNMDGYPDALVILKNTSGSNQQAFLLENVPCNNASCEEARRMFKVYWELTDLNQIKDAMVATFFDIYEDGILDIVVLSKGYTKNDFAIHTLKNNFEADAYFVKVIVLSGLCSNDCPRKITPFGVNQPGPYIMYTTVDANGYLKNGSAGQLSQSAHLALQLPYNVLGLGRSANFLDHLYVGIPRPSGEKSIRKQEWTAIIPNSQLIVIPYPHNVPRSSHRCLCFHLGNNWHFTLAGKESR

SEQ ID NO: 7 - protein, 612 aa, Macaca fascicularis
MAAAGRLPSSWALLSPLLTGLALLGVGPVPARALHNVTAELFGAEAWGTLAAFGDLNSDKQTDLFVLRERNDLIVFLADQNAPYFKPKVKVSFKNHSALITSVVPGDYDGDSQMDVLLTYLPKNYAKSELGAVIFWGQNQTLDPNNMTILNRTFQDEPLIMDFNGDLIPDIFGITNESNQPQILLGGNLSWHPALTTTSKMRIPHSHAFIDLTEDFTADLFLTTLNATSSTFQFEIWENLDGNFSVSTILEKPQNMMVVGQSAFADFDGDGHMDHLLPGCEDKNCQKSTIYLVRSGTKQWVPVLQDFSNKGTLWGFVPFVDEQQPTEIPIPITLHIGDYNMDGYPDALVILKNTSGSNQQAFLLENVPCNNASCEEARRMFKVYWELTDLNQIKDAMVATFFDIYEDGILDIVVLSKGYTKNDFAIHTLKNNFEADAYFVKVIVLSGLCSNDCPRKITPFGVNQPGPYIMYTTVDANGYLKNGSAGQLSQSAHLALQLPYNVLGLGRSANFLDHLYVGIPRPSGEKSIRKQEWTAIIPNSQLIVIPYPHNVPRSWSAKLYLTPSNIVLLTAIALIGVCVFILAIIGILHWQEKKADDREKRQEAHRFHFDAM

SEQ ID NO: 8 - protein, 610 aa, Mus musculus
MAAGRLPSARAVLAPLFLGLALLSVGPAPARALHNVTAELFGAEAWGTLAAFGDLNSDKQTDLFVLRERNDLIVFLADQSAPYFKPKVKVSLKTLSALVTSVVPGDYDGDSQMDVLLTYFPQNHTNSELGAVIFWGQNQTLDPKNMTILNRTFHDQPLIMDFNGDLIPDVFGITNESSQPQILLGGDLSWHPALTTKSKMRDPHSHAFIDLTEDFTADLFLTTLTASNAFQFEIWENLGGNFSIHSVFEKPKNLVVVGQSAFADFDGDGHMDHLLPGCEDKDCQKSAIYLMRSGTGQWVPVLQDFSNKGTLWGFVPFVHEEQPTTIPIPLTLHIGDYNMDGYPDALAILKNTSGSNQQAFLLENVPCNNASCEEVHRMFKVYWDLAGLNLIKDAIVATFFDIYEDGILDIIVLSKGYTKNDVAIHTLKNNFEADAYFVKVIVLSGLCSNDCPRKITPFGVNQPGPYIMYTTVDANGYLKNGSAGQLSQSAHLALQLPYNVLGLGRSANFLDHLFVGIPRPSGEKSIRKQEWTAIIPNSQLIVIPYPHNVPRSWSAKLYLTPSNIVLLTAVALIGVCIFILAIIAILHWQEKKADDREKRQEAHRFHFDAM

SEQ ID NO: 9 - RNA, 3185 nt, Homo sapiens
  source 1..3185: mol_type = other RNA
gggggctgaggggctgccatggcggcggcgggccggctcccgagctcctgggccctcttctcgccgctcctcgcagggcttgcactactgggagtcgggccggtcccagcgcgggcgctgcacaacgtcacggccgagctctttggggccgaggcctggggcacccttgcggctttcggggacctcaactccgacaagcagacggatctcttcgtgctgcgggaaagaaatgacttaatcgtctttttggcagaccagaatgcaccctattttaaacccaaagtaaaggtatctttcaagaatcacagtgcattgataacaagtgtagtccctggggattatgatggagattctcaaatggatgtccttctgacatatcttcccaaaaattatgccaagagtgaattaggagctgttatcttctggggacaaaatcaaacattagatcctaacaatatgaccatactcaataggacttttcaagatgagccactaattatggatttcaatggtgatctaattcctgatatttttggtatcacaaatgaatccaaccagccacagatactattaggagggaatttatcatggcatccagcattgaccactacaagtaaaatgcgaattccacattctcatgcatttattgatctgactgaagattttacagcagatttattcctgacgacattgaatgccaccactagtaccttccagtttgaaatatgggaaaatttggatggaaacttctctgtcagtactatattggaaaaacctcaaaatatgatggtggttggacagtcagcatttgcagactttgatggagatggacacatggatcatttactgccaggctgtgaagataaaaattgccaaaagagtaccatctacttagtgagatctgggatgaagcagtgggttccagtcctacaagatttcagcaataagggcacactctggggctttgtgccatttgtggatgaacagcaaccaactgaaataccaattccaattacccttcatattggagactacaatatggatggctatccagacgctctggtcatactaaagaacacatctggaagcaaccagcaggcctttttactggagaacgtcccttgtaataatgcaagctgtgaagaggcgcgtcgaatgtttaaagtctactgggagctgacagacctaaatcaaattaaggatgccatggttgccaccttctttgacatttacgaagatggaatcttggacattgtagtgctaagtaaaggatatacaaagaatgattttgccattcatacactaaaaaataactttgaagcagatgcttattttgttaaagttattgttcttagtggtctgtgttctaatgactgtcctcgtaagataacaccctttggagtgaatcaacctggaccttatatcatgtatacaactgtagatgcaaatgggtatctgaaaaatggatcagctggccaactcagccaatccgcacatttagctctccaactaccatacaacgtgcttggtttaggtcggagcgcaaattttcttgaccatctctacgttggtattccccgtccatctggagaaaaatctatacgaaaacaagagtggactgcaatcattccaaattcccagctaattgtcattccataccctcacaatgtccctcgaagttggagtgccaaactgtatcttacaccaagtaatattgttctgcttactgctatagctctcatcggtgtctgtgttttcatcttggcaataattggcattttacattggcaggaaaagaaagcagatgatagagaaaaacgacaagaagcccaccggtttcattttgatgctatgtgacttgcctttaatattacataatggaatggctgttcacttgattagttgaaacacaaattctggcttgaaaaaataggggagattaaatattatttataaatgatgtatcccatggtaattattggaaagtattcaaataaatatggtttgaatatgtcacaaggtctttttttttaaagcactttgtatataaaaatttgggttctctattctgtagtgctgtacatttttgttcctttgtggaatgtgttgcatgtactccagtgtttgtgtatttataatcttatttgcatcatgatgatggaaaaagttgtgtaaataaaaataattaaatgagcaggaatttttgtgtccacttgacttggtcttgcttcttattctaatgatgcaaattatacttttgtgaatatatcacggagtcattaggcattcagcttcatcacagcaggtcaggggtctcactgatggcatacaatatagtgatcgggtactctgacttggtagcacagtaagacagacttgccttaaactcctaattcaaccacttacaaagtcattgtttgaacttggctcttgtttaacctctgtaaacctcagttttcttgtttattcagtggggctaatacttgagttactgtaaacattaaatgggatgatgtatgtgaagtgcttagcttggtgcctagcacagagtaagtggtcaatatgtggtagttgtcattattaatattttagatgatcttattagacttatacatctaattatagaaatacatagacttgatagaattttattttcaggcatgaagaaatattctttggaaaagctaaatttttggtgattgacataaagatttacttgctcatattaactaaaaattatagtactctccaagaattaatgtgccctaaaaattttcctccaaaaacttatccttatcatgtgataatgaagaacatttgatttcttgaaaggaaactgctgtaggcagcatctgggaatgcaaatcttcaatcacatttctattctcaaacacttggagaagtctataatttacattcagacttcaatgcaaattttgtattgtgaacttcacatttccaaaaagttactttaaaaagactttaagactgaaaaaaaaaagtttatcaatgctaataattttctagtatgcaaatggacatgtgatgcctataaaacacaaaaatttctctgaaaacaattttgttcttatttttttctttatagttcactgagattggcatgtgtttttactttgtatctaagcatgttaacatgtcttcttaataaatattccttattgaaa

SEQ ID NO: 10 - RNA, 3035 nt, Homo sapiens
  source 1..3035: mol_type = other RNA
gagcgtgagggcggctttcaccctagctccactgcttttttagcgtcggaagggcagccctagtgggagagagaaaaaggagccggcagcggctcttacgcgtcccggggctgcgcgccactctctcggccggaaatgacttaatcgtctttttggcagaccagaatgcaccctattttaaacccaaagtaaaggtatctttcaagaatcacagtgcattgataacaagtgtagtccctggggattatgatggagattctcaaatggatgtccttctgacatatcttcccaaaaattatgccaagagtgaattaggagctgttatcttctggggacaaaatcaaacattagatcctaacaatatgaccatactcaataggacttttcaagatgagccactaattatggatttcaatggtgatctaattcctgatatttttggtatcacaaatgaatccaaccagccacagatactattaggagggaatttatcatggcatccagcattgaccactacaagtaaaatgcgaattccacattctcatgcatttattgatctgactgaagattttacagcagatttattcctgacgacattgaatgccaccactagtaccttccagtttgaaatatgggaaaatttggatggaaacttctctgtcagtactatattggaaaaacctcaaaatatgatggtggttggacagtcagcatttgcagactttgatggagatggacacatggatcatttactgccaggctgtgaagataaaaattgccaaaagagtaccatctacttagtgagatctgggatgaagcagtgggttccagtcctacaagatttcagcaataagggcacactctggggctttgtgccatttgtggatgaacagcaaccaactgaaataccaattccaattacccttcatattggagactacaatatggatggctatccagacgctctggtcatactaaagaacacatctggaagcaaccagcaggcctttttactggagaacgtcccttgtaataatgcaagctgtgaagaggcgcgtcgaatgtttaaagtctactgggagctgacagacctaaatcaaattaaggatgccatggttgccaccttctttgacatttacgaagatggaatcttggacattgtagtgctaagtaaaggatatacaaagaatgattttgccattcatacactaaaaaataactttgaagcagatgcttattttgttaaagttattgttcttagtggtctgtgttctaatgactgtcctcgtaagataacaccctttggagtgaatcaacctggaccttatatcatgtatacaactgtagatgcaaatgggtatctgaaaaatggatcagctggccaactcagccaatccgcacatttagctctccaactaccatacaacgtgcttggtttaggtcggagcgcaaattttcttgaccatctctacgttggtattccccgtccatctggagaaaaatctatacgaaaacaagagtggactgcaatcattccaaattcccagctaattgtcattccataccctcacaatgtccctcgaagctctcatcggtgtctgtgttttcatcttggcaataattggcattttacattggcaggaaaagaaagcagatgatagagaaaaacgacaagaagcccaccggtttcattttgatgctatgtgacttgcctttaatattacataatggaatggctgttcacttgattagttgaaacacaaattctggcttgaaaaaataggggagattaaatattatttataaatgatgtatcccatggtaattattggaaagtattcaaataaatatggtttgaatatgtcacaaggtctttttttttaaagcactttgtatataaaaatttgggttctctattctgtagtgctgtacatttttgttcctttgtggaatgtgttgcatgtactccagtgtttgtgtatttataatcttatttgcatcatgatgatggaaaaagttgtgtaaataaaaataattaaatgagcaggaatttttgtgtccacttgacttggtcttgcttcttattctaatgatgcaaattatacttttgtgaatatatcacggagtcattaggcattcagcttcatcacagcaggtcaggggtctcactgatggcatacaatatagtgatcgggtactctgacttggtagcacagtaagacagacttgccttaaactcctaattcaaccacttacaaagtcattgtttgaacttggctcttgtttaacctctgtaaacctcagttttcttgtttattcagtggggctaatacttgagttactgtaaacattaaatgggatgatgtatgtgaagtgcttagcttggtgcctagcacagagtaagtggtcaatatgtggtagttgtcattattaatattttagatgatcttattagacttatacatctaattatagaaatacatagacttgatagaattttattttcaggcatgaagaaatattctttggaaaagctaaatttttggtgattgacataaagatttacttgctcatattaactaaaaattatagtactctccaagaattaatgtgccctaaaaattttcctccaaaaacttatccttatcatgtgataatgaagaacatttgatttcttgaaaggaaactgctgtaggcagcatctgggaatgcaaatcttcaatcacatttctattctcaaacacttggagaagtctataatttacattcagacttcaatgcaaattttgtattgtgaacttcacatttccaaaaagttactttaaaaagactttaagactgaaaaaaaaaagtttatcaatgctaataattttctagtatgcaaatggacatgtgatgcctataaaacacaaaaatttctctgaaaacaattttgttcttatttttttctttatagttcactgagattggcatgtgtttttactttgtatctaagcatgttaacatgtcttcttaataaatattccttattgaaa

SEQ ID NO: 11 - RNA, 2206 nt, Macaca fascicularis
  source 1..2206: mol_type = other RNA
gggggccgaggggctgccatggcggcggcgggccggctgccgagctcctgggcgctcctgtcgccgctcctcacggggcttgcactactgggagtcgggccggtcccagcgcgggcgctgcacaacgtcacggccgagctctttggggccgaggcctggggcacccttgcggctttcggggacctcaactccgacaagcagacggacctcttcgtgctgcgggaaagaaacgacttaatcgtctttttggcagaccagaatgcaccctattttaaacctaaagtaaaggtatctttcaagaatcacagtgcattgataacaagtgtagtccctggggattatgatggagattctcaaatggatgtccttctgacatatcttcccaaaaattatgccaagagtgaattaggagctgttatcttctggggacaaaatcaaacgttagatcctaacaatatgaccatactcaataggacttttcaagatgagccactaattatggatttcaatggtgatctaattcctgatatttttggtatcacaaatgaatccaaccagccacagatactattaggagggaatttatcatggcatcctgcattgaccactacaagtaaaatgcgaattccacattctcatgcatttattgatctgactgaagattttacagcagatttatttctgacgacattgaatgccacctctagtaccttccagtttgagatatgggaaaatttggatggaaacttctctgtcagtactatattggaaaaacctcaaaatatgatggtggttggacagtcagcatttgcagactttgatggagatggacacatggatcatttactgccaggctgtgaagataaaaattgccaaaagagtaccatctacttagtgagatctgggacaaagcagtgggttccagtcctacaagatttcagtaataagggcacactctggggctttgtgccgtttgtggatgaacagcaaccaactgaaataccaattccaattacccttcatattggagactacaatatggatggctatccagatgctctggtcatactaaagaacacatctggaagcaaccagcaggcctttttactggagaacgtcccttgtaataatgcaagctgtgaagaggcgcgtcgaatgttcaaagtctactgggagctgacagacctaaatcaaattaaggatgccatggttgccaccttctttgacatttacgaagatggaatcttggacattgtagtgctaagtaaaggatatacaaagaatgattttgccatccatacactaaaaaataactttgaagcagatgcttattttgttaaagttattgttcttagtggtctgtgttctaatgactgtcctcgtaagataacaccctttggagtgaatcaacctggaccttatatcatgtatacaactgttgatgcaaatgggtatctgaaaaatggatcagctggtcaactcagccaatccgcacatttagctctccaactaccatacaacgtgcttggtttaggtcggagcgcaaattttcttgaccatctgtatgttggtattccccgtccatctggagaaaaatctatacgaaaacaagagtggactgcaatcattccaaattcccagctaattgtcattccataccctcacaatgtccctcgaagttggagtgccaaactatatcttacaccaagtaacattgttctgcttactgccatagctctcatcggtgtctgtgttttcatcttggcaataattggcattttacattggcaggaaaagaaagcagatgatagagaaaaacgacaagaagcccaccggtttcattttgatgctatgtgacttgcctttaatgttacataatggaatggctgttcacttgattagttgaaacacaaattctggcttaaaaaataggagagattaaatattatttgtaaatgatgtatcccatggtaattattggaaagtattcaaataaatatggtttgaatatgtcacaaggtcctttttttttttaaaagcactttgtatataaaaagtttgggttctttattccgtagtgctgtacatttctgttcctttgtggaatgtgttgcatgtactcccgtgtgtgtgtatttataatcttattagtgtcatgatgatggaaaaagatgtgtgtaaataaaaataattaaatgagcaggaa

SEQ ID NO: 12 - RNA, 3355 nt, Mus musculus
  source 1..3355: mol_type = other RNA
cacgacagccggaagtgagttcccgggcagacgccgccctagctcctcggcgtcttggtcgtcggagaaacagccccagggcagcgtgagagtgcagcgaggggctgcccgtgaggccgcaccccgagtcttttctcggtcggccggtcagtcgcacggtgctttgcggctgccgtcaagcgcggcggaggccggccgggccgaaggcgccatggcggcaggccggcttcctagcgccagggcggtgctggccccgctgttcttggggctcgcgctgctgagcgttgggcctgccccggcgcgggctctgcacaacgtcacggccgagctctttggggctgaggcctggggcaccctggcggccttcggtgacctcaactccgacaaacagacggacctcttcgtgctgcgggaaagaaatgacttaattgtcttcttggcagatcagagtgcaccctattttaaacccaaggtaaaggtatccctgaagactttgagtgcgttggtaacaagtgtggtccctggtgattatgatggggattcacaaatggatgtcctgctcacatactttccccaaaatcataccaacagtgaactaggagctgttatcttttggggacaaaatcaaacattagatcctaaaaacatgaccatactcaataggacttttcatgaccaaccactgattatggacttcaatggtgatctaattcctgatgtgtttggtatcacaaatgaatccagccaaccgcagatactgctaggaggagatttatcatggcatcctgccttgaccactaagagtaaaatgcgagatccacattcccatgcatttattgatctgactgaagattttacagcagatttattcctcacgacattgactgcctctaatgccttccagtttgagatatgggaaaatttgggtggaaacttctctatccatagtgtctttgaaaaacctaaaaatctggtggtggttggacagtcagcatttgcagactttgatggagatggacacatggatcacttgctgccaggctgtgaagataaggactgtcagaaaagtgccatatatttaatgagatctggaacaggacagtgggttcctgtgcttcaggattttagcaataagggtacactctggggctttgtgccatttgtgcatgaagaacaaccaaccacaataccaatcccactcacacttcacattggagactacaacatggacggctacccagatgctctggctatattgaagaatacatctggaagcaatcagcaagcctttttactggagaacgtcccatgcaataatgcgagctgcgaagaagtgcatcggatgtttaaagtctactgggacctagcaggcctaaatctaatcaaagacgccatagttgccaccttttttgatatttatgaagatggaatcttggacattatagtcctcagtaaaggatacaccaagaatgatgttgccatccatacactcaaaaataactttgaggcagatgcttactttgttaaagtcattgttcttagtggtctatgttctaatgactgtcctcgtaagataacaccttttggagtaaatcagcctggaccttatatcatgtacacaacagtagatgcaaatggatatctgaaaaacggctcagctggacagctcagccagtcagcacacttagctcttcagctaccttacaatgtacttggcttaggccgaagtgccaatttcctggatcatctttttgttggtattccccgtccatctggagagaagtctataagaaaacaagagtggacagcaatcattccaaattcccagctgattgtcattccatatcctcacaatgtccctcgaagctggagtgccaaattgtacctgacaccaagtaacattgtcctgctgactgcggtagccctcattggagtctgcatttttatcctggcaataatcgccattttacattggcaggaaaagaaagccgatgacagagaaaaacgacaggaagcacatcggtttcattttgatgctatgtgactttgctgaagtagtacatcctggaaatcacttgattagtgaaacattaaattttgactcctagtttaaaaaaaatgatggatctgagatatttataaattatgccttccttgtcaattattggaaaatactgaaataaatatggcctgaatatctcataaagtctgttttaagcactttgtatataataatttaggttcttaatttagcagcactgagcatttgtgttcctttgtggaatgtgttgcatgtacttggtgtatctgtgtttacaatgttgttagactgaggatggtgaaaggacgtgtaaataaaaactatttaaatgacctgtggccagggcgttgttctgcctaaattgatcttgtttttatctttaaaatgcatactttcacaaatatgtgccagataaagttattcaagtaacattcttcactgcagatcagactgctcatgcgtggcacagaatgaaactaaatagccaggctttggaatcataggaggacagcttgccttagatgcctggttctacaacttagagttatctgaatatggctgctgcttagcgtctccgtaagcctcagttttgtttgtatgatgaggctaatatgcttgttcacttgagttgtcatgagcatacattgtatgatgtacatgatgtacttagctcagtgtctgagcatacagaaagcactcgtcaagcagctgctgtcatgattgctactgtaggtgatcttattctactactgtatacaatggattgtcaccgaagacataaggactctttggaaaacttaaattattggtggtacatatcttacatagttaacttgtttaactagctctaatactatccaagagtaaatgtatcctgaaagctggtatttgaatactaatccttgcacagtaacataaatgatttgctctcttgaaagaaagctgatagaggcaaactcttcaggtgccagcatgccatacagttctgtctcaaaagacgggaaaggtctgtcccatttctacagacctcaggtttcatgttagaaacttcacattgccaacatttactttaaaattatttaaatctagatttcaggagagaggtgataaaattattatcaaagctaacaatatgctgattgttaagacatgctgtggctgtaaaatataaaattcctctggaacagttttgtttgtgttattttcctcactgcgattcactggagggcatgtgctcctttttatttctatctaagcatgttaatataccttcttaataaatactccttactgaataaaaacgctgtggcaagc

SEQ ID NO: 13 - RNA, 21 nt, synthetic construct
  source 1..21: mol_type = other RNA
  mRNA 1..21
ctgactgaagattttacagca

SEQ ID NO: 14 - RNA, 21 nt, synthetic construct
  source 1..21: mol_type = other RNA
  mRNA 1..21
aatgactgtcctcgtaagata

SEQ ID NO: 15 - RNA, 21 nt, synthetic construct
  source 1..21: mol_type = other RNA
  mRNA 1..21
atgactgtcctcgtaagataa

SEQ ID NO: 16 - RNA, 21 nt, synthetic construct
  source 1..21: mol_type = other RNA
  mRNA 1..21
ttacattggcaggaaaagaaa

SEQ ID NO: 17 - RNA, 21 nt, synthetic construct
  source 1..21: mol_type = other RNA
  mRNA 1..21
cttcgtgctgcgggaaagaaa

SEQ ID NO: 18 - RNA, 21 nt, synthetic construct
  source 1..21: mol_type = other RNA
  mRNA 1..21
ttctaatgactgtcctcgtaa

SEQ ID NO: 19 - RNA, 21 nt, synthetic construct
  source 1..21: mol_type = other RNA
  mRNA 1..21
gaagattttacagcagattta

SEQ ID NO: 20 - RNA, 21 nt, synthetic construct
  source 1..21: mol_type = other RNA
  mRNA 1..21
gtggttggacagtcagcattt

SEQ ID NO: 21 - RNA, 21 nt, synthetic construct
  source 1..21: mol_type = other RNA
  mRNA 1..21
actgaagattttacagcagat

SEQ ID NO: 22 - RNA, 21 nt, synthetic construct
  source 1..21: mol_type = other RNA
  mRNA 1..21
ttgtggaatgtgttgcatgta

SEQ ID NO: 23 - RNA, 21 nt, synthetic construct
  source 1..21: mol_type = other RNA
  mRNA 1..21
cagtcagcatttgcagacttt

SEQ ID NO: 24 - RNA, 21 nt, synthetic construct
  source 1..21: mol_type = other RNA
  mRNA 1..21
ttgatctgactgaagatttta

SEQ ID NO: 25 - RNA, 21 nt, synthetic construct
  source 1..21: mol_type = other RNA
  mRNA 1..21
ctgaagattttacagcagatt

SEQ ID NO: 26 - RNA, 21 nt, synthetic construct
  source 1..21: mol_type = other RNA
  mRNA 1..21
aatggtgatctaattcctgat

SEQ ID NO: 27 - RNA, 21 nt, synthetic construct
  source 1..21: mol_type = other RNA
  mRNA 1..21
gactgaagattttacagcaga

SEQ ID NO: 28 - RNA, 21 nt, synthetic construct
  source 1..21: mol_type = other RNA
  mRNA 1..21
gtggaatgtgttgcatgtact

SEQ ID NO: 29 - RNA, 21 nt, synthetic construct
  source 1..21: mol_type = other RNA
  mRNA 1..21
ttggacagtcagcatttgcag

SEQ ID NO: 30 - RNA, 21 nt, synthetic construct
  source 1..21: mol_type = other RNA
  mRNA 1..21
tttacattggcaggaaaagaa

SEQ ID NO: 31 - RNA, 21 nt, synthetic construct
  source 1..21: mol_type = other RNA
  mRNA 1..21
tttcattttgatgctatgtga

SEQ ID NO: 32 - RNA, 21 nt, synthetic construct
  source 1..21: mol_type = other RNA
  mRNA 1..21
atggagatggacacatggatc

SEQ ID NO: 33 - RNA, 21 nt, synthetic construct
  source 1..21: mol_type = other RNA
  mRNA 1..21
aagattttacagcagatttat

SEQ ID NO: 34 - RNA, 21 nt, synthetic construct
  source 1..21: mol_type = other RNA
  mRNA 1..21
tgcatttattgatctgactga

SEQ ID NO: 35 - RNA, 21 nt, synthetic construct
  source 1..21: mol_type = other RNA
  mRNA 1..21
tggtggttggacagtcagcat

SEQ ID NO: 36 - RNA, 21 nt, synthetic construct
  source 1..21: mol_type = other RNA
  mRNA 1..21
gcagactttgatggagatgga

SEQ ID NO: 37 - RNA, 21 nt, synthetic construct
  source 1..21: mol_type = other RNA
  mRNA 1..21
attgatctgactgaagatttt

SEQ ID NO: 38 - RNA, 21 nt, synthetic construct
  source 1..21: mol_type = other RNA
  mRNA 1..21
caatggtgatctaattcctga

SEQ ID NO: 39 - RNA, 21 nt, synthetic construct
  source 1..21: mol_type = other RNA
  mRNA 1..21
ttcaatggtgatctaattcct

SEQ ID NO: 40 - RNA, 21 nt, synthetic construct
  source 1..21: mol_type = other RNA
  mRNA 1..21
cattttacattggcaggaaaa

SEQ ID NO: 41 - RNA, 21 nt, synthetic construct
  source 1..21: mol_type = other RNA
  mRNA 1..21
ttggtatcacaaatgaatcca

SEQ ID NO: 42 - RNA, 21 nt, synthetic construct
  source 1..21: mol_type = other RNA
  mRNA 1..21
cagcatttgcagactttgatg

SEQ ID NO: 43 - RNA, 21 nt, synthetic construct
  source 1..21: mol_type = other RNA
  mRNA 1..21
tggagatggacacatggatca

SEQ ID NO: 44 - RNA, 21 nt, synthetic construct
  source 1..21: mol_type = other RNA
  mRNA 1..21
tcctttgtggaatgtgttgca

SEQ ID NO: 45 - RNA, 23 nt, synthetic construct
  source 1..23: mol_type = other RNA
  ncRNA 1..23: ncRNA_class = siRNA
tgctgtaaaatcttcagtcagtt

SEQ ID NO: 46 - RNA, 23 nt, synthetic construct
  source 1..23: mol_type = other RNA
  ncRNA 1..23: ncRNA_class = siRNA
tatcttacgaggacagtcatttt

SEQ ID NO: 47 - RNA, 23 nt, synthetic construct
  source 1..23: mol_type = other RNA
  ncRNA 1..23: ncRNA_class = siRNA
ttatcttacgaggacagtcattt

SEQ ID NO: 48 - RNA, 23 nt, synthetic construct
  source 1..23: mol_type = other RNA
  ncRNA 1..23: ncRNA_class = siRNA
tttcttttcctgccaatgtaatt

SEQ ID NO: 49 - RNA, 23 nt, synthetic construct
  source 1..23: mol_type = other RNA
  ncRNA 1..23: ncRNA_class = siRNA
tttctttcccgcagcacgaagtt

SEQ ID NO: 50 - RNA, 23 nt, synthetic construct
  source 1..23: mol_type = other RNA
  ncRNA 1..23: ncRNA_class = siRNA
ttacgaggacagtcattagaatt

SEQ ID NO: 51 - RNA, 23 nt, synthetic construct
  source 1..23: mol_type = other RNA
  ncRNA 1..23: ncRNA_class = siRNA
taaatctgctgtaaaatcttctt

SEQ ID NO: 52 - RNA, 23 nt, synthetic construct
  source 1..23: mol_type = other RNA
  ncRNA 1..23: ncRNA_class = siRNA
aaatgctgactgtccaaccactt

SEQ ID NO: 53 - RNA, 23 nt, synthetic construct
  source 1..23: mol_type = other RNA
  ncRNA 1..23: ncRNA_class = siRNA
atctgctgtaaaatcttcagttt

SEQ ID NO: 54 - RNA, 23 nt, synthetic construct
  source 1..23: mol_type = other RNA
  ncRNA 1..23: ncRNA_class = siRNA
tacatgcaacacattccacaatt

SEQ ID NO: 55 - RNA, 23 nt, synthetic construct
  source 1..23: mol_type = other RNA
  ncRNA 1..23: ncRNA_class = siRNA
aaagtctgcaaatgctgactgtt

SEQ ID NO: 56 - RNA, 23 nt, synthetic construct
  source 1..23: mol_type = other RNA
  ncRNA 1..23: ncRNA_class = siRNA
taaaatcttcagtcagatcaatt

SEQ ID NO: 57 - RNA, 23 nt, synthetic construct
  source 1..23: mol_type = other RNA
  ncRNA 1..23: ncRNA_class = siRNA
aatctgctgtaaaatcttcagtt

SEQ ID NO: 58 - RNA, 23 nt, synthetic construct
  source 1..23: mol_type = other RNA
  ncRNA 1..23: ncRNA_class = siRNA
atcaggaattagatcaccatttt

SEQ ID NO: 59 - RNA, 23 nt, synthetic construct
  source 1..23: mol_type = other RNA
  ncRNA 1..23: ncRNA_class = siRNA
tctgctgtaaaatcttcagtctt

SEQ ID NO: 60 - RNA, 23 nt, synthetic construct
  source 1..23: mol_type = other RNA
  ncRNA 1..23: ncRNA_class = siRNA
agtacatgcaacacattccactt

SEQ ID NO: 61 - RNA, 23 nt, synthetic construct
  source 1..23: mol_type = other RNA
  ncRNA 1..23: ncRNA_class = siRNA
ctgcaaatgctgactgtccaatt

SEQ ID NO: 62 - RNA, 23 nt, synthetic construct
  source 1..23: mol_type = other RNA
  ncRNA 1..23: ncRNA_class = siRNA
ttcttttcctgccaatgtaaatt

SEQ ID NO: 63 - RNA, 23 nt, synthetic construct
  source 1..23: mol_type = other RNA
  ncRNA 1..23: ncRNA_class = siRNA
tcacatagcatcaaaatgaaatt

SEQ ID NO: 64 - RNA, 23 nt, synthetic construct
  source 1..23: mol_type = other RNA
  ncRNA 1..23: ncRNA_class = siRNA
gatccatgtgtccatctccattt

SEQ ID NO: 65 - RNA, 23 nt, synthetic construct
  source 1..23: mol_type = other RNA
  ncRNA 1..23: ncRNA_class = siRNA
ataaatctgctgtaaaatctttt

SEQ ID NO: 66 - RNA, 23 nt, synthetic construct
  source 1..23: mol_type = other RNA
  ncRNA 1..23: ncRNA_class = siRNA
tcagtcagatcaataaatgcatt

SEQ ID NO: 67 - RNA, 23 nt, synthetic construct
  source 1..23: mol_type = other RNA
  ncRNA 1..23: ncRNA_class = siRNA
atgctgactgtccaaccaccatt

SEQ ID NO: 68 - RNA, 23 nt, synthetic construct
  source 1..23: mol_type = other RNA
  ncRNA 1..23: ncRNA_class = siRNA
tccatctccatcaaagtctgctt

SEQ ID NO: 69 - RNA, 23 nt, synthetic construct
  source 1..23: mol_type = other RNA
  ncRNA 1..23: ncRNA_class = siRNA
aaaatcttcagtcagatcaattt

SEQ ID NO: 70 - RNA, 23 nt, synthetic construct
  source 1..23: mol_type = other RNA
  ncRNA 1..23: ncRNA_class = siRNA
tcaggaattagatcaccattgtt

SEQ ID NO: 71 - RNA, 23 nt, synthetic construct
  source 1..23: mol_type = other RNA
  ncRNA 1..23: ncRNA_class = siRNA
aggaattagatcaccattgaatt

SEQ ID NO: 72 - RNA, 23 nt, synthetic construct
  source 1..23: mol_type = other RNA
  ncRNA 1..23: ncRNA_class = siRNA
ttttcctgccaatgtaaaatgtt

SEQ ID NO: 73 - RNA, 23 nt, synthetic construct
  source 1..23: mol_type = other RNA
  ncRNA 1..23: ncRNA_class = siRNA
tggattcatttgtgataccaatt

SEQ ID NO: 74 - RNA, 23 nt, synthetic construct
  source 1..23: mol_type = other RNA
  ncRNA 1..23: ncRNA_class = siRNA
catcaaagtctgcaaatgctgtt

SEQ ID NO: 75 - RNA, 23 nt, synthetic construct
  source 1..23: mol_type = other RNA
  ncRNA 1..23: ncRNA_class = siRNA
tgatccatgtgtccatctccatt

SEQ ID NO: 76 - RNA, 23 nt, synthetic construct
  source 1..23: mol_type = other RNA
  ncRNA 1..23: ncRNA_class = siRNA
tgcaacacattccacaaaggatt

SEQ ID NO: 77 - RNA, 21 nt, synthetic construct
  source 1..21: mol_type = other RNA
ctgactgaagattttacagca

SEQ ID NO: 78 - RNA, 21 nt, synthetic construct
  source 1..21: mol_type = other RNA
aatgactgtcctcgtaagata

SEQ ID NO: 79 - RNA, 21 nt, synthetic construct
  source 1..21: mol_type = other RNA
atgactgtcctcgtaagataa

SEQ ID NO: 80 - RNA, 21 nt, synthetic construct
  source 1..21: mol_type = other RNA
ttacattggcaggaaaagaaa

SEQ ID NO: 81 - RNA, 21 nt, synthetic construct
  source 1..21: mol_type = other RNA
cttcgtgctgcgggaaagaaa

SEQ ID NO: 82 - RNA, 21 nt, synthetic construct
  source 1..21: mol_type = other RNA
ttctaatgactgtcctcgtaa

SEQ ID NO: 83 - RNA, 21 nt, synthetic construct
  source 1..21: mol_type = other RNA
gaagattttacagcagattta

SEQ ID NO: 84 - RNA, 21 nt, synthetic construct
  source 1..21: mol_type = other RNA
gtggttggacagtcagcattt

SEQ ID NO: 85 - RNA, 21 nt, synthetic construct
  source 1..21: mol_type = other RNA
actgaagattttacagcagat

SEQ ID NO: 86 - RNA, 21 nt, synthetic construct
  source 1..21: mol_type = other RNA
ttgtggaatgtgttgcatgta

SEQ ID NO: 87 - RNA, 21 nt, synthetic construct
  source 1..21: mol_type = other RNA
cagtcagcatttgcagacttt

SEQ ID NO: 88 - RNA, 21 nt, synthetic construct
  source 1..21: mol_type = other RNA
ttgatctgactgaagatttta

SEQ ID NO: 89 - RNA, 21 nt, synthetic construct
  source 1..21: mol_type = other RNA
ctgaagattttacagcagatt

SEQ ID NO: 90 - RNA, 21 nt, synthetic construct
  source 1..21: mol_type = other RNA
aatggtgatctaattcctgat

SEQ ID NO: 91 - RNA, 21 nt, synthetic construct
  source 1..21: mol_type = other RNA
gactgaagattttacagcaga

SEQ ID NO: 92 - RNA, 21 nt, synthetic construct
  source 1..21: mol_type = other RNA
gtggaatgtgttgcatgtact

SEQ ID NO: 93 - RNA, 21 nt, synthetic construct
  source 1..21: mol_type = other RNA
ttggacagtcagcatttgcag

SEQ ID NO: 94 - RNA, 21 nt, synthetic construct
  source 1..21: mol_type = other RNA
tttacattggcaggaaaagaa

SEQ ID NO: 95 - RNA, 21 nt, synthetic construct
  source 1..21: mol_type = other RNA
tttcattttgatgctatgtga

SEQ ID NO: 96 - RNA, 21 nt, synthetic construct
  source 1..21: mol_type = other RNA
atggagatggacacatggatc

SEQ ID NO: 97 - RNA, 21 nt, synthetic construct
  source 1..21: mol_type = other RNA
aagattttacagcagatttat

SEQ ID NO: 98 - RNA, 21 nt, synthetic construct
  source 1..21: mol_type = other RNA
tgcatttattgatctgactga

SEQ ID NO: 99 - RNA, 21 nt, synthetic construct
  source 1..21: mol_type = other RNA
tggtggttggacagtcagcat

SEQ ID NO: 100 - RNA, 21 nt, synthetic construct
  source 1..21: mol_type = other RNA
gcagactttgatggagatgga

SEQ ID NO: 101 - RNA, 21 nt, synthetic construct
  source 1..21: mol_type = other RNA
attgatctgactgaagatttt

SEQ ID NO: 102 - RNA, 21 nt, synthetic construct
  source 1..21: mol_type = other RNA
caatggtgatctaattcctga

SEQ ID NO: 103 - RNA, 21 nt, synthetic construct
  source 1..21: mol_type = other RNA
ttcaatggtgatctaattcct

SEQ ID NO: 104 - RNA, 21 nt, synthetic construct
  source 1..21: mol_type = other RNA
cattttacattggcaggaaaa

SEQ ID NO: 105 - RNA, 21 nt, synthetic construct
  source 1..21: mol_type = other RNA
ttggtatcacaaatgaatcca

SEQ ID NO: 106 - RNA, 21 nt, synthetic construct
  source 1..21: mol_type = other RNA
cagcatttgcagactttgatg

SEQ ID NO: 107 - RNA, 21 nt, synthetic construct
  source 1..21: mol_type = other RNA
tggagatggacacatggatca

SEQ ID NO: 108 - RNA, 21 nt, synthetic construct
  source 1..21: mol_type = other RNA
tcctttgtggaatgtgttgca

SEQ ID NO: 109 - RNA, 21 nt, synthetic construct
  source 1..21: mol_type = other RNA
aatggtgatgtaattcctgat

SEQ ID NO: 110 - RNA, 21 nt, synthetic construct
  source 1..21: mol_type = other RNA
aatggtgatataattcctgat

SEQ ID NO: 111 - RNA, 21 nt, synthetic construct
  source 1..21: mol_type = other RNA
aatggtgatttaattcctgat

SEQ ID NO: 112 - RNA, 21 nt, synthetic construct
  source 1..21: mol_type = other RNA
aagattttagagcagatttat

SEQ ID NO: 113 - RNA, 21 nt, synthetic construct
  source 1..21: mol_type = other RNA
aagattttaaagcagatttat

SEQ ID NO: 114 - RNA, 21 nt, synthetic construct
  source 1..21: mol_type = other RNA
aagattttatagcagatttat

SEQ ID NO: 115 - RNA, 27 nt, synthetic construct
  source 1..27: mol_type = other RNA
  mRNA 1..27
atctgactgaagattttacagcagatt

SEQ ID NO: 116 - RNA, 27 nt, synthetic construct
  source 1..27: mol_type = other RNA
  mRNA 1..27
ctaatgactgtcctcgtaagataacac

SEQ ID NO: 117 - RNA, 27 nt, synthetic construct
  source 1..27: mol_type = other RNA
  mRNA 1..27
taatgactgtcctcgtaagataacacc

SEQ ID NO: 118 - RNA, 27 nt, synthetic construct
  source 1..27: mol_type = other RNA
  mRNA 1..27
ttttacattggcaggaaaagaaagcag

SEQ ID NO: 119 - RNA, 27 nt, synthetic construct
  source 1..27: mol_type = other RNA
  mRNA 1..27
ctcttcgtgctgcgggaaagaaatgac

SEQ ID NO: 120 - RNA, 27 nt, synthetic construct
  source 1..27: mol_type = other RNA
  mRNA 1..27
tgttctaatgactgtcctcgtaagata

SEQ ID NO: 121 - RNA, 27 nt, synthetic construct
  source 1..27: mol_type = other RNA
  mRNA 1..27
ctgaagattttacagcagatttattcc

SEQ ID NO: 122 - RNA, 27 nt, synthetic construct
  source 1..27: mol_type = other RNA
  mRNA 1..27
tggtggttggacagtcagcatttgcag

SEQ ID NO: 123 - RNA, 27 nt, synthetic construct
  source 1..27: mol_type = other RNA
  mRNA 1..27
tgactgaagattttacagcagatttat

SEQ ID NO: 124 - RNA, 27 nt, synthetic construct
  source 1..27: mol_type = other RNA
  mRNA 1..27
ctttgtggaatgtgttgcatgtactcc

SEQ ID NO: 125 - RNA, 27 nt, synthetic construct
  source 1..27: mol_type = other RNA
  mRNA 1..27
gacagtcagcatttgcagactttgatg

SEQ ID NO: 126 - RNA, 27 nt, synthetic construct
  source 1..27: mol_type = other RNA
  mRNA 1..27
tattgatctgactgaagattttacagc

SEQ ID NO: 127 - RNA, 27 nt, synthetic construct
  source 1..27: mol_type = other RNA
  mRNA 1..27
gactgaagattttacagcagatttatt

SEQ ID NO: 128 - RNA, 27 nt, synthetic construct
  source 1..27: mol_type = other RNA
  mRNA 1..27
tcaatggtgatctaattcctgatattt

SEQ ID NO: 129 - RNA, 27 nt, synthetic construct
  source 1..27: mol_type = other RNA
  mRNA 1..27
ctgactgaagattttacagcagattta

SEQ ID NO: 130 - RNA, 27 nt, synthetic construct
  source 1..27: mol_type = other RNA
  mRNA 1..27
ttgtggaatgtgttgcatgtactccag

SEQ ID NO: 131 - RNA, 27 nt, synthetic construct
  source 1..27: mol_type = other RNA
  mRNA 1..27
ggttggacagtcagcatttgcagactt

SEQ ID NO: 132 - RNA, 27 nt, synthetic construct
  source 1..27: mol_type = other RNA
  mRNA 1..27
attttacattggcaggaaaagaaagca

SEQ ID NO: 133 - RNA, 27 nt, synthetic construct
  source 1..27: mol_type = other RNA
  mRNA 1..27
ggtttcattttgatgctatgtgacttg

SEQ ID NO: 134 - RNA, 27 nt, synthetic construct
  source 1..27: mol_type = other RNA
  mRNA 1..27
tgatggagatggacacatggatcattt

SEQ ID NO: 135 - RNA, 27 nt, synthetic construct
  source 1..27: mol_type = other RNA
  mRNA 1..27
tgaagattttacagcagatttattcct

SEQ ID NO: 136 - RNA, 27 nt, synthetic construct
  source 1..27: mol_type = other RNA
  mRNA 1..27
catgcatttattgatctgactgaagat

SEQ ID NO: 137 - RNA, 27 nt, synthetic construct
  source 1..27: mol_type = other RNA
  mRNA 1..27
gatggtggttggacagtcagcatttgc

SEQ ID NO: 138 - RNA, 27 nt, synthetic construct
  source 1..27: mol_type = other RNA
  mRNA 1..27
ttgcagactttgatggagatggacaca

SEQ ID NO: 139 - RNA, 27 nt, synthetic construct
  source 1..27: mol_type = other RNA
  mRNA 1..27
ttattgatctgactgaagattttacag

SEQ ID NO: 140 - RNA, 27 nt, synthetic construct
  source 1..27: mol_type = other RNA
  mRNA 1..27
ttcaatggtgatctaattcctgatatt

SEQ ID NO: 141 - RNA, 27 nt, synthetic construct
  source 1..27: mol_type = other RNA
  mRNA 1..27
atttcaatggtgatctaattcctgata

SEQ ID NO: 142 - RNA, 27 nt, synthetic construct
  source 1..27: mol_type = other RNA
  mRNA 1..27
ggcattttacattggcaggaaaagaaa

SEQ ID NO: 143 - RNA, 27 nt, synthetic construct
  source 1..27: mol_type = other RNA
  mRNA 1..27
ttttggtatcacaaatgaatccaacca

SEQ ID NO: 144 - RNA, 27 nt, synthetic construct
  source 1..27: mol_type = other RNA
  mRNA 1..27
gtcagcatttgcagactttgatggaga

SEQ ID NO: 145 - RNA, 27 nt, synthetic construct
  source 1..27: mol_type = other RNA
  mRNA 1..27
gatggagatggacacatggatcattta

SEQ ID NO: 146 - RNA, 27 nt, synthetic construct
  source 1..27: mol_type = other RNA
  mRNA 1..27
gttcctttgtggaatgtgttgcatgta

SEQ ID NO: 147 - RNA, 27 nt, synthetic construct
  source 1..27: mol_type = other RNA
  ncRNA 1..27: ncRNA_class = siRNA
aatctgctgtaaaatcttcagtcagat

SEQ ID NO: 148 - RNA, 27 nt, synthetic construct
  source 1..27: mol_type = other RNA
  ncRNA 1..27: ncRNA_class = siRNA
gtgttatcttacgaggacagtcattag

SEQ ID NO: 149 - RNA, 27 nt, synthetic construct
  source 1..27: mol_type = other RNA
  ncRNA 1..27: ncRNA_class = siRNA
ggtgttatcttacgaggacagtcatta

SEQ ID NO: 150 - RNA, 27 nt, synthetic construct
  source 1..27: mol_type = other RNA
  ncRNA 1..27: ncRNA_class = siRNA
ctgctttcttttcctgccaatgtaaaa

SEQ ID NO: 151 - RNA, 27 nt, synthetic construct
  source 1..27: mol_type = other RNA
  ncRNA 1..27: ncRNA_class = siRNA
gtcatttctttcccgcagcacgaagag

SEQ ID NO: 152 - RNA, 27 nt, synthetic construct
  source 1..27: mol_type = other RNA
  ncRNA 1..27: ncRNA_class = siRNA
tatcttacgaggacagtcattagaaca

SEQ ID NO: 153 - RNA, 27 nt, synthetic construct
  source 1..27: mol_type = other RNA
  ncRNA 1..27: ncRNA_class = siRNA
ggaataaatctgctgtaaaatcttcag

SEQ ID NO: 154 - RNA, 27 nt, synthetic construct
  source 1..27: mol_type = other RNA
  ncRNA 1..27: ncRNA_class = siRNA
ctgcaaatgctgactgtccaaccacca

SEQ ID NO: 155 - RNA, 27 nt, synthetic construct
  source 1..27: mol_type = other RNA
  ncRNA 1..27: ncRNA_class = siRNA
ataaatctgctgtaaaatcttcagtca

SEQ ID NO: 156 - RNA, 27 nt, synthetic construct
  source 1..27: mol_type = other RNA
  ncRNA 1..27: ncRNA_class = siRNA
ggagtacatgcaacacattccacaaag

SEQ ID NO: 157 - RNA, 27 nt, synthetic construct
  source 1..27: mol_type = other RNA
  ncRNA 1..27: ncRNA_class = siRNA
catcaaagtctgcaaatgctgactgtc

SEQ ID NO: 158 - RNA, 27 nt, synthetic construct
  source 1..27: mol_type = other RNA
  ncRNA 1..27: ncRNA_class = siRNA
gctgtaaaatcttcagtcagatcaata

SEQ ID NO: 159 - RNA, 27 nt, synthetic construct
  source 1..27: mol_type = other RNA
  ncRNA 1..27: ncRNA_class = siRNA
aataaatctgctgtaaaatcttcagtc

SEQ ID NO: 160 - RNA, 27 nt, synthetic construct
  source 1..27: mol_type = other RNA
  ncRNA 1..27: ncRNA_class = siRNA
aaatatcaggaattagatcaccattga

SEQ ID NO: 161 - RNA, 27 nt, synthetic construct
  source 1..27: mol_type = other RNA
  ncRNA 1..27: ncRNA_class = siRNA
taaatctgctgtaaaatcttcagtcag

SEQ ID NO: 162 - RNA, 27 nt, synthetic construct
  source 1..27: mol_type = other RNA
  ncRNA 1..27: ncRNA_class = siRNA
ctggagtacatgcaacacattccacaa

SEQ ID NO: 163 - RNA, 27 nt, synthetic construct
  source 1..27: mol_type = other RNA
  ncRNA 1..27: ncRNA_class = siRNA
aagtctgcaaatgctgactgtccaacc

SEQ ID NO: 164 - RNA, 27 nt, synthetic construct
  source 1..27: mol_type = other RNA
  ncRNA 1..27: ncRNA_class = siRNA
tgctttcttttcctgccaatgtaaaat

SEQ ID NO: 165 - RNA, 27 nt, synthetic construct
  source 1..27: mol_type = other RNA
  ncRNA 1..27: ncRNA_class = siRNA
caagtcacatagcatcaaaatgaaacc

SEQ ID NO: 166 - RNA, 27 nt, synthetic construct
  source 1..27: mol_type = other RNA
  ncRNA 1..27: ncRNA_class = siRNA
aaatgatccatgtgtccatctccatca

SEQ ID NO: 167 - RNA, 27 nt, synthetic construct
  source 1..27: mol_type = other RNA
  ncRNA 1..27: ncRNA_class = siRNA
aggaataaatctgctgtaaaatcttca

SEQ ID NO: 168 - RNA, 27 nt, synthetic construct
  source 1..27: mol_type = other RNA
  ncRNA 1..27: ncRNA_class = siRNA
atcttcagtcagatcaataaatgcatg

SEQ ID NO: 169 - RNA, 27 nt, synthetic construct
  source 1..27: mol_type = other RNA
  ncRNA 1..27: ncRNA_class = siRNA
gcaaatgctgactgtccaaccaccatc

SEQ ID NO: 170 - RNA, 27 nt, synthetic construct
  source 1..27: mol_type = other RNA
  ncRNA 1..27: ncRNA_class = siRNA
tgtgtccatctccatcaaagtctgcaa

SEQ ID NO: 171 - RNA, 27 nt, synthetic construct
  source 1..27: mol_type = other RNA
  ncRNA 1..27: ncRNA_class = siRNA
ctgtaaaatcttcagtcagatcaataa

SEQ ID NO: 172 - RNA, 27 nt, synthetic construct
  source 1..27: mol_type = other RNA
  ncRNA 1..27: ncRNA_class = siRNA
aatatcaggaattagatcaccattgaa

SEQ ID NO: 173 - RNA, 27 nt, synthetic construct
  source 1..27: mol_type = other RNA
  ncRNA 1..27: ncRNA_class = siRNA
tatcaggaattagatcaccattgaaat

SEQ ID NO: 174 - RNA, 27 nt, synthetic construct
  source 1..27: mol_type = other RNA
  ncRNA 1..27: ncRNA_class = siRNA
tttcttttcctgccaatgtaaaatgcc

SEQ ID NO: 175 - RNA, 27 nt, synthetic construct
  source 1..27: mol_type = other RNA
  ncRNA 1..27: ncRNA_class = siRNA
tggttggattcatttgtgataccaaaa

SEQ ID NO: 176 - RNA, 27 nt, synthetic construct
  source 1..27: mol_type = other RNA
  ncRNA 1..27: ncRNA_class = siRNA
tctccatcaaagtctgcaaatgctgac

SEQ ID NO: 177 - RNA, 27 nt, synthetic construct
  source 1..27: mol_type = other RNA
  ncRNA 1..27: ncRNA_class = siRNA
taaatgatccatgtgtccatctccatc

SEQ ID NO: 178 - RNA, 27 nt, synthetic construct
  source 1..27: mol_type = other RNA
  ncRNA 1..27: ncRNA_class = siRNA
tacatgcaacacattccacaaaggaac

SEQ ID NO: 179 - RNA, 25 nt, synthetic construct
  source 1..25: mol_type = other RNA
ctgactgaagattttacagcagatt

SEQ ID NO: 180 - RNA, 25 nt, synthetic construct
  source 1..25: mol_type = other RNA
aatgactgtcctcgtaagataacac

SEQ ID NO: 181 - RNA, 25 nt, synthetic construct
  source 1..25: mol_type = other RNA
atgactgtcctcgtaagataacacc

SEQ ID NO: 182 - RNA, 25 nt, synthetic construct
  source 1..25: mol_type = other RNA
ttacattggcaggaaaagaaagcag

SEQ ID NO: 183 - RNA, 25 nt, synthetic construct
  source 1..25: mol_type = other RNA
cttcgtgctgcgggaaagaaatgac

SEQ ID NO: 184 - RNA, 25 nt, synthetic construct
  source 1..25: mol_type = other RNA
ttctaatgactgtcctcgtaagata

SEQ ID NO: 185 - RNA, 25 nt, synthetic construct
  source 1..25: mol_type = other RNA
gaagattttacagcagatttattcc

SEQ ID NO: 186 - RNA, 25 nt, synthetic construct
  source 1..25: mol_type = other RNA
gtggttggacagtcagcatttgcag

SEQ ID NO: 187 - RNA, 25 nt, synthetic construct
  source 1..25: mol_type = other RNA
actgaagattttacagcagatttat

SEQ ID NO: 188 - RNA, 25 nt, synthetic construct
  source 1..25: mol_type = other RNA
ttgtggaatgtgttgcatgtactcc

SEQ ID NO: 189 - RNA, 25 nt, synthetic construct
  source 1..25: mol_type = other RNA
cagtcagcatttgcagactttgatg

SEQ ID NO: 190 - RNA, 25 nt, synthetic construct
  source 1..25: mol_type = other RNA
ttgatctgactgaagattttacagc

SEQ ID NO: 191 - RNA, 25 nt, synthetic construct
  source 1..25: mol_type = other RNA
ctgaagattttacagcagatttatt

SEQ ID NO: 192 - RNA, 25 nt, synthetic construct
  source 1..25: mol_type = other RNA
aatggtgatctaattcctgatattt

SEQ ID NO: 193 - RNA, 25 nt, synthetic construct
  source 1..25: mol_type = other RNA
gactgaagattttacagcagattta

SEQ ID NO: 194 - RNA, 25 nt, synthetic construct
  source 1..25: mol_type = other RNA
gtggaatgtgttgcatgtactccag

SEQ ID NO: 195 - RNA, 25 nt, synthetic construct
  source 1..25: mol_type = other RNA
ttggacagtcagcatttgcagactt

SEQ ID NO: 196 - RNA, 25 nt, synthetic construct
  source 1..25: mol_type = other RNA
tttacattggcaggaaaagaaagca

SEQ ID NO: 197 - RNA, 25 nt, synthetic construct
  source 1..25: mol_type = other RNA
tttcattttgatgctatgtgacttg

SEQ ID NO: 198 - RNA, 25 nt, synthetic construct
  source 1..25: mol_type = other RNA
atggagatggacacatggatcattt

SEQ ID NO: 199 - RNA, 25 nt, synthetic construct
  source 1..25: mol_type = other RNA
aagattttacagcagatttattcct

SEQ ID NO: 200 - RNA, 25 nt, synthetic construct
  source 1..25: mol_type = other RNA
tgcatttattgatctgactgaagat

SEQ ID NO: 201 - RNA, 25 nt, synthetic construct
  source 1..25: mol_type = other RNA
tggtggttggacagtcagcatttgc

SEQ ID NO: 202 - RNA, 25 nt, synthetic construct
  source 1..25: mol_type = other RNA
gcagactttgatggagatggacaca

SEQ ID NO: 203 - RNA, 25 nt, synthetic construct
  source 1..25: mol_type = other RNA
attgatctgactgaagattttacag

SEQ ID NO: 204 - RNA, 25 nt, synthetic construct
  source 1..25: mol_type = other RNA
caatggtgatctaattcctgatatt

SEQ ID NO: 205 - RNA, 25 nt, synthetic construct
  source 1..25: mol_type = other RNA
ttcaatggtgatctaattcctgata

SEQ ID NO: 206 - RNA, 25 nt, synthetic construct
  source 1..25: mol_type = other RNA
cattttacattggcaggaaaagaaa

SEQ ID NO: 207 - RNA, 25 nt, synthetic construct
  source 1..25: mol_type = other RNA
ttggtatcacaaatgaatccaacca

SEQ ID NO: 208 - RNA, 25 nt, synthetic construct
  source 1..25: mol_type = other RNA
cagcatttgcagactttgatggaga

SEQ ID NO: 209 - RNA, 25 nt, synthetic construct
  source 1..25: mol_type = other RNA
tggagatggacacatggatcattta

SEQ ID NO: 210 - RNA, 25 nt, synthetic construct
  source 1..25: mol_type = other RNA
tcctttgtggaatgtgttgcatgta

SEQ ID NO: 211 - RNA, 23 nt, synthetic construct
  source 1..23: mol_type = other RNA
  modified_base order(3,5,7,9,11,13,15,17..20,23): mod_base = OTHER; note = 2-Prime-O-methyl nucleotide-3-Prime-phosphate
  modified_base order(1,21,22): mod_base = OTHER; note = 2-Prime-O-methyl nucleotide-3-Prime-phosphorothioate
  modified_base order(4,6,8,10,12,14,16): mod_base = OTHER; note = 2-Prime-fluoro nucleotide-3-Prime-phosphate
  modified_base 2: mod_base = OTHER; note = 2-Prime-fluoro nucleotide-3-Prime-phosphorothioate
atcaggaattagatcaccatttt

SEQ ID NO: 212 - RNA, 23 nt, synthetic construct
  source 1..23: mol_type = other RNA
  modified_base order(3,5,7,9..11,13,15,17..20,23): mod_base = OTHER; note = 2-Prime-O-methyl nucleotide-3-Prime-phosphate
  modified_base order(1,21,22): mod_base = OTHER; note = 2-Prime-O-methyl nucleotide-3-Prime-phosphorothioate
  modified_base order(4,6,8,12,14,16): mod_base = OTHER; note = 2-Prime-fluoro nucleotide-3-Prime-phosphate
  modified_base 2: mod_base = OTHER; note = 2-Prime-fluoro nucleotide-3-Prime-phosphorothioate
atcaggaattagatcaccatttt

SEQ ID NO: 213 - RNA, 23 nt, synthetic construct
  source 1..23: mol_type = other RNA
  modified_base order(3,5,7,9,11..13,15,17..20,23): mod_base = OTHER; note = 2-Prime-O-methyl nucleotide-3-Prime-phosphate
  modified_base order(1,21,22): mod_base = OTHER; note = 2-Prime-O-methyl nucleotide-3-Prime-phosphorothioate
  modified_base order(4,6,8,10,14,16): mod_base = OTHER; note = 2-Prime-fluoro nucleotide-3-Prime-phosphate
  modified_base 2: mod_base = OTHER; note = 2-Prime-fluoro nucleotide-3-Prime-phosphorothioate
atcaggaattagatcaccatttt

SEQ ID NO: 214 - RNA, 23 nt, synthetic construct
  source 1..23: mol_type = other RNA
  modified_base order(3,5,7,9..13,15,17..20,23): mod_base = OTHER; note = 2-Prime-O-methyl nucleotide-3-Prime-phosphate
  modified_base order(1,21,22): mod_base = OTHER; note = 2-Prime-O-methyl nucleotide-3-Prime-phosphorothioate
  modified_base order(4,6,8,14,16): mod_base = OTHER; note = 2-Prime-fluoro nucleotide-3-Prime-phosphate
  modified_base 2: mod_base = OTHER; note = 2-Prime-fluoro nucleotide-3-Prime-phosphorothioate
atcaggaattagatcaccatttt

SEQ ID NO: 215 - RNA, 23 nt, synthetic construct
  source 1..23: mol_type = other RNA
  modified_base order(3..5,7,9,11,13,15,17..20,23): mod_base = OTHER; note = 2-Prime-O-methyl nucleotide-3-Prime-phosphate
  modified_base order(1,21,22): mod_base = OTHER; note = 2-Prime-O-methyl nucleotide-3-Prime-phosphorothioate
  modified_base order(6,8,10,12,14,16): mod_base = OTHER; note = 2-Prime-fluoro nucleotide-3-Prime-phosphate
  modified_base 2: mod_base = OTHER; note = 2-Prime-fluoro nucleotide-3-Prime-phosphorothioate
atcaggaattagatcaccatttt

SEQ ID NO: 216 - RNA, 23 nt, synthetic construct
  source 1..23: mol_type = other RNA
  modified_base order(3,5,7..9,11,13,15,17..20,23): mod_base = OTHER; note = 2-Prime-O-methyl nucleotide-3-Prime-phosphate
  modified_base order(1,21,22): mod_base = OTHER; note = 2-Prime-O-methyl nucleotide-3-Prime-phosphorothioate
  modified_base order(4,6,10,12,14,16): mod_base = OTHER; note = 2-Prime-fluoro nucleotide-3-Prime-phosphate
  modified_base 2: mod_base = OTHER; note = 2-Prime-fluoro nucleotide-3-Prime-phosphorothioate
atcaggaattagatcaccatttt

SEQ ID NO: 217 - RNA, 23 nt, synthetic construct
  source 1..23: mol_type = other RNA
  modified_base order(3..7,9,11,13,15,17..20,23): mod_base = OTHER; note = 2-Prime-O-methyl nucleotide-3-Prime-phosphate
  modified_base order(1,21,22): mod_base = OTHER; note = 2-Prime-O-methyl nucleotide-3-Prime-phosphorothioate
  modified_base order(8,10,12,14,16): mod_base = OTHER; note = 2-Prime-fluoro nucleotide-3-Prime-phosphate
  modified_base 2: mod_base = OTHER; note = 2-Prime-fluoro nucleotide-3-Prime-phosphorothioate
atcaggaattagatcaccatttt

SEQ ID NO: 218 - RNA, 23 nt, synthetic construct
  source 1..23: mol_type = other RNA
  modified_base order(3..5,7..9,11,13,15,17..20,23): mod_base = OTHER; note = 2-Prime-O-methyl nucleotide-3-Prime-phosphate
  modified_base order(1,21,22): mod_base = OTHER; note = 2-Prime-O-methyl nucleotide-3-Prime-phosphorothioate
  modified_base order(6,10,12,14,16): mod_base = OTHER; note = 2-Prime-fluoro nucleotide-3-Prime-phosphate
  modified_base 2: mod_base = OTHER; note = 2-Prime-fluoro nucleotide-3-Prime-phosphorothioate
atcaggaattagatcaccatttt

SEQ ID NO: 219 - RNA, 23 nt, synthetic construct
  source 1..23: mol_type = other RNA
  modified_base order(3,4,6,8,9,11,13,15,17..20,23): mod_base = OTHER; note = 2-Prime-O-methyl nucleotide-3-Prime-phosphate
  modified_base order(1,2,21,22): mod_base = OTHER; note = 2-Prime-O-methyl nucleotide-3-Prime-phosphorothioate
  modified_base order(5,7,10,12,14,16): mod_base = OTHER; note = 2-Prime-fluoro nucleotide-3-Prime-phosphate
atcaggaattagatcaccatttt

SEQ ID NO: 220 - RNA, 23 nt, synthetic construct
  source 1..23: mol_type = other RNA
  modified_base order(3..9,11,13,15,17..20,23): mod_base = OTHER; note = 2-Prime-O-methyl nucleotide-3-Prime-phosphate
  modified_base order(1,21,22): mod_base = OTHER; note = 2-Prime-O-methyl nucleotide-3-Prime-phosphorothioate
  modified_base order(10,12,14,16): mod_base = OTHER; note = 2-Prime-fluoro nucleotide-3-Prime-phosphate
  modified_base 2: mod_base = OTHER; note = 2-Prime-fluoro nucleotide-3-Prime-phosphorothioate
atcaggaattagatcaccatttt

SEQ ID NO: 221 - RNA, 23 nt, synthetic construct
  source 1..23: mol_type = other RNA
  modified_base order(3,5,7,9,11,13,15,17..20,23): mod_base = OTHER; note = 2-Prime-O-methyl nucleotide-3-Prime-phosphate
  modified_base order(1,21,22): mod_base = OTHER; note = 2-Prime-O-methyl nucleotide-3-Prime-phosphorothioate
  modified_base order(4,6,8,10,12,14,16): mod_base = OTHER; note = 2-Prime-fluoro nucleotide-3-Prime-phosphate
  modified_base 2: mod_base = OTHER; note = 2-Prime-fluoro nucleotide-3-Prime-phosphorothioate
ataaatctgctgtaaaatctttt

SEQ ID NO: 222 - RNA, 23 nt, synthetic construct
  source 1..23: mol_type = other RNA
  modified_base order(3,5,7,9..11,13,15,17..20,23): mod_base = OTHER; note = 2-Prime-O-methyl nucleotide-3-Prime-phosphate
  modified_base order(1,21,22): mod_base = OTHER; note = 2-Prime-O-methyl nucleotide-3-Prime-phosphorothioate
  modified_base order(4,6,8,12,14,16): mod_base = OTHER; note = 2-Prime-fluoro nucleotide-3-Prime-phosphate
  modified_base 2: mod_base = OTHER; note = 2-Prime-fluoro nucleotide-3-Prime-phosphorothioate
ataaatctgctgtaaaatctttt

SEQ ID NO: 223 - RNA, 23 nt, synthetic construct
  source 1..23: mol_type = other RNA
  modified_base order(3,5,7,9,11..13,15,17..20,23): mod_base = OTHER; note = 2-Prime-O-methyl nucleotide-3-Prime-phosphate
  modified_base order(1,21,22): mod_base = OTHER; note = 2-Prime-O-methyl nucleotide-3-Prime-phosphorothioate
  modified_base order(4,6,8,10,14,16): mod_base = OTHER; note = 2-Prime-fluoro nucleotide-3-Prime-phosphate
  modified_base 2: mod_base = OTHER; note = 2-Prime-fluoro nucleotide-3-Prime-phosphorothioate
ataaatctgctgtaaaatctttt

SEQ ID NO: 224 - RNA, 23 nt, synthetic construct
  source 1..23: mol_type = other RNA
  modified_base order(3,5,7,9..13,15,17..20,23): mod_base = OTHER; note = 2-Prime-O-methyl nucleotide-3-Prime-phosphate
  modified_base order(1,21,22): mod_base = OTHER; note = 2-Prime-O-methyl nucleotide-3-Prime-phosphorothioate
  modified_base order(4,6,8,14,16): mod_base = OTHER; note = 2-Prime-fluoro nucleotide-3-Prime-phosphate
  modified_base 2: mod_base = OTHER; note = 2-Prime-fluoro nucleotide-3-Prime-phosphorothioate
ataaatctgctgtaaaatctttt

SEQ ID NO: 225 - RNA, 23 nt, synthetic construct
  source 1..23: mol_type = other RNA
  modified_base order(3..5,7,9,11,13,15,17..20,23): mod_base = OTHER; note = 2-Prime-O-methyl nucleotide-3-Prime-phosphate
  modified_base order(1,21,22): mod_base = OTHER; note = 2-Prime-O-methyl nucleotide-3-Prime-phosphorothioate
  modified_base order(6,8,10,12,14,16): mod_base = OTHER; note = 2-Prime-fluoro nucleotide-3-Prime-phosphate
  modified_base 2: mod_base = OTHER; note = 2-Prime-fluoro nucleotide-3-Prime-phosphorothioate
ataaatctgctgtaaaatctttt

SEQ ID NO: 226 - RNA, 23 nt, synthetic construct
  source 1..23: mol_type = other RNA
  modified_base order(3,5,7..9,11,13,15,17..20,23): mod_base = OTHER; note = 2-Prime-O-methyl nucleotide-3-Prime-phosphate
  modified_base order(1,21,22): mod_base = OTHER; note = 2-Prime-O-methyl nucleotide-3-Prime-phosphorothioate
  modified_base order(4,6,10,12,14,16): mod_base = OTHER; note = 2-Prime-fluoro nucleotide-3-Prime-phosphate
  modified_base 2: mod_base = OTHER; note = 2-Prime-fluoro nucleotide-3-Prime-phosphorothioate
ataaatctgctgtaaaatctttt

SEQ ID NO: 227 - RNA, 23 nt, synthetic construct
  source 1..23: mol_type = other RNA
  modified_base order(3..7,9,11,13,15,17..20,23): mod_base = OTHER; note = 2-Prime-O-methyl nucleotide-3-Prime-phosphate
  modified_base order(1,21,22): mod_base = OTHER; note = 2-Prime-O-methyl nucleotide-3-Prime-phosphorothioate
  modified_base order(8,10,12,14,16): mod_base = OTHER; note = 2-Prime-fluoro nucleotide-3-Prime-phosphate
  modified_base 2: mod_base = OTHER; note = 2-Prime-fluoro nucleotide-3-Prime-phosphorothioate
ataaatctgctgtaaaatctttt

SEQ ID NO: 228 - RNA, 23 nt, synthetic construct
  source 1..23: mol_type = other RNA
  modified_base order(3..5,7..9,11,13,15,17..20,23): mod_base = OTHER; note = 2-Prime-O-methyl nucleotide-3-Prime-phosphate
  modified_base order(1,21,22): mod_base = OTHER; note = 2-Prime-O-methyl nucleotide-3-Prime-phosphorothioate
  modified_base order(6,10,12,14,16): mod_base = OTHER; note = 2-Prime-fluoro nucleotide-3-Prime-phosphate
  modified_base 2: mod_base = OTHER; note = 2-Prime-fluoro nucleotide-3-Prime-phosphorothioate
ataaatctgctgtaaaatctttt

SEQ ID NO: 229 - RNA, 23 nt, synthetic construct
  source 1..23: mol_type = other RNA
  modified_base order(3,4,6,8,9,11,13,15,17..20,23): mod_base = OTHER; note = 2-Prime-O-methyl nucleotide-3-Prime-phosphate
  modified_base order(1,2,21,22): mod_base = OTHER; note = 2-Prime-O-methyl nucleotide-3-Prime-phosphorothioate
  modified_base order(5,7,10,12,14,16): mod_base = OTHER; note = 2-Prime-fluoro nucleotide-3-Prime-phosphate
ataaatctgctgtaaaatctttt

SEQ ID NO: 230 - RNA, 23 nt, synthetic construct
  source 1..23: mol_type = other RNA
  modified_base order(3..9,11,13,15,17..20,23): mod_base = OTHER; note = 2-Prime-O-methyl nucleotide-3-Prime-phosphate
  modified_base order(1,21,22): mod_base = OTHER; note = 2-Prime-O-methyl nucleotide-3-Prime-phosphorothioate
  modified_base order(10,12,14,16): mod_base = OTHER; note = 2-Prime-fluoro nucleotide-3-Prime-phosphate
  modified_base 2: mod_base = OTHER; note = 2-Prime-fluoro nucleotide-3-Prime-phosphorothioate
ataaatctgctgtaaaatctttt

SEQ ID NO: 231 - RNA, 21 nt, synthetic construct
  source 1..21: mol_type = other RNA
  modified_base order(3..6,8,12..18,21): mod_base = OTHER; note = 2-Prime-O-methyl nucleotide-3-Prime-phosphate
  modified_base order(1,2,19,20): mod_base = OTHER; note = 2-Prime-O-methyl nucleotide-3-Prime-phosphorothioate
  modified_base order(7,9..11): mod_base = OTHER; note = 2-Prime-fluoro nucleotide-3-Prime-phosphate
aatggtgatctaattcctgat

SEQ ID NO: 232 - RNA, 21 nt, synthetic construct
  source 1..21: mol_type = other RNA
  modified_base order(1..6,8,12..21): mod_base = OTHER; note = 2-Prime-O-methyl nucleotide-3-Prime-phosphate
  modified_base order(7,9..11): mod_base = OTHER; note = 2-Prime-fluoro nucleotide-3-Prime-phosphate
  modified_base order(1..3,19..21): mod_base = OTHER; note = Phosphorothioate linkage
aatggtgatgtaattcctgat

SEQ ID NO: 233 - RNA, 21 nt, synthetic construct
  source 1..21: mol_type = other RNA
  modified_base order(1..6,8,12..21): mod_base = OTHER; note = 2-Prime-O-methyl nucleotide-3-Prime-phosphate
  modified_base order(7,9..11): mod_base = OTHER; note = 2-Prime-fluoro nucleotide-3-Prime-phosphate
  modified_base order(1..3,19..21): mod_base = OTHER; note = Phosphorothioate linkage
aatggtgatataattcctgat

SEQ ID NO: 234 - RNA, 21 nt, synthetic construct
  source 1..21: mol_type = other RNA
  modified_base order(1..6,8,12..21): mod_base = OTHER; note = 2-Prime-O-methyl nucleotide-3-Prime-phosphate
  modified_base order(7,9..11): mod_base = OTHER; note = 2-Prime-fluoro nucleotide-3-Prime-phosphate
  modified_base order(1..3,19..21): mod_base = OTHER; note = Phosphorothioate linkage
aatggtgatttaattcctgat

SEQ ID NO: 235 - RNA, 21 nt, synthetic construct
  source 1..21: mol_type = other RNA
  modified_base order(1..6,8,12..21): mod_base = OTHER; note = 2-Prime-O-methyl nucleotide-3-Prime-phosphate
  modified_base order(7,9..11): mod_base = OTHER; note = 2-Prime-fluoro nucleotide-3-Prime-phosphate
  modified_base 1..3: mod_base = OTHER; note = Phosphorothioate linkage
aatggtgatttaattcctgat

SEQ ID NO: 236 - RNA, 21 nt, synthetic construct
  source 1..21: mol_type = other RNA
  modified_base order(3..6,8,12..18,21): mod_base = OTHER; note = 2-Prime-O-methyl nucleotide-3-Prime-phosphate
  modified_base order(7,9..11): mod_base = OTHER; note = 2-Prime-fluoro nucleotide-3-Prime-phosphate
  modified_base order(1,2,19,20): mod_base = OTHER; note = 2-Prime-O-methyl nucleotide-3-Prime-phosphorothioate
aagattttacagcagatttat

SEQ ID NO: 237 - RNA, 21 nt, synthetic construct
  source 1..21: mol_type = other RNA
  modified_base order(1..6,8,12..21): mod_base = OTHER; note = 2-Prime-O-methyl nucleotide-3-Prime-phosphate
  modified_base order(7,9..11): mod_base = OTHER; note = 2-Prime-fluoro nucleotide-3-Prime-phosphate
  modified_base order(1..3,19..21): mod_base = OTHER; note = Phosphorothioate linkage
aagattttagagcatatttat

SEQ ID NO: 238 - RNA, 21 nt, synthetic construct
  source 1..21: mol_type = other RNA
  modified_base order(1..6,8,12..21): mod_base = OTHER; note = 2-Prime-O-methyl nucleotide-3-Prime-phosphate
  modified_base order(7,9..11): mod_base = OTHER; note = 2-Prime-fluoro nucleotide-3-Prime-phosphate
  modified_base order(1..3,19..21): mod_base = OTHER; note = Phosphorothioate linkage
aagattttaaagcatatttat

SEQ ID NO: 239 - RNA, 21 nt, synthetic construct
  source 1..21: mol_type = other RNA
  modified_base order(1..6,8,12..21): mod_base = OTHER; note = 2-Prime-O-methyl nucleotide-3-Prime-phosphate
  modified_base order(7,9..11): mod_base = OTHER; note = 2-Prime-fluoro nucleotide-3-Prime-phosphate
  modified_base order(1..3,19..21): mod_base = OTHER; note = Phosphorothioate linkage
aagattttatagcatatttat

SEQ ID NO: 240 - RNA, 21 nt, synthetic construct
  source 1..21: mol_type = other RNA
  modified_base order(1..6,8,12..21): mod_base = OTHER; note = 2-Prime-O-methyl nucleotide-3-Prime-phosphate
  modified_base order(7,9..11): mod_base = OTHER; note = 2-Prime-fluoro nucleotide-3-Prime-phosphate
  modified_base 1..3: mod_base = OTHER; note = Phosphorothioate linkage
aagattttacagcagatttat

SEQ ID NO: 241 - RNA, 21 nt, synthetic construct
  source 1..21: mol_type = other RNA
  modified_base order(1..6,8,12..21): mod_base = OTHER; note = 2-Prime-O-methyl nucleotide-3-Prime-phosphate
  modified_base order(7,9..11): mod_base = OTHER; note = 2-Prime-fluoro nucleotide-3-Prime-phosphate
  modified_base 1..3: mod_base = OTHER; note = Phosphorothioate linkage
aatggtgatttaattcctgat

SEQ ID NO: 242 - DNA, 20 bp, synthetic construct
  source 1..20: mol_type = other DNA
tgatggagatggacacatgg

SEQ ID NO: 243 - DNA, 20 bp, synthetic construct
  source 1..20: mol_type = other DNA
catccacaaatggcacaaag

SEQ ID NO: 244 - DNA, 21 bp, synthetic construct
  source 1..21: mol_type = other DNA
caatgtccctcgaagctggag

SEQ ID NO: 245 - DNA, 24 bp, synthetic construct
  source 1..24: mol_type = other DNA
ttctctgtcatcggctttcttttc

SEQ ID NO: 246 - DNA, 20 bp, synthetic construct
  source 1..20: mol_type = other DNA
agaaacgccaaccaagagga

SEQ ID NO: 247 - DNA, 20 bp, synthetic construct
  source 1..20: mol_type = other DNA
gactggggccgttccttgat

SEQ ID NO: 248 - DNA, 20 bp, synthetic construct
  source 1..20: mol_type = other DNA
ctaaggccaaccgggagaaa

SEQ ID NO: 249 - DNA, 20 bp, synthetic construct
  source 1..20: mol_type = other DNA
gagtccagcacaataccagt

SEQ ID NO: 250 - DNA, 21 bp, synthetic construct
  source 1..21: mol_type = other DNA
gccgcagatactgttaggagg

SEQ ID NO: 251 - DNA, 23 bp, synthetic construct
  source 1..23: mol_type = other DNA
ggatagagaagtttccacccaaa

SEQ ID NO: 252 - DNA, 20 bp, synthetic construct
  source 1..20: mol_type = other DNA
gagcgcaaattttcttgacc

SEQ ID NO: 253 - DNA, 20 bp, synthetic construct
  source 1..20: mol_type = other DNA
gggacattgtgagggtatgg

SEQ ID NO: 254 - RNA, 21 nt, synthetic construct
  source 1..21: mol_type = other RNA
  modified_base order(1..6,8,12..21): mod_base = OTHER; note = 2-Prime-O-methyl nucleotide-3-Prime-phosphate
  modified_base order(7,9..11): mod_base = OTHER; note = 2-Prime-fluoro nucleotide-3-Prime-phosphate
  modified_base 1..3: mod_base = OTHER; note = Phosphorothioate linkage
aatggtgatttaattcctgat

SEQ ID NO: 255 - RNA, 21 nt, synthetic construct
  source 1..21: mol_type = other RNA
  modified_base order(1..6,8,12..21): mod_base = OTHER; note = 2-Prime-O-methyl nucleotide-3-Prime-phosphate
  modified_base order(7,9..11): mod_base = OTHER; note = 2-Prime-fluoro nucleotide-3-Prime-phosphate
  modified_base 1..3: mod_base = OTHER; note = Phosphorothioate linkage
aatggtgatttaattcctgat

SEQ ID NO: 256 - RNA, 21 nt, synthetic construct
  source 1..21: mol_type = other RNA
  modified_base order(1..6,8,12..21): mod_base = OTHER; note = 2-Prime-O-methyl nucleotide-3-Prime-phosphate
  modified_base order(7,9..11): mod_base = OTHER; note = 2-Prime-fluoro nucleotide-3-Prime-phosphate
  modified_base 1..3: mod_base = OTHER; note = Phosphorothioate linkage
aatggtgatttaattcctgat

SEQ ID NO: 257 - RNA, 21 nt, synthetic construct
  source 1..21: mol_type = other RNA
  modified_base order(1..6,8,12..21): mod_base = OTHER; note = 2-Prime-O-methyl nucleotide-3-Prime-phosphate
  modified_base order(7,9..11): mod_base = OTHER; note = 2-Prime-fluoro nucleotide-3-Prime-phosphate
  modified_base 1..3: mod_base = OTHER; note = Phosphorothioate linkage
aatggtgatttaattcctgat